SEQUENCE LISTING (34 sequences)

SEQ ID NO: 1 - protein, 846 aa, synthetic construct
  REGION 1..846: note = Synthetic polypeptide
MLFTVSCSKMSSIVDRDDSSIFDGLVEEDDKDKAKRVSRNKSEKKRRDQFNVLIKELGSMLPGNARKMDKSTVLQKSIDFLRKHKEITAQSDASEIRQDWKPTFLSNEEFTQLMLEALDGFFLAIMTDGSIIYVSESVTSLLEHLPSDLVDQSIFNFIPEGEHSEVYKILSTHLLESDSLTPEYLKSKNQLEFCCHMLRGTIDPKEPSTYEYVKFIGNFKSLNSVSSSAHNGFEGTIQRTHRPSYEDRVCFVATVRLATPQFIKEMCTVEEPNEEFTSRHSLEWKFLFLDHRAPPIIGYLPFEVLGTSGYDYYHVDDLENLAKCHEHLMQYGKGKSCYYRFLTKGQQWIWLQTHYYITYHQWNSRPEFIVCTHTVVSYAEVRAERRRELGIEESLPETAADKSQDSGSDNRINTVSLKEALERFDHSPTPSASSRSSRKSSHTAVSDPSSTPTKIPTDTSTPPRQHLPAHEKMVQRRSSFSSQSINSQSVGSSLTQPVMSQATNLPIPQGMSQFQFSAQLGAMQHLKDQLEQRTRMIEANIHRQQEELRKIQEQLQMVHGQGLQMFLQQSNPGLNFGSVQLSSGNSSNIQQLAPINMQGQVVPTNQIQSGMNTGHIGTTQHMIQQQTLQSTSTQSQQNVLSGHSQQTSLPSQTQSTLTAPLYNTMVISQPAAGSMVQIPSSMPQNSTQSAAVTTFTQDRQIRFSQGQQLVTKLVTAPVACGAVMVPSTMLMGQVVTAYPTFATQQQQSQTLSVTQQQQQQSSQEQQLTSVQQPSQAQLTQPPQQFLQTSRLLHGNPSTQLILSAAFPLQQSTFPQSHHQQHQSQQQQQLSRHRTDSLPDPSKVQPQ

SEQ ID NO: 2 - protein, 626 aa, synthetic construct
  REGION 1..626: note = Synthetic polypeptide
MADQRMDISSTISDFMSPGPTDLLSSSLGTSGVDCNRKRKGSSTDYQESMDTDKDDPHGRLEYTEHQGRIKNAREAHSQIEKRRRDKMNSFIDELASLVPTCNAMSRKLDKLTVLRMAVQHMKTLRGATNPYTEANYKPTFLSDDELKHLILRAADGFLFVVGCDRGKILFVSESVFKILNYSQNDLIGQSLFDYLHPKDIAKVKEQLSSSDTAPRERLIDAKTGLPVKTDITPGPSRLCSGARRSFFCRMKCNRPSVKVEDKDFPSTCSKKKADRKSFCTIHSTGYLKSWPPTKMGLDEDNEPDNEGCNLSCLVAIGRLHSHVVPQPVNGEIRVKSMEYVSRHAIDGKFVFVDQRATAILAYLPQELLGTSCYEYFHQDDIGHLAECHRQVLQTREKITTNCYKFKIKDGSFITLRSRWFSFMNPWTKEVEYIVSTNTVVLANVLEGGDPTFPQLTASPHSMDSMLPSGEGGPKRTHPTVPGIPGGTRAGAGKIGRMIAEEIMEIHRIRGSSPSSCGSSPLNITSTPPPDASSPGGKKILNGGTPDIPSSGLLSGQAQENPGYPYSDSSSILGENPHIGIDMIDNDQGSSSPSNDEAAMAVIMSLLEADAGLGGPVDFSDLPWPL

SEQ ID NO: 3 - DNA, 21 bp, synthetic construct
  misc_feature 1..21: note = Synthetic polynucleotide
  source 1..21: mol_type = other DNA
cgctttctggagggtgtccgc

SEQ ID NO: 4 - DNA, 20 bp, synthetic construct
  misc_feature 1..20: note = Synthetic polynucleotide
  source 1..20: mol_type = other DNA
tgccaggacgcgcttgtacc

SEQ ID NO: 5 - DNA, 19 bp, synthetic construct
  misc_feature 1..19: note = Synthetic polynucleotide
  source 1..19: mol_type = other DNA
ttgctccacgggaatcctt

SEQ ID NO: 6 - DNA, 23 bp, synthetic construct
  misc_feature 1..23: note = Synthetic polynucleotide
  source 1..23: mol_type = other DNA
ggagggaaagtgctctgttgtag

SEQ ID NO: 7 - DNA, 20 bp, synthetic construct
  misc_feature 1..20: note = Synthetic polynucleotide
  source 1..20: mol_type = other DNA
tggcatccggtgcactgcag

SEQ ID NO: 8 - DNA, 22 bp, synthetic construct
  misc_feature 1..22: note = Synthetic polynucleotide
  source 1..22: mol_type = other DNA
ccctccagaagggtagcacgct

SEQ ID NO: 9 - DNA, 24 bp, synthetic construct
  misc_feature 1..24: note = Synthetic polynucleotide
  source 1..24: mol_type = other DNA
ggagttcatgcttgtgaaggctgt

SEQ ID NO: 10 - DNA, 24 bp, synthetic construct
  misc_feature 1..24: note = Synthetic polynucleotide
  source 1..24: mol_type = other DNA
cagacacttcttaaagcggcactg

SEQ ID NO: 11 - DNA, 19 bp, synthetic construct
  misc_feature 1..19: note = Synthetic polynucleotide
  source 1..19: mol_type = other DNA
ctggcgtggaagtcatcgt

SEQ ID NO: 12 - DNA, 22 bp, synthetic construct
  misc_feature 1..22: note = Synthetic polynucleotide
  source 1..22: mol_type = other DNA
ctgtccgccattgagttctatg

SEQ ID NO: 13 - DNA, 21 bp, synthetic construct
  misc_feature 1..21: note = Synthetic polynucleotide
  source 1..21: mol_type = other DNA
tgtcccttcctgtgtggaaga

SEQ ID NO: 14 - DNA, 18 bp, synthetic construct
  misc_feature 1..18: note = Synthetic polynucleotide
  source 1..18: mol_type = other DNA
gctcccagcttggcttga

SEQ ID NO: 15 - DNA, 20 bp, synthetic construct
  misc_feature 1..20: note = Synthetic polynucleotide
  source 1..20: mol_type = other DNA
ctgccatggaggaagaagag

SEQ ID NO: 16 - DNA, 20 bp, synthetic construct
  misc_feature 1..20: note = Synthetic polynucleotide
  source 1..20: mol_type = other DNA
agctggggcagtttcctatt

SEQ ID NO: 17 - DNA, 19 bp, synthetic construct
  misc_feature 1..19: note = Synthetic polynucleotide
  source 1..19: mol_type = other DNA
atgctcgccatccacaaga

SEQ ID NO: 18 - DNA, 20 bp, synthetic construct
  misc_feature 1..20: note = Synthetic polynucleotide
  source 1..20: mol_type = other DNA
gcggaatcgaatgggagaat

SEQ ID NO: 19 - DNA, 23 bp, synthetic construct
  misc_feature 1..23: note = Synthetic polynucleotide
  source 1..23: mol_type = other DNA
ggtttctactacgttgctactgg

SEQ ID NO: 20 - DNA, 21 bp, synthetic construct
  misc_feature 1..21: note = Synthetic polynucleotide
  source 1..21: mol_type = other DNA
ggaatccgtcaacaggttcgt

SEQ ID NO: 21 - DNA, 20 bp, synthetic construct
  misc_feature 1..20: note = Synthetic polynucleotide
  source 1..20: mol_type = other DNA
caacgcgtgcgctctgggta

SEQ ID NO: 22 - DNA, 20 bp, synthetic construct
  misc_feature 1..20: note = Synthetic polynucleotide
  source 1..20: mol_type = other DNA
tggctcaagccctttccgcg

SEQ ID NO: 23 - DNA, 19 bp, synthetic construct
  misc_feature 1..19: note = Synthetic polynucleotide
  source 1..19: mol_type = other DNA
catgcccaaccttcttggc

SEQ ID NO: 24 - DNA, 21 bp, synthetic construct
  misc_feature 1..21: note = Synthetic polynucleotide
  source 1..21: mol_type = other DNA
cagcgggtagaactgatgcac

SEQ ID NO: 25 - DNA, 21 bp, synthetic construct
  misc_feature 1..21: note = Synthetic polynucleotide
  source 1..21: mol_type = other DNA
aatcatgcagggggccccgaa

SEQ ID NO: 26 - DNA, 23 bp, synthetic construct
  misc_feature 1..23: note = Synthetic polynucleotide
  source 1..23: mol_type = other DNA
cgacaagctaggtacctgtggcg

SEQ ID NO: 27 - DNA, 21 bp, synthetic construct
  misc_feature 1..21: note = Synthetic polynucleotide
  source 1..21: mol_type = other DNA
tcagtttgcgggtgtgcgcag

SEQ ID NO: 28 - DNA, 21 bp, synthetic construct
  misc_feature 1..21: note = Synthetic polynucleotide
  source 1..21: mol_type = other DNA
ttcgtctcgcctacaccaccg

SEQ ID NO: 29 - DNA, 21 bp, synthetic construct
  misc_feature 1..21: note = Synthetic polynucleotide
  source 1..21: mol_type = other DNA
ggccgcagcgccttctcttta

SEQ ID NO: 30 - DNA, 22 bp, synthetic construct
  misc_feature 1..22: note = Synthetic polynucleotide
  source 1..22: mol_type = other DNA
accatcattgactcccccgagg

SEQ ID NO: 31 - DNA, 19 bp, synthetic construct
  misc_feature 1..19: note = Synthetic polynucleotide
  source 1..19: mol_type = other DNA
cgcttggctgaaccatcac

SEQ ID NO: 32 - DNA, 21 bp, synthetic construct
  misc_feature 1..21: note = Synthetic polynucleotide
  source 1..21: mol_type = other DNA
gttccgcgtcatcctgatagt

SEQ ID NO: 33 - DNA, 21 bp, synthetic construct
  misc_feature 1..21: note = Synthetic polynucleotide
  source 1..21: mol_type = other DNA
cgctttctggagggtgtccgc

SEQ ID NO: 34 - DNA, 20 bp, synthetic construct
  misc_feature 1..20: note = Synthetic polynucleotide
  source 1..20: mol_type = other DNA
tgccaggacgcgcttgtacc

(19) United States
(12) Patent Application Publication
(10) Pub. No.: US 2025/0100962 A1
(43) Pub. Date: Mar. 27, 2025

(54) CLOCK ACTIVATORS AND USES THEREOF

(71) Applicant: CITY OF HOPE, Duarte, CA (US)

(72) Inventors: Ke Ma, La Verne, CA (US); David Horne, Altadena, CA (US); Yuelong Ma, Glendora, CA (US)

(21) Appl. No.: 18/892,055

(22) Filed: Sep. 20, 2024

Related U.S. Application Data
(60) Provisional application No. 63/539,765, filed on Sep. 21, 2023.

(51) Int. Cl. C07C 279/18; A61K 31/155; A61P 21/00; A61P 43/00

(52) U.S. Cl. CPC C07C 279/18; A61K 31/155; A61P 21/00; A61P 43/00

(57) ABSTRACT

Described herein, inter alia, are clock activators and uses thereof.

### CROSS-REFERENCE TO RELATED APPLICATIONS

This application claims the benefit of U.S. Provisional Application No. 63/539,765 filed Sep. 21, 2023, the contents of which is hereby incorporated herein in its entirety and for all purposes.

### STATEMENT AS TO RIGHTS TO INVENTIONS MADE UNDER FEDERALLY SPONSORED RESEARCH AND DEVELOPMENT

This invention was made with government support under R01 DK112794, and R56 AG080294 awarded by the National Institutes of Health. The government has certain rights in the invention.

### REFERENCE TO AN ELECTRONIC SEQUENCE LISTING

The contents of the electronic sequence listing (048440-843001US_Sequence_Listing_ST26.xml; Size: 45,562 bytes; and Date of Creation: Aug. 27, 2024) are hereby incorporated by reference in their entirety.

### BACKGROUND

The circadian clock, driven by a molecular machinery that generates a ˜24-hour rhythm, imparts pervasive temporal controls in diverse biological processes (1). Oscillations in metabolism, cell cycle and stem cell behavior are required for metabolic tissue growth and remodeling to maintain homeostasis in response to daily entrainment cues. Disruption of clock control, increasingly prevalent in our modern lifestyle, leads to the development of metabolic disorders (2,3). Therapeutic targeting of circadian clock and its biological output pathways may thus have potential applications in tissue remodeling and treatment and prevention of metabolic diseases. Disclosed herein, inter alia, are solutions to these and other problems in the art.

### BRIEF SUMMARY

In an aspect is provided a compound, or a pharmaceutically acceptable salt thereof, having the formula:

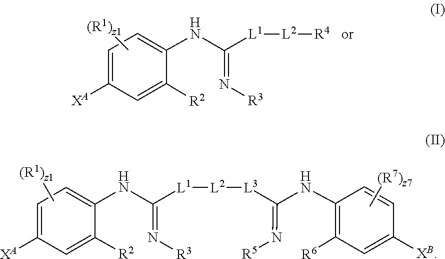

X^{A} and X^{B} are independently —Cl, —Br, —I, or —F.

L¹ is a bond, —C(O)—, —C(O)O—, —OC(O)—, —O—, —S—, —NR¹⁰—, —C(O)NR¹⁰—, —NR¹⁰C(O)—, —NR¹⁰C(O)O—, —OC(O)NR¹⁰—, —NR¹⁰C(O)NR¹⁰—, —NR¹⁰C(NH)NR¹⁰—, —S(O)₂—, —NR¹⁰S(O)₂—, —S(O)₂NR¹⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L² is a bond, —C(O)—, —C(O)O—, —OC(O)—, —O—, —S—, —NR²⁰—, —C(O)NR²⁰—, —NR²⁰C(O)—, —NR²⁰C(O)O—, —OC(O)NR²⁰—, —NR²⁰C(O)NR²⁰—, —NR²⁰C(NH)NR²⁰—, —S(O)₂—, —NR²⁰S(O)₂—, —S(O)₂NR²⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —NR³⁰C(NH)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹ is independently halogen, —CX¹₃, —CHX¹₂, —CH₂X¹₃, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A} C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A} C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² and R³ substituents may optionally be joined to form a substituted or unsubstituted heterocycloalkyl or substituted or unsubstituted heteroaryl.

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —NR^{4A} C(O)OR^{4C}, —NR^{4A}OR^{4C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ is hydrogen, —CCl₃, —CBr₃, —CF₃, —CI₃, —CHCl₂, —CHBr₂, —CHF₂, —CHI₂, —CH₂Cl, —CH₂Br, —CH₂F, —C H₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OC(O)NR^{6A}R^{6B}, —OR^{6D}, —SR^{6D}, —NR^{6A}SO₂R^{6D}, —NR^{6A}C(O)R^{6C}, —NR^{6A} C(O)OR^{6C}, —NR^{6A}OR^{6C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ and R⁶ substituents may optionally be joined to form a substituted or unsubstituted heterocycloalkyl or substituted or unsubstituted heteroaryl.

R⁷ is independently halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NR^{7C}C(O)NR^{7A}R^{7B}, —N(O)ₘ₇, —NR^{7A}R^{7B}, —C(O)R^{7C}, —C(O)OR^{7C}, —OC(O)R^{7C}, —OC(O)OR^{7C}, —C(O)NR^{7A}R^{7B}, —OC(O)NR^{7A}R^{7B}, —OR^{7D}, —SR^{7D}, —NR^{7A}SO₂R^{7D}, —NR^{7A}C(O)R^{7C}, —NR^{7A} C(O)OR^{7C}, —NR^{7A}OR^{7C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Each R¹⁰, R²⁰, and R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{2A}, R^{2B}, R^{2C}, R^{2D}, R^{4A}, R^{4B}, R^{4C}, R^{4D}, R^{6A}, R^{6B}, R^{6C}, R^{6D}, R^{7A}, R^{7B}, R^{7C}, and R^{7D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl.

The symbol z1 is an integer from 0 to 3. The symbol z7 is an integer from 0 to 2.

Each X¹, X², X⁴, X⁶, and X⁷ is independently —Cl, —Br, —I, or —F. The symbols n1, n2, n4, n6, and n7 are independently an integer from 0 to 4. The symbols m1, m2, m4, m6, m7, v1, v2, v4, v6, and v7 are independently 1 or 2.

In an aspect is provided a pharmaceutical composition including a compound described herein, or a pharmaceutically acceptable salt thereof, and a pharmaceutically acceptable excipient.

In an aspect is provided a method of increasing myogenesis in a subject in need thereof, the method including administering to the subject a therapeutically effective amount of a clock activator, or a pharmaceutically acceptable salt thereof. In embodiments, the clock activator is a compound as described herein, including in embodiments.

In an aspect is provided a method of treating a muscle degenerative disease in a subject in need thereof, the method including administering to the subject a therapeutically effective amount of a clock activator described herein, or a pharmaceutically acceptable salt thereof. In embodiments, the clock activator is a compound as described herein, including in embodiments.

In an aspect is provided a method of treating a muscle injury in a subject in need thereof, the method including administering to the subject a therapeutically effective amount of a clock activator, or a pharmaceutically acceptable salt thereof. In embodiments, the clock activator is a compound as described herein, including in embodiments.

In an aspect is provided a method of reducing adipogenesis in a subject in need thereof, the method including administering to the subject a therapeutically effective amount of a clock activator, or a pharmaceutically acceptable salt thereof. In embodiments, the clock activator is a compound as described herein, including in embodiments.

In an aspect is provided a method of treating a metabolic disorder in a subject in need thereof, the method including administering to the subject a therapeutically effective amount of a clock activator described herein, or a pharmaceutically acceptable salt thereof. In embodiments, the clock activator is a compound as described herein, including in embodiments.

In an aspect is provided a method of activating a CLOCK protein, the method including contacting the CLOCK protein with a clock activator, or a pharmaceutically acceptable salt thereof, which contacts at least one amino acid residue forming a palmitoylation site of the CLOCK protein, wherein the at least one amino acid residue is selected from E116, Q261, F262, E270, G345, Q112, F262, and P260. In embodiments, the clock activator is a compound as described herein, including in embodiments.

### BRIEF DESCRIPTION OF THE DRAWINGS

FIGS. 1A-1E. Screening of circadian clock modulators targeting a novel palmitoylation pocket in CLOCK protein. FIG. 1A: Structure modeling of hydrophobic pocket palmitoylation site on CLOCK protein. Crystal structure of heterodimeric CLOCK with Bmal1 is based on PDB: 4f31. Stick structure: palmitoyl-CoA. FIG. 1B: All-around docking poses of 266 hit compounds identified targeting CLOCK palmitoylation pocket. FIG. 1C: Screening strategy to identify clock modulators using NCI/DTP-FDA chemical libraries with functional validation. FIG. 1D: Structural modeling of chlorhexidine within the CLOCK-Bmal1 interaction interface. FIG. 1E: Predicted interactions of chlorhexidine with residues within CLOCK palmitoylation pocket.

FIGS. 2A-2F. Identification of chlorhexidine as a circadian clock activator. FIG. 2A: Average tracing of bioluminescence activity monitoring of U2OS cells containing PER2::LUC reporter, treated by chlorhexidine (CHX) at indicated concentrations as shown in baseline-adjusted plot. FIGS. 2B-2C: Quantitative analysis of circadian period length (FIG. 2B) and cycling amplitude (FIG. 2C) at CHX concentrations indicated. Data are presented as Mean±SD. =4 each concentration. *,**,***: p<0.05, 0.01 and 0.001, CHX vs. DMSO by Student's t test. CHX=Chlorhexidine. FIG. 2D: Analysis of chlorhexidine direct effect on clock transcriptional activity by luciferase assay. 293A cells were transfected with Bmal1 and CLOCK cDNA constructs to activate Period2-driven luciferase reporter, with bioluminescence activity determined at 6 hours following CHX or SR8278 treatment as compared to DMSO control. Cry2 plasmid was used as a negative control to inhibit Bmal1/CLOCK-mediated transcription activity, and Rev-erbα antagonist SR8278 as a positive control. PGL2: Per2-Luciferase reporter construct. n=4/treatment. *,**,***: p<0.05, 0.01 and 0.001 respectively, CM1/SR8278 vs. DMSO by Student's t test. FIGS. 2E-2F: Chlorhexidine promotes interaction of Bmal1 with CLOCK through analysis of Bmal1 and Myc-CLOCK interaction in the presence or absence of chlorhexidine (CHX) or CHX-derivative CM001. Co-immunoprecipitation of Bmal1 with Myc-CLOCK (FIG. 2E) or with a Myc-CLOCK mutant (FIG. 2F) in the presence of indicated concentrations of CHX or CM001.

FIGS. 3A-3E. Induction of clock gene protein expression by chlorhexidine. FIGS. 3A-3B: Immunoblot analysis of core clock protein expression after treatment with CHX at indicated concentration for 24 h in U2OS cells (FIG. 3A), and quantification (FIG. 3B). FIGS. 3C-3E: RT-qPCR analysis of core clock genes after CHX treatment for 24 h of U2OS cells—core clock activators (FIG. 3C), Bmal1/CLOCK target genes (FIG. 3D), and clock repressor genes (FIG. 3E). *,**,***: p<0.05, 0.01 and 0.001, CHX vs. DMSO by Student's t test.

FIGS. 4A-4C. Chlorhexidine induces clock activation in myoblasts. FIG. 4A: Western blot analysis of chlorhexidine on clock protein expression in C2C12 myoblast during day 2, 4, and 6 of myogenic differentiation at indicated concentration. FIGS. 4B-4C: Chlorhexidine modulation of clock is dependent on Bmal1. RT-qPCR analysis of CHX treatment on expression of CLOCK and Bmal1 with its transcription targets (FIG. 4B) and negative clock components (FIG. 4C) on day 0, 3 and 5 of myogenic differentiation. *,**, p<0.05, 0.01 and 0.001, CHX vs. DMSO by Student's t test. RT-qPCR analysis of expression of CLOCK and Bmal1 (FIG. 4A), its clock target genes (FIG. 4B) and negative clock components (FIG. 4C) in C2C12 myoblasts with stable expression of scrambled control shRNA (SC) or Bmal1 shRNA (BMKD) in response to chlorhexidine (CHX). Treatment before and at day 4 of C2C12 myogenic differentiation.

FIGS. 5A-5D. Chlorhexidine promotes myogenic differentiation. FIG. 5A: Representative images of C2C12 morphological myogenic progression on Day 0, 3, 6 and 9 after CHX treatment. FIG. 5B: Immunofluorescence staining by myosin heavy chain at differentiation day 5 and day 7. 10 representative fields each concentration. Scale bar: 100 μM. FIGS. 5C-5D: Immunoblot analysis (FIG. 5C) and RT-qPCR analysis (FIG. 5D) of myogenic gene expression at indicated CHX concentrations during C2C12 myogenic differentiation. *,**: p<0.05 or 0.01 CHX vs. DMSO by Student's t test.

FIGS. 6A-6D. Effect of chlorhexidine on myogenesis is dependent on clock. FIG. 6A: Representative images of morphological myogenic progression at differentiation Day 0, 3, 6 and 9 in response to CHL treatment in C2C12 myoblasts with stable scrambled control shRNA (SC) or Bmal1 shRNA (BMKD). 10 representative fields each concentration. Scale bar: 100 μM. FIG. 6B: RT-qPCR analysis of myogenic gene expression in C2C12 myoblasts with stable expression of scrambled control shRNA (SC) or Bmal1 shRNA (BMKD) in response to chlorhexidine (CHX) treatment before and at day 4 of C2C12 myogenic differentiation. Clock-dependent effect of chlorhexidine on inducing Wnt pathway. RT-qPCR analysis of expression of Wnt ligands (FIG. 6A), Wnt signaling components (FIG. 6B), and Wnt transcription effectors (FIG. 6C) in C2C12 myoblasts with stable expression of scrambled control shRNA (SC) or Bmal1 shRNA (BMKD) in response to chlorhexidine (CHX) treatment before and at day 4 of C2C12 myogenic differentiation. FIG. 6D: Consistent with previous findings of circadian clock transcriptional control of the Wnt signaling pathway that promotes myogenesis, expressions of key signaling components in this cascade, including Wnt10a, Fzd5 and beta-catenin, were significantly induced by CHX at 0.5 μM with tendency toward induction at 0.2 μM. Notably, this effect was completely abolished in Bmal1-deficient myoblasts, suggesting that CHX effect on inducing Wnt signaling is dependent on clock modulation.

FIGS. 7A-7D. CHX promotes myoblast proliferation and migration. FIGS. 7A-7B: Representative images of EdU staining of C2C12 myoblasts with CHX or SR2878 treatment for 24 hours (FIG. 7A), with quantification (FIG. 7B). 10 representative fields was used for quantitative analysis. *,**: p<0.05 and 0.01 treated vs. DMSO by Student's t test. Scale bar: 100 μM. FIGS. 7C-7D: Representative images of wound healing assay of C2C12 myoblasts for 24 h with CHX treatment at indicated concentrations (FIG. 7C), with quantification (FIG. 7D). 10 representative fields for each concentration. *,**: p<0.05, 0.01 and 0.001, CHX vs. DMSO by Student's t test. Scale bar: 230 μM.

FIG. 8. Quantification of FIG. 4A of chlorhexidine effect on clock protein expression levels by immunoblot analysis.

FIGS. 9A-9D. Clock-dependent effect of chlorhexidine on activation of Wnt signaling. RT-qPCR analysis of key components involved in Wnt pathway signaling transduction in C2C12 myoblasts with stable expression of scrambled control shRNA (SC) or Bmal1 shRNA (BMKD) before and at day 4 of myogenic differentiation, with or without treatment by 0.2 or 0.5 μM of chlorhexidine (CHX).

FIGS. 10A-10B. Chlorhexidine interactions with CLOCK protein hydrophobic pocket. FIG. 10A: Docking pose of CHX, represented as the stick molecule, on the CLOCK hydrophobic pocket structure with interacting amino acid residues listed. FIG. 10B: List of predicted CHX interactions with amino acid residues within the CLOCK hydrophobic pocket by all-around docking modeling.

FIGS. 11A-11C. Analysis of CLOCK protein S-palmitoylation. FIGS. 11A-11B: Identification of CLOCK palmitoylation by acyl-biotin exchange method using hydroxylamine (HAM). Flag-tagged CLOCK is expressed in HEK 293T cells, and total protein extract subjected to palmitoylation assay followed by anti-Flag Western for CLOCK (FIG. 11A), and loss of CLOCK palmitoylation with 2-BrP inhibition of palmitoylation (FIG. 11B). FIG. 11C: Structural modeling of the palmitoylation pocket and the docking pose within the hydrophobic pocket that lies between the interface of CLOCK interaction with Bmal1.

FIGS. 12A-12C. Palmitoylation modulates CLOCK protein stability and interaction with Bmal1. FIG. 12A: Treatment of pan-palmitoylation inhibitor 2-BrP and palmitoylation synthesis inhibitor Orlistat reduces CLOCK protein stability. FIG. 12B: CLOCK palmitoylation cysteine mutations inhibit its interaction with heterodimer partner Bmal1 and Bmal1 stability by single amino acid mutations. FIG. 12C: CLOCK palmitoylation site mutations attenuated CLOCK-mediated transcription activation.

FIGS. 13A-13E. Modulation of palmitoylation impacts circadian clock oscillation. FIGS. 13A-13C: Effect of pan-palmitoylation inhibitor 2-BrP effect on clock properties as shown by Per2 promoter-luciferase monitoring for 5 consecutive days (FIG. 13A), with quantification of clock period length and amplitude (FIG. 13C). FIG. 13D: Effect of palmitate treatment on inducing clock gene expression. FIG. 13E: Proposed working model of CLOCK protein palmitoylation on circadian clock oscillation.

FIGS. 14A-14D. ZDHHC17 mediates CLOCK palmitoylation. FIG. 14A: Co-immunoprecipitation analysis of ZDHHC17 interaction with CLOCK. FIG. 14B: Overexpression of ZDHHC augments CLOCK palmitoylation. FIG. 14C: Inhibition of ZDHHC by shRNA reduced clock oscillation amplitude with period lengthening.

FIGS. 15A-15D. ABHD17C mediates CLOCK de-palmitoylation. FIG. 15A: Co-immunoprecipitation analysis of ABHD17C interaction with CLOCK. FIG. 15B: Overexpression of ABHD17C reduced CLOCK palmitoylation level. FIG. 15C: Inhibition of ABHD17C by shRNA knockdown augments clock oscillation.

FIGS. 16A-16B. Pro-myogenic effect of chlorhexidine on dystrophin-deficient mdx primary myoblasts. FIG. 16A: Representative images of immunofluorescence staining of myosin heavy chain (MyHC) of chlorhexidine treatment on mdx and Bmal1-deficient (BMKO) mdx primary myoblasts. Primary myoblasts were isolated from male mdx mice and differentiated for 3 days in 2% FBS. FIG. 16B: Representative images of EdU fluorescence staining of chlorhexidine treatment of mdx primary myoblasts for 6 hours at indicated concentrations with quantitative analysis (n=5). Scale bar: 100 μm.

FIGS. 17A-17C. In vivo effect of chlorhexidine on inducing regenerative repair of mdx dystrophic muscle. FIG. 17A: Immunoblot analysis of CHX effect on inducting clock and myogenic factor protein expression. CHX was delivered into the Tibialis Anterior (TA) muscle of mdx mice via direct muscular injection. Single and double denotes the number of injections with muscle sample collected 1 day after final injection. FIG. 17B: Immunoblot analysis of effect of triple CHX injection on clock and myogenic factor proteins using pooled (FIG. 17A, n=4) and individual mdx muscle (FIG. 17C, n=3).

FIG. 18. In vivo effect of chlorhexidine on inducing nascent myofiber regeneration of mdx dystrophic muscle. Representative images of Immunofluorescence staining of embryonic myosin heavy chain (eMyHC) to identify nascent regenerated myofiber and Laminin to delineate myofiber were performed following three CHX injections in mdx TA muscle. CHX was dissolved in PBS. Scale bar: 100 μm.

FIGS. 19A-19C. Structure-activity relationship analysis of CHX analogs. FIG. 19A: Clock-activating compounds in assays tested. FIG. 19B: Compounds without significant effect on clock modulation in assays tested. FIG. 19C: Clock-inhibitory compounds in assays tested.

FIGS. 20A-20F. Effect of CM002 on activating circadian clock function with broader effective concentration range. FIGS. 20A-20D: Representative average luminescence recordings of Per2-dLuc reporter over 6 days following treatment of CM002 at indicated concentrations (FIGS. 20A-20B), with quantitative analysis of period length (FIG. 20C) and amplitude (FIG. 20D). FIGS. 20E-20F: Analysis of effect of CHX and CM002 on stimulating Wnt activity via TOPFlash luciferase reporter in U2OS cells at indicated concentrations.

FIGS. 21A-21D. CM002 displayed enhanced pro-myogenic activity than CHX. FIG. 21A: Representative phase-contrast images, and immunofluorescence images of myosin heavy chain (MyHC) staining (FIG. 21B), of differentiated C2C12 myoblasts at day 6 of myogenic differentiation treated by CHX or CM002 at indicated concentrations. FIGS. 21C-21D: Representative images of EdU incorporation to assess proliferation of C2C12 myoblasts treated at indicated concentrations of CHX or CM002 for 4 hour (FIG. 21C), with quantitative analysis (FIG. 21D). n=7-8/treatment group.

FIGS. 22A-22B. CM002 display pro-myogenic activity in mdx primary myoblasts. Representative immunofluorescence images of myosin heavy chain (MyHC) staining of differentiated mdx primary myoblasts at day 3 of myogenic differentiation treated at indicated concentration of CHX or CM002. FIG. 22B: Representative immunofluorescence myosin heavy chain (MyHC) staining of CM002-treated mdx primary myoblasts seeded at low density at day 3 of myogenic differentiation. Scale bar: 100 μm.

FIGS. 23A-23C. Chlorhexidine inhibits terminal differentiation of 3T3-L1 preadipocytes. FIG. 23A: Phase-contrast and Oil-red-O staining of dosage-dependent effect of CHX on inhibiting 3T3-L1 differentiation at Day 6. FIG. 23B: Bodipy fluorescence staining of 3T3-L1 differentiation at Day 6. FIG. 23C: Immunoblot analysis of adipogenic gene expression before and at Day 6 of differentiation. Scale bar: 100 μm.

FIG. 24. Bodipy fluorescence staining of 3T3-L1 differentiation at Day 6 at 20× magnification. Scale bar: 50 μm.

FIGS. 25A-25B. Chlorhexidine inhibits adipogenic differentiation of primary preadipocytes. FIG. 25A: Phase-contrast and Oil-red-O staining of primary preadipocytes at Day 4 and Day 6 of adipogenic differentiation. FIG. 25B: Bodipy fluorescence staining of primary preadipocyte differentiation at Day 4 and Day 6. Scale bar: 100 am.

FIGS. 26A-26D. Chlorhexidine inhibits adipogenic lineage commitment and differentiation in C3H10T1/2 cells. FIG. 26A: Oil-red-O staining of 10T1/2 cells at Day 5 of adipogenic differentiation treated at indicated chlorhexidine concentration. FIG. 26B: Phase-contrast and Oil-red-O staining of 10T1/2 cells at Day 8 of adipogenic differentiation. FIG. 26C: Bodipy fluorescence staining of 10T1/2 cell differentiation at Day 8. Scale bar: 100 am. FIG. 26D: Immunoblot analysis of adipogenic program before and at Day 8 of differentiation treated with 1 μM of chlorhexidine.

FIGS. 27A-27H. Chlorhexidine activates clock in adipocyte progenitors. FIGS. 27A, 27D: Baseline-adjusted tracing plots of average bioluminescence activity of Per2::dLuc reporter-containing C3H10T1/2 (FIGS. 27A-27C) and primary preadipocytes (FIGS. 27D-27F) for 5 days, with quantitative analysis of clock period length (FIGS. 27B, 27E) and cycling amplitude (FIGS. 27C, 27F), with chlorhexidine (CHX) treatment at indicated concentrations. Data are presented as Mean±SD of n=4 replicates for each concentration tested, for three independent repeat experiments. FIGS. 27G, 27H: RT-qPCR analysis of clock gene expression at indicated concentrations of chlorhexidine in C3H10T/2 (FIG. 27G), and 3T3-L1 cells (FIG. 27H). Data are presented as Mean±SD of n=3 replicates. *, **: p<0.05 and 0.01 CHX vs. DMSO by Student's t test.

FIGS. 28A-28F. Chlorhexidine inhibition of lineage commitment and differentiation of adipogenic mesenchymal progenitors. FIG. 28A: Representative images of Oil-red-O staining of 10T1/2 cells at day 5 following adipogenic differentiation treated with chlorhexidine at indicated concentrations. FIG. 28B: Representative images of phase-contrast and Oil-red-O staining of 10T1/2 cells at day 8 of differentiation treated by chlorhexidine at indicated concentration. FIGS. 28C-28D: Representative images of Bodipy fluorescence staining of adipogenic differentiation at Day 8 (FIG. 28C) with quantification (FIG. 28D). Average intensity of BODIPY fluorescence of three representative fields was obtained using Image J and normalized to DAPI signal. Scale bars: 100 μm. FIGS. 28E-28F: Representative images of immunoblot analysis of adipogenic program before and after 8 days of differentiation treated with or without 1 μM of chlorhexidine (FIG. 28E), with quantification normalized to HSP90 level (FIG. 28F). *, **: p<0.05 and 0.01 CHX vs. DMSO by Student's t test.

FIGS. 29A-29E. Chlorhexidine inhibitory effect on preadipocyte terminal differentiation. FIGS. 29A-29C: Representative images of phase-contrast and oil-red-O staining (FIG. 29A), and Bodipy fluorescence staining (FIG. 29B) with quantitative analysis (FIG. 29C), of effect CHX of on adipogenic differentiation of 3T3-L1 preadipocytes at 6 days of differentiation at indicated concentrations. Scale bar: 100 μm. *, **: p<0.05 and 0.01 CHX vs. DMSO by Student's t test. FIGS. 29D-29E: Representative images of immunoblot analysis of adipogenic gene expression before (day 0) and after day 6 of differentiation (FIG. 29D) with quantification normalized to HSP90 level (FIG. 29E).

FIGS. 30A-30E. Effect of chlorhexidine on suppressing primary preadipocyte differentiation. FIGS. 30A-30C: Representative images of phase-contrast and oil-red-O staining (FIG. 30A) and Bodipy fluorescence staining (FIG. 30B) with quantitative analysis (FIG. 30C) of primary preadipocytes at day 6 of adipogenic differentiation treated with CHX at indicated concentrations. Scale bar: 100 μm. FIGS. 30D-30E: Representative images of immunoblot analysis of adipogenic gene expression before (day 0) and after 6 days of primary adipocyte differentiation (FIG. 30D), with quantitative analysis normalized to HSP90 level (FIG. 30E). **: p<0.01 CHX vs. DMSO by Student's t test.

FIGS. 31A-31E. Chlorhexidine promotes Wnt signaling in adipogenic progenitors. FIGS. 31A-31B: RT-qPCR analysis of CHX effect on expression of key Wnt signaling pathways components in C3H10T1/2 cells (FIG. 31A), and 3T3-L1 preadipocytes (FIG. 31B), treated with indicated concentrations of chlorhexidine for 6 hours. Data are presented as Mean±SD of n=3 replicates. *, **: p<0.05 and 0.01 CHX vs. DMSO by Student's t test. FIGS. 31C-31D: Representative images of immunoblot analysis of CHX effect (1 μM) on β-catenin protein level in C3H10T1/2 cells before and after adipogenic induction for 8 days (FIG. 31C), with quantitative analysis normalized to HSP90 level (FIG. 31D). FIG. 31E: Analysis of Wnt signaling pathway activity by a Wnt-responsive TOPFlash luciferase reporter. Data are presented as Mean±SD of n=4 replicates. *, **: p<0.05 or 0.01 CHX vs. DMSO by Student's t test.

FIGS. 32A-32H. CM002 as a chlorhexidine analog with clock-activating properties. FIG. 32A: Docking conformation of CM002 within the CLOCK protein hydrophobic pocket. Crystal structure shown for CLOCK (surface mode) with Bmal1 (cartoon mode) is based on PDB: 4f31. FIG. 32B: Predicted CM002 interactions with CLOCK protein residues within 3-4 Å distance. FIGS. 32C-32D: Baseline-adjusted tracing plots of average bioluminescence activity of U2OS cell lien with stable expression of a Per2::dLuc reporter for 5 days (FIG. 32C) with quantitative analysis of clock period length (FIG. 32D) and cycling amplitude (FIG. 32E), with CM002 treatment at indicated concentrations. Data are presented as Mean±SD of n=4 replicates for each concentration tested. FIG. 32F: RT-qPCR analysis of clock gene expression of C3H10T/2 cells treated by CM002 at indicated concentrations for 6 hours. Data are presented as Mean±SD of n=3 replicates. *, **: p<0.05 and 0.01 CHX vs. DMSO by Student's t test. FIGS. 32G-32H: Analysis of CM002 effect on Wnt signaling activity using TOPFlash luciferase reporter. Data are presented as Mean±SD of n=4 replicates. *, **: p<0.05 or 0.01 CM002 vs. DMSO by Student's t test.

FIGS. 33A-33B. FIG. 33A: Chemical structure of CM002. FIG. 33B: Molecular docking model of CM002 interaction with CLOCK protein residues together with chlorhexidine fitting within the shared hydrophobic pocket structure.

FIGS. 34A-34H. Effect of CM002 on inhibiting C3H10T1/2 mesenchymal adipogenic progenitor differentiation. FIGS. 34A-34C: Representative images of oil-red-O staining (FIG. 34A), and Bodipy fluorescence staining (FIG. 34B) with quantitative analysis (FIG. 34C), of 10T1/2 cells at Day 8 of adipogenic differentiation treated with CM002 at indicated concentrations. Scale bar: 100 μm. FIGS. 34D-34E: Representative images of immunoblot analysis of adipogenic program before and after 8 days of C3H10T1/2 differentiation treated with or without 1 μM of CM002 (FIG. 34D), with quantification using normalization to HSP90 level (FIG. 34E). Each lane represents pooled samples from 3 independent experiments. FIG. 34F: RT-qPCR analysis of clock gene expression of C3H10T/2 cells treated by CM002 at indicated concentrations for 6 hours. FIGS. 34G-34H: Representative immunoblot images of total β-catenin level before and at day 7 of C3H10T1/2 differentiation treated with 1 μM of CM002 (FIG. 34G), with quantification using normalization to HSP90 level (FIG. 34H).

FIGS. 35A-35C. Effect of CM002 on inhibiting primary preadipocyte differentiation. Representative images of oil-red-O staining (FIG. 35A), and Bodipy fluorescence staining (FIG. 35B) with quantitative analysis (FIG. 35C), of primary preadipocytes from control (BMCtr) or Bmal1-null mice (BMKO) after six days of adipogenic differentiation, with CM002 at indicated concentrations. Scale bars: 100 μm. *, **: p<0.05 or 0.01 CM002 vs. DMSO by Student's t test.

FIGS. 36A-36C. Effect of chlorhexidine as a clock activator in 3T3-L1 preadipocytes. FIG. 36A: Baseline-adjusted tracing plots of average bioluminescence activity of Per2:: dLuc reporter-containing 3T3-L1 preadipocytes for 5 days, with quantitative analysis of clock period length (FIG. 36B) and cycling amplitude (FIG. 36C). Chlorhexidine (CHX) treatment was added to culture media at indicated concentrations. Data are presented as Mean±SD of n=4 replicates for each concentration tested, for three independent repeat experiments.

FIGS. 37A-37B. Bodipy fluorescence staining of C3H10T1/2 mesenchymal adipogenic progenitor at day 4 of adipogenic differentiation at 100× (FIG. 37A) and 200× (FIG. 37B) magnification. Scale bar: 50 μm.

FIGS. 38A-38B. Effect of chlorhexidine on inhibiting early adipogenesis of primary preadipocytes. Phase-contrast and oil-red-O staining (FIG. 38A) and Bodipy fluorescence staining (FIG. 38B) of primary preadipocyte at day 4 of early differentiation at indicated chlorhexidine (CHX) concentration.

FIGS. 39A-39B. Effect of CM002 on early differentiation of primary preadipocytes. Phase-contrast and oil-red-O staining (FIG. 39A) and Bodipy fluorescence staining (FIG. 39B) of primary preadipocyte at day 4 of early differentiation with indicated concentration of CM002. Scale bar: 100 μm.

FIGS. 40A-40B. Effect of Chlorhexidine and CM002 on promoting myogenic induction of human DMD primary myoblasts. Immunoblot analysis of myogenic factor induction by CM002 (FIG. 40A) and chlorhexidine (FIG. 40B) treatment in human DMD primary myoblasts at indicated concentrations following 3 days of differentiation (each lane represent pooled three samples). Quantification is based on three repeats. *p<0.05 and ** p<0.01 CM002 or CHX treatment vs. DMSO control by Student's t test.

FIGS. 41A-41D. Effect of Chlorhexidine (CHX) and CM002 effect on viability of mouse and human myoblasts. FIG. 41A: Analysis of mouse C2C12 myoblast cell line viability as determined by MTT assay at indicated concentrations of CHX or CM002. n=3/group. FIGS. 41B-41C: Analysis of cell viability by MTT assay of C2C12 myoblasts (FIG. 41B), and mdx mouse primary myoblasts (FIG. 41C), following treatment with cytokine cocktail together with CHX or CM002 at indicated concentrations. N=4-5/group. FIG. 41D: Analysis of cell line viability as determined by MTT assay of human primary myoblasts derived from Duchene Muscular Dystrophy (DMD) patients, following treatment with cytokine cocktail together with CHX or CM002 at indicated concentrations. N=4-8/group. *p<0.05 and ** p<0.01 CM002 or CHX treatment vs. CC control by Student's t test. CC: cytokine cocktail: 10 ng/mL IL-1β, 100 ng/mL IFN-γ, 25 ng/mL TNF-α.

FIGS. 42A-42B. CM002 and CHX promotes regenerative repair after cardiotoxin-induced acute muscle injury. FIG. 42A: Immunoblot analysis of embryonic myosin heavy chain (eMyHC) and laminin to examine new myofiber synthesis in TA muscle at 7 days after cardiotoxin injury, with injection of CHX (3 days 0.5 mM) or CM002 (3 days 0.5 mM). FIG. 42B: Immunoblot analysis of myogenic factor expression following injections with CHX or CM002 in CTX-injected TA muscle at day 7 of regeneration. Each lane represent pooled sample of 4-6 mice/group.

FIGS. 43A-43D. Effect of Chlorhexidine and CM002 on promoting regenerative myogenesis in mdx mice. Immunoblot analysis of clock regulators (FIG. 43A) and myogenic factor induction (FIG. 43B) by three daily doses of chlorhexidine (50 μl of 2 μM) and CM002 (50 μl of 2 μM) intramuscular injection in TA muscle of mdx mice. Each lane represent pooled samples of 3-4 mice/lane. FIG. 43C: Representative images of immunofluorescence staining of embryonic myosin heavy chain and laminin of mdx mice TA cross section following three daily chlorhexidine or CM002 treatment in mdx mice. FIG. 43D: Quantification of TA cross section area distribution of chlorhexidine or CM002-treated mdx mice. *p<0.05 and ** p<0.01 CM002 or CHX treatment vs. DMSO control by Student's t test.

FIGS. 44A-44D. Intramuscular injections of CM002 and CHX promotes satellite cell proliferation and regenerative myogenesis in mdx mice. FIG. 44A: Representative images of EdU and Pax7 immunofluorescence staining of mdx mice TA muscle cross sections, following daily direction intra-muscular injection of 150 μl of 2 μM of CM002 or CHX for three days. FIG. 44B: Quantitative analysis of proliferative satellite cells as a percentage of the satellite cell pool or total nuclei present. Quantification is based on n=7-9/group. FIGS. 44C-44D: RT-qPCR analysis of expression of clock genes (FIG. 44C), and myogenic factors (FIG. 44D). N=6-7/group. *p<0.05 and ** p<0.01 CM002 or CHX treatment vs. PBS injection control by Student's t test.

FIGS. 45A-45D. Effect of CHX and CM002 intramuscular injection on muscle injury in mdx mice. Representative images of Evans Blue Dye fluorescence of mdx mice TA cross section (FIG. 45A), or stitched images of entire TA section (FIG. 45B) following daily 150 ul of 2 uM of CM002 or CHX injection in TA for 3 days. N=6-10/group. FIG. 45C: Quantitative analysis of Evans blue dye staining in entire TA sections of mdx mice with CM002 or CHX intramuscular injections. FIG. 45D: Analysis of creatine kinase activity from secreted media of TA explant from mdx mice following intramuscular injections of CM002 or CHX. Wild-type C57/BL6 mice were included as negative control. TA were dissected and cultured in cell culture media for 24 hours. *p<0.05 and ** p<0.01 CM002 or CHX treatment vs. PBS control by Student's t test. *p<0.01 mdx vs. PBS control. N=6-13/group.

FIGS. 46A-46D. CM002 and CHX ameliorate inflammation in mdx mice and display anti-inflammasome activity. FIG. 46A: RT-qPCR analysis of inflammatory cytokine gene expression of mdx TA muscle injected with CM002 or CHX for 3 daily doses. N=6-7/group. *p<0.05 and ** p<0.01 CM002 or CHX treatment vs. PBS control by Student's t test. *p<0.01 mdx vs. PBS control. FIG. 46B: Immunoblot analysis of effect of CHX and CM002 on NLRP3 inflammasome expression and induction of CHOP in primary bone marrow-derived macrophages. FIGS. 46C-46D: Immunoblot analysis of effect of CHX (FIG. 46C), and CM002 (FIG. 46D) treatment on NLRP3 and CHOP with LPS stimulation in bone marrow-derived macrophages at indicated concentrations.

FIGS. 47A-47C. In vivo pharmacokinetics analysis of CM002 in mice. Analysis of CM002 plasma concentration at 6 hours (FIG. 47A) and 24 hours (FIG. 47B) following intravenous injection via tail vein at 1 mg/kg of body weight. Three mice of C57/BL6 at 12 weeks of age were used. FIG. 47C: Quantitative analysis of pharmacokinetic parameters of CM002 based on 1 mg/kg/day intravenous administration.

FIGS. 48A-48C. Effect of CM002 by intraperitoneal injection in mdx mice. FIG. 48A: Determination of maximal lethal dose in mice with intraperitoneal injection of CM002 consecutively for 3 days. FIG. 48B: Analysis of grip strength of mdx mice following intraperitoneal injection of DMSO or CM002. CM002 was injected daily at 10 mg/kg for 3 days and grip strength measured at day 4 after last injection. Male mdx mice at 10-14 weeks of age were used. *p<0.05 and ** p<0.01 by one-way ANOVA with Tukey's post-hoc analysis. FIG. 48C: Analysis of body weight and muscle tissue weight of mdx mice following IP injection of CM002 (10 mg/kg) for 3 days. TA: Tibialis Anterior; GN: Gastrocnemius.

FIGS. 49A-49C. Systemic delivery of CM002 by in normal C57/BL6 wild-type mice reduces food consumption coupled with increased energy expenditure. C57/BL6 wild-type mice were administered with daily intraperitoneal injection of DMSO or CM002 (5 mg/Kg) and energy balance were monitored using metabolic cage for 3 days with data show for two consecutive daily cycles following 1 day of acclimation. Analysis of accumulative food consumption (FIG. 49A), hourly energy consumption (FIG. 49B) and average tracing of oxygen consumption (FIG. 49C) over 2 days. N=3/group.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O—is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

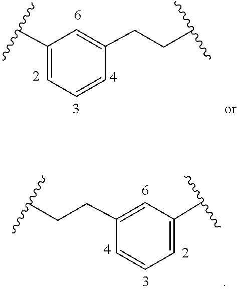 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, =O, =NR′, =N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)=NR″″, —NRC(NR′R″)=NR′″″, —S(O)R′, —S(O)₂R′, —S(O)₂NR′R″, —NRSO₂R′, —NR′NR″R″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′ C(O)NR″R′″, —NR″C(O)₂R′, —NR—C(NR′R″R′″)=NR″″, —NR—C(NR′R″)=NR′″″, —S(O)R′, —S(O)₂R′, —S(O)₂NR′R″, —NRSO₂R′, —NR′NR″R′″″, —ONR′R″, —NR′C(O) NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and/or
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and/or
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and/or
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

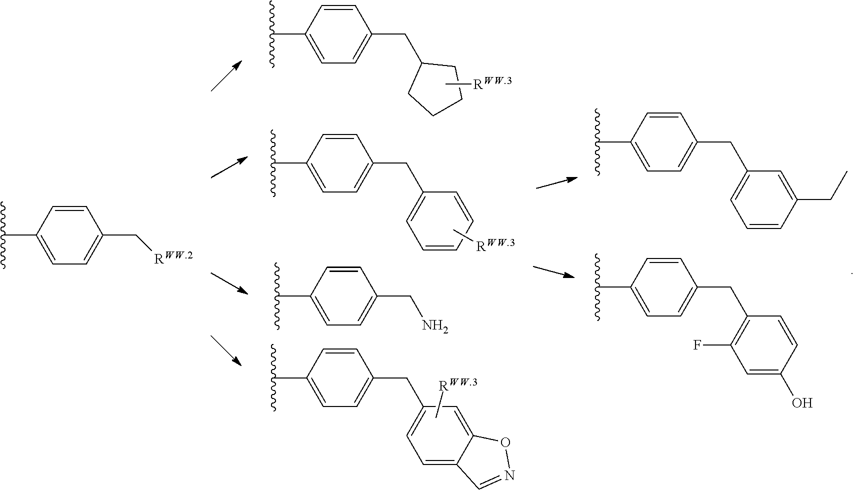

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R′″-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist. The term “clock activator” as used herein refers to a substance capable of increasing the expression or activity of a CLOCK protein or CLOCK/Bmal1 transcriptional activity relative to the absence of the substance.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments, inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient in need thereof is human. In embodiments, a subject is human. In embodiments, a subject in need thereof is human.

“Disease” or “condition” refers to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a muscle degenerative disease. In embodiments, the muscle degenerative disease is muscular dystrophy. In embodiments, the muscle degenerative disease is aging-induced sarcopenia. In embodiments, the muscle degenerative disease is cachexia.

The term “cachexia” is used in accordance with its plain ordinary meaning and refers to a syndrome causing muscle loss. A range of diseases can cause cachexia, including cancer, congestive heart failure, chronic obstructive pulmonary disease, chronic kidney disease, and acquired immunodeficiency syndrome (AIDS). As used herein, the term “cancer-associated cachexia” refers to cachexia associated with cancer. As used herein, the term “chronic inflammatory disease-associated cachexia” refers to cachexia associated with chronic inflammatory disease.

As used herein, the term “inflammatory disease” refers to a disease or condition characterized by aberrant inflammation (e.g., an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, autoimmune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, the term “chronic inflammatory disease” refers to a disease or condition characterized by prolonged aberrant inflammation. Examples of chronic inflammatory diseases include Crohn's disease, ulcerative colitis, rheumatoid arthritis, psoriatic arthritis, psoriasis, lupus, asthma, and chronic obstructive pulmonary disease.

The term “metabolic disorder” refers to a disorder characterized by one or more abnormal metabolic processes in a subject. In embodiments, a metabolic disorder may be associated with, related to, or may be diabetes (e.g., type 1 diabetes or type 2 diabetes), insulin resistance, metabolic syndrome, obesity, aging-related obesity, sarcopenic obesity, hyperlipidemia, hyperglycemia, high serum triglycerides, and/or high blood pressure. In embodiments, a metabolic disorder may be associated with, related to, or may be a diabetes associated disease selected from nephropathy, retinopathy, neuropathy, cardiovascular disease, or inflammation. In embodiments, a metabolic disorder may be associated with, related to, or may be nephropathy, retinopathy, neuropathy, cardiovascular disease, or inflammation.

The term “myogenesis” is used in accordance with its plain ordinary meaning and refers to the formation of muscular tissue.

The term “adipogenesis” is used in accordance with its plain ordinary meaning and refers to the formation of adipocytes, or fat cells, from stem cells.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁵Rh ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., muscle degenerative disease) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to E116 of the CLOCK protein when the selected residue occupies the same essential spatial or other structural relationship as E116 of the CLOCK protein. In some embodiments, where a selected protein is aligned for maximum homology with the CLOCK protein, the position in the aligned selected protein aligning with E116 is said to correspond to E116. Instead of a primary sequence alignment, a three dimensional structural alignment can also be used, e.g., where the structure of the selected protein is aligned for maximum correspondence with the CLOCK protein and the overall structures compared. In this case, an amino acid that occupies the same essential position as E116 in the structural model is said to correspond to the E116 residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “CLOCK” or “circadian locomotor output cycles kaput” refers to a protein (including homologs, isoforms, and functional fragments thereof) that plays a role in activating downstream elements in the pathway critical to the generation of circadian rhythms. The term includes any recombinant or naturally-occurring form of CLOCK variants thereof that maintain CLOCK activity (e.g., within at least 30%, 40%, 50%, 60%, 70%, 80%, 90%, 95%, or 100% activity compared to wildtype CLOCK). In embodiments, the CLOCK protein encoded by the CLOCK gene has the amino acid sequence set forth in or corresponding to Entrez 9575, UniProt O15516, RefSeq (protein) NP_001254772.1, or RefSeq (protein) NP_004889.1, or homolog thereof. In embodiments, the amino acid sequence or nucleic acid sequence is the sequence known at the time of filing of the present application. In embodiments, the CLOCK protein has the following amino acid sequence:

(SEQ ID NO: 1)
MLFTVSCSKMSSIVDRDDSSIFDGLVEEDDKDKAKRVSRNKSEKKRRDQF
NVLIKELGSMLPGNARKMDKSTVLQKSIDFLRKHKEITAQSDASEIRQDW
KPTFLSNEEFTQLMLEALDGFFLAIMTDGSIIYVSESVTSLLEHLPSDLV
DQSIFNFIPEGEHSEVYKILSTHLLESDSLTPEYLKSKNQLEFCCHMLRG
TIDPKEPSTYEYVKFIGNFKSLNSVSSSAHNGFEGTIQRTHRPSYEDRVC
FVATVRLATPQFIKEMCTVEEPNEEFTSRHSLEWKFLFLDHRAPPIIGYL
PFEVLGTSGYDYYHVDDLENLAKCHEHLMQYGKGKSCYYRFLTKGQQWIW
LQTHYYITYHQWNSRPEFIVCTHTVVSYAEVRAERRRELGIEESLPETAA
DKSQDSGSDNRINTVSLKEALERFDHSPTPSASSRSSRKSSHTAVSDPSS
TPTKIPTDTSTPPRQHLPAHEKMVQRRSSFSSQSINSQSVGSSLTQPVMS
QATNLPIPQGMSQFQFSAQLGAMQHLKDQLEQRTRMIEANIHRQQEELRK
IQEQLQMVHGQGLQMFLQQSNPGLNFGSVQLSSGNSSNIQQLAPINMQGQ
VVPTNQIQSGMNTGHIGTTQHMIQQQTLQSTSTQSQQNVLSGHSQQTSLP
SQTQSTLTAPLYNTMVISQPAAGSMVQIPSSMPQNSTQSAAVTTFTQDRQ
IRFSQGQQLVTKLVTAPVACGAVMVPSTMLMGQVVTAYPTFATQQQQSQT
LSVTQQQQQQSSQEQQLTSVQQPSQAQLTQPPQQFLQTSRLLHGNPSTQL
ILSAAFPLQQSTFPQSHHQQHQSQQQQQLSRHRTDSLPDPSKVQPQ.

The term “BMAL1” or “brain and muscle ARNT-Like 1” refers to a protein (including homologs, isoforms, and functional fragments thereof) that plays a role in generating circadian rhythms. The term includes any recombinant or naturally-occurring form of BMAL1 variants thereof that maintain BMAL1 activity (e.g., within at least 30%, 40%, 50%, 60%, 70%, 80%, 90%, 95%, or 100% activity compared to wildtype BMAL1). In embodiments, the BMAL1 protein encoded by the ARNTL gene has the amino acid sequence set forth in or corresponding to Entrez 406, UniProt 000327, RefSeq (protein) NP_001025443.1, RefSeq (protein) NP_001025444.1, RefSeq (protein) NP_001169.3, RefSeq (protein) NP_001284648.1, RefSeq (protein) NP_001284651.1, or RefSeq (protein) NP_001284653.1, or homolog thereof. In embodiments, the amino acid sequence or nucleic acid sequence is the sequence known at the time of filing of the present application. In embodiments, the BMAL1 protein has the following amino acid sequence:

(SEQ ID NO: 2)
MADQRMDISSTISDFMSPGPTDLLSSSLGTSGVDCNRKRKGSSTDYQESM
DTDKDDPHGRLEYTEHQGRIKNAREAHSQIEKRRRDKMNSFIDELASLVP
TCNAMSRKLDKLTVLRMAVQHMKTLRGATNPYTEANYKPTFLSDDELKHL
ILRAADGFLFVVGCDRGKILFVSESVFKILNYSQNDLIGQSLFDYLHPKD
IAKVKEQLSSSDTAPRERLIDAKTGLPVKTDITPGPSRLCSGARRSFFCR
MKCNRPSVKVEDKDFPSTCSKKKADRKSFCTIHSTGYLKSWPPTKMGLDE
DNEPDNEGCNLSCLVAIGRLHSHVVPQPVNGEIRVKSMEYVSRHAIDGKF
VFVDQRATAILAYLPQELLGTSCYEYFHQDDIGHLAECHRQVLQTREKIT
TNCYKFKIKDGSFITLRSRWFSFMNPWTKEVEYIVSTNTVVLANVLEGGD
PTFPQLTASPHSMDSMLPSGEGGPKRTHPTVPGIPGGTRAGAGKIGRMIA
EEIMEIHRIRGSSPSSCGSSPLNITSTPPPDASSPGGKKILNGGTPDIPS
SGLLSGQAQENPGYPYSDSSSILGENPHIGIDMIDNDQGSSSPSNDEAAM
AVIMSLLEADAGLGGPVDESDLPWPL.

The term “ZDHHC17” or “zinc finger DHHC-type palmitoyltransferase 17” refers to a protein (including homologs, isoforms, and functional fragments thereof) that plays a role in mediating CLOCK protein palmitoylation. The term includes any recombinant or naturally-occurring form of ZDHHC17 variants thereof that maintain ZDHHC17 activity (e.g., within at least 30%, 40%, 50%, 60%, 70%, 80%, 90%, 95%, or 100% activity compared to wildtype ZDHHC17). In embodiments, the ZDHHC17 protein encoded by the ZDHHC17 gene has the amino acid sequence set forth in or corresponding to Entrez 23390, UniProt Q8IUH5, RefSeq (protein) NP_056151.2, or RefSeq (protein) NP_001359626, or homolog thereof. In embodiments, the amino acid sequence or nucleic acid sequence is the sequence known at the time of filing of the present application.

The term “ABHD17C” or “alpha/beta hydrolase domain-containing protein 17C” refers to a protein (including homologs, isoforms, and functional fragments thereof) that plays a role in mediating CLOCK protein palmitoylation. The term includes any recombinant or naturally-occurring form of ABHD17C variants thereof that maintain ABHD17C activity (e.g., within at least 30%, 40%, 50%, 60%, 70%, 80%, 90%, 95%, or 100% activity compared to wildtype ABHD17C). In embodiments, the ABHD17C protein encoded by the ABHD17C gene has the amino acid sequence set forth in or corresponding to UniProt Q6PCB6 or RefSeq (protein) NP_067037.1, or homolog thereof. In embodiments, the amino acid sequence or nucleic acid sequence is the sequence known at the time of filing of the present application.

The term “palmitoylation site” or “palmitoylation pocket” as used herein refers to a hydrophobic pocket of a CLOCK protein, formed by residues corresponding to residues 112-347 of the CLOCK protein. In embodiments, the clock activator (e.g., compound described herein) binds or contacts one amino acid that forms the palmitoylation site. In embodiments, the clock activator (e.g., compound described herein) binds or contacts multiple amino acids that form the palmitoylation site.

In embodiments, the clock activator (e.g., compound described herein) binds or contacts one palmitoylation site amino acid selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts multiple palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts two palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts three palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts four palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts five palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts six palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts seven palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts eight palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts nine palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts ten palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 11 palmitoylation site amino acids selected from Q112, L113, L115, E116, A117, L118, D119, L141, R256, P260, Q261, F262, 1263, K264, M266, T268, E270, E271, E274, P294, P295, I296, G298, T343, K344, G345, Q346, or Q347. In embodiments, the clock activator (e.g., compound described herein) binds or contacts 12 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 13 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 14 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 15 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 16 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 17 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 18 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 19 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 20 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 21 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 22 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 23 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 24 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 25 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 26 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 27 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347. In embodiments, the clock activator (e.g., compound described herein) binds or contacts 28 palmitoylation site amino acids selected from Q112, L113, L115, E116, A117, L118, D119, L141, R256, P260, Q261, F262, I263, K264, M266, T268, E270, E271, E274, P294, P295, I296, G298, T343, K344, G345, Q346, or Q347.

### II. Compounds

In an aspect is provided a compound, or a pharmaceutically acceptable salt thereof, having the formula:

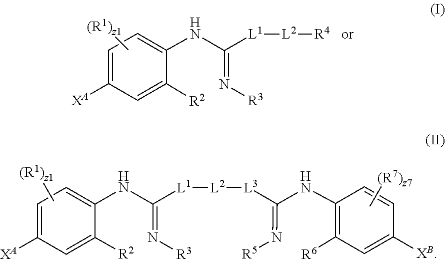

X^{A} and X^{B} are independently —Cl, —Br, —I, or —F.

L¹ is a bond, —C(O)—, —C(O)O—, —OC(O)—, —O—, —S—, —NR¹⁰—, —C(O)NR¹⁰—, —NR¹⁰C(O)—, —NR¹⁰C(O)O—, —OC(O)NR¹⁰—, —NR¹⁰C(O)NR¹⁰—, —NR¹⁰C(NH)NR¹⁰—, —S(O)₂—, —NR¹⁰S(O)₂—, —S(O)₂NR¹⁰—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L² is a bond, —C(O)—, —C(O)O—, —OC(O)—, —O—, —S—, —NR²⁰—, —C(O)NR²⁰—, —NR²⁰C(O)—, —NR²⁰C(O)O—, —OC(O)NR²⁰—, —NR²⁰C(O)NR²⁰—, —NR²⁰C(NH)NR²⁰—, —S(O)₂—, —NR²⁰S(O)₂—, —S(O)₂NR²⁰—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —NR³⁰C(NH)NR³⁰—, —S(O)₂—, —NR³⁰S(O)₂—, —S(O)₂NR³⁰—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² and R³ substituents may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OC(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ and R⁶ substituents may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

R⁷ is independently halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NR^{7C}C(O)NR^{7A}R^{7B}, —N(O)ₘ₇, —NR^{7A}R^{7B}, —C(O)R^{7C}, —C(O)OR^{7C}, —OC(O)R^{7C}, —OC(O)OR^{7C}, —C(O)NR^{7A}R^{7B}, —OC(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

Each R¹⁰, R²⁰, and R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A}, R^{1B}, R^{1C}, R^{1D}, R^{2A}, R^{2B}, R^{2C}, R^{2D}, R^{4A}, R^{4B}, R^{4C}, R^{4D}, R^{6A}, R^{6B}, R^{6C}, R^{6D}, R^{7A}, R^{7B}, R^{7C}, and R^{7D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{6A} and R^{6B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{7A} and R^{7B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

The symbol z1 is an integer from 0 to 3.

The symbol z7 is an integer from 0 to 2.

Each X¹, X², X⁴, X⁶, and X⁷ is independently —Cl, —Br, —I, or —F.

The symbols n1, n2, n4, n6, and n7 are independently an integer from 0 to 4.

The symbols m1, m2, m4, m6, m7, v1, v2, v4, v6, and v7 are independently 1 or 2.

In embodiments, the compound is not

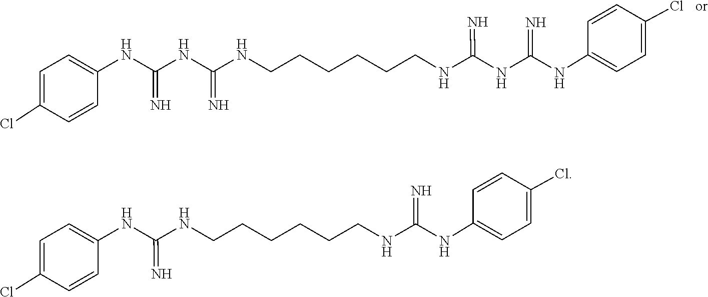 [SMILES: N=C(NCCCCCCNC(=N)NC(=N)Nc1ccc(Cl)cc1)NC(=N)Nc1ccc(Cl)cc1 ; N=C(NCCCCCCNC(=N)Nc1ccc(Cl)cc1)Nc1ccc(Cl)cc1]

In embodiments, the compound is not

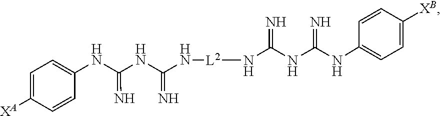

wherein X^{A} and X^{B} are independently —Cl, —Br, —I, or —F; and L² is unsubstituted C₂-C₁₀ alkylene. In embodiments, the compound is not

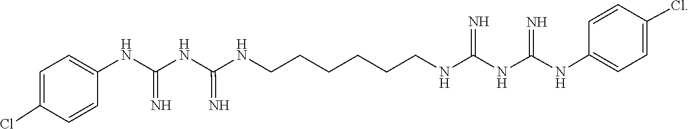 [SMILES: N=C(NCCCCCCNC(=N)NC(=N)Nc1ccc(Cl)cc1)NC(=N)Nc1ccc(Cl)cc1]

In embodiments, the compound is not

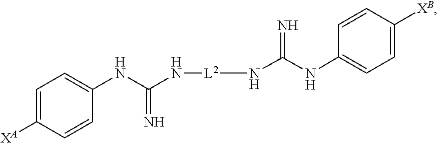

wherein X^{A} and X^{B} are independently —Cl, —Br, —I, or —F; and L² is unsubstituted C₂-C₁₀ alkylene. In embodiments, the compound is not

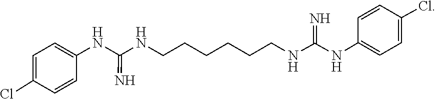 [SMILES: N=C(NCCCCCCNC(=N)Nc1ccc(Cl)cc1)Nc1ccc(Cl)cc1]

In embodiments, the compound has the formula:

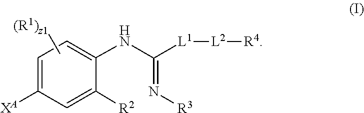

X^{A}, L¹, L², R¹, z1, R², R³, and R⁴ are as described herein, including in embodiments.

In embodiments, the compound has the formula:

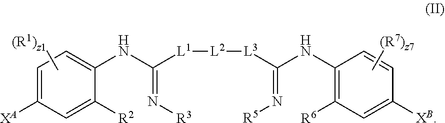

X^{A}, X^{B} L¹, L², L³, R¹, z1, R², R³, R⁵, R⁶, R⁷, and z7 are as described herein, including in embodiments.

In embodiments, the compound has the formula:

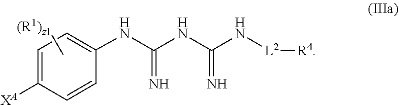

X^{A}, L², R¹, z1, and R⁴ are as described herein, including in embodiments.

In embodiments, the compound has the formula:

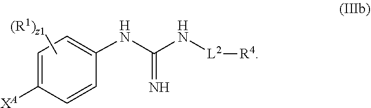

X^{A}, L², R¹, z1, and R⁴ are as described herein, including in embodiments.

In embodiments, the compound has the formula:

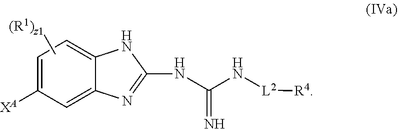

X^{A}, L², R¹, z1, and R⁴ are as described herein, including in embodiments.

In embodiments, the compound has the formula:

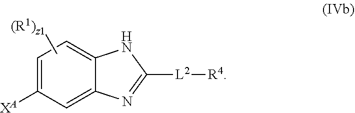

X^{A}, L², R¹, z1, and R⁴ are as described herein, including in embodiments.

In embodiments, the compound has the formula:

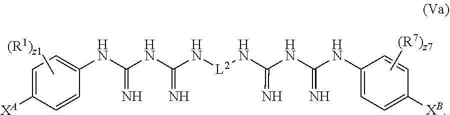

X^{A}, X^{B}, L², R¹, z1, R⁷, and z7 are as described herein, including in embodiments.

In embodiments, the compound has the formula:

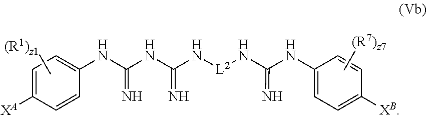

X^{A}, X^{B}, L², R¹, z1, R⁷, and z7 are as described herein, including in embodiments.

In embodiments, the compound has the formula:

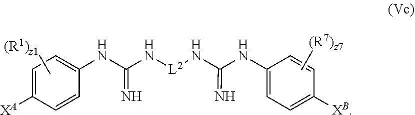

X^{A}, X^{B}, L², R¹, z1, R⁷, and z7 are as described herein, including in embodiments.

In embodiments, the compound has the formula:

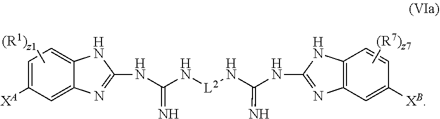

X^{A}, X^{B}, L², R¹, z1, R⁷, and z7 are as described herein, including in embodiments.

In embodiments, the compound has the formula:

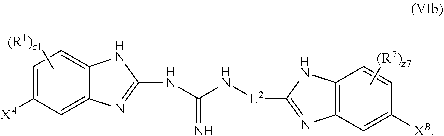

X^{A}, X^{B}, L², R¹, z1, R⁷, and z7 are as described herein, including in embodiments.

In embodiments, the compound has the formula:

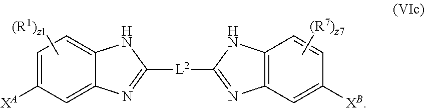

X^{A}, X^{B}, L², R¹, z1, R⁷, and z7 are as described herein, including in embodiments.

In an aspect is provided a compound, or a pharmaceutically acceptable salt thereof, having the formula:

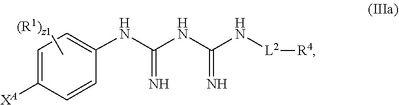

wherein X^{A} is —Cl, —Br, —I, or —F; L² is a bond; R⁴ is unsubstituted C₄-C₂₀ alkenyl; R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₅, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); z1 is an integer from 0 to 3; each X¹ is independently —Cl, —Br, —I, or —F; n1 is independently an integer from 0 to 4; and m1 and v1 are independently 1 or 2.

In embodiments, X^{A} is —Cl. In embodiments, X^{A} is —Br. In embodiments, X^{A} is —I. In embodiments, X^{A} is —F.

In embodiments, X^{B} is —Cl. In embodiments, X^{B} is —Br. In embodiments, X^{B} is —I. In embodiments, X^{B} is —F.

In embodiments, a substituted L¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond, —C(O)—, —C(O)O—, —OC(O)—, —O—, —S—, —NH—, —C(O)NH—, —NHC(O)—, —NHC(O)O—, —OC(O)N H—, —NHC(O)NH—, —NHC(NH)NH—, —S(O)₂—, —NHS(O)₂—, —S(O)₂NH—, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L¹ is a bond, —NH—, —NHC(NH)NH—, or substituted or unsubstituted 2 to 10 membered heteroalkylene.

In embodiments, L¹ is a bond. In embodiments, L¹ is —C(O)—. In embodiments, L¹ is —C(O)O—. In embodiments, L¹ is —OC(O)—. In embodiments, L¹ is —O—. In embodiments, L¹ is —S—. In embodiments, L¹ is —NH—. In embodiments, L¹ is —C(O)NH—. In embodiments, L¹ is —NHC(O)—. In embodiments, L¹ is —NHC(O)O—. In embodiments, L¹ is —OC(O)NH—. In embodiments, L¹ is —NHC(O)NH—. In embodiments, L¹ is —NHC(NH)NH—. In embodiments, L¹ is —S(O)₂—. In embodiments, L¹ is —NHS(O)₂—. In embodiments, L¹ is —S(O)₂NH—. In embodiments, L¹ is substituted or unsubstituted 2 to 10 membered heteroalkylene. In embodiments, L¹ is unsubstituted 2 to 10 membered heteroalkylene.

In embodiments, a substituted L² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is a bond, —C(O)—, —C(O)O—, —OC(O)—, —O—, —S—, —NH—, —C(O)NH—, —NHC(O)—, —NHC(O)O—, —OC(O)N H—, —NHC(O)NH—, —NHC(NH)NH—, —S(O)₂—, —NHS(O)₂—, —S(O)₂NH—, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

In embodiments, L² is a bond. In embodiments, L² is —C(O)—. In embodiments, L² is —C(O)O—. In embodiments, L² is —OC(O)—. In embodiments, L² is —O—. In embodiments, L² is —S—. In embodiments, L² is —NH—. In embodiments, L² is —C(O)NH—. In embodiments, L² is —NHC(O)—. In embodiments, L² is —NHC(O)O—. In embodiments, L² is —OC(O)NH—. In embodiments, L² is —NHC(O)NH—. In embodiments, L² is —NHC(NH)NH—. In embodiments, L² is —S(O)₂—. In embodiments, L² is —NHS(O)₂—. In embodiments, L² is —S(O)₂NH—. In embodiments, L² is substituted or unsubstituted C₂-C₂₀ alkylene. In embodiments, L² is unsubstituted C₁ alkylene. In embodiments, L² is unsubstituted C₂ alkylene. In embodiments, L² is unsubstituted C₃ alkylene. In embodiments, L² is unsubstituted C₄ alkylene. In embodiments, L² is unsubstituted C₅ alkylene. In embodiments, L² is unsubstituted C₆ alkylene. In embodiments, ^{L} is unsubstituted C₇ alkylene. In embodiments, ^{L} is unsubstituted C₈ alkylene. In embodiments, L² is unsubstituted C₉ alkylene. In embodiments, L² is unsubstituted C₁₀ alkylene. In embodiments, L² is unsubstituted C₁₁ alkylene. In embodiments, L² is unsubstituted C₁₂ alkylene. In embodiments, L² is unsubstituted C₁₃ alkylene. In embodiments, L² is unsubstituted C₁₄ alkylene. In embodiments, L² is unsubstituted C₁₅ alkylene. In embodiments, L² is unsubstituted C₁₆ alkylene. In embodiments, L² is unsubstituted C₁₇ alkylene. In embodiments, L² is unsubstituted C₁₈ alkylene. In embodiments, L² is unsubstituted C₁₉ alkylene. In embodiments, L² is unsubstituted C₂₀ alkylene. In embodiments, L² is substituted or unsubstituted C₂-C₂₀ alkenylene. In embodiments, L² is unsubstituted C₂ alkenylene. In embodiments, L² is unsubstituted C₃ alkenylene. In embodiments, L² is unsubstituted C₄ alkenylene. In embodiments, L² is unsubstituted C₅ alkenylene. In embodiments, L² is unsubstituted C₆ alkenylene. In embodiments, L² is unsubstituted C₇ alkenylene. In embodiments, L² is unsubstituted C₈ alkenylene. In embodiments, L² is unsubstituted C₉ alkenylene. In embodiments, L² is unsubstituted C₁₀ alkenylene. In embodiments, L² is unsubstituted C₁₁ alkenylene. In embodiments, L² is unsubstituted C₁₂ alkenylene. In embodiments, L² is unsubstituted C₁₃ alkenylene. In embodiments, L² is unsubstituted C₁₄ alkenylene. In embodiments, L² is unsubstituted C₁₅ alkenylene. In embodiments, L² is unsubstituted C₁₆ alkenylene. In embodiments, L² is unsubstituted C₁₇ alkenylene. In embodiments, L² is unsubstituted C₁₈ alkenylene. In embodiments, L² is unsubstituted C₁₉ alkenylene. In embodiments, L² is unsubstituted C₂₀ alkenylene. In embodiments, L² is

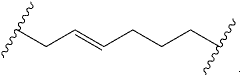 [SMILES: *C/C=C/CCC*]

In embodiments, L² is

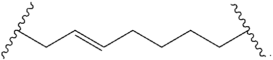 [SMILES: *C/C=C/CCCC*]

In embodiments, L² is substituted or unsubstituted 2 to 20 membered heteroalkylene.

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L³ is a bond, —C(O)—, —C(O)O—, —OC(O)—, —O—, —S—, —NH—, —C(O)NH—, —NHC(O)—, —NHC(O)O—, —OC(O)N H—, —NHC(O)NH—, —NHC(NH)NH—, —S(O)₂—, —NHS(O)₂—, —S(O)₂NH—, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L³ is a bond, —NH—, —NHC(NH)NH—, or substituted or unsubstituted 2 to 10 membered heteroalkylene.

In embodiments, L³ is a bond. In embodiments, L³ is —C(O)—. In embodiments, L³ is —C(O)O—. In embodiments, L³ is —OC(O)—. In embodiments, L³ is —O—. In embodiments, L³ is —S—. In embodiments, L³ is —NH—. In embodiments, L³ is —C(O)NH—. In embodiments, L³ is —NHC(O)—. In embodiments, L³ is —NHC(O)O—. In embodiments, L³ is —OC(O)NH—. In embodiments, L³ is —NHC(O)NH—. In embodiments, L³ is —NHC(NH)NH—. In embodiments, L³ is —S(O)₂—. In embodiments, L³ is —NHS(O)₂—. In embodiments, L³ is —S(O)₂NH—. In embodiments, L³ is substituted or unsubstituted 2 to 10 membered heteroalkylene. In embodiments, L³ is unsubstituted 2 to 10 membered heteroalkylene.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently —CCl₃. In embodiments, R¹ is independently —CBr₃. In embodiments, R¹ is independently —CF₃. In embodiments, R¹ is independently —Cl₃. In embodiments, R¹ is independently —CH₂Cl. In embodiments, R¹ is independently —CH₂Br. In embodiments, R¹ is independently —CH₂F. In embodiments, R¹ is independently —CH₂I. In embodiments, R¹ is independently —CHCl₂. In embodiments, R¹ is independently —CHBr₂. In embodiments, R¹ is independently —CHF₂. In embodiments, R¹ is independently —CHI₂. In embodiments, R¹ is independently —CN. In embodiments, R¹ is independently —OH. In embodiments, R¹ is independently —NH₂. In embodiments, R¹ is independently —COOH. In embodiments, R¹ is independently —CONH₂. In embodiments, R¹ is independently —NO₂. In embodiments, R¹ is independently —SH. In embodiments, R¹ is independently —SO₃H. In embodiments, R¹ is independently —OSO₃H. In embodiments, R¹ is independently —SO₂NH₂. In embodiments, R¹ is independently —NHNH₂. In embodiments, R¹ is independently —ONH₂. In embodiments, R¹ is independently —NHC(O)NH₂. In embodiments, R¹ is independently —NHSO₂H. In embodiments, R¹ is independently —NHC(O)H. In embodiments, R¹ is independently —NHC(O)OH. In embodiments, R¹ is independently —NHOH. In embodiments, R¹ is independently —OCCl₃. In embodiments, R¹ is independently —OCBr₃. In embodiments, R¹ is independently —OCF₃. In embodiments, R¹ is independently —OCI₃. In embodiments, R¹ is independently —OCH₂Cl. In embodiments, R¹ is independently —OCH₂Br. In embodiments, R¹ is independently —OCH₂F. In embodiments, R¹ is independently —OCH₂I. In embodiments, R¹ is independently —OCHCl₂. In embodiments, R¹ is independently —OCHBr₂. In embodiments, R¹ is independently —OCHF₂. In embodiments, R¹ is independently —OCHI₂. In embodiments, R¹ is independently —N₃. In embodiments, R¹ is independently substituted or unsubstituted C₁-C₆ alkyl. In embodiments, R¹ is independently unsubstituted C₁-C₄ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted isobutyl. In embodiments, R¹ is independently unsubstituted tert-butyl. In embodiments, R¹ is independently unsubstituted 2 to 4 membered heteroalkyl. In embodiments, R¹ is independently unsubstituted methoxy. In embodiments, R¹ is independently unsubstituted ethoxy. In embodiments, R¹ is independently unsubstituted propoxy. In embodiments, R¹ is independently unsubstituted n-propoxy. In embodiments, R¹ is independently unsubstituted isopropoxy. In embodiments, R¹ is independently unsubstituted butoxy. In embodiments, R¹ is independently unsubstituted n-butoxy. In embodiments, R¹ is independently unsubstituted isobutoxy. In embodiments, R¹ is independently unsubstituted tert-butoxy. In embodiments, R¹ is independently substituted or unsubstituted 2 to 6 membered heteroalkyl. In embodiments, R¹ is independently substituted or unsubstituted C₃-C₈ cycloalkyl. In embodiments, R¹ is independently substituted or unsubstituted 3 to 8 membered heterocycloalkyl. In embodiments, R¹ is independently substituted or unsubstituted C₆-C₁₀ aryl. In embodiments, R¹ is independently substituted or unsubstituted 5 to 10 membered heteroaryl.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted RD (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when RD is substituted, it is substituted with at least one lower substituent group.

In embodiments, R^{1A} is independently hydrogen. In embodiments, R^{1A} is independently unsubstituted C₁-C₄ alkyl. In embodiments, R^{1A} is independently unsubstituted methyl. In embodiments, R^{1A} is independently unsubstituted ethyl. In embodiments, R^{1A} is independently unsubstituted propyl. In embodiments, R^{1A} is independently unsubstituted n-propyl. In embodiments, R^{1A} is independently unsubstituted isopropyl. In embodiments, R^{1A} is independently unsubstituted butyl. In embodiments, R^{1A} is independently unsubstituted n-butyl. In embodiments, R^{1A} is independently unsubstituted isobutyl. In embodiments, R^{1A} is independently unsubstituted tert-butyl.

In embodiments, R^{1B} is independently hydrogen. In embodiments, R^{1B} is independently unsubstituted C₁-C₄ alkyl. In embodiments, R^{1B} is independently unsubstituted methyl. In embodiments, R^{1B} is independently unsubstituted ethyl. In embodiments, R^{1B} is independently unsubstituted propyl. In embodiments, R^{1B} is independently unsubstituted n-propyl. In embodiments, R^{1B} is independently unsubstituted isopropyl. In embodiments, R^{1B} is independently unsubstituted butyl. In embodiments, R^{1B} is independently unsubstituted n-butyl. In embodiments, R^{1B} is independently unsubstituted isobutyl. In embodiments, R^{1B} is independently unsubstituted tert-butyl.

In embodiments, R^{1C} is independently hydrogen. In embodiments, R^{1C} is independently unsubstituted C₁-C₄ alkyl. In embodiments, R^{1C} is independently unsubstituted methyl. In embodiments, R^{1C} is independently unsubstituted ethyl. In embodiments, R^{1C} is independently unsubstituted propyl. In embodiments, R^{1C} is independently unsubstituted n-propyl. In embodiments, R^{1C} is independently unsubstituted isopropyl. In embodiments, R^{1C} is independently unsubstituted butyl. In embodiments, R^{1C} is independently unsubstituted n-butyl. In embodiments, R^{1C} is independently unsubstituted isobutyl. In embodiments, R^{1C} is independently unsubstituted tert-butyl.

In embodiments, R^{1D} is independently hydrogen. In embodiments, R^{1D} is independently unsubstituted C₁-C₄ alkyl. In embodiments, R^{1D} is independently unsubstituted methyl. In embodiments, R^{1D} is independently unsubstituted ethyl. In embodiments, R^{1D} is independently unsubstituted propyl. In embodiments, R^{1D} is independently unsubstituted n-propyl. In embodiments, R^{1D} is independently unsubstituted isopropyl. In embodiments, R^{1D} is independently unsubstituted butyl. In embodiments, R^{1D} is independently unsubstituted n-butyl. In embodiments, R^{1D} is independently unsubstituted isobutyl. In embodiments, R^{1D} is independently unsubstituted tert-butyl.

In embodiments, z1 is 0. In embodiments, z1 is 1. In embodiments, z1 is 2. In embodiments, z1 is 3.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen. In embodiments, R² is halogen. In embodiments, R² is —F. In embodiments, R² is —Cl. In embodiments, R² is —Br. In embodiments, R² is —I. In embodiments, R² is —CCl₃. In embodiments, R² is —CBr₃. In embodiments, R² is —CF₃. In embodiments, R² is —Cl₃. In embodiments, R² is —CH₂Cl. In embodiments, R² is —CH₂Br. In embodiments, R² is —CH₂F. In embodiments, R² is —CH₂I. In embodiments, R² is —CHCl₂. In embodiments, R² is —CHBr₂. In embodiments, R² is —CHF₂. In embodiments, R² is —CHI₂. In embodiments, R² is —CN. In embodiments, R² is —OH. In embodiments, R² is —NH₂. In embodiments, R² is —COOH. In embodiments, R² is —CONH₂. In embodiments, R² is —NO₂. In embodiments, R² is —SH. In embodiments, R² is —SO₃H. In embodiments, R² is —OSO₃H. In embodiments, R² is —SO₂NH₂. In embodiments, R² is —NHNH₂. In embodiments, R² is —ONH₂. In embodiments, R² is —NHC(O)NH₂. In embodiments, R² is —NHSO₂H. In embodiments, R² is —NHC(O)H. In embodiments, R² is —NHC(O)OH. In embodiments, R² is —NHOH. In embodiments, R² is —OCCl₃. In embodiments, R² is —OCBr₃. In embodiments, R² is —OCF₃. In embodiments, R² is —OCI₃. In embodiments, R² is —OCH₂Cl. In embodiments, R² is —OCH₂Br. In embodiments, R² is —OCH₂F. In embodiments, R² is —OCH₂I. In embodiments, R² is —OCHCl₂. In embodiments, R² is —OCHBr₂. In embodiments, R² is —OCHF₂. In embodiments, R² is —OCHI₂. In embodiments, R² is —N₃. In embodiments, R² is substituted or unsubstituted C₁-C₆ alkyl. In embodiments, R² is unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl. In embodiments, R² is unsubstituted 2 to 4 membered heteroalkyl. In embodiments, R² is unsubstituted methoxy. In embodiments, R² is unsubstituted ethoxy. In embodiments, R² is unsubstituted propoxy. In embodiments, R² is unsubstituted n-propoxy. In embodiments, R² is unsubstituted isopropoxy. In embodiments, R² is unsubstituted butoxy. In embodiments, R² is unsubstituted n-butoxy. In embodiments, R² is unsubstituted isobutoxy. In embodiments, R² is unsubstituted tert-butoxy. In embodiments, R² is substituted or unsubstituted 2 to 6 membered heteroalkyl. In embodiments, R² is substituted or unsubstituted C₃-C₈ cycloalkyl. In embodiments, R² is substituted or unsubstituted 3 to 8 membered heterocycloalkyl. In embodiments, R² is substituted or unsubstituted C₆-C₁₀ aryl. In embodiments, R² is substituted or unsubstituted 5 to 10 membered heteroaryl.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R^{2A} is hydrogen. In embodiments, R^{2A} is unsubstituted C₁-C₄ alkyl. In embodiments, R^{2A} is unsubstituted methyl. In embodiments, R^{2A} is unsubstituted ethyl. In embodiments, R^{2A} is unsubstituted propyl. In embodiments, R^{2A} is unsubstituted n-propyl. In embodiments, R^{2A} is unsubstituted isopropyl. In embodiments, R^{2A} is unsubstituted butyl. In embodiments, R^{2A} is unsubstituted n-butyl. In embodiments, R^{2A} is unsubstituted isobutyl. In embodiments, R^{2A} is unsubstituted tert-butyl.

In embodiments, R^{2B} is hydrogen. In embodiments, R^{2B} is unsubstituted C₁-C₄ alkyl. In embodiments, R^{2B} is unsubstituted methyl. In embodiments, R^{2B} is unsubstituted ethyl. In embodiments, R^{2B} is unsubstituted propyl. In embodiments, R^{2B} is unsubstituted n-propyl. In embodiments, R^{2B} is unsubstituted isopropyl. In embodiments, R^{2B} is unsubstituted butyl. In embodiments, R^{2B} is unsubstituted n-butyl. In embodiments, R^{2B} is unsubstituted isobutyl. In embodiments, R^{2B} is unsubstituted tert-butyl.

In embodiments, R^{2C} is hydrogen. In embodiments, R^{2C} is unsubstituted C₁-C₄ alkyl. In embodiments, R^{2C} is unsubstituted methyl. In embodiments, R^{2C} is unsubstituted ethyl. In embodiments, R^{2C} is unsubstituted propyl. In embodiments, R^{2C} is unsubstituted n-propyl. In embodiments, R^{2C} is unsubstituted isopropyl. In embodiments, R^{2C} is unsubstituted butyl. In embodiments, R^{2C} is unsubstituted n-butyl. In embodiments, R^{2C} is unsubstituted isobutyl. In embodiments, R^{2C} is unsubstituted tert-butyl.

In embodiments, R^{2D} is hydrogen. In embodiments, R^{2D} is unsubstituted C₁-C₄ alkyl. In embodiments, R^{2D} is unsubstituted methyl. In embodiments, R^{2D} is unsubstituted ethyl. In embodiments, R^{2D} is unsubstituted propyl. In embodiments, R^{2D} is unsubstituted n-propyl. In embodiments, R^{2D} is unsubstituted isopropyl. In embodiments, R^{2D} is unsubstituted butyl. In embodiments, R^{2D} is unsubstituted n-butyl. In embodiments, R^{2D} is unsubstituted isobutyl. In embodiments, R^{2D} is unsubstituted tert-butyl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is hydrogen or unsubstituted C₁-C₆ alkyl. In embodiments, R³ is hydrogen. In embodiments, R³ is substituted or unsubstituted C₁-C₆ alkyl. In embodiments, R³ is unsubstituted C₁-C₆ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl. In embodiments, R³ is substituted or unsubstituted 2 to 6 membered heteroalkyl. In embodiments, R³ is substituted or unsubstituted C₃-C₈ cycloalkyl. In embodiments, R³ is substituted or unsubstituted 3 to 8 membered heterocycloalkyl. In embodiments, R³ is substituted or unsubstituted C₆-C₁₀ aryl. In embodiments, R³ is substituted or unsubstituted 5 to 10 membered heteroaryl.

In embodiments, a substituted ring formed when R² and R³ substituents are joined (e.g., substituted heterocycloalkyl and/or substituted heteroaryl) is substituted with at least one substituent group, size-limited substituent group, or lower substituent group; wherein if the substituted ring formed when R² and R³ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R² and R³ substituents are joined is substituted, it is substituted with at least one substituent group. In embodiments, when the substituted ring formed when R² and R³ substituents are joined is substituted, it is substituted with at least one size-limited substituent group. In embodiments, when the substituted ring formed when R² and R³ substituents are joined is substituted, it is substituted with at least one lower substituent group.

In embodiments, R² and R³ substituents are joined to form a substituted or unsubstituted 3 to 8 membered heterocycloalkyl or substituted or unsubstituted 5 to 10 membered heteroaryl. In embodiments, R² and R³ substituents are joined to form a substituted or unsubstituted 3 to 8 membered heterocycloalkyl. In embodiments, R² and R³ substituents are joined to form a substituted or unsubstituted 5 to 10 membered heteroaryl. In embodiments, R² and R³ substituents are joined to form an unsubstituted imidazolyl.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen, —NHC(O)NH₂, or unsubstituted C₁-C₄ alkyl.

In embodiments, R⁴ is hydrogen. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CCl₃. In embodiments, R⁴ is —CBr₃. In embodiments, R⁴ is —CF₃. In embodiments, R⁴ is —Cl₃. In embodiments, R⁴ is —CH₂Cl. In embodiments, R⁴ is —CH₂Br. In embodiments, R⁴ is —CH₂F. In embodiments, R⁴ is —CH₂I. In embodiments, R⁴ is —CHCl₂. In embodiments, R⁴ is —CHBr₂. In embodiments, R⁴ is —CHF₂. In embodiments, R⁴ is —CHI₂. In embodiments, R⁴ is —CN. In embodiments, R⁴ is —OH. In embodiments, R⁴ is —NH₂. In embodiments, R⁴ is —COOH. In embodiments, R⁴ is —CONH₂. In embodiments, R⁴ is —NO₂. In embodiments, R⁴ is —SH. In embodiments, R⁴ is —SO₃H. In embodiments, R⁴ is —OSO₃H. In embodiments, R⁴ is —SO₂NH₂. In embodiments, R⁴ is —NHNH₂. In embodiments, R⁴ is —ONH₂. In embodiments, R⁴ is —NHC(O)NH₂. In embodiments, R⁴ is —NHSO₂H. In embodiments, R⁴ is —NHC(O)H. In embodiments, R⁴ is —NHC(O)OH. In embodiments, R⁴ is —NHOH. In embodiments, R⁴ is —OCCl₃. In embodiments, R⁴ is —OCBr₃. In embodiments, R⁴ is —OCF₃. In embodiments, R⁴ is —OCI₃. In embodiments, R⁴ is —OCH₂Cl. In embodiments, R⁴ is —OCH₂Br. In embodiments, R⁴ is —OCH₂F. In embodiments, R⁴ is —OCH₂I. In embodiments, R⁴ is —OCHCl₂. In embodiments, R⁴ is —OCHBr₂. In embodiments, R⁴ is —OCHF₂. In embodiments, R⁴ is —OCHI₂. In embodiments, R⁴ is —N₃. In embodiments, R⁴ is substituted or unsubstituted C₁-C₆ alkyl. In embodiments, R⁴ is unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl. In embodiments, R⁴ is substituted or unsubstituted 2 to 4 membered heteroalkyl. In embodiments, R⁴ is unsubstituted C₂-C₂₀ alkenyl. In embodiments, R⁴ is unsubstituted C₄-C₂₀ alkenyl. In embodiments, R⁴ is unsubstituted C₄-C₈ alkenyl. In embodiments, R⁴ is

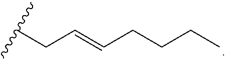 [SMILES: *C/C=C/CCCC]

In embodiments, R⁴ is substituted or unsubstituted C₃-C₈ cycloalkyl. In embodiments, R⁴ is substituted or unsubstituted 3 to 8 membered heterocycloalkyl. In embodiments, R⁴ is substituted or unsubstituted C₅-C₁₀ aryl. In embodiments, R⁴ is substituted or unsubstituted phenyl. In embodiments, R⁴ is substituted or unsubstituted 5 to 10 membered heteroaryl.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R^{4A} is independently hydrogen. In embodiments, R^{4A} is independently unsubstituted C₁-C₄ alkyl. In embodiments, R^{4A} is independently unsubstituted methyl. In embodiments, R^{4A} is independently unsubstituted ethyl. In embodiments, R^{4A} is independently unsubstituted propyl. In embodiments, R^{4A} is independently unsubstituted n-propyl. In embodiments, R^{4A} is independently unsubstituted isopropyl. In embodiments, R^{4A} is independently unsubstituted butyl. In embodiments, R^{4A} is independently unsubstituted n-butyl. In embodiments, R^{4A} is independently unsubstituted isobutyl. In embodiments, R^{4A} is independently unsubstituted tert-butyl.

In embodiments, R^{4B} is independently hydrogen. In embodiments, R^{4B} is independently unsubstituted C₁-C₄ alkyl. In embodiments, R^{4B} is independently unsubstituted methyl. In embodiments, R^{4B} is independently unsubstituted ethyl. In embodiments, R^{4B} is independently unsubstituted propyl. In embodiments, R^{4B} is independently unsubstituted n-propyl. In embodiments, R^{4B} is independently unsubstituted isopropyl. In embodiments, R^{4B} is independently unsubstituted butyl. In embodiments, R^{4B} is independently unsubstituted n-butyl. In embodiments, R^{4B} is independently unsubstituted isobutyl. In embodiments, R^{4B} is independently unsubstituted tert-butyl.

In embodiments, R^{4C} is independently hydrogen. In embodiments, R^{4C} is independently unsubstituted C₁-C₄ alkyl. In embodiments, R^{4C} is independently unsubstituted methyl. In embodiments, R^{4C} is independently unsubstituted ethyl. In embodiments, R^{4C} is independently unsubstituted propyl. In embodiments, R^{4C} is independently unsubstituted n-propyl. In embodiments, R^{4C} is independently unsubstituted isopropyl. In embodiments, R^{4C} is independently unsubstituted butyl. In embodiments, R^{4C} is independently unsubstituted n-butyl. In embodiments, R^{4C} is independently unsubstituted isobutyl. In embodiments, R^{4C} is independently unsubstituted tert-butyl.

In embodiments, R^{4D} is independently hydrogen. In embodiments, R^{4D} is independently unsubstituted C₁-C₄ alkyl. In embodiments, R⁴¹ is independently unsubstituted methyl. In embodiments, R^{4D} is independently unsubstituted ethyl. In embodiments, R^{4D} is independently unsubstituted propyl. In embodiments, R⁴¹ is independently unsubstituted n-propyl. In embodiments, R^{4D} is independently unsubstituted isopropyl. In embodiments, R^{4D} is independently unsubstituted butyl. In embodiments, R⁴¹ is independently unsubstituted n-butyl. In embodiments, R^{4D} is independently unsubstituted isobutyl. In embodiments, R^{4D} is independently unsubstituted tert-butyl.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is hydrogen or unsubstituted C₁-C₆ alkyl. In embodiments, R⁵ is hydrogen. In embodiments, R⁵ is substituted or unsubstituted C₁-C₆ alkyl. In embodiments, R⁵ is unsubstituted C₁-C₆ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl. In embodiments, R⁵ is substituted or unsubstituted 2 to 4 membered heteroalkyl. In embodiments, R⁵ is substituted or unsubstituted C₃-C₅ cycloalkyl. In embodiments, R⁵ is substituted or unsubstituted 3 to 8 membered heterocycloalkyl. In embodiments, R⁵ is substituted or unsubstituted C₅-C₁₀ aryl. In embodiments, R⁵ is substituted or unsubstituted phenyl. In embodiments, R⁵ is substituted or unsubstituted 5 to 10 membered heteroaryl.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is hydrogen. In embodiments, R⁶ is halogen. In embodiments, R⁶ is —F. In embodiments, R⁶ is —Cl. In embodiments, R⁶ is —Br. In embodiments, R⁶ is —I. In embodiments, R⁶ is —CCl₃. In embodiments, R⁶ is —CBr₃. In embodiments, R⁶ is —CF₃. In embodiments, R⁶ is —Cl₃. In embodiments, R⁶ is —CH₆Cl. In embodiments, R⁶ is —CH₂Br. In embodiments, R⁶ is —CH₂F. In embodiments, R⁶ is —CH₂I. In embodiments, R⁶ is —CHCl₂. In embodiments, R⁶ is —CHBr₂. In embodiments, R⁶ is —CHF₂. In embodiments, R⁶ is —CHI₂. In embodiments, R⁶ is —CN. In embodiments, R⁶ is —OH. In embodiments, R⁶ is —NH₂. In embodiments, R⁶ is —COOH. In embodiments, R⁶ is —CONH₂. In embodiments, R⁶ is —NO₂. In embodiments, R⁶ is —SH. In embodiments, R⁶ is —SO₃H. In embodiments, R⁶ is —OSO₃H. In embodiments, R⁶ is —SO₂NH₂. In embodiments, R⁶ is —NHNH₂. In embodiments, R⁶ is —ONH₂. In embodiments, R⁶ is —NHC(O)NH₂. In embodiments, R⁶ is —NHSO₂H. In embodiments, R⁶ is —NHC(O)H. In embodiments, R⁶ is —NHC(O)OH. In embodiments, R⁶ is —NHOH. In embodiments, R⁶ is —OCCl₃. In embodiments, R⁶ is —OCBr₃. In embodiments, R⁶ is —OCF₃. In embodiments, R⁶ is —OCI₃. In embodiments, R⁶ is —OCH₂Cl. In embodiments, R⁶ is —OCH₂Br. In embodiments, R⁶ is —OCH₂F. In embodiments, R⁶ is —OCH₂I. In embodiments, R⁶ is —OCHCl₂. In embodiments, R⁶ is —OCHBr₂. In embodiments, R⁶ is —OCHF₂. In embodiments, R⁶ is —OCHI₂. In embodiments, R⁶ is —N₃. In embodiments, R⁶ is substituted or unsubstituted C₁-C₆ alkyl. In embodiments, R⁶ is unsubstituted C₁-C₄ alkyl. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl. In embodiments, R⁶ is unsubstituted 2 to 4 membered heteroalkyl. In embodiments, R⁶ is unsubstituted methoxy. In embodiments, R⁶ is unsubstituted ethoxy. In embodiments, R⁶ is unsubstituted propoxy. In embodiments, R⁶ is unsubstituted n-propoxy. In embodiments, R⁶ is unsubstituted isopropoxy. In embodiments, R⁶ is unsubstituted butoxy. In embodiments, R⁶ is unsubstituted n-butoxy. In embodiments, R⁶ is unsubstituted isobutoxy. In embodiments, R⁶ is unsubstituted tert-butoxy. In embodiments, R⁶ is substituted or unsubstituted 2 to 4 membered heteroalkyl. In embodiments, R⁶ is substituted or unsubstituted C₃-C₈ cycloalkyl. In embodiments, R⁶ is substituted or unsubstituted 3 to 8 membered heterocycloalkyl. In embodiments, R⁶ is substituted or unsubstituted C₅-C₁₀ aryl. In embodiments, R⁶ is substituted or unsubstituted phenyl. In embodiments, R⁶ is substituted or unsubstituted 5 to 10 membered heteroaryl.

In embodiments, a substituted R^{6A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group.

In embodiments, a substituted R^{6B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6B} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, a substituted ring formed when R^{6A} and R^{6B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{6A} and R^{6B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{6A} and R^{6B} substituents bonded to the same nitrogen atom are joined is substituted, it is substituted with at least one substituent group. In embodiments, when the substituted ring formed when R^{6A} and R^{6B} substituents bonded to the same nitrogen atom are joined is substituted, it is substituted with at least one size-limited substituent group. In embodiments, when the substituted ring formed when R^{6A} and R^{6B} substituents bonded to the same nitrogen atom are joined is substituted, it is substituted with at least one lower substituent group.

In embodiments, a substituted R^{6C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6C} is substituted with a plurality of groups selected from substituent groups, size-limited substituent groups, and lower substituent groups; each substituent group, size-limited substituent group, and/or lower substituent group may optionally be different. In embodiments, when R^{6C} is substituted, it is substituted with at least one substituent group. In embodiments, when R^{6C} is substituted, it is substituted with at least one size-limited substituent group. In embodiments, when R^{6C} is substituted, it is substituted with at least one lower substituent group.

In embodiments, a substituted R^{6D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6D} is substituted with a plurality of groups selected from substituent groups, size-limited substituent groups, and lower substituent groups; each substituent group, size-limited substituent group, and/or lower substituent group may optionally be different. In embodiments, when R^{6D} is substituted, it is substituted with at least one substituent group. In embodiments, when R^{6D} is substituted, it is substituted with at least one size-limited substituent group. In embodiments, when R^{6D} is substituted, it is substituted with at least one lower substituent group.

In embodiments, R^{6A} is hydrogen. In embodiments, R^{6A} is unsubstituted C₁-C₄ alkyl. In embodiments, R^{6A} is unsubstituted methyl. In embodiments, R^{6A} is unsubstituted ethyl. In embodiments, R^{6A} is unsubstituted propyl. In embodiments, R^{6A} is unsubstituted n-propyl. In embodiments, R^{6A} is unsubstituted isopropyl. In embodiments, R^{6A} is unsubstituted butyl. In embodiments, R^{6A} is unsubstituted n-butyl. In embodiments, R^{6A} is unsubstituted isobutyl. In embodiments, R^{6A} is unsubstituted tert-butyl.

In embodiments, R^{6B} is hydrogen. In embodiments, R^{6B} is unsubstituted C₁-C₄ alkyl. In embodiments, R^{6B} is unsubstituted methyl. In embodiments, R^{6B} is unsubstituted ethyl. In embodiments, R^{6B} is unsubstituted propyl. In embodiments, R^{6B} is unsubstituted n-propyl. In embodiments, R^{6B} is unsubstituted isopropyl. In embodiments, R^{6B} is unsubstituted butyl. In embodiments, R^{6B} is unsubstituted n-butyl. In embodiments, R^{6B} is unsubstituted isobutyl. In embodiments, R^{6B} is unsubstituted tert-butyl.

In embodiments, R^{6C} is hydrogen. In embodiments, R^{6C} is unsubstituted C₁-C₄ alkyl. In embodiments, R^{6C} is unsubstituted methyl. In embodiments, R^{6C} is unsubstituted ethyl. In embodiments, R^{6C} is unsubstituted propyl. In embodiments, R^{6C} is unsubstituted n-propyl. In embodiments, R^{6C} is unsubstituted isopropyl. In embodiments, R^{6C} is unsubstituted butyl. In embodiments, R^{6C} is unsubstituted n-butyl. In embodiments, R^{6C} is unsubstituted isobutyl. In embodiments, R^{6C} is unsubstituted tert-butyl.

In embodiments, R^{6D} is hydrogen. In embodiments, R^{6D} is unsubstituted C₁-C₄ alkyl. In embodiments, R^{6D} is unsubstituted methyl. In embodiments, R^{6D} is unsubstituted ethyl. In embodiments, R^{6D} is unsubstituted propyl. In embodiments, R^{6D} is unsubstituted n-propyl. In embodiments, R^{6D} is unsubstituted isopropyl. In embodiments, R^{6D} is unsubstituted butyl. In embodiments, R^{6D} is unsubstituted n-butyl. In embodiments, R^{6D} is unsubstituted isobutyl. In embodiments, R^{6D} is unsubstituted tert-butyl.

In embodiments, a substituted ring formed when R⁵ and R⁶ substituents are joined (e.g., substituted heterocycloalkyl and/or substituted heteroaryl) is substituted with at least one substituent group, size-limited substituent group, or lower substituent group; wherein if the substituted ring formed when R⁵ and R⁶ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R⁵ and R⁶ substituents are joined is substituted, it is substituted with at least one substituent group. In embodiments, when the substituted ring formed when R⁵ and R⁶ substituents are joined is substituted, it is substituted with at least one size-limited substituent group. In embodiments, when the substituted ring formed when R⁵ and R⁶ substituents are joined is substituted, it is substituted with at least one lower substituent group.

In embodiments, R⁵ and R⁶ substituents are joined to form a substituted or unsubstituted 3 to 8 membered heterocycloalkyl or substituted or unsubstituted 5 to 10 membered heteroaryl. In embodiments, R⁵ and R⁶ substituents are joined to form a substituted or unsubstituted 3 to 8 membered heterocycloalkyl. In embodiments, R⁵ and R⁶ substituents are joined to form a substituted or unsubstituted 5 to 10 membered heteroaryl. In embodiments, R⁵ and R⁶ substituents are joined to form an unsubstituted imidazolyl.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is independently halogen. In embodiments, R⁷ is independently —F. In embodiments, R⁷ is independently —Cl. In embodiments, R⁷ is independently —Br. In embodiments, R⁷ is independently —I. In embodiments, R⁷ is independently —CCl₃. In embodiments, R⁷ is independently —CBr₃. In embodiments, R⁷ is independently —CF₃. In embodiments, R⁷ is independently —Cl₃. In embodiments, R⁷ is independently —CH₂Cl. In embodiments, R⁷ is independently —CH₂Br. In embodiments, R⁷ is independently —CH₂F. In embodiments, R⁷ is independently —CH₂I. In embodiments, R⁷ is independently —CHCl₂. In embodiments, R⁷ is independently —CHBr₂. In embodiments, R⁷ is independently —CHF₂. In embodiments, R⁷ is independently —CHI₂. In embodiments, R⁷ is independently —CN. In embodiments, R⁷ is independently —OH. In embodiments, R⁷ is independently —NH₂. In embodiments, R⁷ is independently —COOH. In embodiments, R⁷ is independently —CONH₂. In embodiments, R⁷ is independently —NO₂. In embodiments, R⁷ is independently —SH. In embodiments, R⁷ is independently —SO₃H. In embodiments, R⁷ is independently —OSO₃H. In embodiments, R⁷ is independently —SO₂NH₂. In embodiments, R⁷ is independently —NHNH₂. In embodiments, R⁷ is independently —ONH₂. In embodiments, R⁷ is independently —NHC(O)NH₂. In embodiments, R⁷ is independently —NHSO₂H. In embodiments, R⁷ is independently —NHC(O)H. In embodiments, R⁷ is independently —NHC(O)OH. In embodiments, R′ is independently —NHOH. In embodiments, R⁷ is independently —OCCl₃. In embodiments, R⁷ is independently —OCBr₃. In embodiments, R⁷ is independently —OCF₃. In embodiments, R⁷ is independently —OCI₃. In embodiments, R⁷ is independently —OCH₂Cl. In embodiments, R⁷ is independently —OCH₂Br. In embodiments, R⁷ is independently —OCH₂F. In embodiments, R⁷ is independently —OCH₂I. In embodiments, R⁷ is independently —OCHCl₂. In embodiments, R⁷ is independently —OCHBr₂. In embodiments, R⁷ is independently —OCHF₂. In embodiments, R⁷ is independently —OCHI₂. In embodiments, R⁷ is independently —N₃. In embodiments, R⁷ is independently substituted or unsubstituted C₁-C₆ alkyl. In embodiments, R⁷ is independently unsubstituted C₁-C₄ alkyl. In embodiments, R⁷ is independently unsubstituted methyl. In embodiments, R⁷ is independently unsubstituted ethyl. In embodiments, R⁷ is independently unsubstituted propyl. In embodiments, R⁷ is independently unsubstituted n-propyl. In embodiments, R⁷ is independently unsubstituted isopropyl. In embodiments, R⁷ is independently unsubstituted butyl. In embodiments, R⁷ is independently unsubstituted n-butyl. In embodiments, R⁷ is independently unsubstituted isobutyl. In embodiments, R⁷ is independently unsubstituted tert-butyl. In embodiments, R⁷ is independently unsubstituted 2 to 4 membered heteroalkyl. In embodiments, R⁷ is independently unsubstituted methoxy. In embodiments, R⁷ is independently unsubstituted ethoxy. In embodiments, R⁷ is independently unsubstituted propoxy. In embodiments, R⁷ is independently unsubstituted n-propoxy. In embodiments, R⁷ is independently unsubstituted isopropoxy. In embodiments, R⁷ is independently unsubstituted butoxy. In embodiments, R⁷ is independently unsubstituted n-butoxy. In embodiments, R⁷ is independently unsubstituted isobutoxy. In embodiments, R⁷ is independently unsubstituted tert-butoxy. In embodiments, R⁷ is independently substituted or unsubstituted 2 to 6 membered heteroalkyl. In embodiments, R⁷ is independently substituted or unsubstituted C₃-C₈ cycloalkyl. In embodiments, R⁷ is independently substituted or unsubstituted 3 to 8 membered heterocycloalkyl. In embodiments, R⁷ is independently substituted or unsubstituted C₆-C₁₀ aryl. In embodiments, R⁷ is independently substituted or unsubstituted 5 to 10 membered heteroaryl.

In embodiments, a substituted R^{7A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different. In embodiments, when R^{7A} is substituted, it is substituted with at least one substituent group. In embodiments, when R^{7A} is substituted, it is substituted with at least one size-limited substituent group. In embodiments, when R^{7A} is substituted, it is substituted with at least one lower substituent group.

In embodiments, a substituted R^{7B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7B} is substituted with a plurality of groups selected from substituent groups, size-limited substituent groups, and lower substituent groups; each substituent group, size-limited substituent group, and/or lower substituent group may optionally be different. In embodiments, when R^{7B} is substituted, it is substituted with at least one substituent group. In embodiments, when R^{7B} is substituted, it is substituted with at least one size-limited substituent group. In embodiments, when R^{7B} is substituted, it is substituted with at least one lower substituent group.

In embodiments, a substituted ring formed when R^{7A} and R^{7B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{7A} and R^{7B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{7A} and R^{7B} substituents bonded to the same nitrogen atom are joined is substituted, it is substituted with at least one substituent group. In embodiments, when the substituted ring formed when R^{7A} and R^{7B} substituents bonded to the same nitrogen atom are joined is substituted, it is substituted with at least one size-limited substituent group. In embodiments, when the substituted ring formed when R^{7A} and R^{7B} substituents bonded to the same nitrogen atom are joined is substituted, it is substituted with at least one lower substituent group.

In embodiments, a substituted R^{7C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7C} is substituted with a plurality of groups selected from substituent groups, size-limited substituent groups, and lower substituent groups; each substituent group, size-limited substituent group, and/or lower substituent group may optionally be different. In embodiments, when R^{7C} is substituted, it is substituted with at least one substituent group. In embodiments, when R^{7C} is substituted, it is substituted with at least one size-limited substituent group. In embodiments, when R^{7C} is substituted, it is substituted with at least one lower substituent group.

In embodiments, a substituted R^{7D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7D} is substituted with a plurality of groups selected from substituent groups, size-limited substituent groups, and lower substituent groups; each substituent group, size-limited substituent group, and/or lower substituent group may optionally be different. In embodiments, when R^{7D} is substituted, it is substituted with at least one substituent group. In embodiments, when R^{7D} is substituted, it is substituted with at least one size-limited substituent group. In embodiments, when R^{7D} is substituted, it is substituted with at least one lower substituent group.

In embodiments, R^{7A} is independently hydrogen. In embodiments, R^{7A} is independently unsubstituted C₁-C₄ alkyl. In embodiments, R^{7A} is independently unsubstituted methyl. In embodiments, R^{7A} is independently unsubstituted ethyl. In embodiments, R^{7A} is independently unsubstituted propyl. In embodiments, R^{7A} is independently unsubstituted n-propyl. In embodiments, R^{7A} is independently unsubstituted isopropyl. In embodiments, R^{7A} is independently unsubstituted butyl. In embodiments, R^{7A} is independently unsubstituted n-butyl. In embodiments, R^{7A} is independently unsubstituted isobutyl. In embodiments, R^{7A} is independently unsubstituted tert-butyl.

In embodiments, R^{7B} is independently hydrogen. In embodiments, R^{7B} is independently unsubstituted C₁-C₄ alkyl. In embodiments, R^{7B} is independently unsubstituted methyl. In embodiments, R^{7B} is independently unsubstituted ethyl. In embodiments, R^{7B} is independently unsubstituted propyl. In embodiments, R^{7B} is independently unsubstituted n-propyl. In embodiments, R^{7B} is independently unsubstituted isopropyl. In embodiments, R^{7B} is independently unsubstituted butyl. In embodiments, R^{7B} is independently unsubstituted n-butyl. In embodiments, R^{7B} is independently unsubstituted isobutyl. In embodiments, R^{7B} is independently unsubstituted tert-butyl.

In embodiments, R^{7C} is independently hydrogen. In embodiments, R^{7C} is independently unsubstituted C₁-C₄ alkyl. In embodiments, R^{7C} is independently unsubstituted methyl. In embodiments, R^{7C} is independently unsubstituted ethyl. In embodiments, R^{7C} is independently unsubstituted propyl. In embodiments, R^{7C} is independently unsubstituted n-propyl. In embodiments, R^{7C} is independently unsubstituted isopropyl. In embodiments, R^{7C} is independently unsubstituted butyl. In embodiments, R^{7C} is independently unsubstituted n-butyl. In embodiments, R^{7C} is independently unsubstituted isobutyl. In embodiments, R^{7C} is independently unsubstituted tert-butyl.

In embodiments, R^{7D} is independently hydrogen. In embodiments, R^{7D} is independently unsubstituted C₁-C₄ alkyl. In embodiments, R^{7D} is independently unsubstituted methyl. In embodiments, R^{7D} is independently unsubstituted ethyl. In embodiments, R^{7D} is independently unsubstituted propyl. In embodiments, R^{7D} is independently unsubstituted n-propyl. In embodiments, R^{7D} is independently unsubstituted isopropyl. In embodiments, R^{7D} is independently unsubstituted butyl. In embodiments, R^{7D} is independently unsubstituted n-butyl. In embodiments, R^{7D} is independently unsubstituted isobutyl. In embodiments, R^{7D} is independently unsubstituted tert-butyl.

In embodiments, z7 is 0. In embodiments, z7 is 1. In embodiments, z7 is 2. In embodiments, z7 is 3.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, R¹⁰ is independently hydrogen or unsubstituted C₁-C₆ alkyl. In embodiments, R¹⁰ is independently hydrogen. In embodiments, R¹⁰ is independently substituted or unsubstituted C₁-C₆ alkyl. In embodiments, R¹⁰ is independently unsubstituted C₁-C₆alkyl. In embodiments, R¹⁰ is independently unsubstituted methyl. In embodiments, R¹⁰ is independently unsubstituted ethyl. In embodiments, R¹⁰ is independently unsubstituted propyl. In embodiments, R¹⁰ is independently unsubstituted n-propyl. In embodiments, R¹⁰ is independently unsubstituted isopropyl. In embodiments, R¹⁰ is independently unsubstituted butyl. In embodiments, R¹⁰ is independently unsubstituted n-butyl. In embodiments, R¹⁰ is independently unsubstituted isobutyl. In embodiments, R¹⁰ is independently unsubstituted tert-butyl. In embodiments, R¹⁰ is independently substituted or unsubstituted 2 to 6 membered heteroalkyl. In embodiments, R¹⁰ is independently substituted or unsubstituted C₃-C₅ cycloalkyl. In embodiments, R¹⁰ is independently substituted or unsubstituted 3 to 8 membered heterocycloalkyl. In embodiments, R¹⁰ is independently substituted or unsubstituted C₆-C₁₀ aryl. In embodiments, R¹⁰ is independently substituted or unsubstituted 5 to 10 membered heteroaryl.

In embodiments, a substituted R²⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰ is substituted with a plurality of groups selected from substituent groups, size-limited substituent groups, and lower substituent groups; each substituent group, size-limited substituent group, and/or lower substituent group may optionally be different. In embodiments, when R²⁰ is substituted, it is substituted with at least one substituent group. In embodiments, when R²⁰ is substituted, it is substituted with at least one size-limited substituent group. In embodiments, when R²⁰ is substituted, it is substituted with at least one lower substituent group.

In embodiments, R²⁰ is independently hydrogen or unsubstituted C₁-C₆ alkyl. In embodiments, R²⁰ is independently hydrogen. In embodiments, R²⁰ is independently substituted or unsubstituted C₁-C₆ alkyl. In embodiments, R²⁰ is independently unsubstituted C₁-C₆alkyl. In embodiments, R²⁰ is independently unsubstituted methyl. In embodiments, R²⁰ is independently unsubstituted ethyl. In embodiments, R²⁰ is independently unsubstituted propyl. In embodiments, R²⁰ is independently unsubstituted n-propyl. In embodiments, R²⁰ is independently unsubstituted isopropyl. In embodiments, R²⁰ is independently unsubstituted butyl. In embodiments, R²⁰ is independently unsubstituted n-butyl. In embodiments, R²⁰ is independently unsubstituted isobutyl. In embodiments, R²⁰ is independently unsubstituted tert-butyl. In embodiments, R²⁰ is independently substituted or unsubstituted 2 to 6 membered heteroalkyl. In embodiments, R²⁰ is independently substituted or unsubstituted C₃-C₅ cycloalkyl. In embodiments, R²⁰ is independently substituted or unsubstituted 3 to 8 membered heterocycloalkyl. In embodiments, R²⁰ is independently substituted or unsubstituted C₆-C₁₀ aryl. In embodiments, R²⁰ is independently substituted or unsubstituted 5 to 10 membered heteroaryl.

In embodiments, a substituted R³⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³⁰ is substituted with a plurality of groups selected from substituent groups, size-limited substituent groups, and lower substituent groups; each substituent group, size-limited substituent group, and/or lower substituent group may optionally be different. In embodiments, when R³⁰ is substituted, it is substituted with at least one substituent group. In embodiments, when R³⁰ is substituted, it is substituted with at least one size-limited substituent group. In embodiments, when R³⁰ is substituted, it is substituted with at least one lower substituent group.

In embodiments, R³⁰ is independently hydrogen or unsubstituted C₁-C₆ alkyl. In embodiments, R³⁰ is independently hydrogen. In embodiments, R³⁰ is independently substituted or unsubstituted C₁-C₆ alkyl. In embodiments, R³⁰ is independently unsubstituted C₁-C₆ alkyl. In embodiments, R³⁰ is independently unsubstituted methyl. In embodiments, R³⁰ is independently unsubstituted ethyl. In embodiments, R³⁰ is independently unsubstituted propyl. In embodiments, R³⁰ is independently unsubstituted n-propyl. In embodiments, R³⁰ is independently unsubstituted isopropyl. In embodiments, R³⁰ is independently unsubstituted butyl. In embodiments, R³⁰ is independently unsubstituted n-butyl. In embodiments, R³⁰ is independently unsubstituted isobutyl. In embodiments, R³⁰ is independently unsubstituted tert-butyl. In embodiments, R³⁰ is independently substituted or unsubstituted 2 to 6 membered heteroalkyl. In embodiments, R³⁰ is independently substituted or unsubstituted C₃-C₈ cycloalkyl. In embodiments, R³⁰ is independently substituted or unsubstituted 3 to 8 membered heterocycloalkyl. In embodiments, R³⁰ is independently substituted or unsubstituted C₆-C₁₀ aryl. In embodiments, R³⁰ is independently substituted or unsubstituted 5 to 10 membered heteroaryl.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2} and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2} and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{23.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R² and R³ substituents are optionally joined to form a moiety that is substituted (e.g., a substituted heterocycloalkyl or substituted heteroaryl), the moiety is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R^{2.1}, R^{2.2}, and R^{2.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2.1}, R^{2.2}, and R^{2.3}, respectively.

In embodiments, when R² and R³ substituents are optionally joined to form a moiety that is substituted (e.g., a substituted heterocycloalkyl or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R^{3.1}, R^{3.2}, and R^{3.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3.1}, R^{3.2}, and R^{3.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2} and R^{4A.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4D}, R^{4D.1}, R^{4D.2}, and R^{4D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when R^{6A} is substituted, R^{6A} is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A}, R^{6A.1}, R^{6A.2}, and R^{6A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6A}, R^{6A.1}, R^{6A.2}, and R^{6A.3}, respectively.

In embodiments, when R^{6B} is substituted, R^{6B} is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B}, R^{6B.1}, R^{6B.2}, and R^{6B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6B}, R^{6B.1}, R^{6B.2}, and R^{6B.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A.1}, R^{6A.2}, and R^{6A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6A.1}, R^{6A.2} and R^{6A.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B.1}, R^{6B.2}, and R^{6B.3} have values corresponding to the values of R^{WW}, R^{WW.2}, and R^{WW.3}, respectively, as explained in the definitions section above in the description of “first substituent group(s)”, wherein R^{WW.1}, R^{WW.2}, and R^{WW.3} correspond to R^{6B.1}, R^{6B.2} and R^{6B.3}, respectively.

In embodiments, when R^{6C} is substituted, R^{6C} is substituted with one or more first substituent groups denoted by R^{6C.1} as explained in the definitions section above in the description of “first substituent group(s)”. In embodiments, when an R^{6C.1} substituent group is substituted, the R^{6C.1} substituent group is substituted with one or more second substituent groups denoted by R^{6C.2} as explained in the definitions section above in the description of “first substituent group(s)”. In embodiments, when an R^{6C.2} substituent group is substituted, the R^{6C.2} substituent group is substituted with one or more third substituent groups denoted by R^{6C.3} as explained in the definitions section above in the description of “first substituent group(s)”. In the above embodiments, R^{6C}, R^{6C.1}, R^{6C.2}, and R^{6C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6C}, R^{6C.1}, R^{6C.2}, and R^{6C.3}, respectively.

In embodiments, when R^{6D} is substituted, R^{6D} is substituted with one or more first substituent groups denoted by R^{6D.1} as explained in the definitions section above in the description of “first substituent group(s)”. In embodiments, when an R^{6D.1} substituent group is substituted, the R^{6D.1} substituent group is substituted with one or more second substituent groups denoted by R^{6D.2} as explained in the definitions section above in the description of “first substituent group(s)”. In embodiments, when an R^{6D.2} substituent group is substituted, the R^{6D.2} substituent group is substituted with one or more third substituent groups denoted by R^{6D.3} as explained in the definitions section above in the description of “first substituent group(s)”. In the above embodiments, R^{6D}, R^{6D.1}, R^{6D.2}, and R^{6D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6D}, R^{6D.1}, R^{6D.2}, and R^{6D.3}, respectively.

In embodiments, when R⁵ and R⁶ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R² as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R^{5.1}, R^{5.2}, and R^{5.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5.1}, R^{5.2}, and R^{5.3}, respectively.

In embodiments, when R⁵ and R⁶ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R^{6.1}, R^{6.2}, and R^{6.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6.1}, R^{6.2}, and R⁶³, respectively.

In embodiments, when R⁷ is substituted, R⁷ is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁷, R^{7.1}, R^{7.2}, and R^{7.3}, respectively.

In embodiments, when R^{7A} is substituted, R^{7A} is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A}, R^{7A.1}, R^{7A.2}, and R^{7A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7A}, R^{7A.1}, R^{7A.2}, and R^{7A.3}, respectively.

In embodiments, when R^{7B} is substituted, R^{7B} is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B}, R^{7B.1}, R^{7B.2}, and R^{7B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7B}, R^{7B.1}, R^{7B.2}, and R^{7B.3}, respectively.

In embodiments, when R^{7A} and R^{7B} substituents bonded to the same nitrogen atom are optionally joined to form a moiety that is substituted (e.g., a substituted heterocycloalkyl or substituted heteroaryl), the moiety is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A.1}, R^{7A.2}, and R^{7A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7A.1}, R^{7A.2} and R^{7A.3}, respectively.

In embodiments, when R^{7A} and R^{7B} substituents bonded to the same nitrogen atom are optionally joined to form a moiety that is substituted (e.g., a substituted heterocycloalkyl or substituted heteroaryl), the moiety is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B.1}, R^{7B.2}, and R^{7B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7B.1}, R^{7B.2} and R^{7B.3}, respectively.

In embodiments, when R^{7C} is substituted, R^{7C} is substituted with one or more first substituent groups denoted by R^{7C.1} as explained in the definitions section above in the description of “first substituent group(s)”. In embodiments, when an R^{7C.1} substituent group is substituted, the R^{7C.1} substituent group is substituted with one or more second substituent groups denoted by R^{7C.2} as explained in the definitions section above in the description of “first substituent group(s)”. In embodiments, when an R^{7C.2} substituent group is substituted, the R^{7C.2} substituent group is substituted with one or more third substituent groups denoted by R^{7C.3} as explained in the definitions section above in the description of “first substituent group(s)”. In the above embodiments, R^{7C}, R^{7C.1}, R^{7C.2}, and R^{7C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7C}, R^{7C.1}, R^{7C.2}, and R^{7C.3}, respectively.

In embodiments, when R^{7D} is substituted, R^{7D} is substituted with one or more first substituent groups denoted by R^{7D.1} as explained in the definitions section above in the description of “first substituent group(s)”. In embodiments, when an R^{7D.1} substituent group is substituted, the R^{7D.1} substituent group is substituted with one or more second substituent groups denoted by R^{7D.2} as explained in the definitions section above in the description of “first substituent group(s)”. In embodiments, when an R^{7D.2} substituent group is substituted, the R^{7D.2} substituent group is substituted with one or more third substituent groups denoted by R^{7D.3} as explained in the definitions section above in the description of “first substituent group(s)”. In the above embodiments, R^{7D}, R^{7D.1}, R^{7D.2}, and R^{7D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7D}, R^{7D.1}, R^{7D.2}, and R^{7D.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R¹⁰¹, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰, R^{10.1}, R^{10.2}, and R^{10.3}, respectively.

In embodiments, when R²⁰ is substituted, R²⁰ is substituted with one or more first substituent groups denoted by R^{20.1} as explained in the definitions section above in the description of “first substituent group(s)”. In embodiments, when an R^{20.1} substituent group is substituted, the R^{20.1} substituent group is substituted with one or more second substituent groups denoted by R^{20.2} as explained in the definitions section above in the description of “first substituent group(s)”. In embodiments, when an R^{20.2} substituent group is substituted, the R^{20.2} substituent group is substituted with one or more third substituent groups denoted by R^{20.3} as explained in the definitions section above in the description of “first substituent group(s)”. In the above embodiments, R²⁰, R^{20.1}, R^{20.2}, and R^{20.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁰, R^{20.1}, R^{20.2}, and R^{20.3}, respectively.

In embodiments, when R³⁰ is substituted, R³⁰ is substituted with one or more first substituent groups denoted by R^{30.1} as explained in the definitions section above in the description of “first substituent group(s)”. In embodiments, when an R^{30.1} substituent group is substituted, the R^{30.1} substituent group is substituted with one or more second substituent groups denoted by R^{30.2} as explained in the definitions section above in the description of “first substituent group(s)”. In embodiments, when an R^{30.2} substituent group is substituted, the R^{30.2} substituent group is substituted with one or more third substituent groups denoted by R^{30.3} as explained in the definitions section above in the description of “first substituent group(s)”. In the above embodiments, R³⁰, R³⁰¹, R^{30.2}, and R³⁰³ have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³⁰, R^{30.1}, R^{30.2}, and R^{30.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3}, respectively.

In embodiments, when L² is substituted, L² is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L2, R^{L2.1}, R^{L2.2}, and R^{L2.3}, respectively.

In embodiments, when L³ is substituted, L³ is substituted with one or more first substituent groups denoted by R^{L3.1} as explained in the definitions section above in the description of “first substituent group(s)”. In embodiments, when an R^{L3.1} substituent group is substituted, the R^{L3.1} substituent group is substituted with one or more second substituent groups denoted by R^{L3.2} as explained in the definitions section above in the description of “first substituent group(s)”. In embodiments, when an R^{L3.2} substituent group is substituted, the R^{L3.2} substituent group is substituted with one or more third substituent groups denoted by R^{L3.3} as explained in the definitions section above in the description of “first substituent group(s)”. In the above embodiments, L³, R^{L3.1}, R^{L3.2}, and R^{L3.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L³, R^{L3.1}, R^{L3.2}, and R^{L3.3}, respectively.

In embodiments, the compound has the formula:

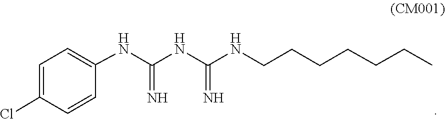 [SMILES: CCCCCCCNC(=N)NC(=N)Nc1ccc(Cl)cc1]

In embodiments, the compound has the formula:

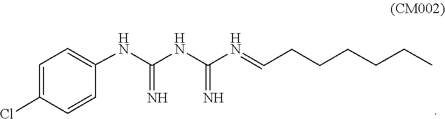 [SMILES: CCCCCC/C=N/C(=N)NC(=N)Nc1ccc(Cl)cc1]

In embodiments, the compound has the formula:

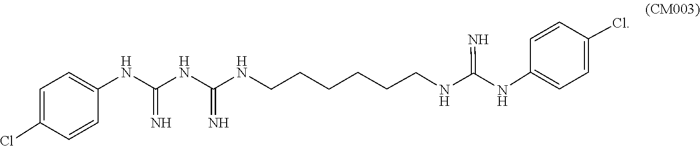 [SMILES: N=C(NCCCCCCNC(=N)Nc1ccc(Cl)cc1)NC(=N)Nc1ccc(Cl)cc1]

In embodiments, the compound has the formula:

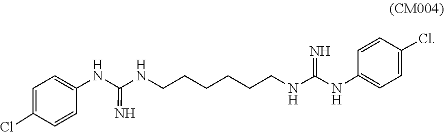 [SMILES: N=C(NCCCCCCNC(=N)Nc1ccc(Cl)cc1)Nc1ccc(Cl)cc1]

In embodiments, the compound has the formula:

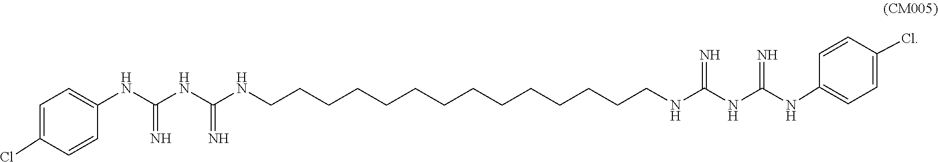 [SMILES: N=C(NCCCCCCCCCCCCCCNC(=N)NC(=N)Nc1ccc(Cl)cc1)NC(=N)Nc1ccc(Cl)cc1]

In embodiments, the compound has the formula:

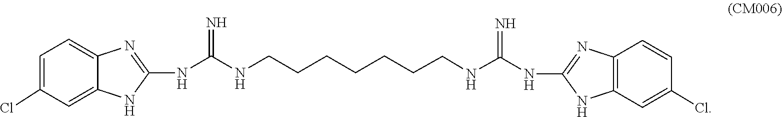 [SMILES: N=C(NCCCCCCCNC(=N)Nc1nc2ccc(Cl)cc2[nH]1)Nc1nc2ccc(Cl)cc2[nH]1]

In embodiments, the compound has the formula:

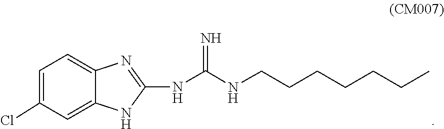 [SMILES: CCCCCCCNC(=N)Nc1nc2ccc(Cl)cc2[nH]1]

In embodiments, the compound has the formula:

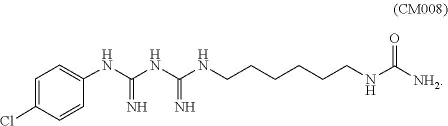 [SMILES: N=C(NCCCCCCNC(N)=O)NC(=N)Nc1ccc(Cl)cc1]

In embodiments, the compound contacts an amino acid corresponding to E116, Q261, F262, E270, G345, Q112, or P260 of a CLOCK (e.g., human CLOCK) protein. In embodiments, the compound contacts an amino acid corresponding to E116 of a CLOCK (e.g., human CLOCK) protein. In embodiments, the compound contacts an amino acid corresponding to Q261 of a CLOCK (e.g., human CLOCK) protein. In embodiments, the compound contacts an amino acid corresponding to F262 of a CLOCK (e.g., human CLOCK) protein. In embodiments, the compound contacts an amino acid corresponding to E270 of a CLOCK (e.g., human CLOCK) protein. In embodiments, the compound contacts an amino acid corresponding to G345 of a CLOCK (e.g., human CLOCK) protein. In embodiments, the compound contacts an amino acid corresponding to Q112 of a CLOCK (e.g., human CLOCK) protein. In embodiments, the compound contacts an amino acid corresponding to F262 of a CLOCK (e.g., human CLOCK) protein. In embodiments, the compound contacts an amino acid corresponding to P260 of a CLOCK (e.g., human CLOCK) protein.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound. In embodiments, the compound is a compound of formula (I), (II), (IIIa), (IIIb), (IVa), (IVb), (Va), (Vb), (Vc), (VIa), (VIb), or (VIc), including embodiments thereof.

### IV. Methods of Use

In an aspect is provided a method of increasing myogenesis in a subject in need thereof, the method including administering to the subject a therapeutically effective amount of a clock activator, or a pharmaceutically acceptable salt thereof. In embodiments, the clock activator is a compound as described herein, including in embodiments.

In embodiments, myogenesis is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the clock activator). In embodiments, myogenesis is increased by about 1.5-fold relative to a control (e.g., absence of the clock activator). In embodiments, myogenesis is increased by about 2-fold relative to a control (e.g., absence of the clock activator). In embodiments, myogenesis is increased by about 5-fold relative to a control (e.g., absence of the clock activator). In embodiments, myogenesis is increased by about 10-fold relative to a control (e.g., absence of the clock activator). In embodiments, myogenesis is increased by about 25-fold relative to a control (e.g., absence of the clock activator). In embodiments, myogenesis is increased by about 50-fold relative to a control (e.g., absence of the clock activator). In embodiments, myogenesis is increased by about 100-fold relative to a control (e.g., absence of the clock activator). In embodiments, myogenesis is increased by about 250-fold relative to a control (e.g., absence of the clock activator). In embodiments, myogenesis is increased by about 500-fold relative to a control (e.g., absence of the clock activator). In embodiments, myogenesis is increased by about 1000-fold relative to a control (e.g., absence of the clock activator).

In embodiments, myogenesis is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the clock activator). In embodiments, myogenesis is increased by at least 1.5-fold relative to a control (e.g., absence of the clock activator). In embodiments, myogenesis is increased by at least 2-fold relative to a control (e.g., absence of the clock activator). In embodiments, myogenesis is increased by at least 5-fold relative to a control (e.g., absence of the clock activator). In embodiments, myogenesis is increased by at least 10-fold relative to a control (e.g., absence of the clock activator). In embodiments, myogenesis is increased by at least 25-fold relative to a control (e.g., absence of the clock activator). In embodiments, myogenesis is increased by at least 50-fold relative to a control (e.g., absence of the clock activator). In embodiments, myogenesis is increased by at least 100-fold relative to a control (e.g., absence of the clock activator). In embodiments, myogenesis is increased by at least 250-fold relative to a control (e.g., absence of the clock activator). In embodiments, myogenesis is increased by at least 500-fold relative to a control (e.g., absence of the clock activator). In embodiments, myogenesis is increased by at least 1000-fold relative to a control (e.g., absence of the clock activator).

In an aspect is provided a method of treating a muscle degenerative disease in a subject in need thereof, the method including administering to the subject a therapeutically effective amount of a clock activator described herein, or a pharmaceutically acceptable salt thereof. In embodiments, the clock activator is a compound as described herein, including in embodiments.

In embodiments, the muscle degenerative disease is muscular dystrophy, aging-induced sarcopenia, or cachexia. In embodiments, the muscle degenerative disease is muscular dystrophy. In embodiments, the muscular dystrophy is Duchene muscular dystrophy or Becker muscular dystrophy. In embodiments, the muscular dystrophy is Duchene muscular dystrophy. In embodiments, the muscular dystrophy is Becker muscular dystrophy. In embodiments, the muscle degenerative disease is cardiomyopathy. In embodiments, the cardiomyopathy is caused by muscular dystrophy.

In embodiments, the muscle degenerative disease is aging-induced sarcopenia. In embodiments, the aging-induced sarcopenia includes loss of muscle mass due to advanced age. In embodiments, the aging-induced sarcopenia is in combination with other concomitant chronic conditions (e.g., obesity or diabetes). In embodiments, the muscle degenerative disease is aging-associated sarcopenic obesity.

In embodiments, the muscle degenerative disease is cachexia. In embodiments, the cachexia is cancer-associated cachexia. In embodiments, the cancer-associated cachexia is associated with pancreatic cancer, gastric cancer, liver cancer, lung cancer, breast cancer, esophageal cancer, colorectal cancer, or head and neck cancer. In embodiments, the cachexia is chronic inflammatory disease-associated cachexia. In embodiments, the chronic inflammatory disease-associated cachexia is associated with chronic heart failure or chronic obstructive pulmonary disease. In embodiments, the cachexia is associated with chronic kidney disease. In embodiments, the cachexia is associated with acquired immunodeficiency syndrome (AIDS).

In an aspect is provided a method of treating a muscle injury in a subject in need thereof, the method including administering to the subject a therapeutically effective amount of a clock activator, or a pharmaceutically acceptable salt thereof. In embodiments, the clock activator is a compound as described herein, including in embodiments.

In embodiments, the muscle injury is a muscle tear. In embodiments, the muscle injury is a muscle strain. In embodiments, the muscle injury is a cardiac muscle injury. In embodiments, the cardiac muscle injury is caused by a heart attack. In embodiments, the muscle injury is a chemical toxin-induced injury. In embodiments, the muscle injury is a physical trauma. In embodiments, the muscle injury is a wound-associated loss of muscle tissue. In embodiments, the muscle injury is an injury caused by tissue freezing. In embodiments, the muscle injury is an injury caused by tissue burning.

In an aspect is provided a method of reducing adipogenesis in a subject in need thereof, the method including administering to the subject a therapeutically effective amount of a clock activator, or a pharmaceutically acceptable salt thereof. In embodiments, the clock activator is a compound as described herein, including in embodiments.

In embodiments, adipogenesis is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the clock activator). In embodiments, adipogenesis is reduced by about 1.5-fold relative to a control (e.g., absence of the clock activator). In embodiments, adipogenesis is reduced by about 2-fold relative to a control (e.g., absence of the clock activator). In embodiments, adipogenesis is reduced by about 5-fold relative to a control (e.g., absence of the clock activator). In embodiments, adipogenesis is reduced by about 10-fold relative to a control (e.g., absence of the clock activator). In embodiments, adipogenesis is reduced by about 25-fold relative to a control (e.g., absence of the clock activator). In embodiments, adipogenesis is reduced by about 50-fold relative to a control (e.g., absence of the clock activator). In embodiments, adipogenesis is reduced by about 100-fold relative to a control (e.g., absence of the clock activator). In embodiments, adipogenesis is reduced by about 250-fold relative to a control (e.g., absence of the clock activator). In embodiments, adipogenesis is reduced by about 500-fold relative to a control (e.g., absence of the clock activator). In embodiments, adipogenesis is reduced by about 1000-fold relative to a control (e.g., absence of the clock activator).

In embodiments, adipogenesis is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the clock activator). In embodiments, adipogenesis is reduced by at least 1.5-fold relative to a control (e.g., absence of the clock activator). In embodiments, adipogenesis is reduced by at least 2-fold relative to a control (e.g., absence of the clock activator). In embodiments, adipogenesis is reduced by at least 5-fold relative to a control (e.g., absence of the clock activator). In embodiments, adipogenesis is reduced by at least 10-fold relative to a control (e.g., absence of the clock activator). In embodiments, adipogenesis is reduced by at least 25-fold relative to a control (e.g., absence of the clock activator). In embodiments, adipogenesis is reduced by at least 50-fold relative to a control (e.g., absence of the clock activator). In embodiments, adipogenesis is reduced by at least 100-fold relative to a control (e.g., absence of the clock activator). In embodiments, adipogenesis is reduced by at least 250-fold relative to a control (e.g., absence of the clock activator). In embodiments, adipogenesis is reduced by at least 500-fold relative to a control (e.g., absence of the clock activator). In embodiments, adipogenesis is reduced by at least 1000-fold relative to a control (e.g., absence of the clock activator).

In an aspect is provided a method of treating a metabolic disorder in a subject in need thereof, the method including administering to the subject a therapeutically effective amount of a clock activator described herein, or a pharmaceutically acceptable salt thereof. In embodiments, the clock activator is a compound as described herein, including in embodiments.

In embodiments, the metabolic disorder is obesity. In embodiments, the metabolic disorder is sarcopenic obesity. In embodiments, the metabolic disorder is diabetes. In embodiments, the metabolic disorder is type 2 diabetes. In embodiments, the clock activator inhibits food consumption and increases energy expenditure relative to a control (e.g., absence of the clock activator). In embodiments, the clock activator crosses the blood-brain barrier. In embodiments, the clock activator controls appetite, food intake, physical activity, and/or oxygen consumption.

In embodiments, the clock activator (e.g., compound described herein) is capable of contacting at least one amino acid residue forming a palmitoylation site of a CLOCK protein, wherein the at least one amino acid residue is selected from E116, Q261, F262, E270, G345, Q112, and P260. In embodiments, the at least one amino acid residue is selected from E116, Q261, F262, E270, G345, and Q112. In embodiments, the clock activator (e.g., compound described herein) is capable of contacting E116 of the CLOCK protein. In embodiments, the clock activator (e.g., compound described herein) is capable of contacting Q261 of the CLOCK protein. In embodiments, the clock activator (e.g., compound described herein) is capable of contacting F262 of the CLOCK protein. In embodiments, the clock activator (e.g., compound described herein) is capable of contacting E270 of the CLOCK protein. In embodiments, the clock activator (e.g., compound described herein) is capable of contacting G345 of the CLOCK protein. In embodiments, the clock activator (e.g., compound described herein) is capable of contacting Q112 of the CLOCK protein. In embodiments, the clock activator (e.g., compound described herein) is capable of contacting P260 of the CLOCK protein.

In an aspect is provided a method of activating a CLOCK protein, the method including contacting the CLOCK protein with a clock activator, or a pharmaceutically acceptable salt thereof, which contacts at least one amino acid residue forming a palmitoylation site of the CLOCK protein, wherein the at least one amino acid residue is selected from E116, Q261, F262, E270, G345, Q112, F262, and P260. In embodiments, the clock activator is a compound as described herein, including in embodiments.

In embodiments, the clock activator is chlorhexidine. In embodiments, the clock activator has the formula:

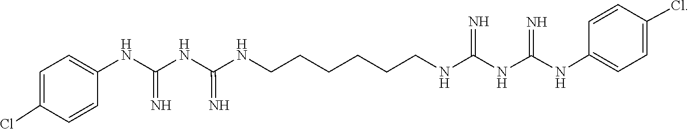 [SMILES: N=C(NCCCCCCNC(=N)NC(=N)Nc1ccc(Cl)cc1)NC(=N)Nc1ccc(Cl)cc1]

### V. Embodiments

Embodiment P1. A method of increasing myogenesis in a subject in need thereof, said method comprising administering a therapeutically effective amount of a clock activator, or a pharmaceutically acceptable salt thereof.

Embodiment P2. A method of treating a muscle degenerative disease in a subject in need thereof, said method comprising administering a therapeutically effective amount of a clock activator, or a pharmaceutically acceptable salt thereof.

Embodiment P3. The method of embodiment P2, wherein the muscle degenerative disease is muscular dystrophy, aging-induced sarcopenia, or cachexia.

Embodiment P4. The method of embodiment P3, wherein the muscular dystrophy is Duchene muscular dystrophy or Becker muscular dystrophy.

Embodiment P5. The method of embodiment P3, wherein the cachexia is chronic inflammatory disease-associated cachexia.

Embodiment P6. A method of reducing adipogenesis in a subject in need thereof, said method comprising administering a therapeutically effective amount of a clock activator, or a pharmaceutically acceptable salt thereof.

Embodiment P7. A method of treating a metabolic disorder in a subject in need thereof, said method comprising administering a therapeutically effective amount of a clock activator, or a pharmaceutically acceptable salt thereof.

Embodiment P8. The method of embodiment P7, wherein the metabolic disorder is obesity.

Embodiment P9. The method of embodiment P7, wherein the metabolic disorder is sarcopenic obesity.

Embodiment P10. The method of embodiment P7, wherein the metabolic disorder is type 2 diabetes.

Embodiment P11. The method of one of embodiments P1 to P10, wherein the clock activator is capable of contacting at least one amino acid residue forming a palmitoylation site of a CLOCK protein, wherein said at least one amino acid residue is selected from E116, Q261, F262, E270, G345, Q112, and P260.

Embodiment P12. The method of embodiment P11, wherein said at least one amino acid residue is selected from E116, Q261, F262, E270, G345, and Q112.

Embodiment P13. A method of activating a CLOCK protein, said method comprising contacting said CLOCK protein with a clock activator, or a pharmaceutically acceptable salt thereof, which contacts at least one amino acid residue forming a palmitoylation site of said CLOCK protein, wherein said at least one amino acid residue is selected from E116, Q261, F262, E270, G345, Q112, F262, and P260.

Embodiment P14. The method of embodiment P13, wherein said at least one amino acid residue is selected from E116, Q261, F262, E270, G345, and Q112.

Embodiment P15. The method of one of embodiments P1 to P14, wherein the clock activator has the formula:

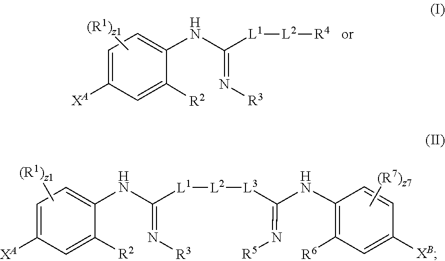

wherein
X^{A} and X^{B} are independently —Cl, —Br, —I, or —F;
L¹ is a bond, —C(O)—, —C(O)O—, —OC(O)—, —O—, —S—, —NR¹⁰—, —C(O)NR¹⁰—, —NR¹⁰C(O)—, —NR¹⁰C(O)O—, —OC(O)NR¹⁰—, —NR¹⁰C(O)NR¹⁰—, —NR¹⁰C(NH)NR¹⁰—, —S(O)₂—, —NR¹⁰S(O)₂—, —S(O)₂NR¹⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —C(O)—, —C(O)O—, —OC(O)—, —O—, —S—, —NR²⁰—, —C(O)NR²⁰—, —NR²⁰C(O)—, —NR²⁰C(O)O—, —OC(O)NR²⁰—, —NR²⁰C(O)NR²⁰—, —NR²⁰C(NH)NR²⁰—, —S(O)₂—, —NR²⁰S(O)₂—, —S(O)₂NR²⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —NR³⁰C(NH)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted heterocycloalkyl or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SO₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SO₆NR^{6A}R^{6B}, —NR^{6C}NR^{6A}R^{6B}, —ONR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OC(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ and R⁶ substituents may optionally be joined to form a substituted or unsubstituted heterocycloalkyl or substituted or unsubstituted heteroaryl;
R⁷ is independently halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NR^{7C}C(O)NR^{7A}R^{7B}, —N(O)ₘ₇, —NR^{7A}R^{7B}, —C(O)R^{7C}, —C(O)OR^{7C}, —OC(O)R^{7C}, —OC(O)OR^{7C}, —C(O)NR^{7A}R^{7B}, —OC(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰, R²⁰, and R³⁰ are independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{4A}, R^{4B}, R^{4C}, R^{4D}, R^{6A}, R^{6B}, R^{6C}, R^{6D}, R^{7A}, R^{7B}, R^{7C}, and R^{7D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl;
z1 is an integer from 0 to 3;
z7 is an integer from 0 to 3;
each X¹, X², X⁴, X⁶, and X⁷ are independently —Cl, —Br, —I, or —F;
n1, n2, n4, n6, and n7 are independently an integer from 0 to 4; and
m1, m2, m4, m6, m7, v1, v2, v4, v6, and v7 are independently 1 or 2.

Embodiment P16. The method of embodiment P15, wherein L¹ is a bond, —NH—, —NHC(NH)NH—, or substituted or unsubstituted 2 to 10 membered heteroalkylene.

Embodiment P17. The method of one of embodiments P15 to P16, wherein L³ is a bond, —NH—, —NHC(NH)NH—, or substituted or unsubstituted 2 to 10 membered heteroalkylene.

Embodiment P18. The method of embodiment P15, wherein the clock activator has the formula:

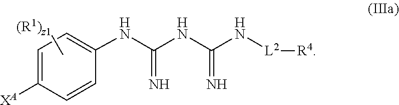

Embodiment P19. The method of embodiment P15, wherein the clock activator has the formula:

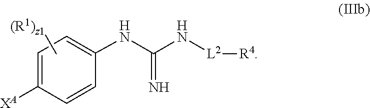

Embodiment P20. The method of embodiment P15, wherein the clock activator has the formula:

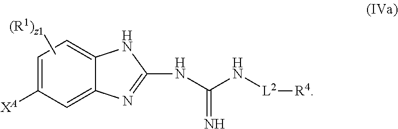

Embodiment P21. The method of embodiment P15, wherein the clock activator has the formula:

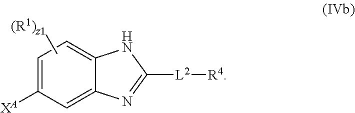

Embodiment P22. The method of embodiment P15, wherein the clock activator has the formula:

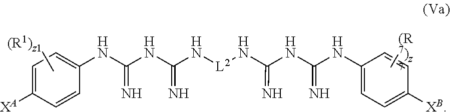

Embodiment P23. The method of embodiment P15, wherein the clock activator has the formula:

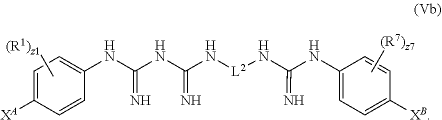

Embodiment P24. The method of embodiment P15, wherein the clock activator has the formula:

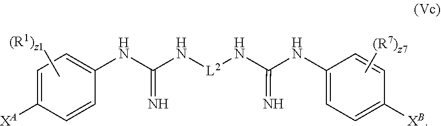

Embodiment P25. The method of embodiment P15, wherein the clock activator has the formula:

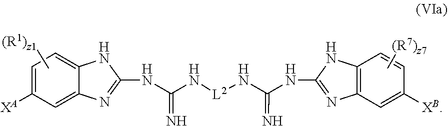

Embodiment P26. The method of embodiment P15, wherein the clock activator has the formula:

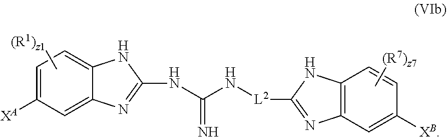

Embodiment P27. The method of embodiment P15, wherein the clock activator has the formula:

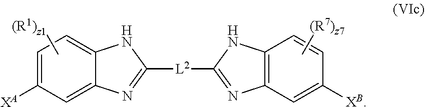

Embodiment P28. The method of one of embodiments P15 to P27, wherein X^{A} is —Cl.

Embodiment P29. The method of one of embodiments P15 to P17 and P22 to P28, wherein X^{B} is —Cl.

Embodiment P30. The method of one of embodiments P15 to P29, wherein R¹ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P31. The method of one of embodiments P15 to P29, wherein z1 is 0.

Embodiment P32. The method of one of embodiments P15 to P17 and P22 to P31, wherein R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P33. The method of one of embodiments P15 to P17 and P22 to P31, wherein z7 is 0.

Embodiment P34. The method of one of embodiments P15 to P33, wherein L² is unsubstituted C₂-C₂₀ alkylene.

Embodiment P35. The method of one of embodiments P15 to P33, wherein L² is unsubstituted C₂-C₂₀ alkenylene.

Embodiment P36. The method of one of embodiments P15 to P35, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P37. The method of one of embodiments P15 to P35, wherein R⁴ is hydrogen, —NHC(O)NH₂, or unsubstituted C₁-C₄ alkylene.

Embodiment P38. The method of one of embodiments P1 to P14, wherein the clock activator is chlorhexidine.

Embodiment P39. The method of one of embodiments P1 to P14, wherein the clock activator has the formula:

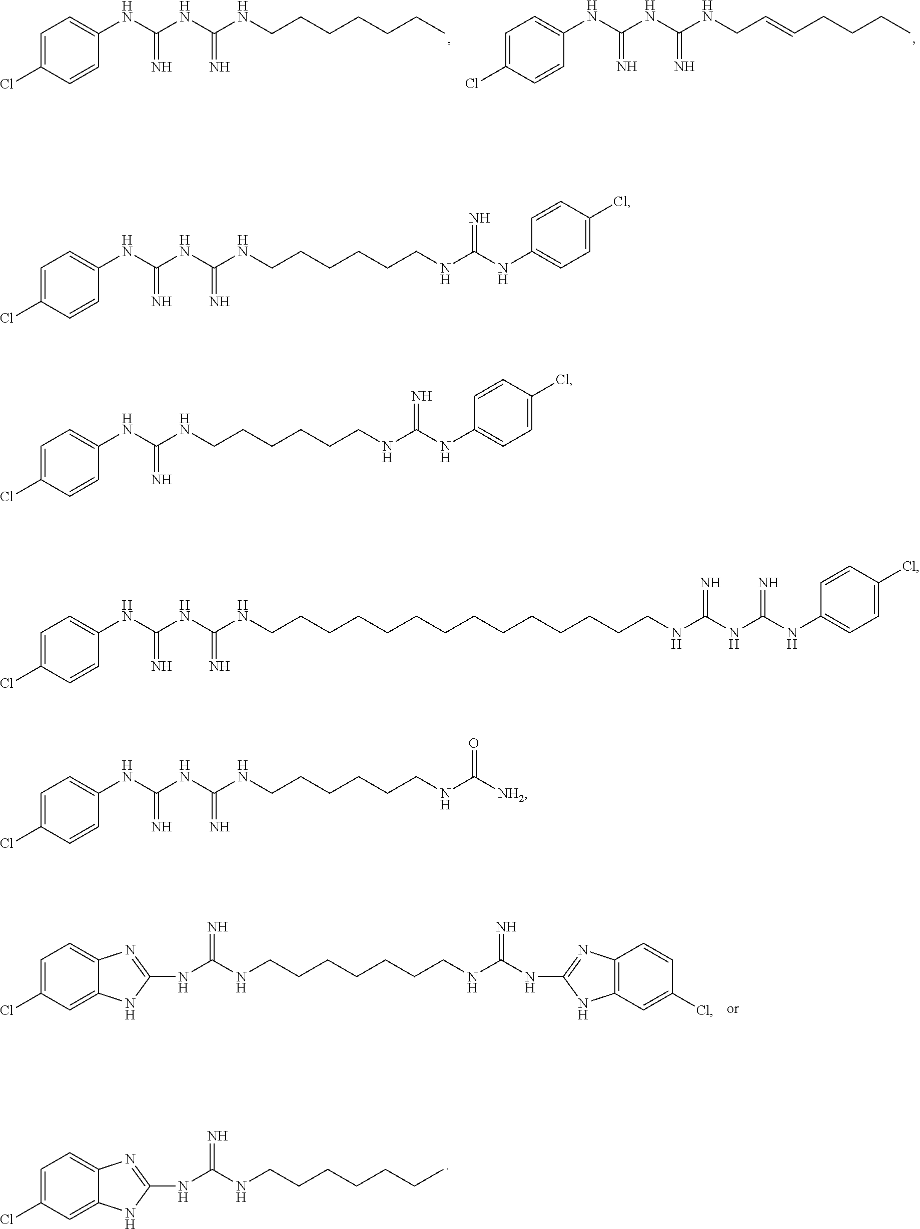

Embodiment P40. A compound, having the formula:

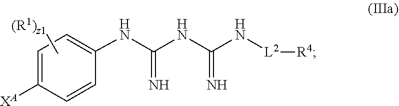

wherein
X^{A} is —Cl, —Br, —I, or —F;
L² is a bond;
R⁴ is unsubstituted C₄-C₂₀ alkenyl;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
z1 is an integer from 0 to 3;
each X¹ is independently —Cl, —Br, —I, or —F;
n1 is independently an integer from 0 to 4; and
m1 and v1 are independently 1 or 2.

Embodiment P41. The compound of embodiment P40, wherein X^{A} is —Cl.

Embodiment P42. The compound of embodiment P40 or embodiment P41, wherein R⁴ is unsubstituted C₄-C₈ alkenyl.

Embodiment P43. The compound of embodiment P40 or embodiment P41, wherein R⁴ is

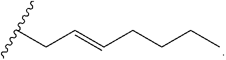 [SMILES: *C/C=C/CCCC]

Embodiment P44. The compound of one of embodiments P40 to P43, wherein R¹ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P45. The compound of one of embodiments P40 to P43, wherein z1 is 0.

Embodiment P46. The compound of embodiment P40, having the formula:

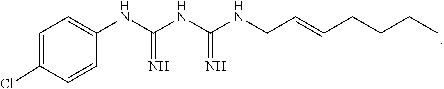 [SMILES: CCCC/C=C/CNC(=N)NC(=N)Nc1ccc(Cl)cc1]

Embodiment P47. A pharmaceutical composition comprising a pharmaceutically acceptable excipient and a compound, or a pharmaceutically acceptable salt thereof, having the formula:

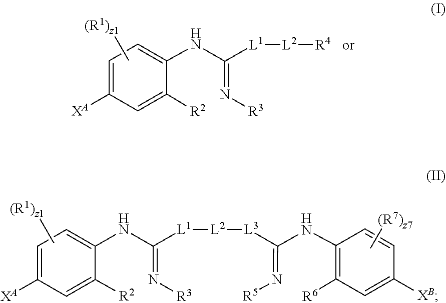

wherein
X^{A} and X^{B} are independently —Cl, —Br, —I, or —F;
L¹ is a bond, —C(O)—, —C(O)O—, —OC(O)—, —O—, —S—, —NR¹⁰—, —C(O)NR¹⁰—, —NR¹⁰C(O)—, —NR¹⁰C(O)O—, —OC(O)NR¹⁰—, —NR¹⁰C(O)NR¹⁰—, —NR¹⁰C(NH)NR¹⁰—, —S(O)₂—, —NR¹⁰S(O)₂—, —S(O)₂NR¹⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —C(O)—, —C(O)O—, —OC(O)—, —O—, —S—, —NR²⁰—, —C(O)NR²⁰—, —NR²⁰C(O)—, —NR²⁰C(O)O—, —OC(O)NR²⁰—, —NR²⁰C(O)NR²⁰—, —NR²⁰C(NH)NR²⁰—, —S(O)₂—, —NR²⁰S(O)₂—, —S(O)₂NR²⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —NR³⁰C(NH)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted heterocycloalkyl or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SO₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OC(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ and R⁶ substituents may optionally be joined to form a substituted or unsubstituted heterocycloalkyl or substituted or unsubstituted heteroaryl;
R⁷ is independently halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NR^{7C}C(O)NR^{7A}R^{7B}, —N(O)ₘ₇, —NR^{7A}R^{7B}, —C(O)R^{7C}, —C(O)OR^{7C}, —OC(O)R^{7C}, —OC(O)OR^{7C}, —C(O)NR^{7A}R^{7B}, —OC(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰, R²⁰, and R³⁰ are independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{4A}, R^{4B}, R^{4C}, R^{4D}, R^{6A}, R^{6B}, R^{6C}, R^{6D}, R^{7A}, R^{7B}, R^{7C}, and R^{7D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl;
z1 is an integer from 0 to 3;
z7 is an integer from 0 to 3;
each X¹, X², X⁴, X⁶, and X⁷ are independently —Cl, —Br, —I, or —F;
n1, n2, n4, n6, and n7 are independently an integer from 0 to 4; and
m1, m2, m4, m6, m7, v1, v2, v4, v6, and v7 are independently 1 or 2.

### VI. Additional Embodiments

Embodiment 1. A method of increasing myogenesis in a subject in need thereof, said method comprising administering a therapeutically effective amount of a clock activator, or a pharmaceutically acceptable salt thereof.

Embodiment 2. A method of treating a muscle degenerative disease in a subject in need thereof, said method comprising administering a therapeutically effective amount of a clock activator, or a pharmaceutically acceptable salt thereof.

Embodiment 3. The method of embodiment 2, wherein the muscle degenerative disease is muscular dystrophy, aging-induced sarcopenia, or cachexia.

Embodiment 4. The method of embodiment 3, wherein the muscular dystrophy is Duchene muscular dystrophy or Becker muscular dystrophy.

Embodiment 5. The method of embodiment 3, wherein the cachexia is chronic inflammatory disease-associated cachexia.

Embodiment 6. The method of embodiment 2, wherein the muscle degenerative disease is cardiomyopathy.

Embodiment 7. The method of embodiment 6, wherein the cardiomyopathy is caused by muscular dystrophy.

Embodiment 8. A method of treating a muscle injury in a subject in need thereof, said method comprising administering a therapeutically effective amount of a clock activator, or a pharmaceutically acceptable salt thereof.

Embodiment 9. The method of embodiment 8, wherein the muscle injury is a muscle tear.

Embodiment 10. The method of embodiment 8, wherein the muscle injury is a cardiac muscle injury.

Embodiment 11. The method of embodiment 10, wherein the cardiac muscle injury is caused by a heart attack.

Embodiment 12. A method of reducing adipogenesis in a subject in need thereof, said method comprising administering a therapeutically effective amount of a clock activator, or a pharmaceutically acceptable salt thereof.

Embodiment 13. A method of treating a metabolic disorder in a subject in need thereof, said method comprising administering a therapeutically effective amount of a clock activator, or a pharmaceutically acceptable salt thereof.

Embodiment 14. The method of embodiment 13, wherein the metabolic disorder is obesity.

Embodiment 15. The method of embodiment 13, wherein the metabolic disorder is sarcopenic obesity.

Embodiment 16. The method of embodiment 13, wherein the metabolic disorder is type 2 diabetes.

Embodiment 17. The method of one of embodiments 1 to 16, wherein the clock activator is capable of contacting at least one amino acid residue forming a palmitoylation site of a CLOCK protein, wherein said at least one amino acid residue is selected from E116, Q261, F262, E270, G345, Q112, and P260.

Embodiment 18. The method of embodiment 17, wherein said at least one amino acid residue is selected from E116, Q261, F262, E270, G345, and Q112.

Embodiment 19. A method of activating a CLOCK protein, said method comprising contacting said CLOCK protein with a clock activator, or a pharmaceutically acceptable salt thereof, which contacts at least one amino acid residue forming a palmitoylation site of said CLOCK protein, wherein said at least one amino acid residue is selected from E116, Q261, F262, E270, G345, Q112, F262, and P260.

Embodiment 20. The method of embodiment 19, wherein said at least one amino acid residue is selected from E116, Q261, F262, E270, G345, and Q112.

Embodiment 21. The method of one of embodiments 1 to 20, wherein the clock activator has the formula:

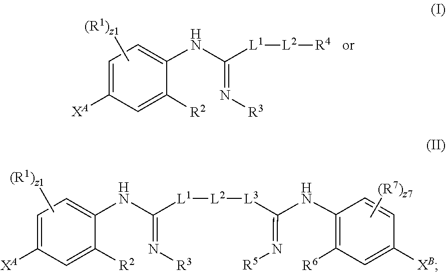

wherein
X^{A} and X^{B} are independently —Cl, —Br, —I, or —F;
L¹ is a bond, —C(O)—, —C(O)O—, —OC(O)—, —O—, —S—, —NR¹⁰—, —C(O)NR¹⁰—, —NR¹⁰C(O)—, —NR¹⁰C(O)O—, —OC(O)NR¹⁰—, —NR¹⁰C(O)NR¹⁰—, —NR¹⁰C(NH)NR¹⁰—, —S(O)₂—, —NR¹⁰S(O)₂—, —S(O)₂NR¹⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —C(O)—, —C(O)O—, —OC(O)—, —O—, —S—, —NR²⁰—, —C(O)NR²⁰—, —NR²⁰C(O)—, —NR²⁰C(O)O—, —OC(O)NR²⁰—, —NR²⁰C(O)NR²⁰—, —NR²⁰C(NH)NR²⁰—, —S(O)₂—, —NR²⁰S(O)₂—, —S(O)₂NR²⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —NR³⁰C(NH)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted heterocycloalkyl or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SO₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SO₆NR^{6A}R^{6B}, —NR^{6C}NR^{6A}R^{6B}, —ONR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OC(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ and R⁶ substituents may optionally be joined to form a substituted or unsubstituted heterocycloalkyl or substituted or unsubstituted heteroaryl;
R⁷ is independently halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NR^{7C}C(O)NR^{7A}R^{7B}, —N(O)ₘ₇, —NR^{7A}R^{7B}, —C(O)R^{7C}, —C(O)OR^{7C}, —OC(O)R^{7C}, —OC(O)OR^{7C}, —C(O)NR^{7A}R^{7B}, —OC(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰, R²⁰, and R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{4A}, R^{4B}, R^{4C}, R^{4D}, R^{6A}, R^{6B}, R^{6C}, R^{6D}, R^{7A}, R^{7B}, R^{7C}, and R^{7D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl;
z1 is an integer from 0 to 3;
z7 is an integer from 0 to 3;
each X¹, X², X⁴, X⁶, and X⁷ is independently —Cl, —Br, —I, or —F;
n1, n2, n4, n6, and n7 are independently an integer from 0 to 4; and
m1, m2, m4, m6, m7, v1, v2, v4, v6, and v7 are independently 1 or 2.

Embodiment 22. The method of embodiment 21, wherein L¹ is a bond, —NH—, —NHC(NH)NH—, or substituted or unsubstituted 2 to 10 membered heteroalkylene.

Embodiment 23. The method of one of embodiments 21 to 22, wherein L³ is a bond, —NH—, —NHC(NH)NH—, or substituted or unsubstituted 2 to 10 membered heteroalkylene.

Embodiment 24. The method of embodiment 21, wherein the clock activator has the formula:

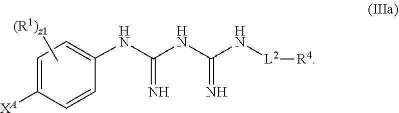

Embodiment 25. The method of embodiment 21, wherein the clock activator has the formula:

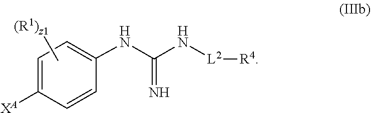

Embodiment 26. The method of embodiment 21, wherein the clock activator has the formula:

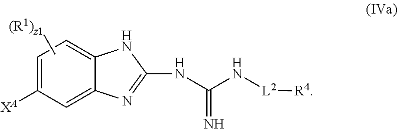

Embodiment 27. The method of embodiment 21, wherein the clock activator has the formula:

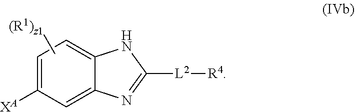

Embodiment 28. The method of embodiment 21, wherein the clock activator has the formula:

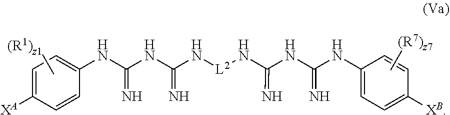

Embodiment 29. The method of embodiment 21, wherein the clock activator has the formula:

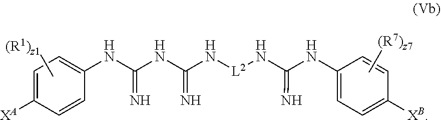

Embodiment 30. The method of embodiment 21, wherein the clock activator has the formula:

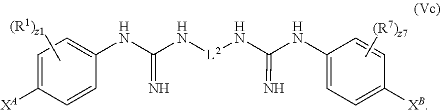

Embodiment 31. The method of embodiment 21, wherein the clock activator has the formula:

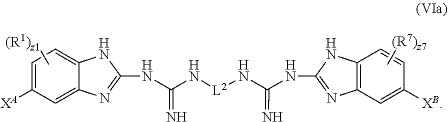

Embodiment 32. The method of embodiment 21, wherein the clock activator has the formula:

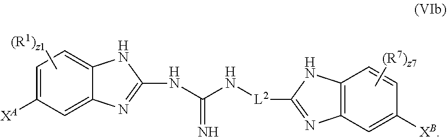

Embodiment 33. The method of embodiment 21, wherein the clock activator has the formula:

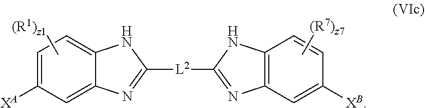

Embodiment 34. The method of one of embodiments 21 to 33, wherein X^{A} is —Cl.

Embodiment 35. The method of one of embodiments 21 to 23 and 28 to 34, wherein X^{B} is —Cl.

Embodiment 36. The method of one of embodiments 21 to 35, wherein R¹ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 37. The method of one of embodiments 21 to 35, wherein z1 is 0.

Embodiment 38. The method of one of embodiments 21 to 23 and 28 to 37, wherein R⁷ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 39. The method of one of embodiments 21 to 23 and 28 to 37, wherein z7 is 0.

Embodiment 40. The method of one of embodiments 21 to 39, wherein L² is unsubstituted C₂-C₂₀ alkylene.

Embodiment 41. The method of one of embodiments 21 to 39, wherein L² is unsubstituted C₂-C₂₀ alkenylene.

Embodiment 42. The method of one of embodiments 21 to 41, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 43. The method of one of embodiments 21 to 41, wherein R⁴ is hydrogen, —NHC(O)NH₂, or unsubstituted C₁-C₄ alkylene.

Embodiment 44. The method of one of embodiments 1 to 20, wherein the clock activator is chlorhexidine.

Embodiment 45. The method of one of embodiments 1 to 20, wherein the clock activator has the formula:

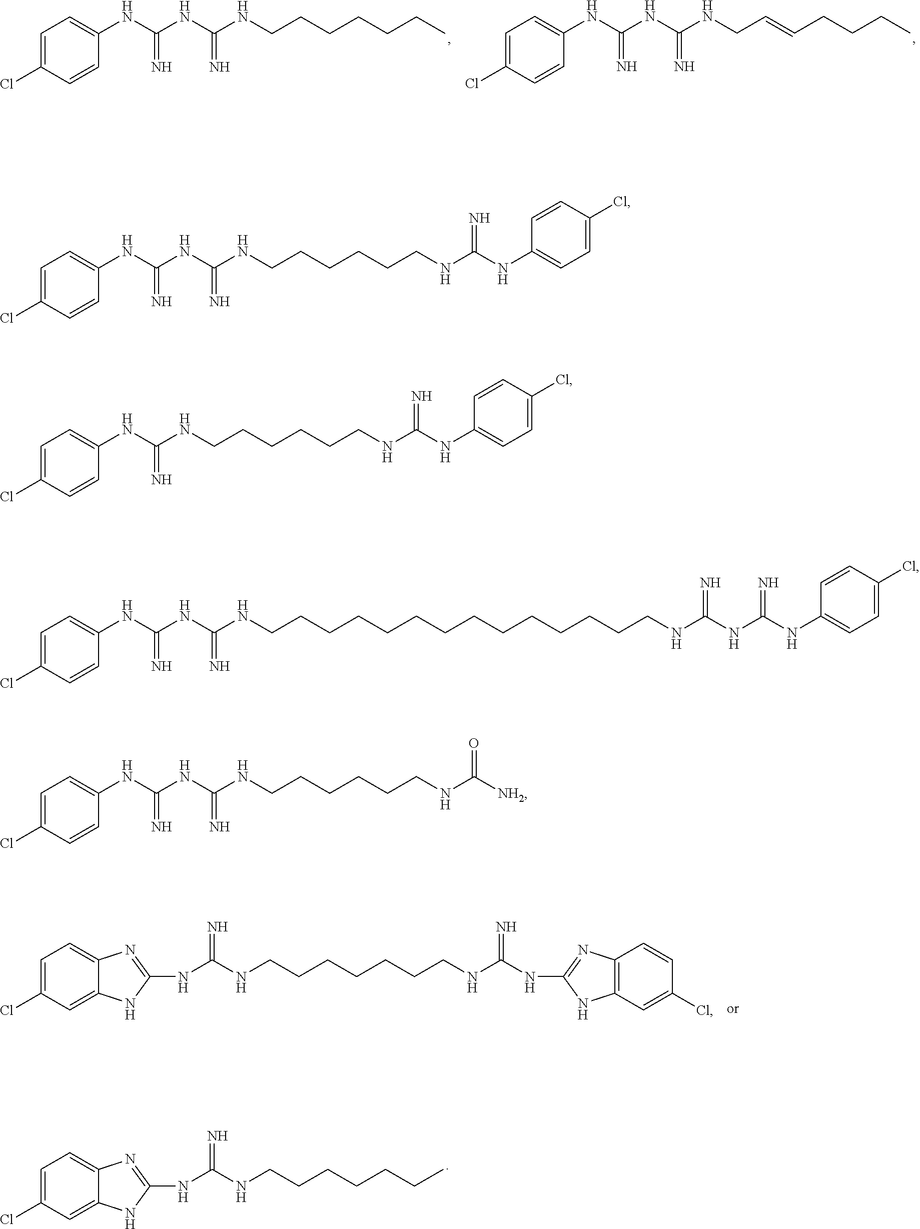

Embodiment 46. A compound, having the formula:

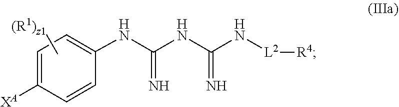

wherein
X^{A} is —Cl, —Br, —I, or —F;
L² is a bond;
R⁴ is unsubstituted C₄-C₂₀ alkenyl;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
z1 is an integer from 0 to 3;
each X¹ is independently —Cl, —Br, —I, or —F;
n1 is independently an integer from 0 to 4; and
m1 and v1 are independently 1 or 2.

Embodiment 47. The compound of embodiment 46, wherein X^{A} is —Cl.

Embodiment 48. The compound of embodiment 46 or embodiment 47, wherein R⁴ is unsubstituted C₄-C₈ alkenyl.

Embodiment 49. The compound of embodiment 46 or embodiment 47, wherein R⁴ is

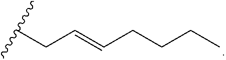 [SMILES: *C/C=C/CCCC]

Embodiment 50. The compound of one of embodiments 46 to 49, wherein R¹ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 51. The compound of one of embodiments 46 to 49, wherein z1 is 0.

Embodiment 52. The compound of embodiment 46, having the formula:

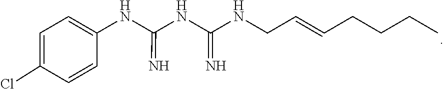 [SMILES: CCCC/C=C/CNC(=N)NC(=N)Nc1ccc(Cl)cc1]

Embodiment 53. A pharmaceutical composition comprising a pharmaceutically acceptable excipient and a compound, or a pharmaceutically acceptable salt thereof, having the formula:

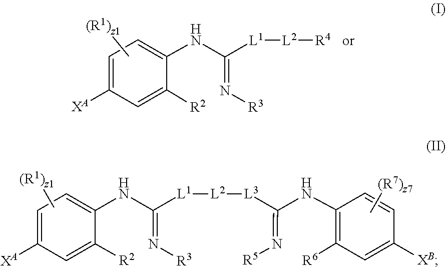

wherein
X^{A} and X^{B} are independently —Cl, —Br, —I, or —F;
L¹ is a bond, —C(O)—, —C(O)O—, —OC(O)—, —O—, —S—, —NR¹⁰—, —C(O)NR¹⁰—, —NR¹⁰C(O)—, —NR¹⁰C(O)O—, —OC(O)NR¹⁰—, —NR¹⁰C(O)NR¹⁰—, —NR¹⁰C(NH)NR¹⁰—, —S(O)₂—, —NR¹⁰S(O)₂—, —S(O)₂NR¹⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —C(O)—, —C(O)O—, —OC(O)—, —O—, —S—, —NR²⁰—, —C(O)NR²⁰—, —NR²⁰C(O)—, —NR²⁰C(O)O—, —OC(O)NR²⁰—, —NR²⁰C(O)NR²⁰—, —NR²⁰C(NH)NR²⁰—, —S(O)₂—, —NR²⁰S(O)₂—, —S(O)₂NR²⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —C(O)—, —C(O)O—, —OC(O)—, —O—, —S—, —NR³⁰—, —C(O)NR³⁰—, —NR³⁰C(O)—, —NR³⁰C(O)O—, —OC(O)NR³⁰—, —NR³⁰C(O)NR³⁰—, —NR³⁰C(NH)NR³⁰—, —S(O)₂—, —NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted heterocycloalkyl or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SO₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OC(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ and R⁶ substituents may optionally be joined to form a substituted or unsubstituted heterocycloalkyl or substituted or unsubstituted heteroaryl;
R⁷ is independently halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NR^{7C}C(O)NR^{7A}R^{7B}, —N(O)ₘ₇, —NR^{7A}R^{7B}, —C(O)R^{7C}, —C(O)OR^{7C}, —OC(O)R^{7C}, —OC(O)OR^{7C}, —C(O)NR^{7A}R^{7B}, —OC(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰, R²⁰, and R³⁰ is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{4A}, R^{4B}, R^{4C}, R^{4D}, R^{6A}, R^{6B}, R^{6C}, R^{6D}, R^{7A}, R^{7B}, R^{7C}, and R^{7D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl;
z1 is an integer from 0 to 3;
z7 is an integer from 0 to 3;
each X¹, X², X⁴, X⁶, and X⁷ is independently —Cl, —Br, —I, or —F;
n1, n2, n4, n6, and n7 are independently an integer from 0 to 4; and
m1, m2, m4, m6, m7, v1, v2, v4, v6, and v7 are independently 1 or 2.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Identification of Clock-Activating Activity of Chlorhexidine with Pro-Myogenic Effect

Circadian clock exerts temporal control in key aspects of stem cell behavior. Circadian clock components coordinately modulate myogenic differentiation and regenerative myogenesis that impacts muscle damage in dystrophic disease. Through in-silico screening of NCI-DTP together with FDA chemical libraries with biochemical and functional validations, here we report, inter alia, the identification of chlorhexidine as a novel circadian clock activator that promotes myogenesis. Following all-around docking analysis to screen molecules with structural fit targeting a hydrophobic pocket within the CLOCK protein, secondary functional screening for clock-modulatory activity identified chlorhexidine (CHX) as a clock activator molecule that induced significant clock period length shortening with augmented oscillatory amplitude. Mechanistically, chlorhexidine promotes CLOCK protein heterodimerization with its obligate partner Bmal1, resulting in enhanced CLOCK/Bmal1 transcriptional activation with up-regulation of key clock components. This effect of chlorhexidine is dependent on Cysteine 267 within the targeted hydrophobic pocket of CLOCK protein. Consistent with its clock-activating action, CHX induced core clock genes and enhanced proliferation in U2OS cells. Furthermore, CHX treatment of C2C12 myoblasts enhanced myogenic differentiation with marked induction of the myogenic gene program and mature myocyte markers, together with significant effect on stimulating proliferation and migration. Additionally, we show that CHX activities are clock-dependent, as its effects on clock induction and myogenesis were abolished in Bmal1-deficient myoblasts. The current study is the first report that demonstrated the feasibility of a screening strategy specifically targeting the CLOCK protein that led to the identification of CHX as a novel clock activator. The pro-myogenic activities of CHX may have therapeutic applications in augmenting skeletal muscle regenerative capacity for dystrophic or degenerative diseases.

The molecular machinery that drives circadian rhythm, the molecular clock, is composed of a transcriptional/translational negative feed-back loop (5). The key transcription activators, CLOCK (Circadian Locomotor Output Cycles Kaput) and Bmal1, form an obligatory heterodimer via interaction within the PAS domain to drive clock gene transcription by binding to the canonical E or E′-box sequence (6). Clock repressor proteins, Cryptochrome (Cry1 & 2) and Periods (Per1-3), are direct transcription targets of CLOCK/Bmal1, which forms a negative transcriptional feed-back arm by inhibiting CLOCK/Bmal1 activity. In addition, a Rev-erbα and ROR-controlled mechanism generates Bmal1 transcriptional oscillation that re-enforce the robustness of the clock (7,8). Additional post-transcriptional and post-translational regulatory mechanisms are involved to complete the molecular clock circuit that drives the 24-hour oscillation in gene expression, physiology and behavior (1). Various components of the molecular clock circuit have been targeted for pharmacological modulations, such as Rev-erbα, ROR, and Cry.

Accumulating studies indicate that circadian clock exert important temporal control in various physiological processes in skeletal muscle, including maintenance of nutrient metabolism, structural integrity and tissue growth (9,10). We previously reported that the tissue-intrinsic circadian clock in muscle and adipose tissues are required for the metabolic regulation and tissue growth processes. Key components of the circadian clock circuit, both the positive arm and negative regulatory arms, orchestrate the differentiation of myogenic progenitors into mature multi-nucleated myotubes. Bmal1 deficiency impairs myogenic differentiation and regenerative myogenesis, whereas ablation of its transcription repressor Rev-erbα promotes these processes (11-13). In addition, Per1/Per2 and Cry2, key regulators within the negative molecular clock loop, modulates myogenesis that impacts muscle regeneration (14,15). Moreover, loss of Bmal1 resulted in obesity with reduced muscle mass (11,16), while a functional clock is required in skeletal muscle as a nutrient sensor by responding to feeding signals and facilitating the metabolic fuel switch from fat oxidation to glucose utilization that determines insulin sensitivity (17).

To date, small molecules directly targeting the key transcriptional drivers of the core clock loop, CLOCK and Bmal1, have yet to be uncovered. In the current study, we conducted a screen for compounds specifically targeting the CLOCK protein to modulate circadian clock activity. Herein we report, inter alia, the identification of chlorhexidine (CHX) as a clock activator with pro-myogenic activities that have potential muscle disease applications.

### Cell Culture

The cell lines used were maintained at 37° C. in 10% Fetal bovine serum (FBS) (Cytiva), 1% Penicillin-Streptomycin-Glutamine 100× (PSG) (Gibco—Thermo Fisher) Dulbecco's Modified Eagle Medium (DMEM) (Gibco—Thermo Fisher) and removed from culture plates for experimentation using 0.25% Trypsin—EDTA 1× (Gibco—Thermo Fisher CN: 25200072). 2% FBS supplemented DMEM was used for differentiation of 80-90% confluent cultures on C2C12, C2C12 Bmal1 KD and C2C12 SC cell lines.

### All-Around Docking-Based Virtual Screening

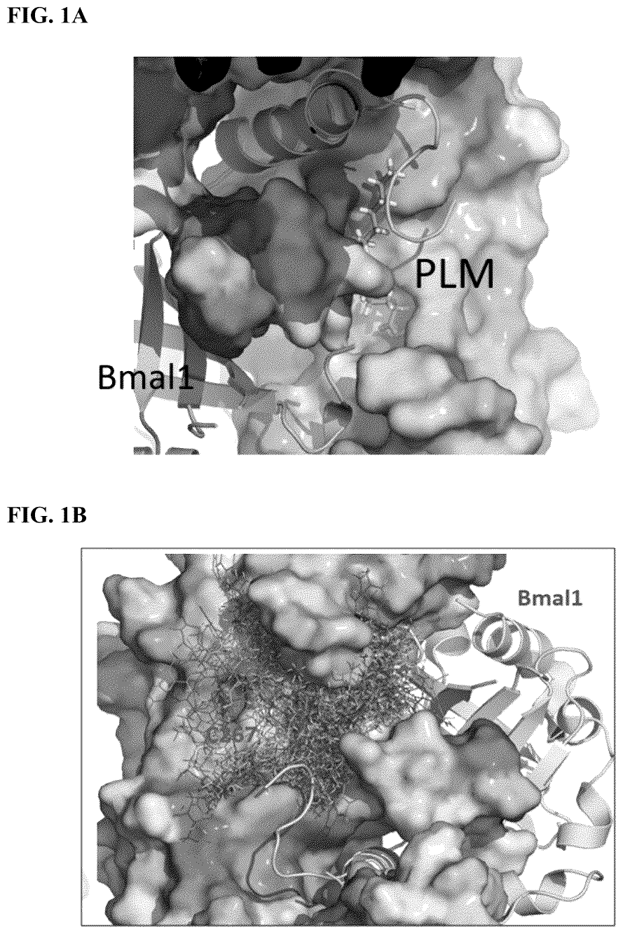

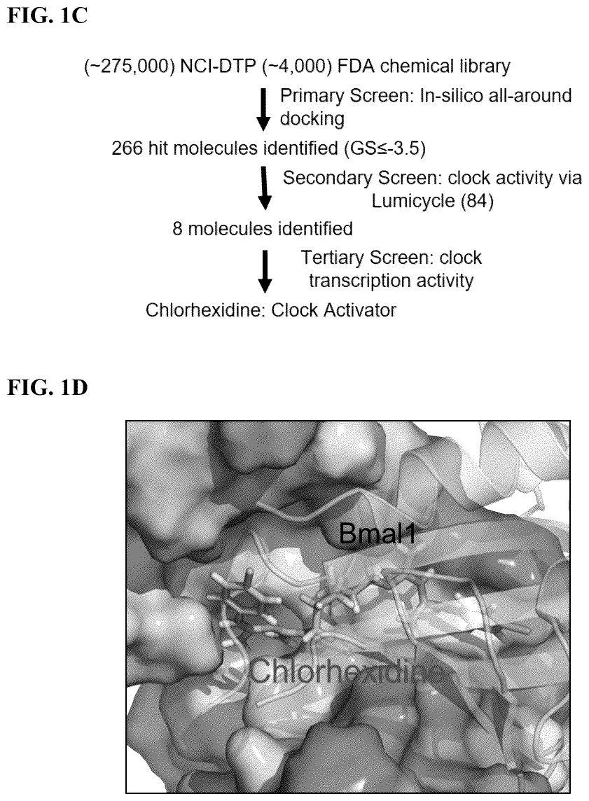

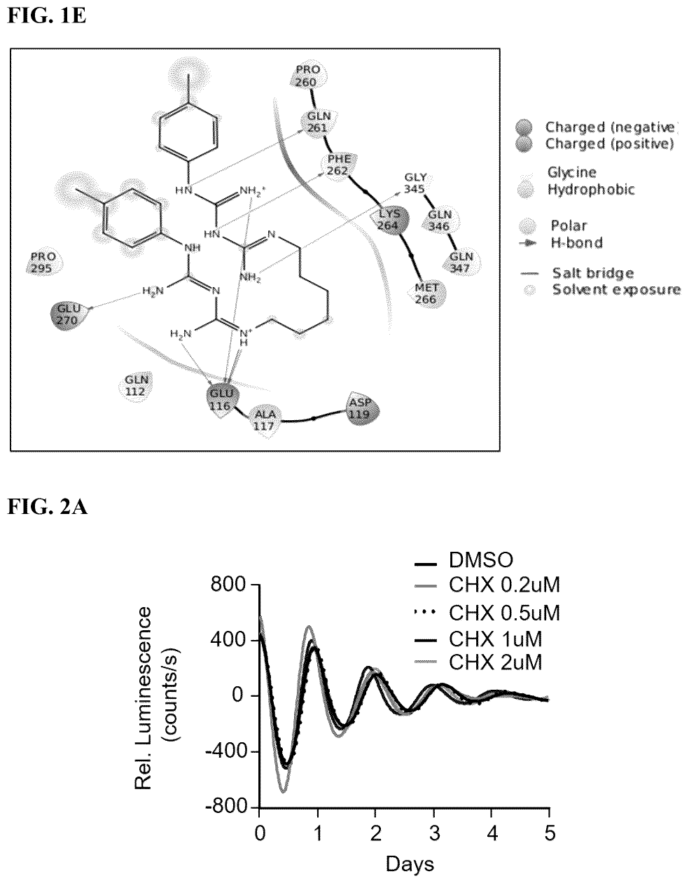

Briefly, the protein crystal structure of CLOCK was obtained from RCSB Protein Data Bank (PDB 4f31) (6). An in-house developed LiVS (Ligand Virtual Screening Pipeline) (Liu et al., 2016) was employed to screen the NCI Developmental Therapeutics Program (DTP) compound library (containing about 260,000 compounds) in silico to identify hits (FIGS. 1A-1C). LiVS method is a multiple-stage and full-coverage pipeline for virtual ligand screening that utilizes the three precision modes (i.e., HTVS, high-throughput virtual screening; SP, standard precision; and XP, extra precision) of Schrodinger Glide software (Friesner et al., 2004) for docking. First, the HTVS precision mode, which is fast but less accurate, was implemented to dock the entire NCI DTP library. The 10,000 top-ranked compounds were next docked and scored by the SP mode. Then the 1,000 top-ranked compounds from SP precision docking were re-docked and re-scored by the XP mode. The 1,000 compounds were further analyzed and filtered by Lipinski's rule of five (Lipinski, 2004), HTS frequent hitter (PAINS) (Baell and Holloway, 2010), protein reactive chemicals such as oxidizer or alkylator (ALARM) (Huth et al., 2005), and maximized the molecule diversity by using UDScore (Universal Diversity Score, developed by us to measure library diversity which is independent of library size). Based on the virtual screening pipeline, we requested the top 255 compounds from NCI DTP and obtained 83 available for secondary functional screening.

### Real-Time Monitoring of Bioluminescence of Per2::Luciferase Reporter Cell Lines

U2OS cells or mouse fibroblasts (MF) containing a Per2-luciferase reporter (Yoo et al., 2004) were seeded at 4×10⁵ concentration on 24 well plates and treated at 90% confluence with 1 ml 1×Fresh explant medium with desired compounds and controls. The seeding density was decided after trying several densities and choosing the one that after 24 h lead to 90% confluence. 1× Fresh explant medium was produced using 50% 2×DMEM buffer stock [DMEM powder (Gibco—Thermo Fisher CN: 12800-017), Sterile MilliQ water, pH7 1 M HEPES (Gibco—Thermo Fisher CN:15630080) and 7.5% Sodium Bicarbonate (Gibco—Thermo Fisher CN: 25080094)], 10% FBS (Cytiva), 37.9% Sterile MiliQ water, 1% PSG 100× (Gibco—Thermo Fisher CN: 10378016), 0.1% Sodium Hydroxixe (NaOH) 100 mM (Fishel Chemical CN: S318-500), 1% XenoLight D-Luciferin—Monopotassium Salt Bioluminiscent Substrate 100 mM (PerkinElmer CN: 122799). Treated cells were introduced to the LumiCycle96 (ActiMetrics) were they were cultured and Per2-luciferafe reporter luminescence was measured for 6 days. Raw and subtracted results for 5 days were exported and data was calculated as luminescence counts per second.

### Luciferase Assay

Cells were seeded at 4×10⁵ concentration on 24 well plates. The seeding density was decided after trying several densities and choosing the one that after 24 h lead to 90% confluence. At 90% confluence cell transfection was performed using Opti-MEM™ Reduced Serum Medium (Gibco—Thermo Fisher CN:31985062) with PGL2, PRL, pcDNA3.0-BMAL1-His, PcDNA3.0-6×Myc-CLOCK (AddGene CN: 47334) pcDNA3.0-3×FLAG-cry2 plasmids and PEI max 40 k (Polysciences Inc CN: 24765-1). After 24 h of transfection, cells were treated with desired compounds and controls in 10% FBS 1% PSG DMEM media for desired times. Cells were extracted with 1×PLB provided in the Dual-Luciferase® Reporter Assay Kit (Promega CN: E1910) and transferred to 96-well black plate to be treated with LARII and Stop & Glo components according to protocol of the Dual-Luciferase® Reporter Assay Kit (Promega CN: E1910). Luminescence was measured on microplate (ELISA) reader (TECAN infinite M200pro). The mean and standard deviation values were calculated for each well and graphed.

### Western Blot Analysis

Total protein (20-30 μg) was extracted using standard IP Lysis buffer (3% NaCl, 5% Tris-HCl, 10% Glycerol, 0.5% Triton X-10 in Sterile MilliQ water) and resolved on 10% SDS-Page gels followed by western blotting on Immun-Blot PVDF Membranes for Protein Blotting (Bio-rad). Antibodies used were diluted in 5% milk (Labscientific CN: M0841). Membranes were washed with TBS-T [10% TBS, 0.1% Tween™ 20 (Fisher BioReagents CN: BP377-500)]. Images were developed using Luminol solution 1:1 SuperSignal West Pico PLUS Stable Peroxide Solution (Thermo Scientific CN: 1863095) and SuperSignal West Pico PLUS Luminol Enhancer Solution (Thermo Scientific CN: 1863094) on chemiluminescence imager (GE Healthcare Bio-Sciences AB Amersham Imager 680).

### RNA Extraction and Quantitative Reverse-Transcriptase PCR Analysis

Cells extracted with TRIzol™ Reagent (Ambion CN: 15596018) were collected. RNA extraction was performed with Chloroform (Fisher Chemical CN: C298-500) and Isopropanol (Fisher Chemical CN: A451SK-4) extraction followed by two purification steps with 75% Ethanol (Fisherbrand CN: HC-800-1GAL) and elution in Sterile MilliQ water.

The RNA concentration was measured using Nanodrop spectrophotometer (Thermo Fisher). Following RNA extraction, retro transcription was performed using RevertAid RT Reverse Transcription Kit (Thermo Fisher CN: K1691) and run on SimpliAmp Thermal Cycler (Thermo Fisher CN: A24811) according to the kit procedure. Quantitative PCR was performed using PowerUp™ SYBR™ Green Master Mix (Thermo Fisher CN: A25742) using a dilution of 5:2:1 master mix, RNA and H2Omq according to the protocol on ViiA 7 Real-Time PCR System (Applied Biosystems). Relative expression levels were determined using the comparative Ct method to normalize target genes to 36B4 internal controls.

### MTT Assay for Cell Viability and Proliferation

Cells were seeded at 5×10⁴ concentration in 100 μl culture medium into 96 wells flat bottom microplates and treated after 24 h with desired compounds and controls. After desired incubation periods, cells were treated with MTT labeling reagent 1× (MTT (3-[4,5-dimethylthiazol-2-yl]-2,5-diphenyltetrazolium bromide labeling reagent, 5 mg/ml in PBS, Roche Cell Proliferation Kit I (MTT)) and Solubilization buffer 1× (10% SDS in 0.01 M HCl and left overnight. Next morning absorbance was measured at 570 nm and 700 nm on microplate (ELISA) reader (TECAN infinite M200pro). The mean and standard deviation values were calculated for each well and graphed.

### EdU Proliferation Assay

Cells were seeded at 0.2×10⁵ concentration and treated with desired compounds and controls overnight. The seeding density was decided after trying several densities and choosing the one that after 24 h lead to visible, not clumping cells. 10 μM 5-ethynyl-2′-deoxyuridine (EdU) incorporation was performed during 4 h according to the kit protocol and detected by Click-iT Imaging Kit with Alexa 488 Fluorophore (Invitrogen). 1 μg/ml 4′,6′-diamidine-2′-phenylindole dihydrochloride (DAPI) was used to label for nuclei. Total number of EdU+ cells was counted from 8 representative fields at 20× and the rate of proliferation was calculated as percentage of EdU+/DAPI.

### Wound Healing Assay

Cells were seeded at 12×10⁵ concentration on 6 well plates until 90% coverage and treated with compounds as indicated and controls while maintained 10% FBS 1% PSG DMEM. Scratch wounds were made with disposable cells scrapers and pictures were taken at hours 0, 4, 8, 24 and 48 following scratch. The results were measured as percentage coverage over original wound area based on image quantification. Area of wound closure was measured using Image J software.

### Myogenic Differentiation

C2C12, C2C12 Bmal1 KD and C2C12 Scrambled control (SC) myoblasts were seeded at Day −1 at 1.2×10⁶ concentration on 6 wells plates and maintained in 10% FBS until 90% confluency and treated with indicated compounds and controls in 2% FBS DMEM media to induce differentiation on Day 0. Pictures were taken every day to monitor morphological progression, and proteins and RNA samples were collected on days indicated.

### MyHC and MyoD Immunostaining

C2C12, C2C12 Bmal1 KD and C2C12 Scrambled control (SC) myoblasts were seeded at Day −1 at 2×105 concentration on 12 wells plates and maintained in 10% FBS until 80% confluency and treated with indicated compounds and controls in 2% FBS DMEM media to induce differentiation on Day 0. On days 3, 5 and 7 cells were fixed with 4% paraformaldehyde (from 37% Formaldehyde Baker Analyzed) and kept at 4° C. in PBS. Cells were permeabilized with Permeabilization solution of 0.5% Triton X-100 (Fisher Bioreagents CN: 9002-93-1) in PBS and blocked with Blocking solution of 1% BSA (Fisher Bioreagents CN:9048-46-8) in PBS. Primary antibodies were diluted in Washing solution of 0.01% Triton X-100 (Fisher Bioreagents CN: 9002-93-1) in PBS and left overnight at 4° C. Cells were then incubated with secondary antibodies in Washing solution at room temperature for 1 h. 1 μg/ml 4′,6′-diamidine-2′-phenylindole dihydrochloride (DAPI) was used to label for nuclei. Pictures were taken to prove protein expression changes related to morphological progression. To quantify the proliferation of myoblast, when needed, cells were treated with 1 μg/ml of EdU for 3 h before fixation. Detection of EdU was performed with Click-iT EdU Alexa Fluor 488 Imaging Kit before applying the immunofluorescence staining as described above.

### Statistical Analysis

Data are presented as mean+SE or SD. Each experiment was repeated at minimum twice to validate the result. Sample size were indicated for each experiment in figure legends. Two-tailed Student's t-test or One-way ANOVA with post-hoc analysis for multiple comparisons were performed as appropriate as indicated. P<0.05 was considered statistically significant.

### Identification of Chlorhexidine as a Small Molecule Activator of Circadian Clock

Recent interests for therapeutic targeting of circadian clock are largely focused on repressor proteins of the “druggable” nuclear Rev-erbα (Nr1d1) or Cryptochromes. We thus conducted a screen to identify small molecules targeting the core circadian clock transcription activator, CLOCK protein. The crystal structure of CLOCK protein, PDB 4f31 (Huang et al., 2012), was used for the high throughput ligand virtual screening (20), with the overall strategy shown in FIG. 1C. All-around docking (ADD) analysis was performed to identify molecules with ranked structural fit for a deep hydrophobic pocket within the well-defined PAS-A domain of CLOCK that mediates association with its obligatory heterodimer Bmal1 (6). Compounds were ranked based on Glide Score calculation that predict potential strength of binding with predicted hydrophobic, polar, or hydrogen bond interactions with key residues within this structure. Chemical libraries from NCI Developmental Therapeutic Program (DTP) with (˜275,000 compounds) and the FDA library (4,086 compounds) were selected for screening with access to hit compounds. 266 compounds were identified as hits in these libraries based on Glide Score ranking of ≤3.5, and the docking poses were shown in FIG. 1B. Out of these initial hits via virtual screening, we were able to obtain 84 molecules for secondary screening, which used a gold-standard clock activity phenotypic assay via a U2OS reporter cell line containing a Period2-driven luciferase construct (FIG. 1C). Three molecules with significant clock modulatory activity were identified by this screen, and chlorhexidine (CHX) was the only molecule that functions as a clock activator. The clock-modulatory activity of CHX was subjected to further validation via transient luciferase activity assays, with subsequent analysis of its mechanism of action and biological activity in myogenic assays that are driven by circadian clock regulation. CHX display a strong GS score of −19.5, with a tight docking pose within the targeted CLOCK hydrophobic pocket that lies within CLOCK-Bmal1 interaction interface (FIG. 1D). The predicted charged, hydrophic, polar or hydrogen bond interactions of CHX with CLOCK residues within 10-20 Å of amino acids outlining the hydrophobic pockets were indicated (FIG. 1E).

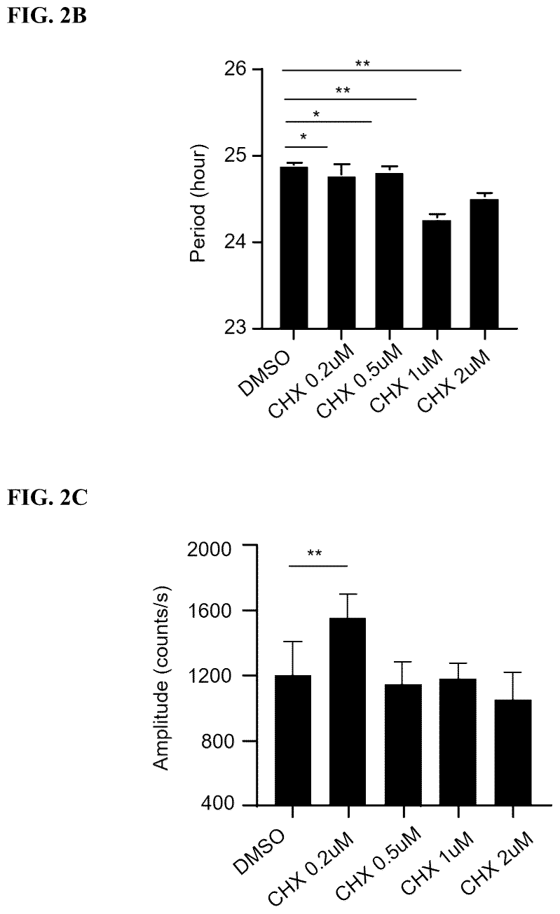

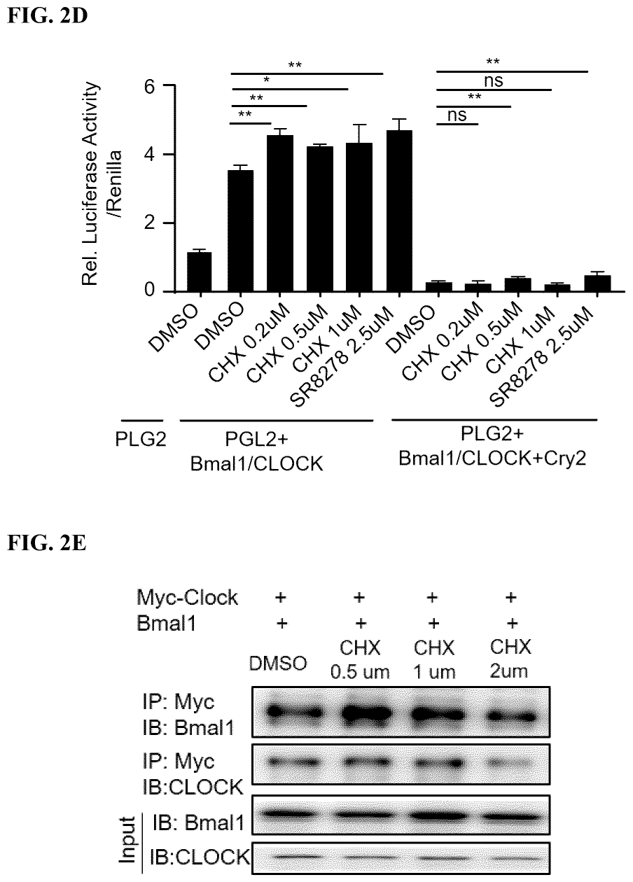

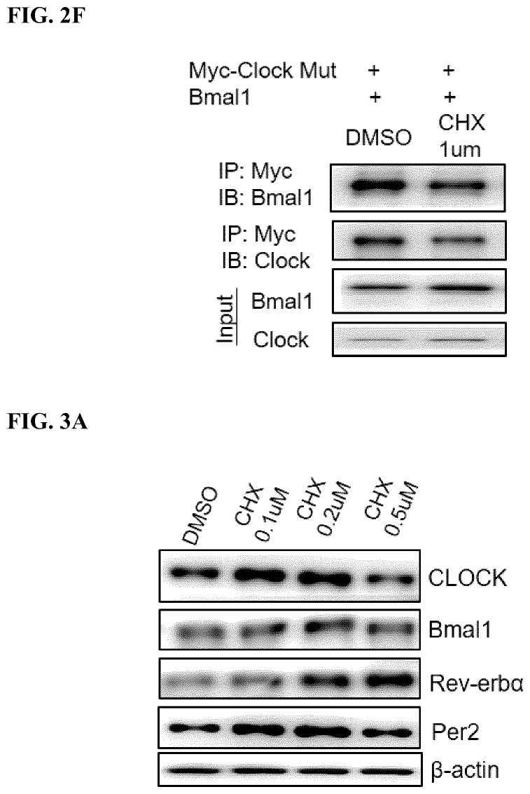

Given our screening strategy targeting the hydrophobic pocket in CLOCK protein involved in heterodimerization with Bmal1, compounds with structural fit may modulate this interaction to influence CLOCK/Bmal1-controlled transcription. We thus tested whether CHX activation of clock is mediated through CLOCK/Bmal1 interaction with resultant enhanced transcriptional activity. Through continuous monitoring of bioluminescence activity of Per2-Luc U2OS reporter line, we determined the activity of CHX on modulating key circadian clock properties, including clock oscillating period length, amplitude, and phase angle (Yoo et al., 2004). Cells were treated with CHX at indicated concentrations from 0.2 to 2 μM at start of the bioluminescence recording, as shown in baseline-subtracted plots for 5 days (FIG. 2A). Quantitative analysis revealed a dose-dependent reduction of period length from 0.2 to 1 μM as compared to the DMSO control with the strongest effect observed at 1 μM (FIG. 2B). This effect of CHX on shortening period length suggests its activity as a clock activator that leads to shortened cycles. In addition, chlorhexidine at a low concentration of 0.2 μM induced clock amplitude, but not at any higher concentrations examined (FIG. 2C). Testing of additional high concentrations of CHX at 5 and 10 μM revealed significant toxicity with reduced amplitude. Using transient transfection of CLOCK/Bmal1 that activates the Per2-luciferase reporter, we further tested whether acute CHX treatment promotes CLOCK/Bmal1-mediated transcription. As indicated in FIG. 2D, Bmal1 and CLOCK co-transfection induced luciferase activity by ˜3-fold, as expected (FIG. 2A). CHX treatment at 0.2 μM for 6 hours was sufficient to stimulated ˜26% induction of CLOCK/Bmal1-activated transcription with similar effects at higher concentration, while Cry2 was sufficient to suppress this effect as expected. We next performed co-immunoprecipitation to test whether CHX promotes CLOCK binding with Bmal1, and found that CHX at 0.5 and 1 μM were sufficient to augment their interaction with increased Bmal1 level immunoprecipitated by Myc-CLOCK, although higher concentration at 2 μM does not further promote this activity (FIG. 2E). To test whether the targeted CLOCK structure mediates CHX effect, we mutated a key residue Cysteine 276 within the hydrophobic pocket to Alanine. While C267 mutation did not affect CLOCK/Bmal1 heterodimer association as compared to wild-type CLOCK, CHX failed to increase Bmal1 interaction with CLOCK C267 mutant, supporting the notion that CHX effect is dependent on the targeted CLOCK protein structure (FIG. 2F). Collectively, these results demonstrate that CHX functions as a clock activator by promoting CLOCK and Bmal1 interaction that enhanced CLOCK/Bmal1 transcription activation.

### Induction of Clock Gene Expression by Chlorhexidine

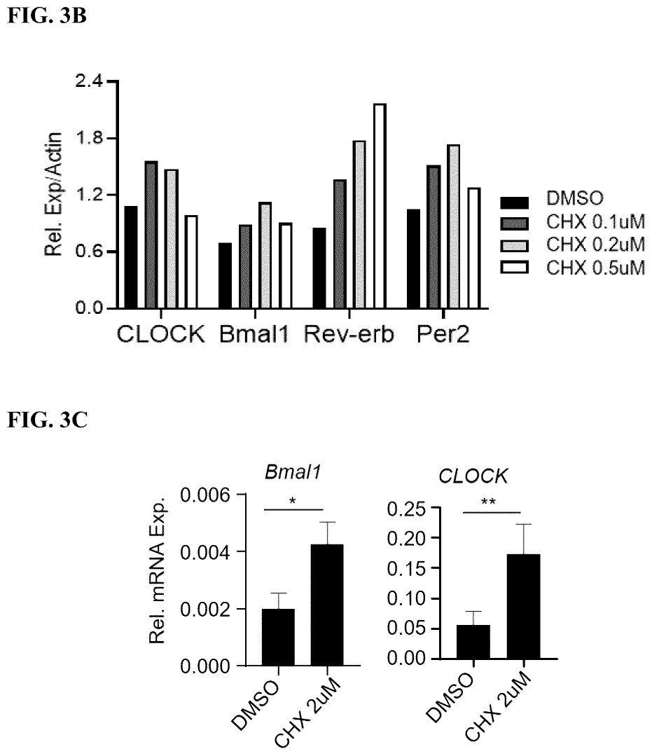

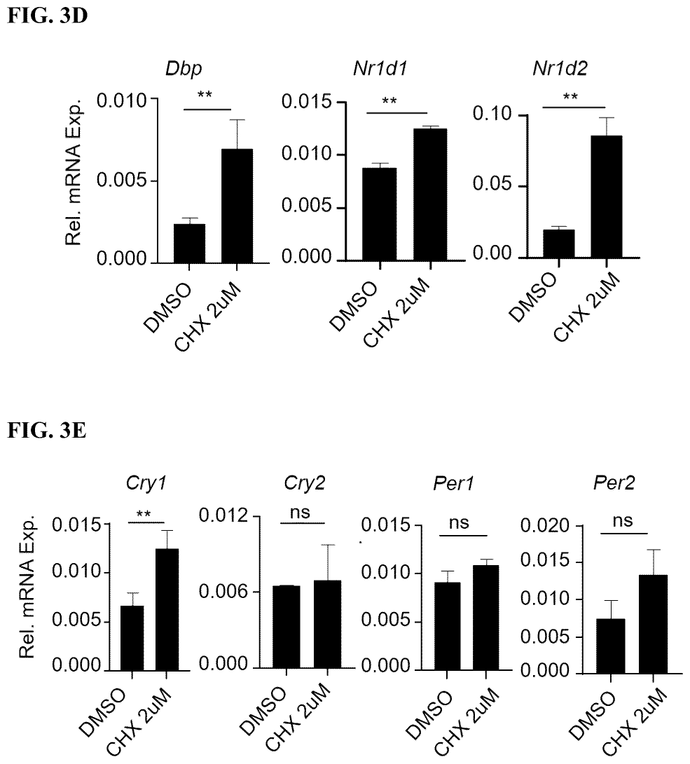

Based on findings of CHX in stimulating CLOCK/Bmal1-mediated transcription, we determined its potential effect on inducing clock gene expression in U2OS cells. 0.1 and 0.2 μM of CHX was sufficient to induce CLOCK protein levels, with elevated Bmal1 expression at 0.2 and 0.5 μM (FIGS. 3A-3B). Rev-erbα (Nr1d1) and Period 2 (Per 2), transcriptional targets of CLOCK/Bmal1 in the core clock circuit, were also induced by CHX in a largely dose-dependent manner. Attenuated stimulation of CHX observed at 0.5 μM could be due to potential toxicity in this cell type. Consistent with this, CLOCK and Bmal1 transcripts were markedly up-regulated by CHX (FIG. 3C), with additional components of molecular clock network, including Dbp, Nr1d1, Nr1d2 and Cry2, also stimulated by CHX treatment (FIGS. 3D-3E).

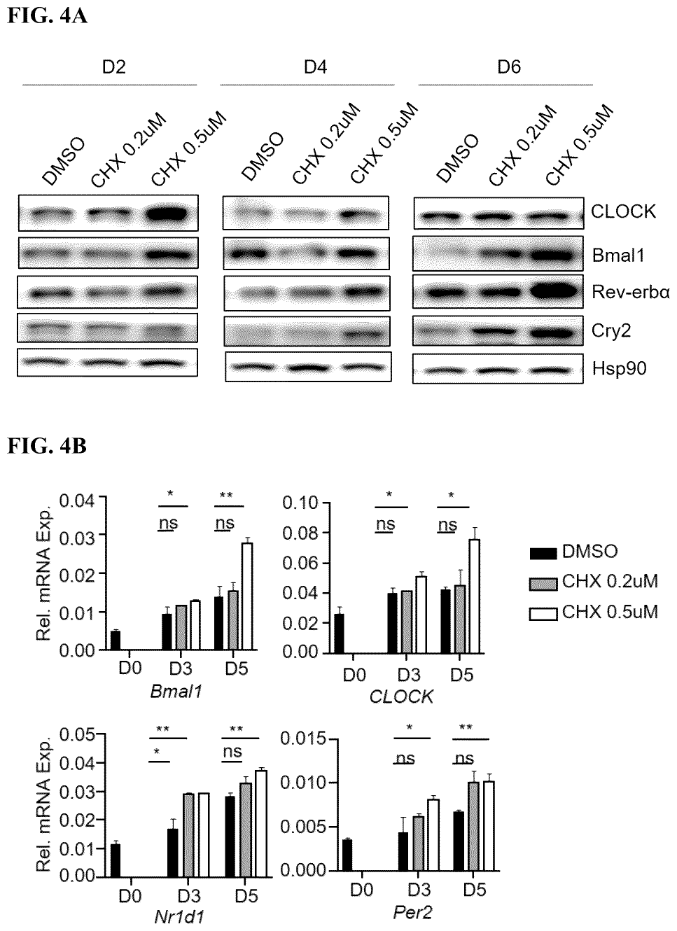

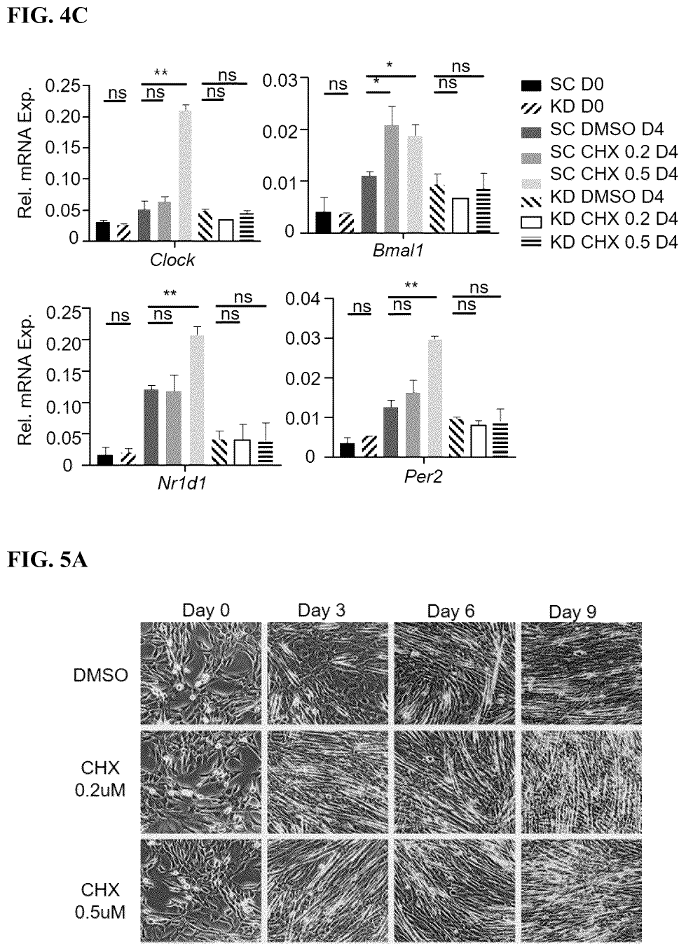

We next tested whether CHX can activate clock using C2C12 mouse myoblasts, and determined its effect during myogenic differentiation. Through 6 days of differentiation, 0.5 uM CHX robustly stimulated CLOCK and Bmal1 protein levels, together with their transcriptional targets Rev-erbα and Cry2 (FIG. 4A and FIG. 8). The augmented expression of clock protein by CHX was corroborated further corroborated by induction of these transcripts, and they displayed a tendency of increased expression by 0.2 μM CHX (FIG. 4B). We next tested whether CHX modulation of molecular clock genes is dependent on a functional clock using myoblasts containing stable shRNA silencing of Bmal1 (BMKD), as compared to cells with scrambled control (SC). As shown in FIG. 4C, the inductions of Clock, Bmal1 and their transcription targets, Rev-erbα and Per2, by CHX at day 4 of myogenic differentiation was completely abolished in the BMKD cells as compared to SC, indicating that indeed CHX effect on activating clock gene transcription is dependent on a functional clock.

### Clock-Dependent Effect of Chlorhexidine in Promoting Myogenesis

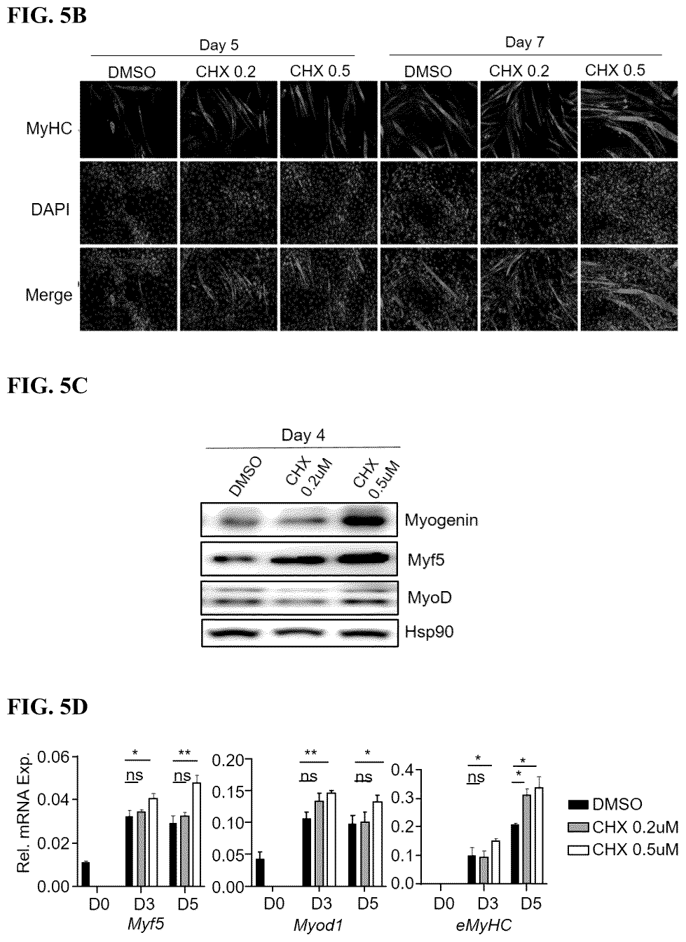

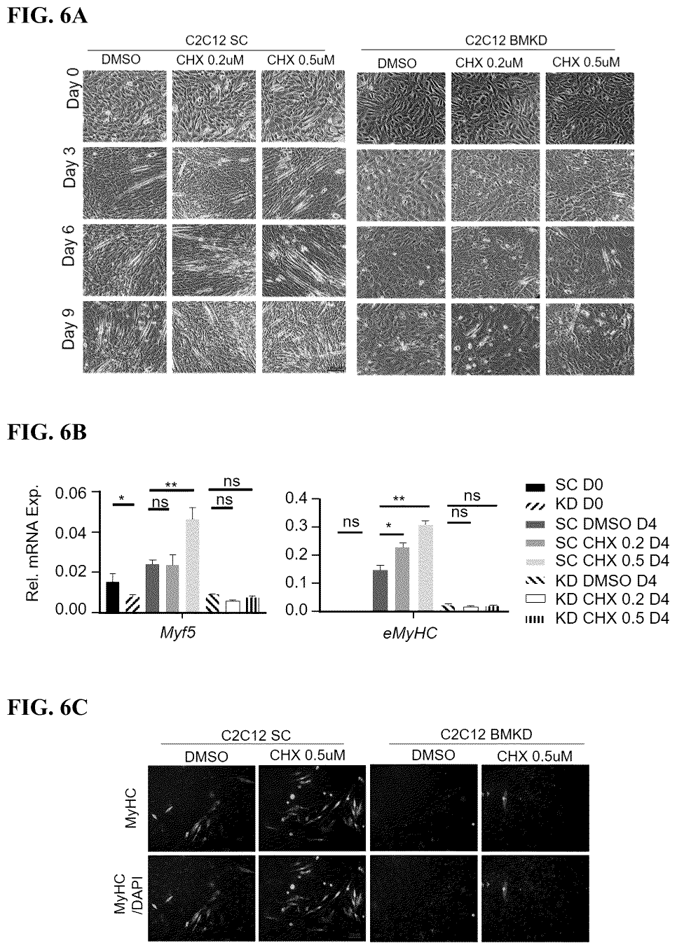

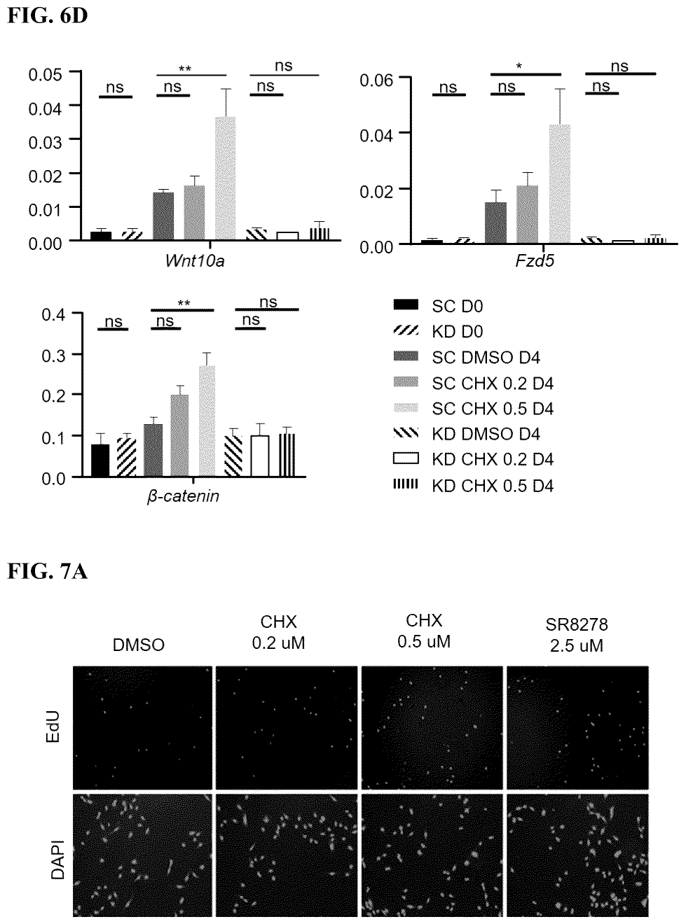

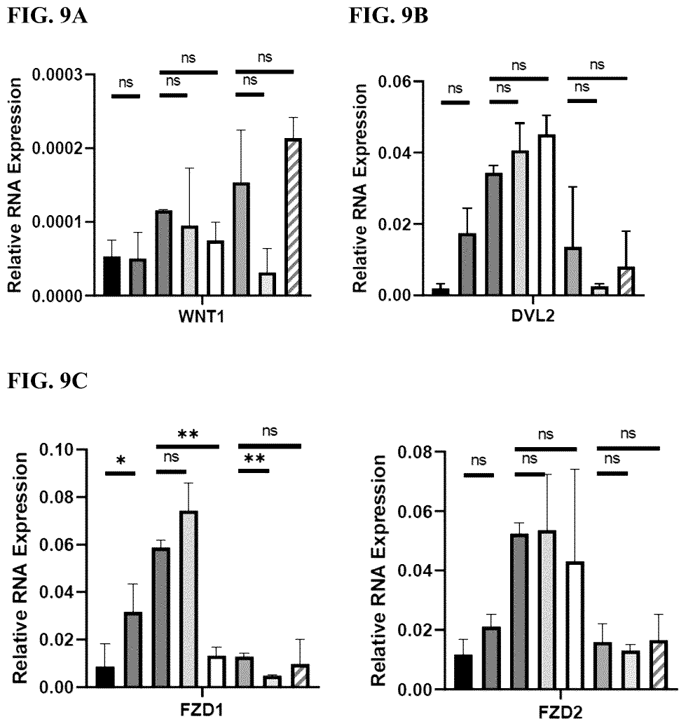

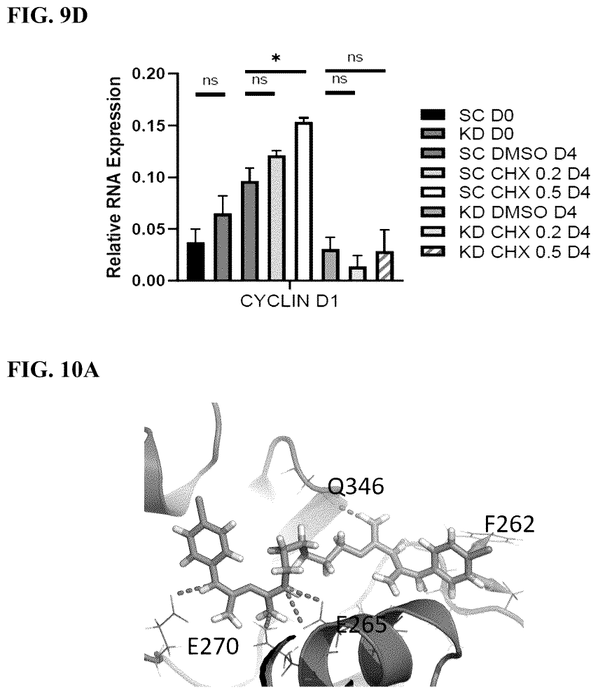

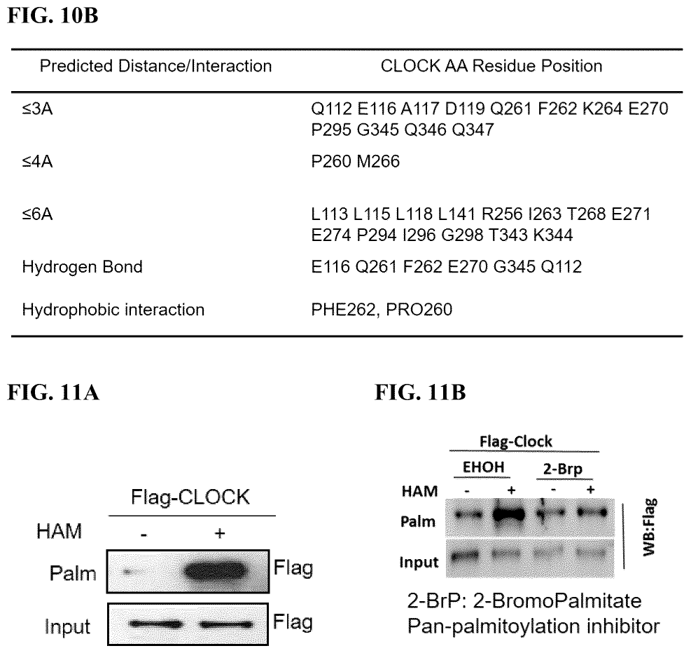

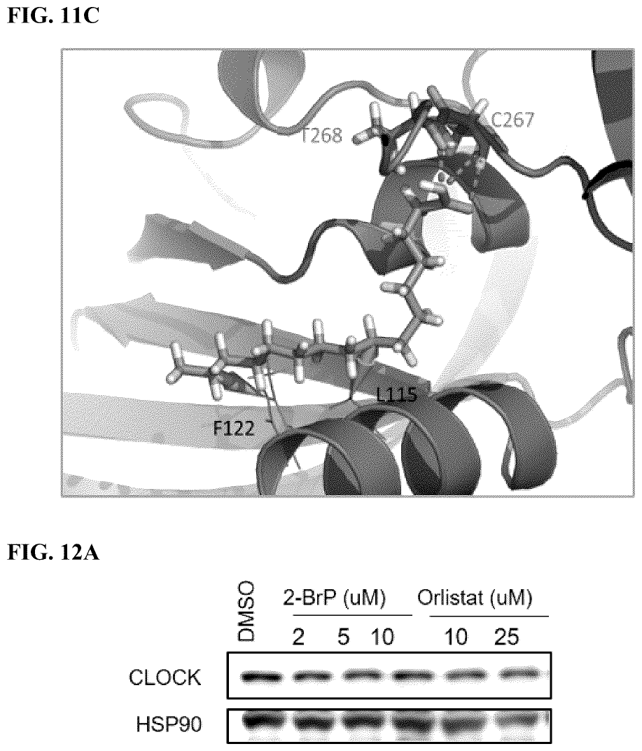

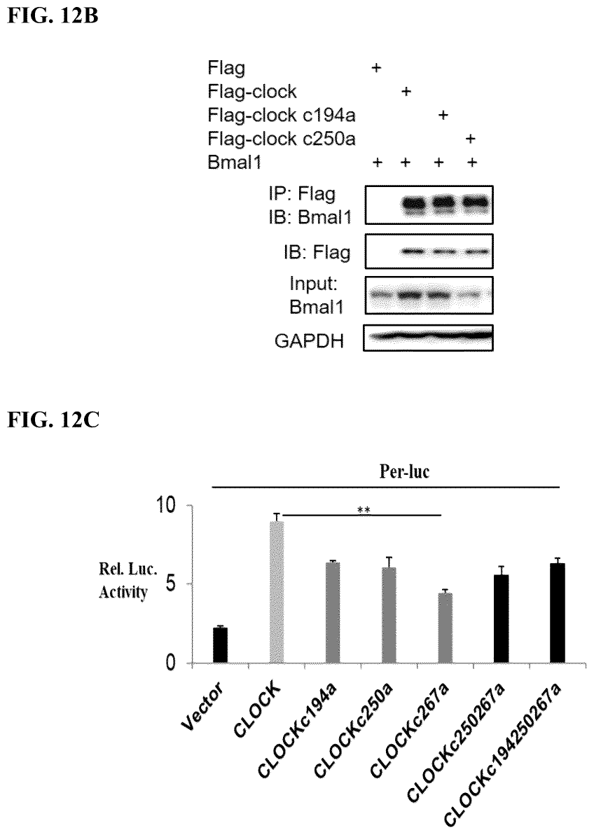

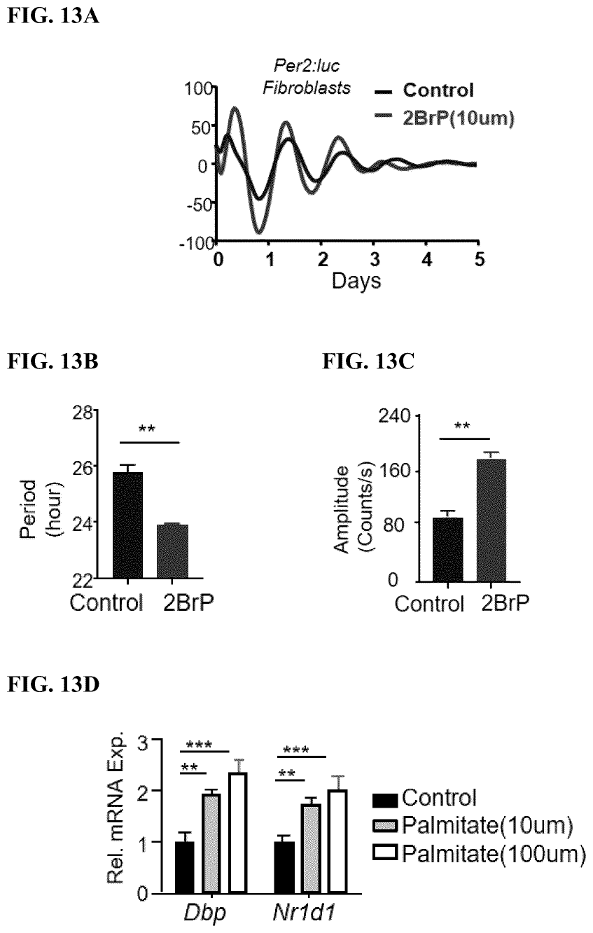

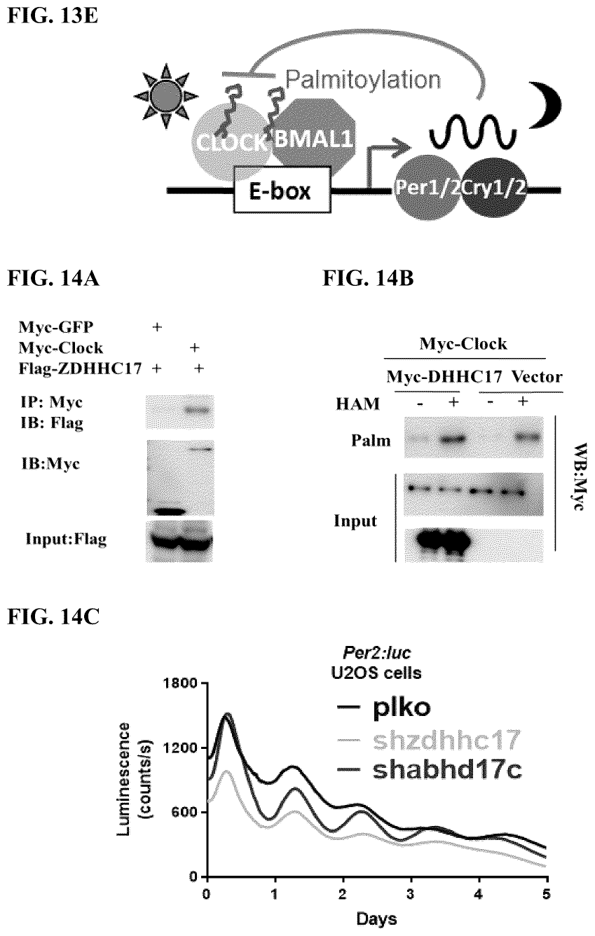

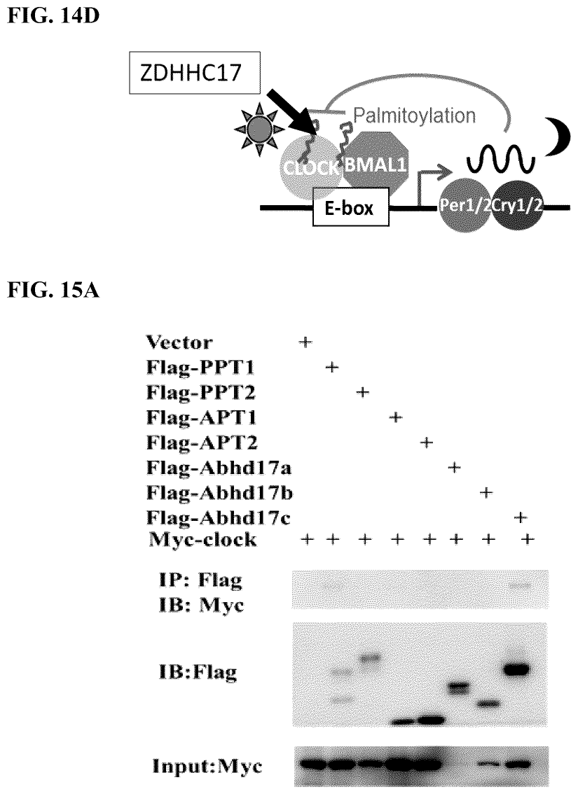

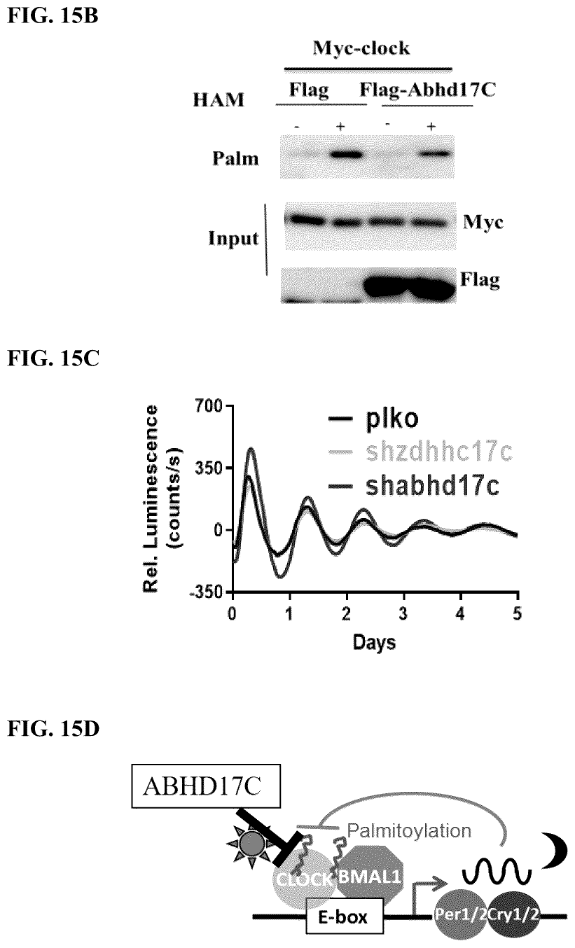

Our previous studies demonstrated that the essential clock activator, Bmal1, promotes myogenesis and muscle regeneration via direct transcriptional control of Wnt signaling cascade (11,13,21). We postulated that CHX, as a clock-activating molecule, may display pro-myogenic activity in myoblast differentiation. As shown by phase-contrast images, normal C2C12 myoblasts differentiate efficiently into mature multi-nucleated myotubes upon 2% serum induction for 9 days (FIG. 5A). In comparison, CHX treatment at 0.2 μM or 0.5 μM markedly accelerated the morphological progression of myotube formation, with abundant mature myotubes formed at early differentiation of day 3 days when barely visible mature myotubes were detected in DMSO-treated cells. Myotube formation in CHX-treated cells at day 3 of differentiation were nearly completed and comparable to the differentiation observed at day 6 of controls, indicating enhanced myocyte maturation. This effect was maintained through differentiation time course, with CHX-treated cells display more advanced myotube elongation at day 6 and 9. However, CHX at 2 and 5 μM appeared to be toxic to myoblasts with cell death during differentiation. This pro-myogenic activity of CHX was further demonstrated by myosin heavy chain (MyHC) immunofluorescence staining for mature myocytes during differentiation. Consistent with morphological maturation, CHX-treated cells at day 5 and day 7 of differentiation displayed increased numbers of MyHC-positive mature myotubes, and 0.5 μM CHX induced notable myotube hypertrophy at Day 7 (FIG. 5B). Consistent with the augmented myogenic differentiation induced by CHX, it led to early induction of myogenic factors Myf5 and Myogenin proteins at day 3 and day 5 along the differentiation time course (FIG. 5C), while Myf5, Myod1 and eMyhc expressions were significantly induced by 0.5 μM CHX with a tendency toward higher expression 0.2 μM (FIG. 5D). We next examined the pro-myogenic activity of CHX in BMKD myoblasts to determined whether this effect is dependent on clock modulation. Due to Bmal1 inhibition, BMKD myogenic differentiation were impaired as compared to SC control (FIG. 6A), as previously reported (11). CHX stimulated myotube formation in SC myoblasts similarly as observed for the parental C2C12 myoblasts, albeit the rate of myogenic progression was attenuated in SC (FIG. 6A). In contrast, CHX failed to augment BMKD myoblast differentiation, indicating that its myogenic activity is indeed clock-dependent. Staining for MyHC to identify mature myocytes revealed similar findings of loss of CHX effect on enhancing the early maturation of myocytes at day 3 of differentiation in the KD cells as compared to its robust effect in SC myoblasts (FIG. 6B). Gene expression analysis further confirmed the clock-dependent effect of CHX on inducing the myogenic program, with loss of CHX induction of Myf5 and eMyhc in Bmal1-deficient myoblasts (FIG. 6B). Based on clock regulation of Wnt signaling during myogenesis, we examined the known transcriptional targets of clock in this pathway and found that CHX was able to stimulate Wnt10a, Fzd5 and β-catenin expression in SC myoblasts, while this effect was abolished in Bmal1-deficient cells (FIG. 6D and FIGS. 9A-9D), further corroborating the clock-dependent action of CHX in activating a key signaling mechanism that drives myogenic differentiation.

### CHX Promotes Myoblast Proliferation and Migration

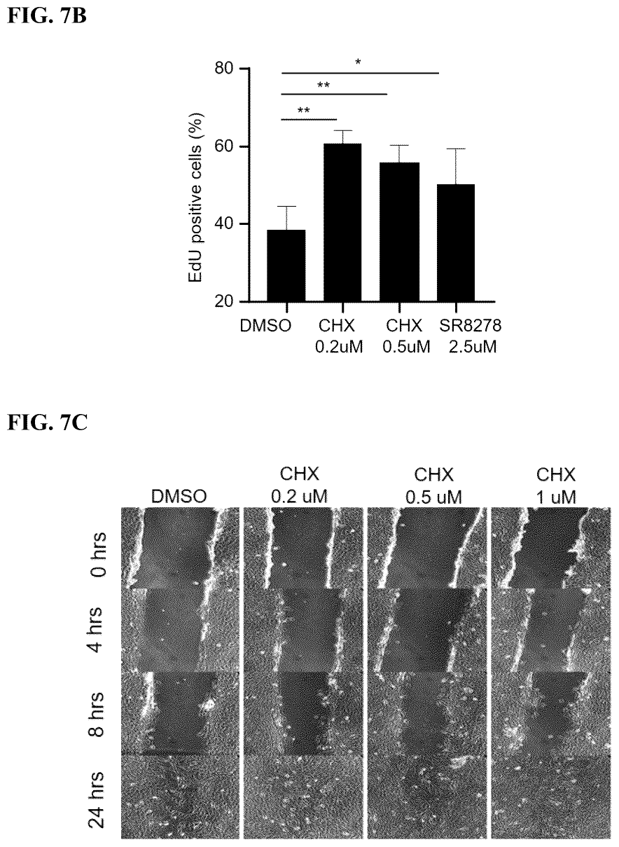

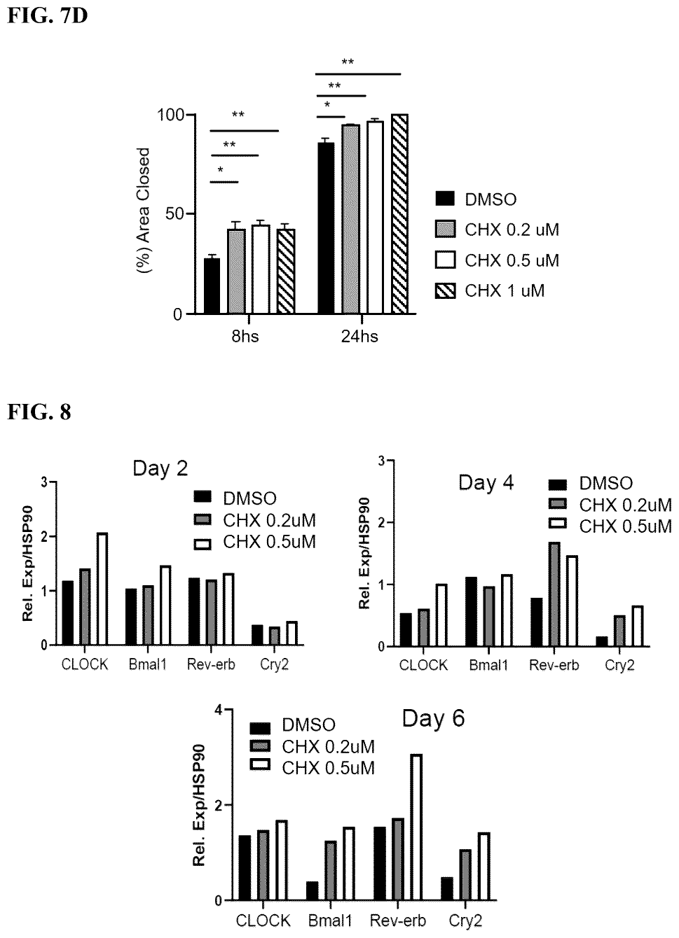

Muscle stem cell provides the major cellular source for muscle regenerative repair, and its activation, proliferative expansion together with migratory activity in response to injury are key processes involved in tissue regeneration (22-24). We thus determined potential effects of CHX on the proliferation and migration in myoblasts. As indicated by EdU incorporation, CHX at both concentrations tested induced ˜40% higher proliferation (FIGS. 7A-7B), and this effect is comparable to that of the Rev-erbα antagonist SR8278 as we demonstrated (12). In addition, we observed a similar degree of augmented proliferation by CHX in U2OS cells (FIGS. 9A-9B). In a wound healing assay of C2C12 myoblasts, area closure at 8 and 24 hours, indicative of the rate of cellular migration, were significantly increased by treatment of CHX ranging from 0.2 to 1 μM as compared with DMSO control (FIGS. 7C-7D). Together these analyses indicate that in addition to its pro-myogenic action, CHX may impact the proliferative and migratory activities of myoblasts to facilitate regenerative repair.

Circadian clock disruption leads to the development of obesity and insulin resistance (25,26). Thus, re-enforcing clock regulation may yield metabolic benefits. Despite current interests in developing clock modulators for disease applications, compounds directly modulate CLOCK activity has yet to be identified. Our screen specifically targeting the CLOCK protein led to the identification of CHX as a novel circadian clock-activating molecule that augments myoblast differentiation, proliferation and migratory activity. Given the role of coordinated circadian clock control in muscle stem cell behavior that is required for regenerative myogenesis and protection against dystrophic muscle damage, these pro-myogenic properties of CHX may have therapeutic applications in dystrophic diseases or muscle-wasting conditions.

We leveraged a powerful high throughput in-silico screening platform based on docking analysis of the NCI-DTP and FDA libraries to identify compounds with a structural fit for a hydrophobic pocket within the PAS-A domain of the CLOCK protein. Secondary functional screening revealed clock-modulatory activities of the top hits through virtual screen, with further biochemical validation for effects on CLOCK/Bmal1-mediated transcription and protein interaction. CLOCK functions as a heterodimer with Bmal1 to activate transcription, and the PAS domains are structural elements critical for CLOCK and Bmal1 interaction. The docking of CHX with the CLOCK crystal structure revealed interactions with several key residues that forms a deep hydrophobic pocket within the PAS-A domain. As CHX displayed clock-activating effect in Per2-Luc reporter cell line, we postulated that its binding within the CLOCK hydrophobic pocket may enhance interaction affinity with Bmal1 to promote CLOCK/Bmal1-activated transcription. Co-immunoprecipitation indeed revealed increased CLOCK and Bmal1 association in cells treated with CHX, with induction of clock genes consistent with its clock-activating activity.

We report herein a CLOCK protein activating molecule with biological activity in promoting myogenic differentiation. Importantly, mutating Cysteine 267 of CLOCK protein abrogated CHX-induced increased CLOCK/Bmal1 association. Additional studies could be needed to further test the specificity of CHX for CLOCK protein, particularly against proteins sharing homology with CLOCK. Nonetheless, our findings indicate that clock gene induction and myogenesis-enhancing activities of CHX are dependent on clock, as they were abolished in Bmal1-deficient myoblasts.

CHX is a cationic bisbiguanide molecule capable of binding to bacterial cell wall with bacteriostatic or bactericidal efficacy against a broad range of bacteria. CHX is currently used as a topical antiseptic agent for skin, and a common ingredient for mouth washes for its anti-plaque and anti-gingivitis activity. It is known to bind to proteins on skin or mucous membranes with limited systemic absorption. Potentially due to its chemical properties, we found CHX at concentrations higher than 1 μM starts to exhibit toxicity to the cells, although distinct cell types appear to have differing sensitivity to CHX. Dose response analysis of bioluminescence activity using Per2-Luc U2OS cells revealed a relatively broader range (0.2-2 μM) than functional assays for its pro-myogenic activities in C2C12 myoblasts (0.2-0.5 μM). We conducted most experiments in the lower concentration range as we determined.

The pro-myogenic activity of CHX was discovered based on our prior findings of circadian clock regulation in myogenesis. Both positive and negative arms of clock transcriptional loop modulate myogenic differentiation, with Bmal1 promoting whereas Rev-erbα inhibiting this process (9,11,12,21,28,29). Notably, these regulations impact chronic dystrophic muscle damage in a preclinical animal model for Duchene Muscular Dystrophy, the dystrophin-deficient mdx mice (21,29). Bmal1 regulation of regenerative repair is required to prevent dystrophic muscle damage (21), whereas loss of Rev-erbα protects against dystrophic muscle damage (29). Moreover, pharmacological inhibition of Rev-erbα by an antagonist ameliorated pathophysiological changes in the mdx model, providing the proof-of-principle validation for targeting clock components for dystrophic disease application (30). In line with these findings, activation of clock by CHX in myoblasts resulted in enhanced differentiation and this effect is dependent on a functional clock. Additional effects of CHX on promoting proliferation and migration of myoblasts further implicate its potential utility in regenerative repair to ameliorate dystrophic disease.

### REFERENCES FOR EXAMPLE 1

1. Partch, C. L., Green, C. B. & Takahashi, J. S. Trends Cell Biol 24, 90-99 (2014). 2. Stenvers, D. J., Scheer, F., Schrauwen, P., la Fleur, S. E. & Kalsbeek, A. Nat Rev Endocrinol 15, 75-89 (2019). 3. Bass, J. & Takahashi, J. S. Science (New York, N.Y 330, 1349-1354 (2010). 4. He, B. & Chen, Z. Curr Drug Metab 17, 503-512 (2016). 5. Takahashi, J. S. Nature reviews. Genetics 18, 164-179 (2017). 6. Huang, N., Chelliah, Y., Shan, Y., Taylor, C. A., Yoo, S. H., Partch, C., Green, C. B., Zhang, H. & Takahashi, J. S. Science (New York, N. Y 337, 189-194 (2012). 7. Preitner, N., Damiola, F., Lopez-Molina, L., Zakany, J., Duboule, D., Albrecht, U. & Schibler, U. Cell 110, 251-260 (2002). 8. Stratmann, M. & Schibler, U. Cell metabolism 15, 791-793 (2012). 9. Chatterjee, S. & Ma, K. F1000Research 5, 1549 (2016). 10. Andrews, J. L., Zhang, X., McCarthy, J. J., McDearmon, E. L., Hornberger, T. A., Russell, B., Campbell, K. S., Arbogast, S., Reid, M. B., Walker, J. R., Hogenesch, J. B., Takahashi, J. S. & Esser, K. A. Proceedings of the National Academy of Sciences of the United States of America 107, 19090-19095 (2010). 11. Chatterjee, S., Nam, D., Guo, B., Kim, J. M., Winnier, G. E., Lee, J., Berdeaux, R., Yechoor, V. K. & Ma, K. Journal of cell science 126, 2213-2224 (2013). 12. Chatterjee, S., Yin, H., Li, W., Lee, J., Yechoor, V. K. & Ma, K. Scientific reports 9, 4585 (2019). 13. Chatterjee, S., Yin, H., Nam, D., Li, Y. & Ma, K. Experimental cell research (2014). 14. Katoku-Kikyo, N., Paatela, E., Houtz, D. L., Lee, B., Munson, D., Wang, X., Hussein, M., Bhatia, J., Lim, S., Yuan, C., Asakura, Y., Asakura, A. & Kikyo, N. J Cell Biol 220(2021). 15. Lowe, M., Lage, J., Paatela, E., Munson, D., Hostager, R., Yuan, C., Katoku-Kikyo, N., Ruiz-Estevez, M., Asakura, Y., Staats, J., Qahar, M., Lohman, M., Asakura, A. & Kikyo, N. Cell reports 22, 2118-2132 (2018). 16. Guo, B., Chatterjee, S., Li, L., Kim, J. M., Lee, J., Yechoor, V. K., Minze, L. J., Hsueh, W. & Ma, K. FASEB journal: official publication of the Federation of American Societies for Experimental Biology 26, 3453-3463 (2012). 17. Yin, H., Li, W., Chatterjee, S., Xiong, X., Saha, P., Yechoor, V. & Ma, K. FASEB journal: official publication of the Federation of American Societies for Experimental Biology 34, 6613-6627 (2020). 18. He, B., Nohara, K., Park, N., Park, Y. S., Guillory, B., Zhao, Z., Garcia, J. M., Koike, N., Lee, C. C., Takahashi, J. S., Yoo, S. H. & Chen, Z. Cell metabolism 23, 610-621 (2016). 19. Nohara, K., Mallampalli, V., Nemkov, T., Wirianto, M., Yang, J., Ye, Y., Sun, Y., Han, L., Esser, K. A., Mileykovskaya, E., D'Alessandro, A., Green, C. B., Takahashi, J. S., Dowhan, W., Yoo, S. H. & Chen, Z. Nat Commun 10, 3923 (2019). 20. Su, R., Dong, L., Li, Y., Gao, M., Han, L., Wunderlich, M., Deng, X., Li, H., Huang, Y., Gao, L., Li, C., Zhao, Z., Robinson, S., Tan, B., Qing, Y., Qin, X., Prince, E., Xie, J., Qin, H., Li, W., Shen, C., Sun, J., Kulkarni, P., Weng, H., Huang, H., Chen, Z., Zhang, B., Wu, X., Olsen, M. J., Muschen, M., Marcucci, G., Salgia, R., Li, L., Fathi, A. T., Li, Z., Mulloy, J. C., Wei, M., Home, D. & Chen, J. Cancer Cell 38, 79-96 eli (2020). 21. Gao, H., Xiong, X., Lin, Y., Chatterjee, S. & Ma, K. Experimental cell research 397, 112348 (2020). 22. Charge, S. B. & Rudnicki, M. A. Physiological reviews 84, 209-238 (2004). 23. Kuang, S. & Rudnicki, M. A. Trends Mol Med 14, 82-91 (2008). 24. Rudnicki, M. A., Le Grand, F., McKinnell, I. & Kuang, S. Cold Spring Harb Symp Quant Biol 73, 323-331 (2008). 25. Fu, L. & Kettner, N. M. Prog Mol Biol Transl Sci 119, 221-282 (2013). 26. Sancar, A. & Van Gelder, R. N. Science (New York, N.Y 371 (2021). 27. Solt, L. A., Kumar, N., Nuhant, P., Wang, Y., Lauer, J. L., Liu, J., Istrate, M. A., Kamenecka, T. M., Roush, W. R., Vidovic, D., Schurer, S. C., Xu, J., Wagoner, G., Drew, P. D., Griffin, P. R. & Burris, T. P. Nature 472, 491-494 (2011). 28. Chatterjee, S., Yin, H., Nam, D., Li, Y. & Ma, K. Experimental cell research 331, 200-210 (2015). 29. Xiong, X., Gao, H., Lin, Y., Yechoor, V. & Ma, K. Experimental cell research 406, 112766 (2021). 30. Welch, R. D., Billon, C., Valfort, A. C., Burris, T. P. & Flaveny, C. A. Scientific reports 7, 17142 (2017).

### Example 2: The Clock Modulatory Activities of Chlorhexidine and its Analogs and Effects on Myogenesis

Using a high through-put virtual screening pipeline coupled with biochemical validations of clock-modulatory activity, we initially identified chlorhexidine (CHX) as a circadian clock activator with pro-myogenic activities in C2C12 myoblasts. Further analysis using Bmal1-deficient cellular models revealed loss of this activity, indicating that the effect of on promoting myogenesis is clock-dependent. We have further investigated the effect of CHX on inducing myogenesis in primary myoblasts isolated from the dystrophin-deficient mdx mice, a pre-clinical model for Duchene Muscular Dystrophy (DMD). Furthermore, initial in vivo testing of CHX via direct muscular injection into mdx dystrophic TA muscle revealed robust effect on induction of myogenic activation with enhanced nascent myofiber formation and satellite cell pool size.

In addition, based on the CHX chemical scaffold, we have synthesized analogs of CHX for analysis of their clock-modulatory activities with myogenic assays. Among these molecules we tested to date, three compounds were identified as clock activators and four compounds displayed inhibitory activity. In particular, CM002 demonstrated enhanced pro-myogenic activities in C2C12 myoblasts and mdx primary myoblasts. Importantly, using human primary myoblasts derived from DMD patients, we obtained results indicating that both CHX and its analog CM002 displayed pro-myogenic efficacy with stronger effect observed for CM002.

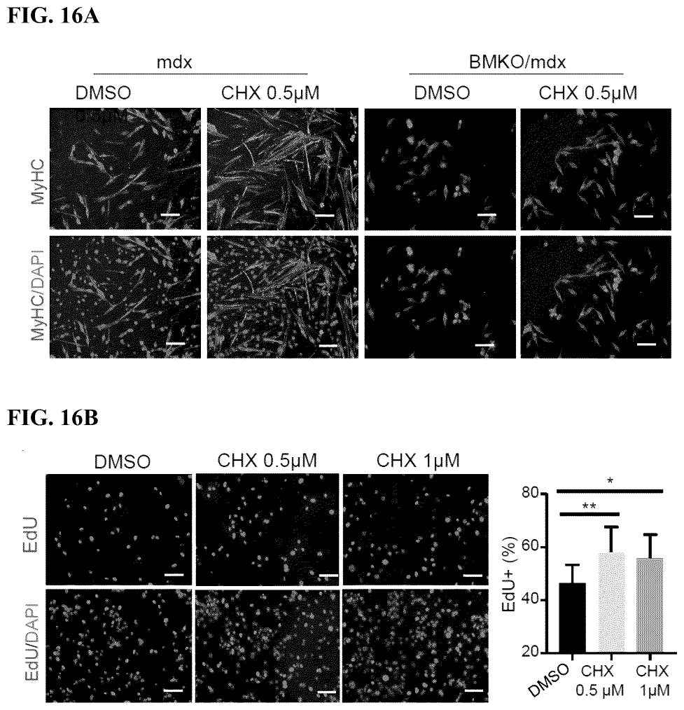

FIGS. 16A-16B. Pro-myogenic effect of chlorhexidine on dystrophin-deficient mdx primary myoblasts. FIG. 16A: We used primary myoblasts isolated from male mdx mice as a cellular model to test the effect of CHX on myogenesis. In cells subjected to myogenic differentiation for 3 days using 2% FBS, immunofluorescence staining of myosin heavy chain (MyHC), a mature myocyte marker, revealed that chlorhexidine treatment led to significantly elevated formation of mature myofibers as compared to DMSO control. Furthermore, this effect was abolished in Bmal1-deficient mdx primary myoblasts isolated from BMKO/mdx double null mice, indicating that the effect of CHX in promoting myogenesis is clock-dependent. FIG. 16B: Using EdU labeling to assess proliferative rate in mdx primary myoblasts, we found that CHX at 0.5-1 μM significantly increased the rate of proliferation by ˜22%. Both of the pro-myogenic and proliferative effects of CHX observed in mdx primary myoblasts were consistent with prior findings from C2C12 myoblasts. Additional validations of these effects are on-going using myoblasts derived from human DMD patients. This pro-myogenic effect of CHX in a cellular model of muscular dystrophy revealed it potential application in dystrophic muscle disease by promoting myogenesis.

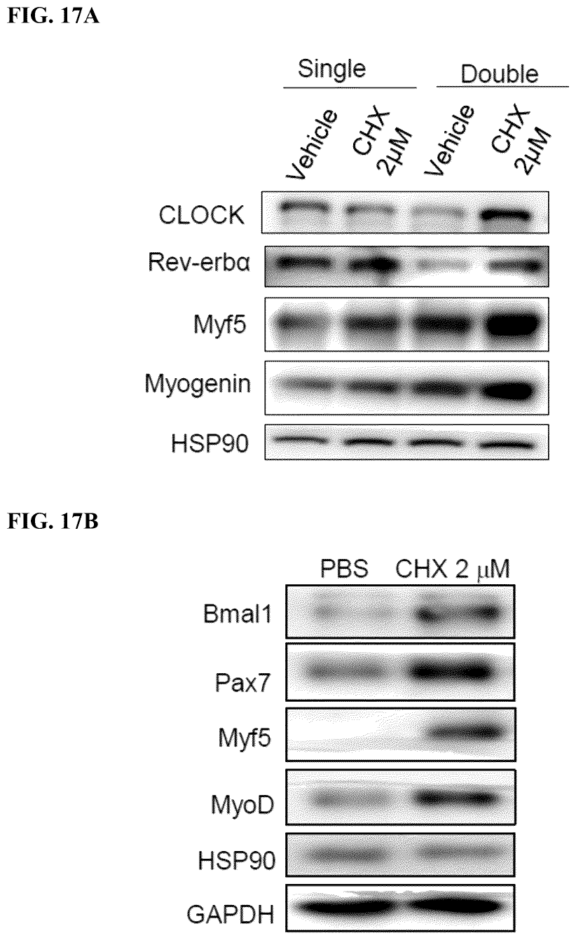

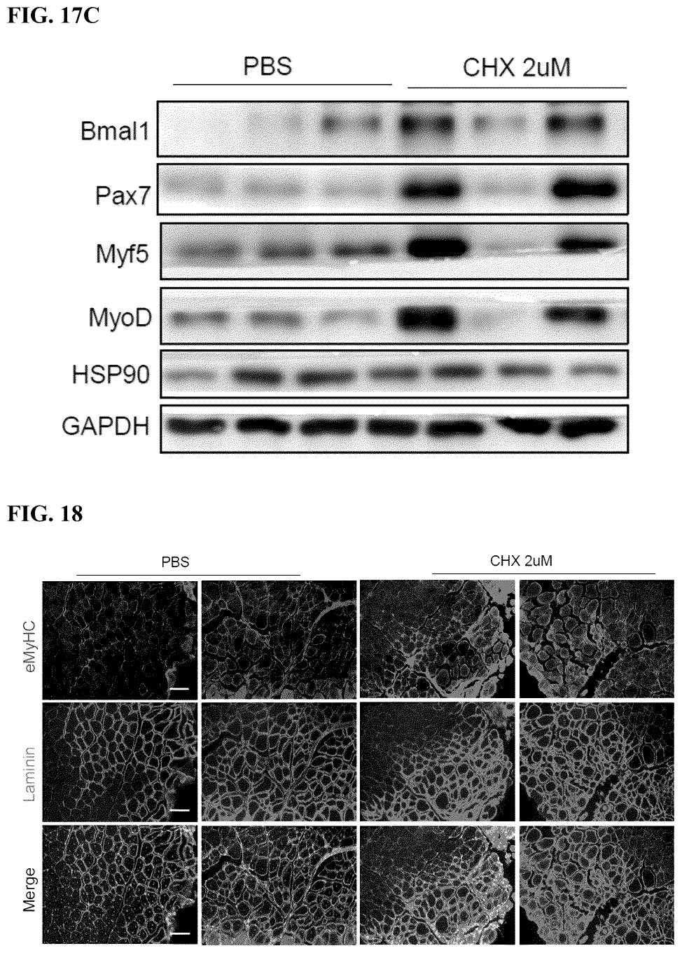

FIGS. 17A-17C. In vivo effect of chlorhexidine on inducing regenerative repair of mdx dystrophic muscle. Based on the effects of CHX on promoting myogenic differentiation, we postulated that it may enhance myogenesis in vivo to promote regenerative repair in dystrophic muscle. To test this, we administered CHX via direct muscular injection (50 μl of 2 μM) into the Tibialis Anterior (TA) muscle in mdx mice. FIG. 17A: Initial preliminary data was obtained from mice injected with one or two injections (one day apart) of CHX dissolved in DMSO. Immunoblot analysis of its effect on protein expression of clock regulators (CLOCK, Rev-erbα) and myogenic factors (Myf5, Myogenin) demonstrated stronger inductions by two doses. FIG. 17B: Next, we further tested the in vivo effect of CHX by three doses using PBS solution to avoid potential membrane damage by DMSO. Pooled samples of 4 mice for control and CHX-treated groups revealed similar effects on inducing myogenic factor expression. Notably, Pax7, a specific marker for muscle stem cells, the satellite cells, was strongly induced by CHX, suggesting potentially increased pool size of satellite cells stimulated by CHX. FIG. 17C: Lastly, we examined the effect of triple CHX injection on clock and myogenic factor proteins in individual and individual mdx muscle. Consistent with results from the pooled sample analysis, two of three mice injected with CHX demonstrated marked inductions of myogenic factors together with up-regulation of Bmal1 protein. Variability observed between individual mice could be due to the in vivo injection. In vivo effect of CHX in augmenting clock genes and its induction of myogenic factor expression in a pre-clinical model of muscular dystrophy revealed it potential utility in dystrophic muscle disease by promoting myogenic repair. Its effect on inducing Pax7 protein suggests increased satellite cell pool that may provide myogenic progenitors to augment repair.

FIG. 18. In vivo effect of chlorhexidine on inducing nascent myofiber regeneration of mdx dystrophic muscle. To directly examine effect of CHX in vivo injection on dystrophic muscle repair, we performed immunofluorescence staining of embryonic myosin heavy chain (eMyHC) to identify nascent regenerated myofiber using frozen TA cross sections from mdx mice following triple injections. Laminin staining was used to delineate basal lamina of myofiber. Compared to PBS-injected controls with modest eMyHC staining, mdx TA muscle receiving CHX displayed robust eMyHC indicative of newly-regenerated myofibers. In vivo administration of CHX in dystrophic muscle led to robust induction of nascent myofiber formation, suggesting enhanced regenerative repair.

TABLE 1
Summary of clock-modulatory activities of CHX analogs screened
using U2OS cells containing Per2::dLuc luciferase reporter.

| | LumiCycle Result: Clock | | Modulatory |
|---|---|---|---|
| Drug | activity | Myogenesis | Property |
| CM001 | Period: NS | No effect | n/a |
| | Amplitude: No effect | | |
| CM002 | Period: Shortening | Enhanced | Activator |
| | Amplitude: Increased | | |
| CM003 | No effect | n/a | n/a |
| CM003-1 | Period: Shortening 0.5-2 μM | Enhanced | Activator |
| | Amplitude: Increased | | |
| CM004 | Period: Shortening | Enhanced | Activator |
| | Amplitude: Increased | | |
| CM005 | Period: Lengthening | Slight inhition | Inhibitor |
| | Amplitude: Increased at low | | |
| | concentration | | |
| CM006-1 | No effect | n/a | n/a |
| CM007 | No effect | n/a | n/a |
| CM008 | Cellular toxicity | n/a | n/a |
| CM008-1 | No effect | n/a | n/a |
| CM009C | Period: Dose-dependent | Inhibition | Inhibitor |
| | lengthening | | |
| | Amplitude: Dose-dependent | | |
| | reduction | | |
| CM10I | Period: Dose-dependent | Not tested | Inhibitor |
| | lengthening | | |
| | Amplitude: Dose-dependent | | |
| | reduction | | |
| CM10B | Period: Dose-dependent | Not tested | Inhibitor |
| | lengthening | | |
| | Amplitude: Dose-dependent | | |
| | reduction | | |

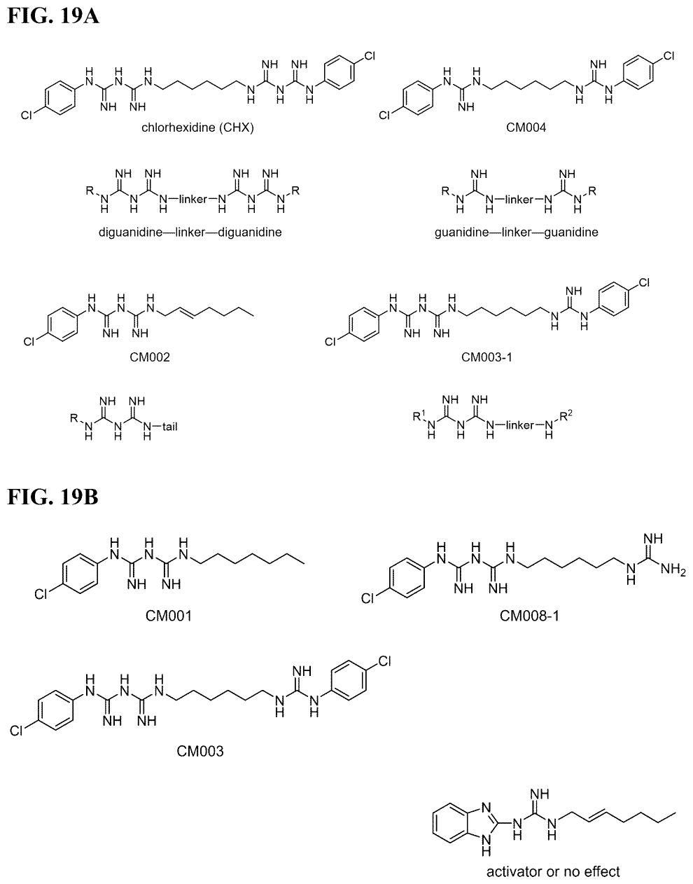

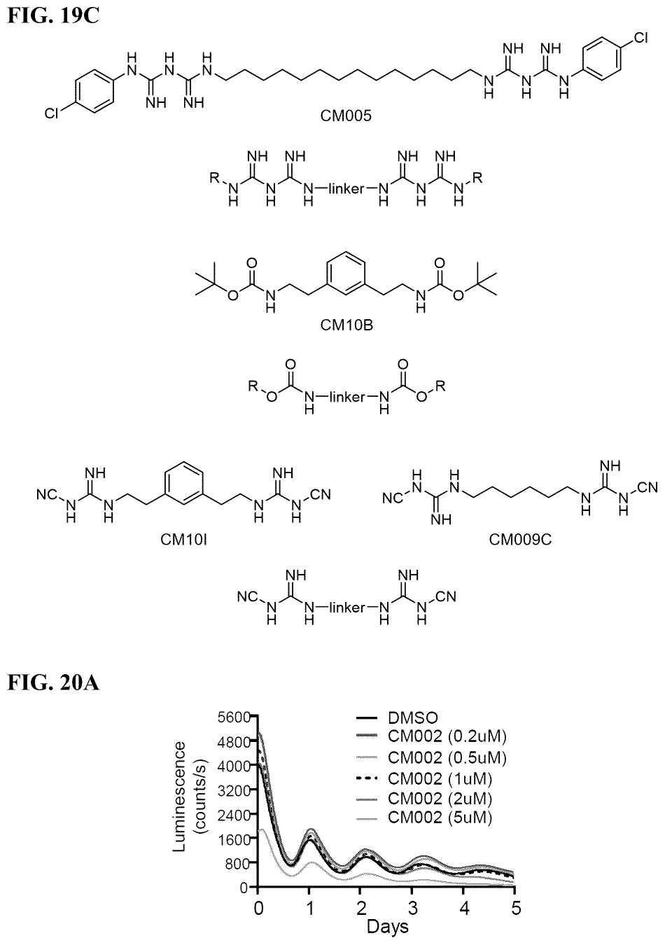

FIGS. 19A-19C. Structure-activity relationship analysis of new CHX analogs. FIG. 19A: Clock-activating compounds in assays tested. FIG. 19B: Compounds without significant effect on clock modulation in assays tested. FIG. 19C: Clock-inhibitory compounds in assays tested.

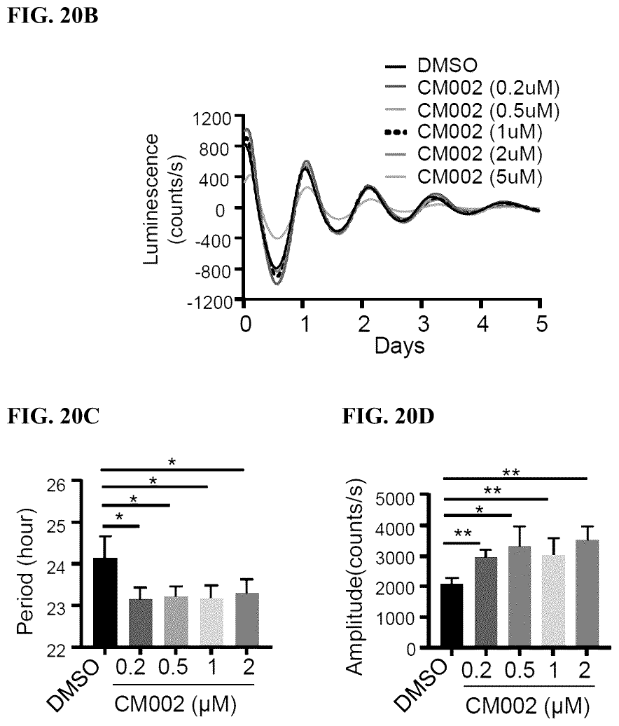

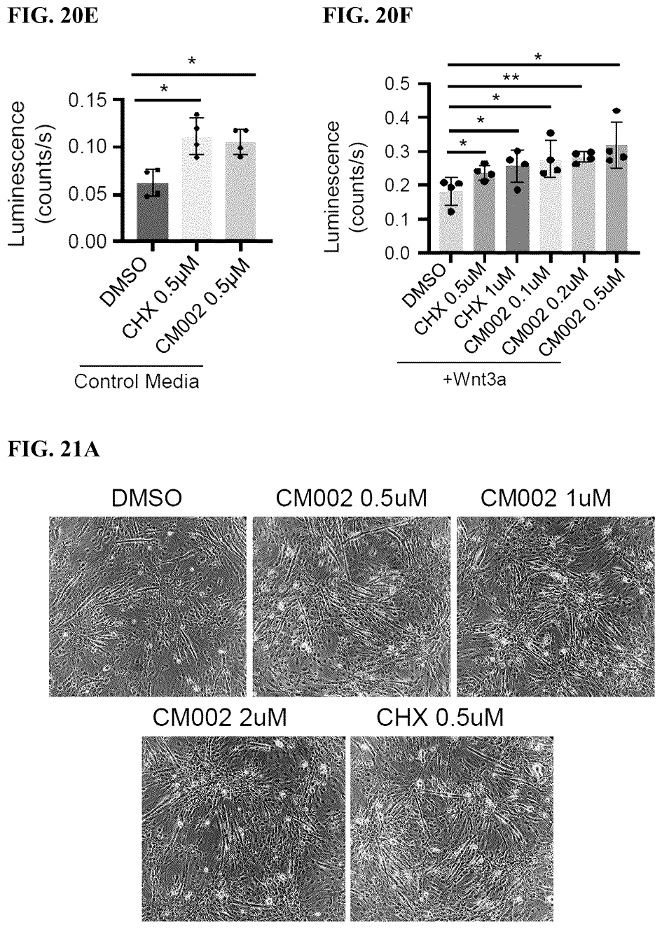

FIGS. 20A-20F. Effect of CM002 on activating circadian clock function. Among 13 new CHX analogs we screened, CM002 demonstrated the strongest effect on activating circadian clock function. We tested the effect of CM002 from 0.2-5 μM on clock modulation in U2O2 cells containing Per2::dLuc reporter using Lumicycle by monitoring chemiluminescence recordings for 6 days. The results were presented using the average original luminescence (FIG. 20A) or baseline-adjusted presentation (FIG. 20B). Quantitative analysis revealed a strong effect of CM002 on shortening of period length starting at 0.2 μM that was maintained at 2 μM (FIG. 20C). A dose-dependent effect of CM002 on increasing clock cycling amplitude was observed at 0.2-2 μM. A reduction of amplitude was found at 5 μM, potentially due to toxicity at this high concentration (FIG. 20D). Based on the known clock effect on stimulating Wnt signaling activity, we used a TCF4 promoter-driven TOPFlash luciferase reporter to determine the effect of CM002 on Wnt pathway and compared this activity with CHX. As shown in FIG. 20E, both CHX and CM002 augmented Wnt activity at basal condition without Wnt stimulation. Notably, when stimulated by a Wnt3a-containing media, TOPFlash luciferase reporter activity was significantly induced by both molecules, with the effect of 0.1 μM of CM002 comparable to that of CHX induction at 1 μM, suggesting stronger effect of CM002 in stimulating clock-controlled Wnt signaling activity. CM002 displayed clock-activating efficacy in luciferase reporter assays with stronger activity than CHX in stimulating Wnt signaling activity.

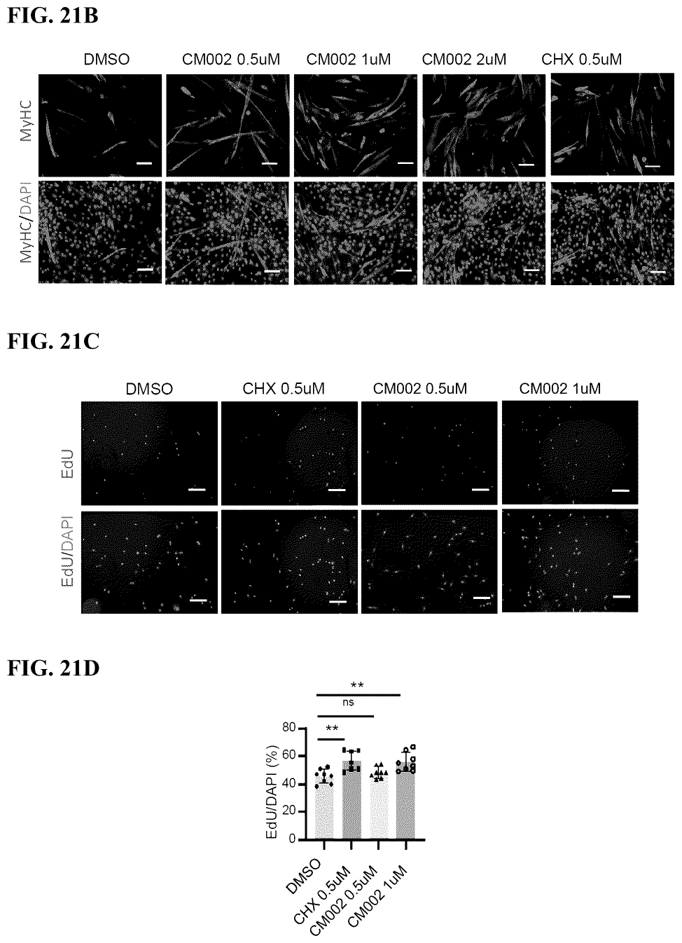

FIGS. 21A-21D. CM002 displayed enhanced pro-myogenic activity than CHX. Using the myogenesis assay, we determined the effect of CM002 on inducing differentiation of C2C12 myoblasts. FIG. 21A: Analysis of morphological progression to mature myotubes at day 7 of differentiation of C2C12 myoblasts revealed a similar or enhanced effect of CM002 at 0.5 μM than CHX. FIG. 21B: Analysis of differentiation via MyHC immunofluorescence staining further demonstrated enhanced differentiation induced by 0.5 μM CM002 than CHX with strongest effect on promoting myotube formation observed at CM002 at 1 μM. FIG. 21C: EdU incorporation significantly increased proliferation of C2C12 myoblasts treated at indicated concentrations of CM002 for 4 hours at 0.5 and 1 μM. This effect was moderately lower than that of CHX at 0.5 μM. Consistent with its stronger efficacy in stimulating Wnt signaling, CM002 displayed stronger activity in inducing myogenic differentiation in C2C12 myoblasts than that of CHX. Interestingly, the effect of CM002 on stimulating proliferation was not as robust as CHX.

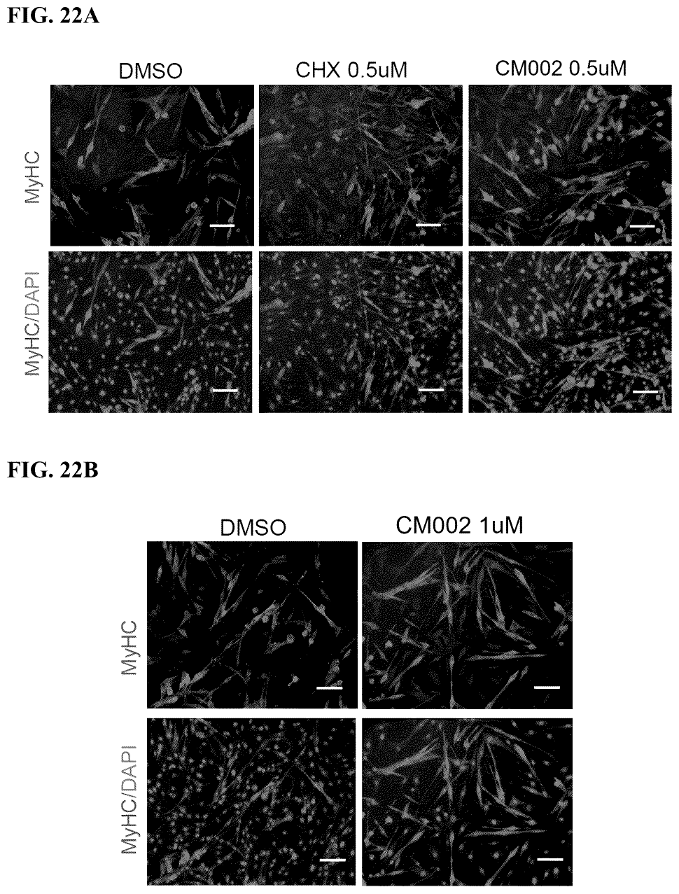

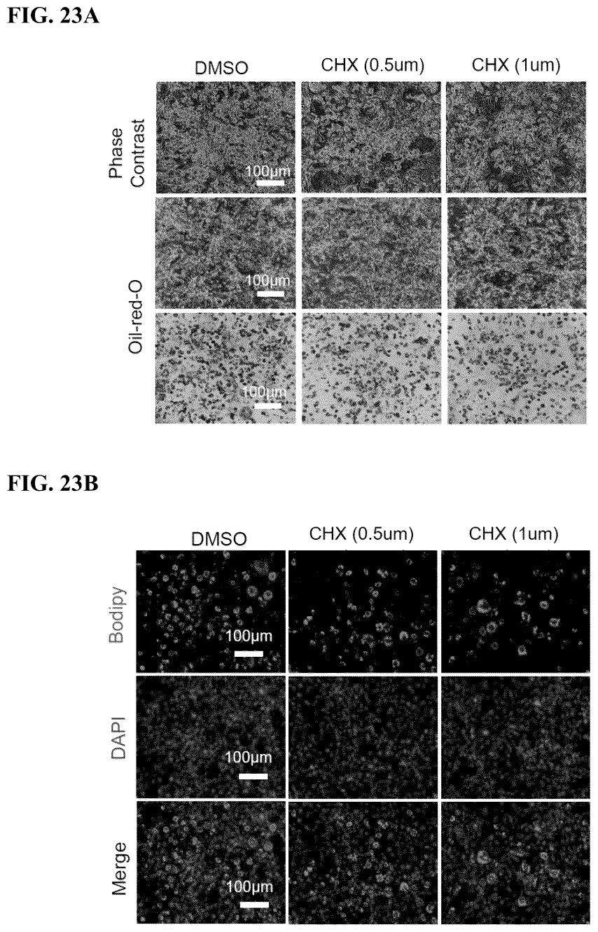

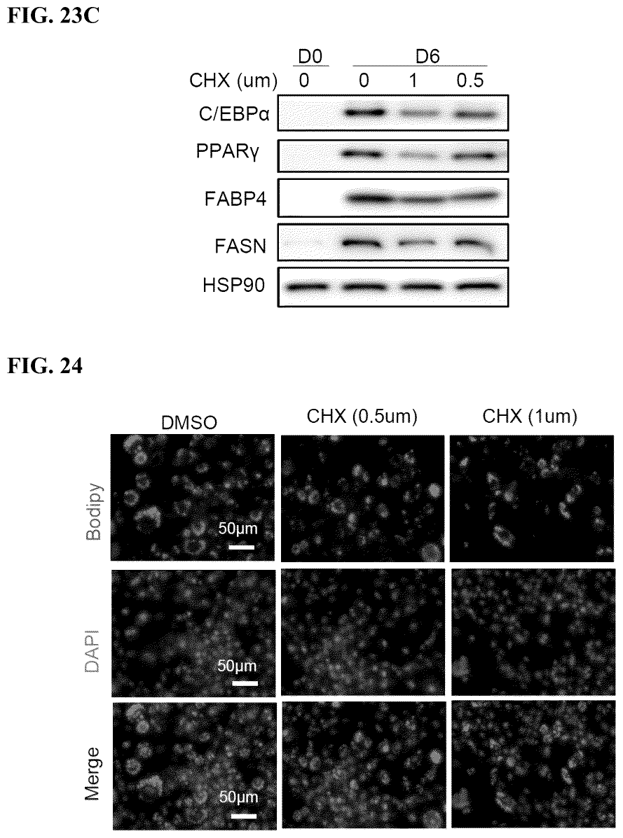

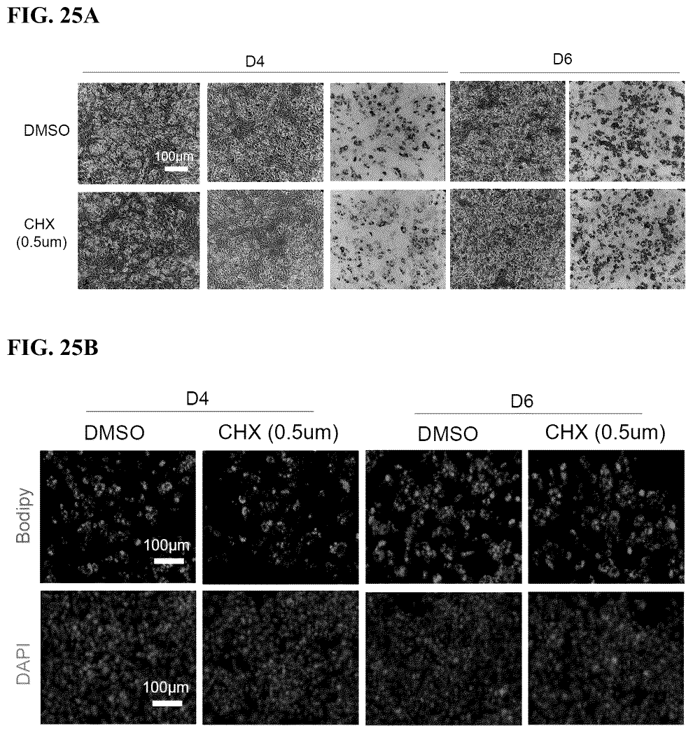

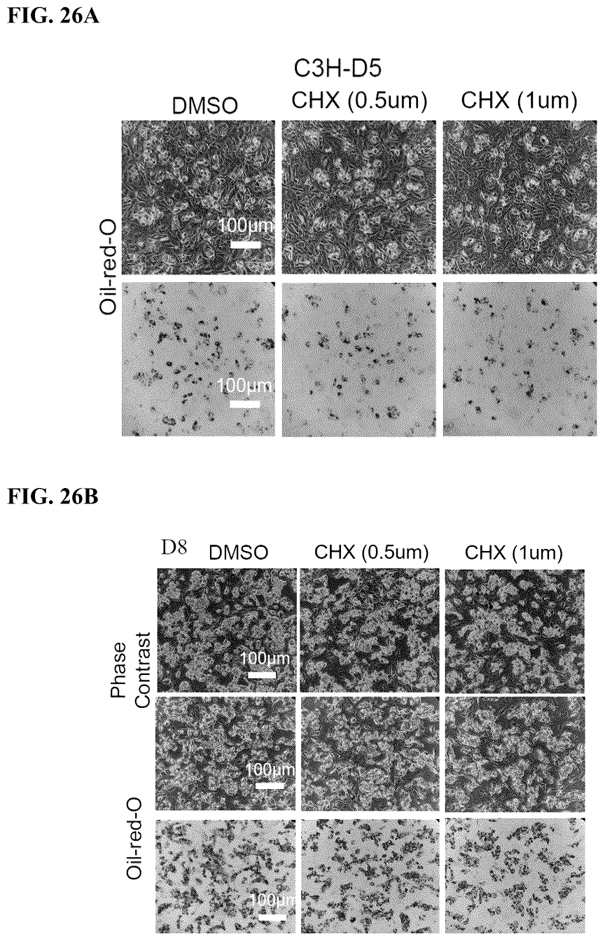

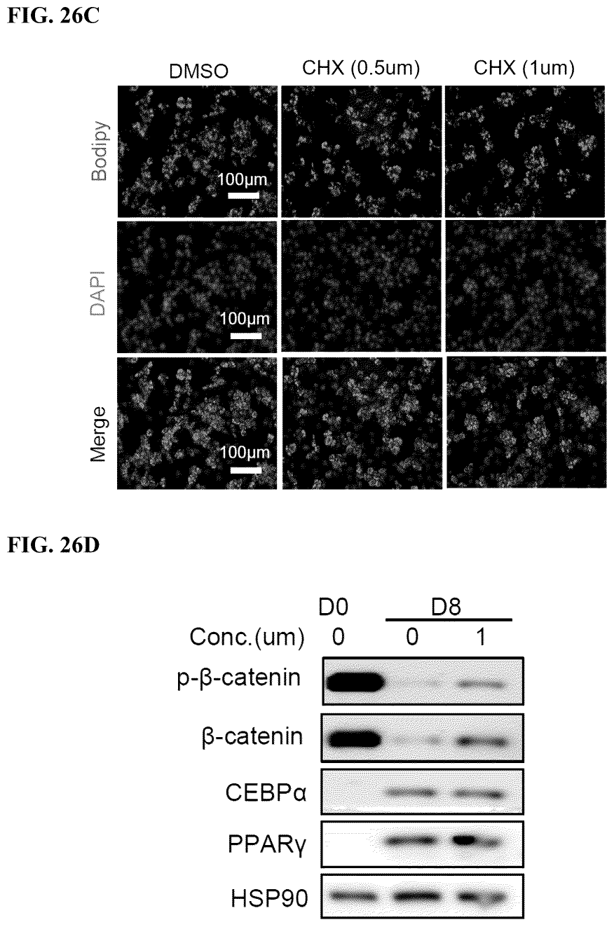

FIGS. 22A-22B. CM002 displays pro-myogenic activity in mdx primary myoblasts. Using primary myoblasts isolated from mdx mice, we determined CM002 effect on myogenesis in this dystrophic disease cellular model. FIG. 22A: In mdx primary myoblasts at day 3 of myogenic differentiation, MyHC immunofluorescence staining revealed significantly enhanced mature myocyte formation induced by 0.5 μM CM002 treatment. Similar as shown in C2C12 myoblasts, this effect was stronger than that of CHX. FIG. 22B: Furthermore, 1 μM of CM002 treatment in mdx primary myoblasts seeded at low density revealed more advanced myotube fusion than that of 0.5 μM, suggesting a dose-dependent effect of CM002 on myogenic differentiation in dystrophin-deficient myoblasts. CM002 displayed stronger activity in stimulating myofiber maturation in mdx primary myoblasts than CHX, with a dose-dependent effect on myogenic fusion process.

### Example 3: Anti-Adipogenic Properties of Clock Activators

Circadian clock exerts temporal control in metabolism, and disruption of circadian regulation leads to the development of obesity. It was previously demonstrated that circadian clock components exert coordinated regulation of adipogenesis that impacts adipose tissue development in vivo. Here we report, inter alia, that a clock activator, chlorhexidine, displays anti-adipogenic activities via clock-controlled Wnt signaling pathway. Chlorhexidine shortened clock period length with induction of core clock components in adipogenic progenitors, indicative of clock activation. Consistent with its clock-activating property, chlorhexidine robustly suppressed the lineage commitment and maturation of adipogenic mesenchymal precursor cells, with similar effects on inhibiting the terminal differentiation of preadipocyte cell line and primary preadipocytes. Mechanistically, we show that chlorhexidine exerts transcriptional modulation of key components of Wnt pathway resulting in activation of Wnt signaling activity. Via modification of its chemical scaffold, we generated chlorhexidine analogs and identified CM002 as a clock-activating molecule with improved anti-adipogenic activity that is dependent on clock modulation. Collectively, our findings demonstrated the anti-adipogenic efficacies of clock-activating molecules chlorhexidine and CM002, implicating their potential application in countering obesity and associated metabolic consequences.

Circadian rhythm, daily oscillations in behavior and physiology, exert pervasive temporal regulation in stem cell behavior and key metabolic processes (Takahashi, 2017; Bass and Takahashi, 2010). Disruption of this timing mechanism, increasingly prevalent in our modern lifestyle, predisposes to the development of metabolic disorders, particularly the incidence of obesity and Type II Diabetes (Bass and Takahashi, 2010; Stenvers, Scheer, Schrauwen et al., 2019; Pan, Schernhammer, Sun et al., 2011; Fonken, Workman, Walton et al., 2010). Potential therapeutic targeting to modulate clock and its biological output pathways may have therapeutic applications for metabolic diseases (Sulli, Manoogian, Taub et al., 2018; Cederroth, Albrecht, Bass et al., 2019).

Circadian temporal control has been implicated in modulating distinct stem cell properties in various tissue compartments (Janich, Pascual, Merlos-Suarez et al., 2011; Janich, Toufighi, Solanas et al., 2013). Notably, clock output pathways were altered in pathological conditions such as obesity and aging. Significant dampening of clock oscillation amplitude occurs with nutritional overload in high-fat diet-induced obesity, and clock disruption could be synergistic in inducing diabetes with the diet challenge (Qian, Yeh, Rakshit et al., 2015; Kohsaka, Laposky, Ramsey et al., 2007). In aged stem cells, clock-controlled pathways were shifted toward responding to stress from growth and developmental processes (Solanas, Peixoto, Perdiguero et al., 2017). In adipogenic progenitors, clock function is required to suppress adipogenesis with its loss promoting development of obesity (Paschos, Ibrahim, Song et al., 2012; Guo, Chatterjee, Li et al., 2012; Turek, Joshu, Kohsaka et al., 2005). It is thus conceivable that pharmacological targeting to maintain or re-enforce clock oscillation may provide new avenues for anti-obesity therapies.

Circadian clock is composed of a transcriptional/translational feed-back loop (Takahashi, 2017) that generates daily oscillations in metabolic processes. Transcription activators CLOCK (Circadian Locomotor Output Cycles Kaput) and Bmal1 initiates clock transcription that are countered by the feedback inhibition of their direct target genes, Periods (Per 1-3) and Cryptochromes (Cry1& 2). Interestingly, current findings suggest both the positive and negative arms of the core clock feedback loop modulates adipogenic process. Either loss of CLOCK or Bmal1 resulted in obesity (Paschos, Ibrahim, Song et al., 2012; Guo, Chatterjee, Li et al., 2012; Turek, Joshu, Kohsaka et al., 2005), while PER2 directly represses PPARγ to inhibit adipocyte development (Grimaldi, Bellet, Katada et al., 2010). Notably, activation of a clock repressor Rev-erbα, a direct target of CLOCK/Bmal1, by specific agonists demonstrated anti-obesity efficacy in vivo with improvement of dyslipidemia (Solt, Wang, Banerjee et al., 2012). In addition, activation of RORα by a natural ligand agonist, Nobiletin, can inhibit adipogenesis with in vivo effects in countering obesity (Xiong, Kiperman, Li et al., 2023). Thus, targeting the clock modulation in adipocyte development may provide novel interventional strategies for obesity and related metabolic consequences.

Chlorhexidine (CHX) was identified as a clock activator through a high throughput screening and demonstrated its pro-myogenic efficacy (Kiperman, Li, Xiong et al., 2023). Here we show that CHX displayed strong activity in inhibiting adipogenic differentiation in multiple adipogenic progenitor cell models, and a new analog CM002 with clock-activating properties has improved anti-adipogenic effects.

### Materials & Methods

### Animal Studies

Cell culture and adipogenic differentiation. 3T3-L1 and C3H10T1/2 cell lines were obtained from ATCC, and maintained in DMEM with 10% fetal bovine serum supplemented with 1% Penicillin-Streptomycin-Glutamine, as previously described (Nam, Guo, Chatterjee et al., 2015; Liu, Xiong, Nam et al., 2020). 0.25% Trypsin was used for digestion and passaging of these cell lines. For adipogenic differentiation induction media (1.6 μM insulin, 1 μM dexamethasone, 0.5 mM IBMX, 0.5 uM Rosiglitazone) was used for 3 days followed by maintenance medium with insulin for 3 days for 3T3-L1 and 5 days for C3H10T1/2 cells, as previously described (Liu et al., 2020).

Generation of stable adipogenic progenitor cell lines containing Per2::dLuc. 3T3-L1 preadipocytes and the C3H10T1/2 mesenchymal stem cells obtained from ATCC were used for Per2:: dLuc lentiviral transduction and stable clone selection using puromycin, as described previously (Guo et al., 2012). Briefly, cells were transfected with lentiviral packaging plasmids (pSPAX.2 and pMD2.G) and lentivirus vectors Per2::dLuc using PEI Max (Polysciences). At 48 hours post-transfection, lentiviruses were collected. 3T3-L1 and C3H10T1/2 U2OS cells were infected using collected lentiviral media supplemented with polybrene. 24 hours following lentiviral infection, stable cell lines were selected in the presence of 2 μg/ml puromycin.

Primary preadipocyte isolation and adipogenic induction. The stromal vascular fraction containing preadipocytes were isolated from subcutaneous fat pads, as described (Chatterjee, Nam, Guo et al., 2013). Briefly, fat pads were cut into pieces and digested using 0.1% collagenase Type 1 with 0.8% BSA at 37° C. in a horizontal shaker for 60 minutes, passed through Nylon mesh and centrifuged to collect the pellet containing the stromal vascular fraction with preadipocytes. Preadipocytes were cultured in F12/DMEM supplemented with bFGF (2.5 ng/ml), expanded for two passages and subjected to differentiation in 6-well plates at 90% confluency. Adipogenic differentiation was induced for 2 days in medium containing 10% FBS, 1.6 μM insulin, 1 μM dexamethasone, 0.5 mM IBMX, 0.5 μM rosiglitazone before switching to maintenance medium for 4 days with insulin only. Nobiletin at indicated concentrations were added for the entire differentiation time course.

Oil-red-O and Bodipy staining. These stainings for neutral lipids during adipogenic differentiation were performed as previously described (Nam et al., 2015). Briefly, for oil-red-O staining, cells were fixed using 10% formalin and incubated using 0.5% oil-red-O solution for 1 hour. Bodipy 493/503 was used at 1 mg/L together with DAPI for 15 minutes, following 4% paraformaldehyde fixation and permeabilization with 0.2% triton-X100.

Continuous Bioluminescence monitoring of Per2:: dLuc luciferase reporter. 3T3-L1 and C3H10T1/2 cells containing a Per2::dLuc luciferase reporter were generated as described previously (Xiong et al., 2023). For bioluminescence recording, cells were seeded at 4×10⁵ density on 24 well plates, and used at 90% confluence following overnight culture with explant medium luciferase recording media. Explant medium contains DMEM buffer stock, 10% FBS, 1% PSG, pH7 1M HEPES, 7.5% Sodium Bicarbonate, Sodium Hydroxide (100 mM) and XenoLight D-Luciferin Bioluminiscent Substrate (100 mM). Raw and subtracted results of real-time bioluminescence recording data for 6 days were exported, and data was calculated as luminescence counts per second, as previously described (Xiong, Li, Nam et al., 2022). LumiCycle Analysis Program (Actimetrics) was used to determine clock oscillation period, length amplitude and phase. Briefly, raw data following the first cycle from day 2 to day were fitted to a linear baseline, and the baseline-subtracted data (polynomial number=1) were fitted to a sine wave, from which period length and goodness of fit and damping constant were determined. For samples that showed persistent rhythms, goodness-of-fit of >80% was usually achieved.

TOPFlash luciferase reporter assay. M50 Super 8×TOPFlash luciferase reporter containing Wnt-responsive TCF bindings sites was a gift from Randall Moon (Veeman, Slusarski, Kaykas et al., 2003) obtained from Addgene (Addgene plasmid #12456). Cells were seeded and grown overnight, and cell transfection was performed at 90% confluency. 24 hours following transfection, cells were treated using 10% Wnt3a conditioned media obtained from L Wnt-3A cell line (ATCC CRL-2647) to induce luciferase activity. Luciferase activity was assayed using Dual-Luciferase Reporter Assay Kit (Promega) in 96-well black plates. TOPFlash luciferase reporter luminescence was measured on microplate reader (TECAN infinite M200pro) and normalized to control FOPFlash activity, as previously described (Guo et al., 2012). The mean and standard deviation values were calculated for each well and graphed.

Immunoblot analysis. Total protein was extracted using lysis buffer containing 3% NaCl, 5% Tris-HCl, 10% Glycerol, 0.5% Triton X-10 in MilliQ water with protease inhibitor cocktail. 20-40 μg of total protein was resolved on 10% SDS-PAGE gels followed by immunoblotting on PVDF membranes (Bio-rad). Membranes were developed by chemiluminescence (SuperSignal West Pico, Pierce Biotechnology) and signals were obtained via a chemiluminescence imager (Amersham Imager 680, GE Biosciences). Primary and secondary antibodies used are listed in Table 2.

TABLE 2
Primary antibodies list.

| Antibody | Source | Cat# | Dilution |
|---|---|---|---|
| C/EBPα | Santa Cruz | SC-61 | 1:1000 |
| PPARγ | Santa Cruz | SC-7273 | 1:1000 |
| FASN | Santa Cruz | SC-48357 | 1:1000 |
| FABP4 | Santa Cruz | SC-271529 | 1:1000 |
| β-catenin | Cell Signaling | 8480S | 1:1000 |
| HSP90 | Cell Signaling | 4874S | 1:3000 |

RNA extraction and RT-qPCR analysis. PureLink RNA Mini Kit (Invitrogen) were used to isolate total RNA from cells. cDNA was generated using Revert Aid RT kit (ThermoFisher) and quantitative PCR was performed using SYBR Green Master Mix (Thermno Fisher) in triplicates on ViiA 7 Real-Time PCR System (Applied Biosystems). Relative expression levels were determined using the comparative Ct method with normalization to 361B4 as internal control. PCR primers sequence are listed in Table 3.

TABLE 3
Primer sequence for qPCR analysis.

| Genes | | Sequences |
|---|---|---|
| Bmal1 | Forward | CGCTTTCTGGAGGGTGTCCGC |
| | | (SEQ ID NO: 3) |
| | Reverse | TGCCAGGACGCGCTTGTACC |
| | | (SEQ ID NO: 4) |
| Clock | Forward | TTGCTCCACGGGAATCCTT |
| | | (SEQ ID NO: 5) |
| | Reverse | GGAGGGAAAGTGCTCTGTTGTAG |
| | | (SEQ ID NO: 6) |
| Nr1d1 | Forward | TGGCATCCGGTGCACTGCAG |
| | | (SEQ ID NO: 7) |
| | Reverse | CCCTCCAGAAGGGTAGCACGCT |
| | | (SEQ ID NO: 8) |
| Nr1d2 | Forward | GGAGTTCATGCTTGTGAAGGCTGT |
| | | (SEQ ID NO: 9) |
| | Reverse | CAGACACTTCTTAAAGCGGCACTG |
| | | (SEQ ID NO: 10) |
| Cry1 | Forward | CTGGCGTGGAAGTCATCGT |
| | | (SEQ ID NO: 11) |
| | Reverse | CTGTCCGCCATTGAGTTCTATG |
| | | (SEQ ID NO: 12) |
| Cry2 | Forward | TGTCCCTTCCTGTGTGGAAGA |
| | | (SEQ ID NO: 13) |
| | Reverse | GCTCCCAGCTTGGCTTGA |
| | | (SEQ ID NO: 14) |
| Per1 | Forward | CTGCCATGGAGGAAGAAGAG |
| | | (SEQ ID NO: 15) |
| | Reverse | AGCTGGGGCAGTTTCCTATT |
| | | (SEQ ID NO: 16) |
| Per2 | Forward | ATGCTCGCCATCCACAAGA |
| | | (SEQ ID NO: 17) |
| | Reverse | GCGGAATCGAATGGGAGAAT |
| | | (SEQ ID NO: 18) |
| Wnt1 | Forward | GGTTTCTACTACGTTGCTACTGG |
| | | (SEQ ID NO: 19) |
| | Reverse | GGAATCCGTCAACAGGTTCGT |
| | | (SEQ ID NO: 20) |
| Wnt10a | Forward | CAACGCGTGCGCTCTGGGTA |
| | | (SEQ ID NO: 21) |
| | Reverse | TGGCTCAAGCCCTTTCCGCG |
| | | (SEQ ID NO: 22) |
| Fzd2 | Forward | CATGCCCAACCTTCTTGGC |
| | | (SEQ ID NO: 23) |
| | Reverse | CAGCGGGTAGAACTGATGCAC |
| | | (SEQ ID NO: 24) |
| Fzd5 | Forward | AATCATGCAGGGGGCCCCGAA |
| | | (SEQ ID NO: 25) |
| | Reverse | CGACAAGCTAGGTACCTGTGGCG |
| | | (SEQ ID NO: 26) |
| Dv12 | Forward | TCAGTTTGCGGGTGTGCGCAG |
| | | (SEQ ID NO: 27) |
| | Reverse | TTCGTCTCGCCTACACCACCG |
| | | (SEQ ID NO: 28) |
| Tcf4 | Forward | GGCCGCAGCGCCTTCTCTTTA |
| | | (SEQ ID NO: 29) |
| | Reverse | ACCATCATTGACTCCCCCGAGG |
| | | (SEQ ID NO: 30) |
| B-catenin | Forward | CGCTTGGCTGAACCATCAC |
| | | (SEQ ID NO: 31) |
| | Reverse | GTTCCGCGTCATCCTGATAGT |
| | | (SEQ ID NO: 32) |
| 36B4 | Forward | CGCTTTCTGGAGGGTGTCCGC |
| | | (SEQ ID NO: 33) |
| | Reverse | TGCCAGGACGCGCTTGTACC |
| | | (SEQ ID NO: 34) |

Statistical analysis. Data are presented as mean±SD. Each experiment was repeated at minimum twice to validate the result. Sample size were indicated for each experiment in figure legends. Two-tailed Student's t-test or One-way ANOVA with post-hoc analysis for multiple comparisons were performed as appropriate as indicated. P<0.05 was considered statistically significant.

### Chlorhexidine Activation of Adipocyte-Intrinsic Circadian Clock

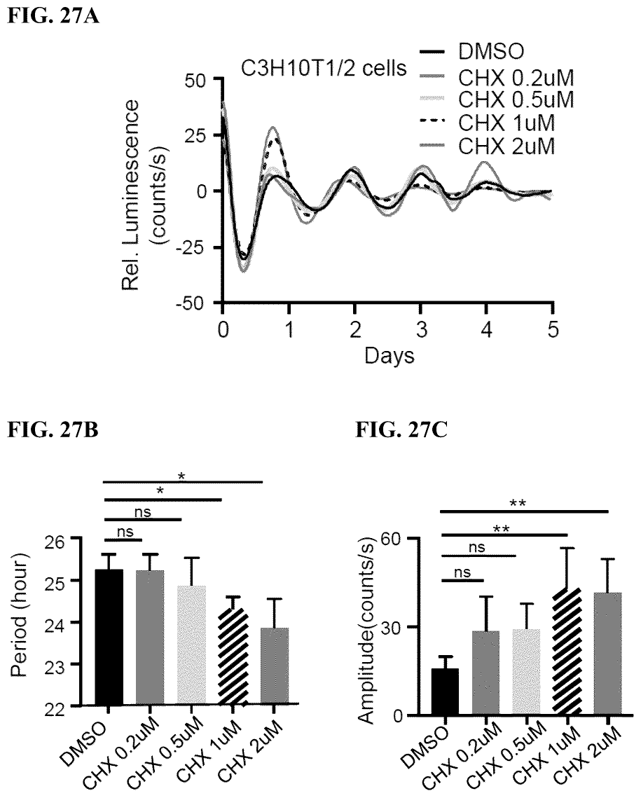

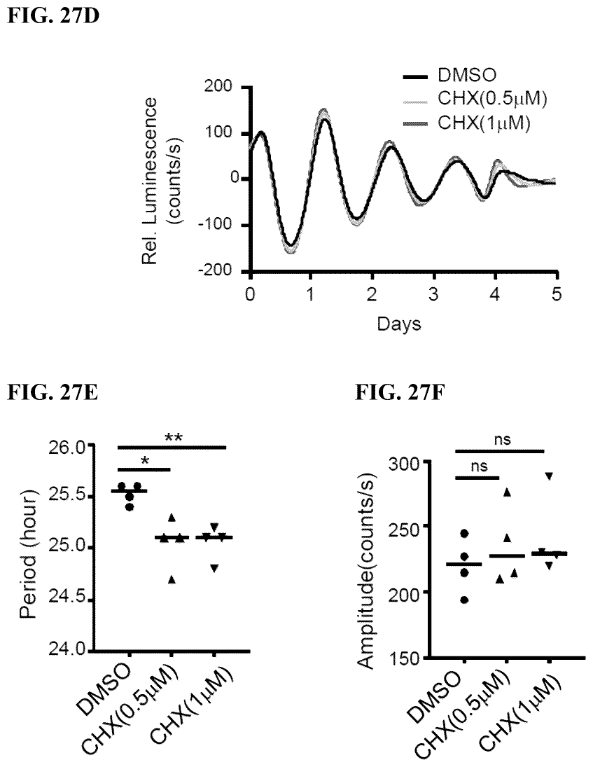

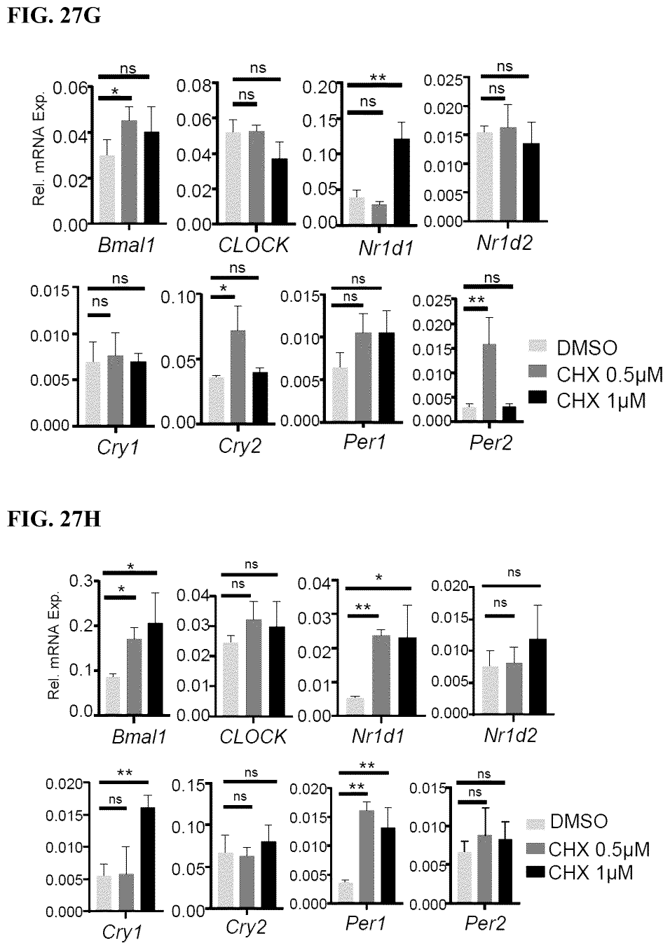

Circadian clock exerts coordinated control in adipogenesis and our previous studies demonstrated that Bmal1, through its transcriptional control of the Wnt signaling pathway genes, inhibits adipocyte development. Based on the activity of CHX that activates CLOCK/Bmal1-mediated transcription, we determined whether it modulates cell-intrinsic clock in distinct adipogenic progenitors. Using the mesenchymal precursor C3H10T1/2 and 3T3-L1 preadipocytes as adipogenic progenitor models, we generated stable lines containing Per2:: dLuc luciferase reporter to examine CHX effect on clock properties. As shown in FIG. 27A, CHX treatment of 10T1/2 cells led to a dose-dependent clock period shortening, with this effect significant only for 1 and 2 μM (FIG. 27B). In addition, CHX at these concentrations we able to augment clock cycling amplitude (FIG. 27C), consistent with it clock activator function. These effects of CHX in reducing period length while increasing amplitude is also evident in lineage-committed 3T3-L1 preadipocytes (FIGS. 27D-27F), although not as robust. Further analysis of clock gene regulation by CHX in 10T1/2 cells revealed inductions of Pers and Crys in line with activation of CLOCK/Bmal1-mediated gene transcription (FIG. 27G). Largely similar effects on inducing core clock genes were observed in 3T3-L1 adipocytes (FIG. 27H).

### Chlorhexidine Inhibition of Lineage Commitment of Adipogenic Mesenchymal Progenitor

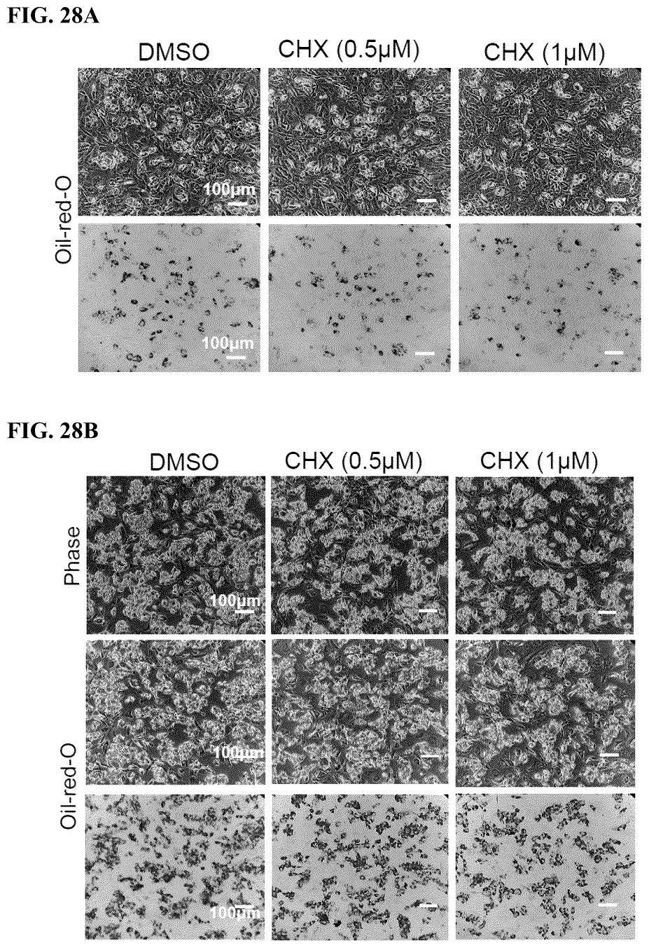

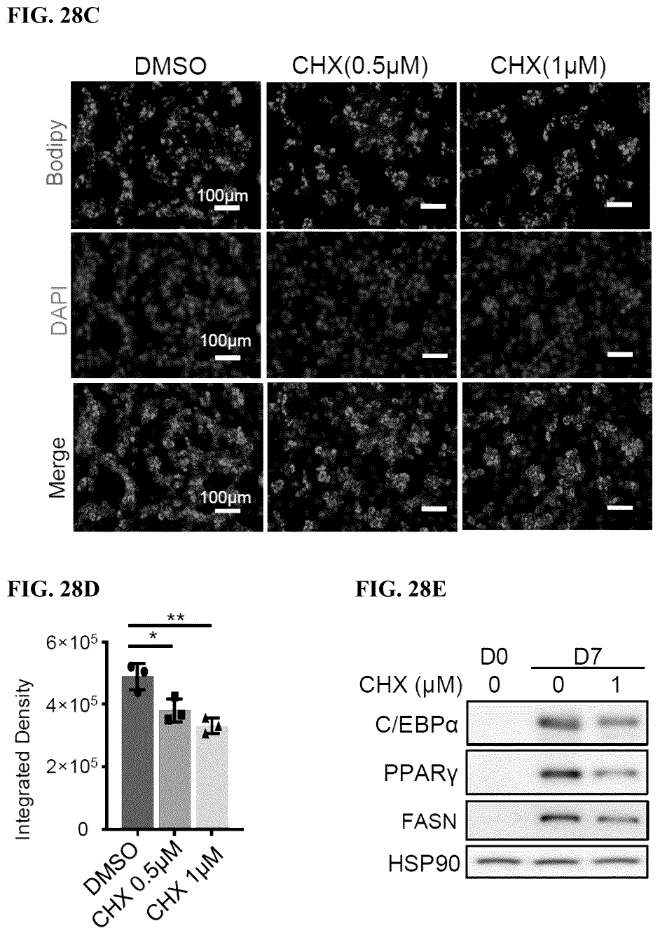

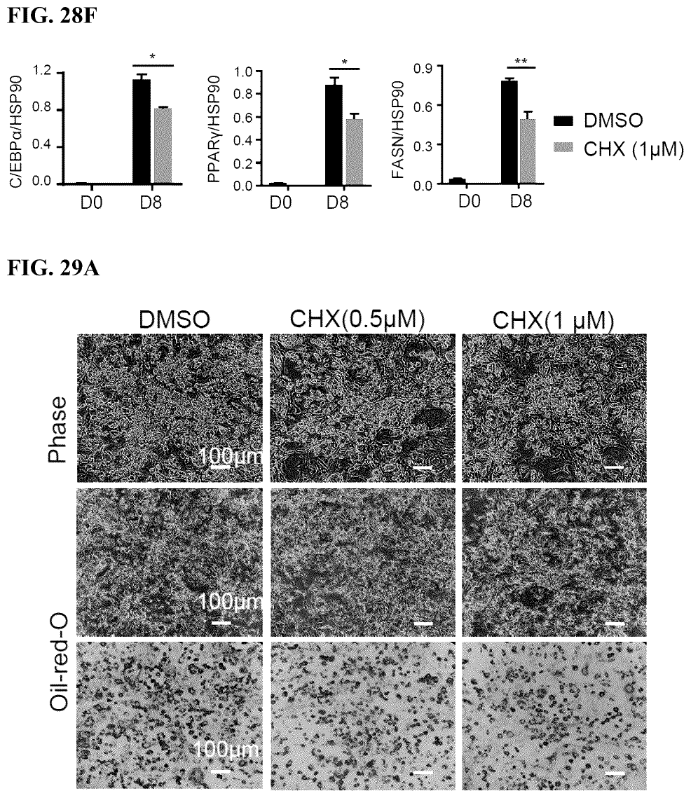

We tested whether CHX activity as a clock activator impacts adipocyte development. Using the 10T1/2 adipogenic mesenchymal precursors, we found that CHX induced a dose-dependent inhibition of adipogenic maturation at the early stage of differentiation 5 days after adipogenic induction, as shown by phase-contrast and oil-red-O staining images of adipocyte morphology (FIG. 28A). At 8 days after differentiation, examination by oil-red-O (FIG. 28B) and Bodipy staining (FIGS. 28C-28D) for lipid accumulation revealed similarly attenuated mature adipocyte formation by CHX treatment. Further analysis of the adipogenic program demonstrated significantly impaired levels of adipogenic factors in day 7-differentiated C3H10T1/2 cells, including CEBP/a and PPARy (FIGS. 28E-28F), with attenuated expression of fatty acid synthase (FASN). These effects of CHX suggest inhibition of the lineage commitment and differentiation program of this adipogenic mesenchymal progenitor cell type.

### Chlorhexidine Inhibition of Preadipocyte Terminal Differentiation

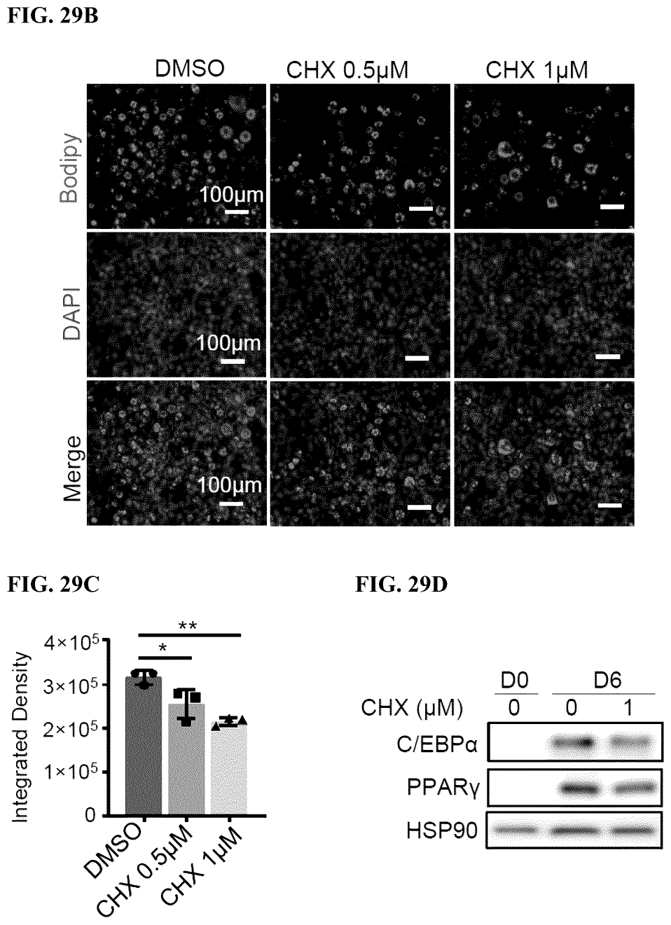

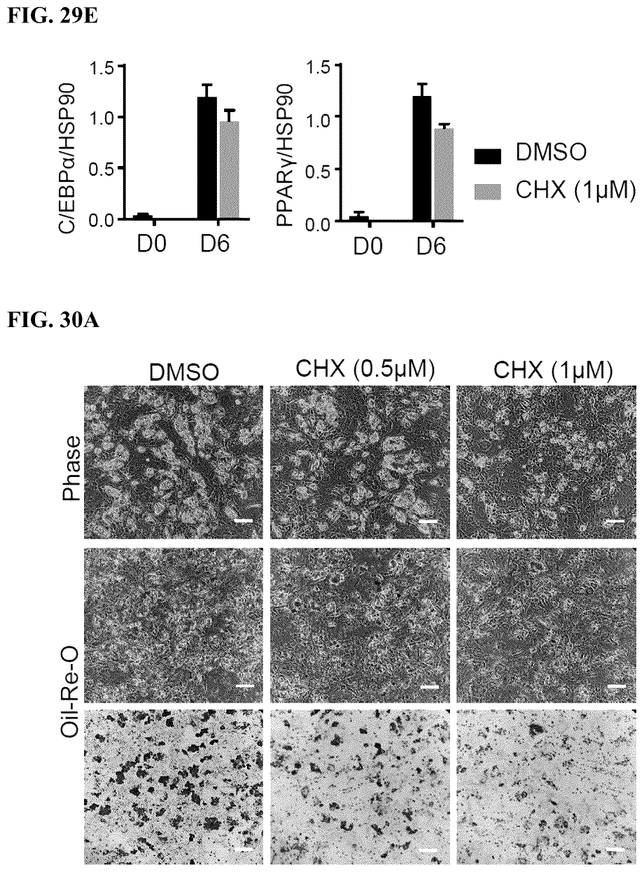

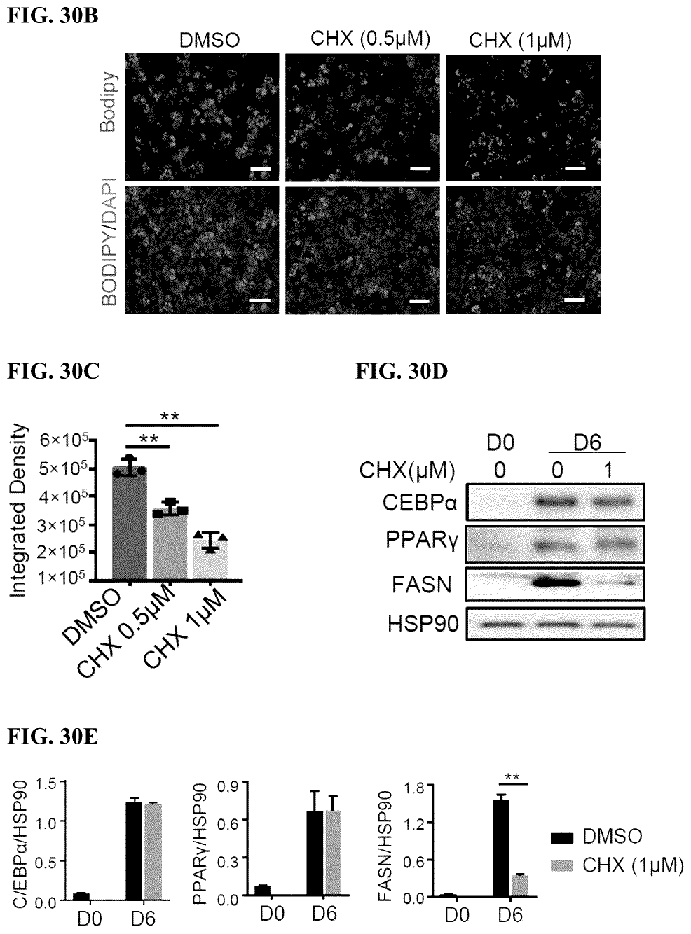

We next determined whether CHX inhibits terminal differentiation of lineage-committed 3T3-L1 preadipocytes. In these adipogenic precursor cells, CHX was sufficient to inhibit the formation of lipid-laden mature adipocyte in a dose-dependent manner, as indicated by phase-contrast and oil-red-O staining (FIG. 29). A similar result of CHX inhibitory effect on adipogenic maturation of preadipocytes was further demonstrated via Bodipy staining (FIGS. 29B-29C). Interestingly, examination of adipogenic factor induction at differentiation day 6 revealed only moderate tendency of lowering of C/EBPα and PPARγ levels by CHX (FIG. 29D) that were not statistically significant (FIG. 29E). We further determined CHX effect on preadipocyte terminal differentiation using primary preadipocytes isolated from the stromal vascular fraction of adipose depot, which may reveal potential relevance of in vivo application of CHX treatment. Following six days of differentiation, CHX at 0.5 and 1 μM displayed a dose-dependent, marked effect on suppressing mature adipocyte formation as revealed by phase-contrast morphology or oil-red-O staining (FIG. 30A). Bodipy staining further validated the inhibition of primary preadipocyte differentiation by CHX (FIGS. 30B-30C). Similar to the observation from 3T3-L1 preadipocytes, CHX only resulted in a tendency toward suppressing adipogenic factor induction at day 6 of differentiation (FIGS. 30D-30E). However, CHX markedly attenuated the terminal differentiation marker of adipocytes, FASN, suggesting its limited modulation of terminal differentiation stage of adipogenesis without altering the adipogenic program. Given the moderate effect of CHX on terminal differentiation of committed adipogenic progenitors, 3T3-L1 and primary preadipocytes, it may predominantly modulate early stages of adipogenesis by influencing lineage commitment of early adipogenic progenitors.

### Chlorhexidine Induction of Wnt Signaling Pathway in Adipogenic Precursors

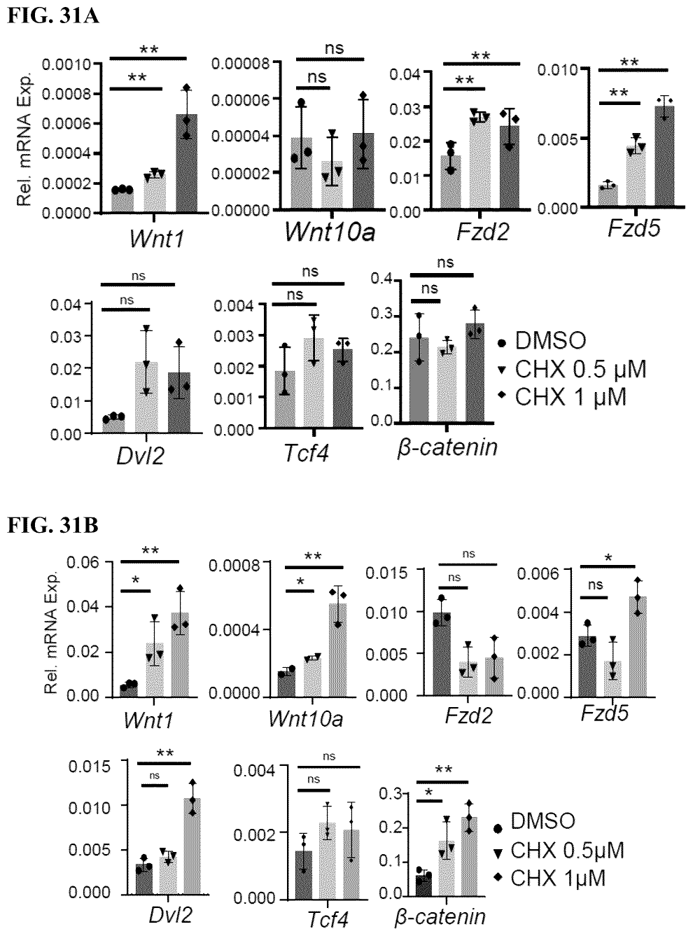

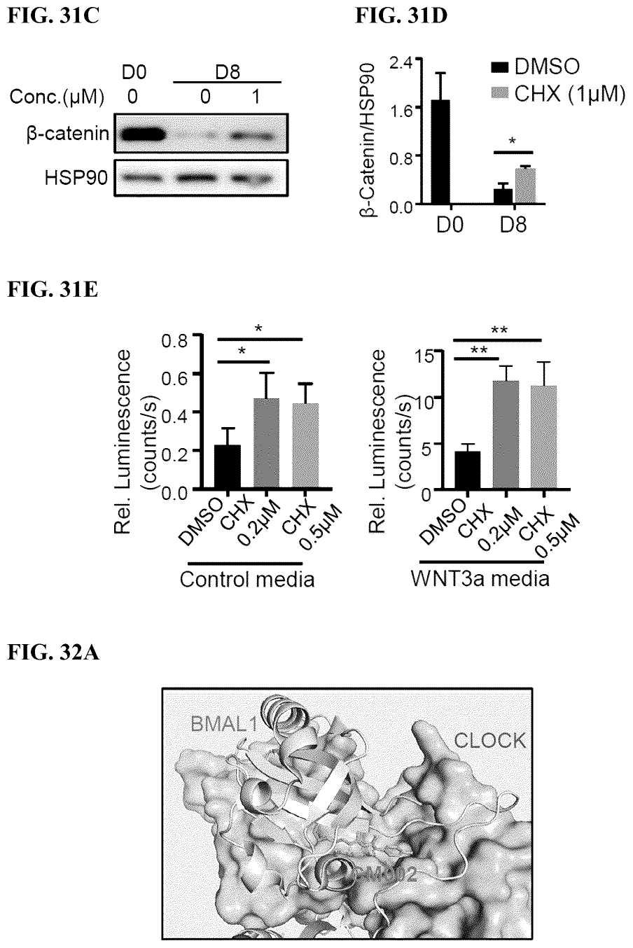

Wnt signaling pathway is a potent inhibitory development signal for adipose tissue development and adipogenic differentiation. Based on prior studies of Bmal1 transcriptional control of the Wnt signaling pathway, we postulated that CHX activation of CLOCK/Bmal1 and induction of direct target genes involved in Wnt pathway may mediates its negative effect on adipogenesis. Via RT-qPCR analysis, we screened various Wnt signaling components that are direct target genes of Bmal1 that we identified previously. Interestingly, CHX induced the mRNA levels of Wnt1, Wnt10a, Fzd2, Fzd5 that are up-stream Wnt ligands and receptors in 10T1/2 cells (FIG. 31A). In 3T3-L1 preadipocytes, similar regulations of Wnt ligands were observed, together with induction of b-catenin expression (FIG. 31B). Consistent with this finding of CHX modulation of Wnt pathway gene transcription, we found that CHX treatment in 10T/2 cells was sufficient to re-activate the lost b-catenin protein level during adipogenic differentiation (FIGS. 31C-31D). Furthermore, using a Wnt-responsive TOPFlash luciferase reporter containing TCF4 bindings sites, we found that CHX at 0.2 and 0.5 μM could induce Wnt signaling activity at either basal or Wnt-stimulated conditions (FIG. 31E), suggesting that CHX was sufficient to augment Wnt signaling activity in adipogenic progenitors.

### Chlorhexidine Structural Analog CM002 Displays Clock-Activating Property

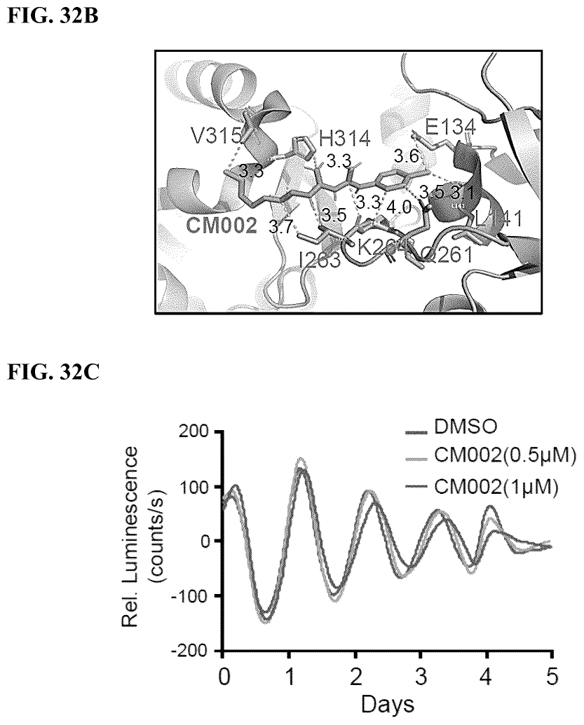

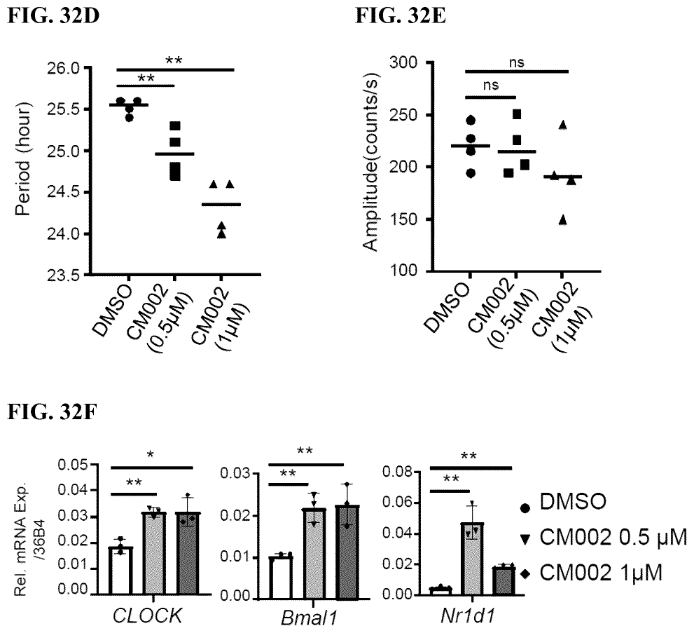

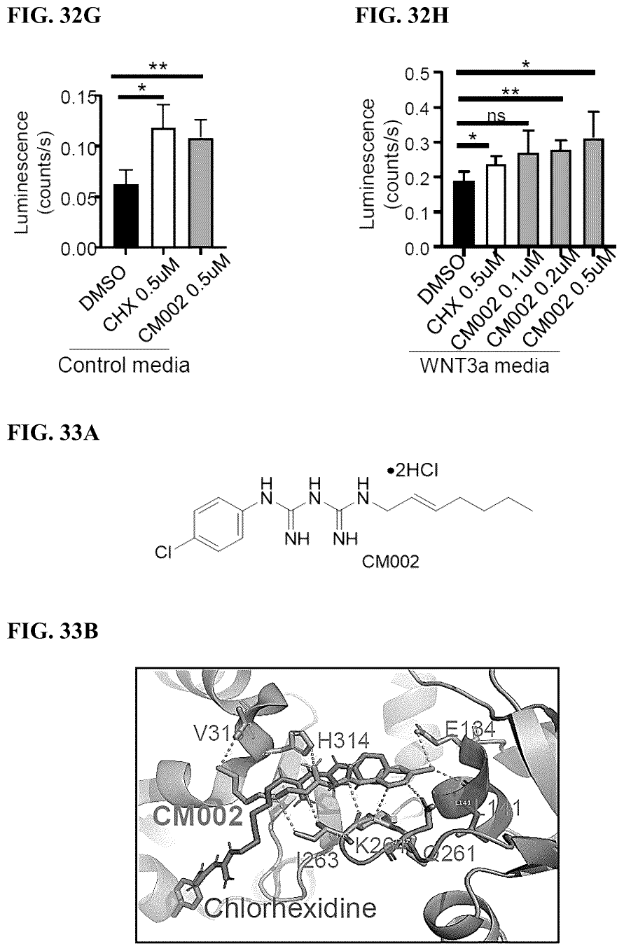

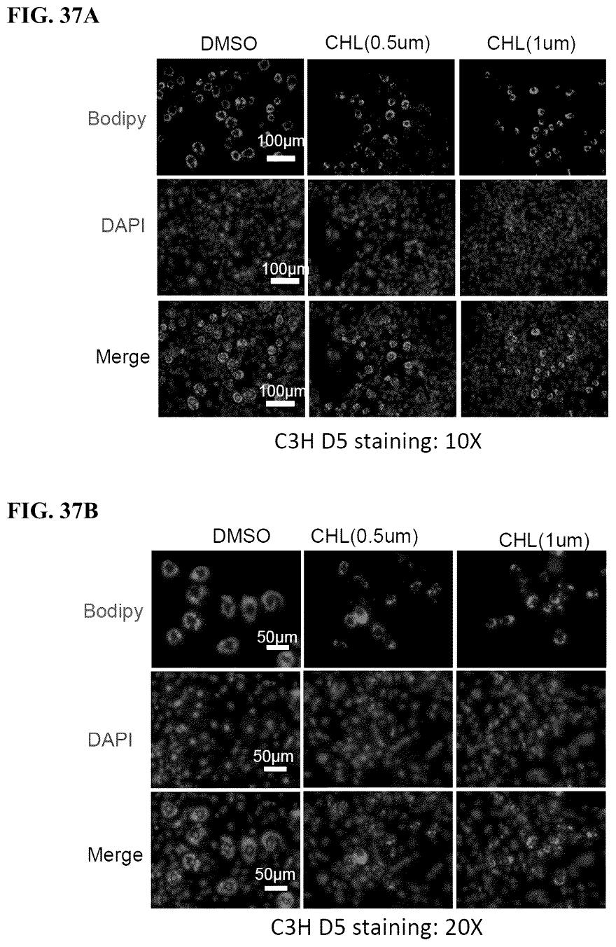

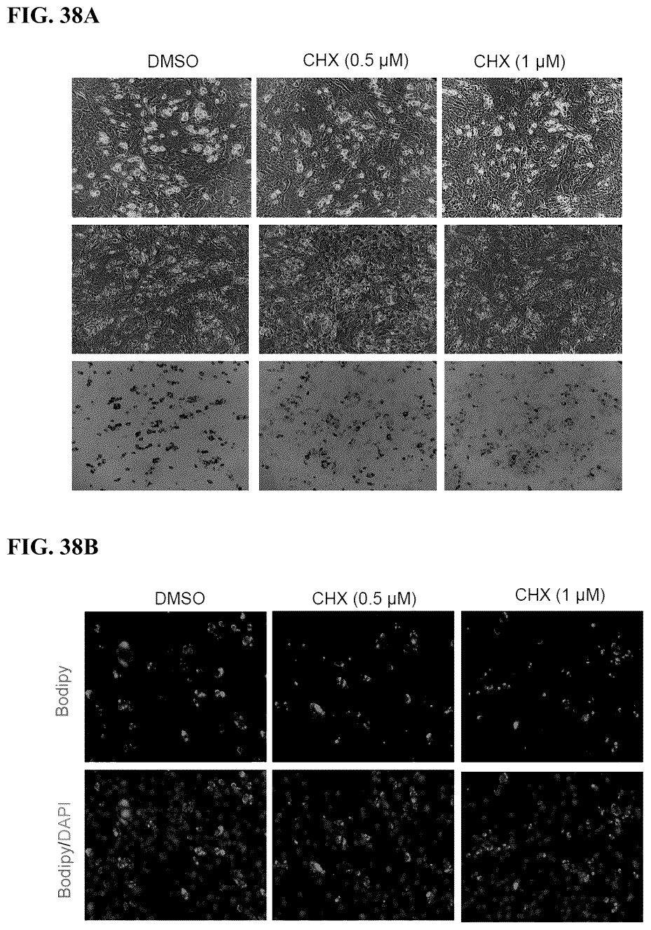

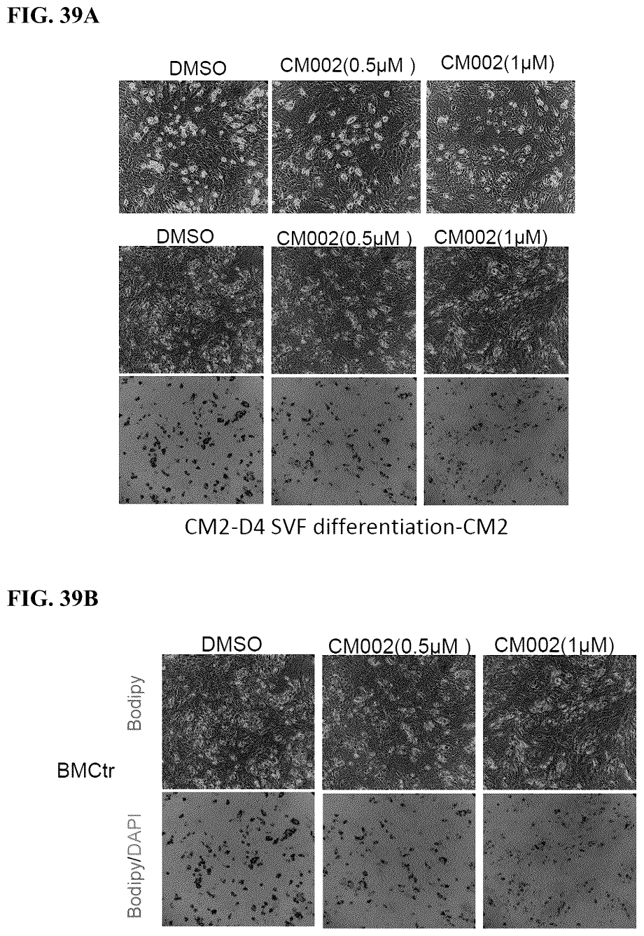

Based on the chemical scaffold of chlorhexidine, we generated twelve structural analogs via modification of various chemical groups within this molecule. Using an established methodology via real-time continuous monitoring of bioluminescence of a U2OS reporter cell line containing Per2:: dLuc luciferase, we determined the clock-modulatory activities of these compounds. This screening led to the identification of CM002, a molecule with approximately half of the structural scaffold of CHX (FIG. 33A), as a strong clock activator. CHX was identified by a high through-put screening platform for clock modulators against a hydrophobic pocket of the key molecular clock activator protein, CLOCK (Kiperman et al., 2023). Molecular docking pose indicated potential CM002 occupation within the shared hydrophobic pocket of CLOCK with CHX (FIGS. 32A, 37B). Consistent with the structural similarity of CM002 with half of CHX molecular scaffold, its predicted binding surface overlapped with half of CHX binding with the CLOCK protein (FIG. 33B). A detailed examination of CLOCK protein residues predicted to be interacting with CM002 within 3-4 Å were illustrated in FIG. 32B. As shown using U2OS Per2::dLuc reporter cell line, CM002 exhibited a dose-dependent effect on inducing clock period length shortening (FIGS. 32C-32D), without affecting cycling amplitude of clock (FIG. 32E), demonstrating its clock-activating property. Furthermore, gene expression analysis revealed CM002 induction of core clock transcription activators, CLOCK and Bmal1, together with up-regulation of its direct transcriptional target within the molecular clock loop, Nr1d1 (FIG. 32F). The essential core clock activator, Bmal1, exerts direct transcriptional control of components of Wnt signaling pathway to inhibit adipogenesis (Guo et al., 2012), and CHX can activate Wnt signaling due to its clock-modulatory activity. We thus examined whether CM002 modulates Wnt activity similarly as CHX using a Wnt-responsive TCF4-driven TOPFlash luciferase reporter (Veeman et al., 2003). Under basal condition, CM002 treatment at 0.5 μM was sufficient to stimulate TOPFlash reporter activity to a similar degree as CHX (FIG. 32G). Under Wnt3a media stimulation, CM002 at lower concentrations at 0.1 and 0.2 μM were sufficient to induce TOPFlash reporter activity comparable to that of CHX at 0.5 μM, suggesting improved Wnt-activating efficacy of CM002 (FIG. 32H).

### CM002 Inhibits Adipogenesis of Mesenchymal Precursor and Committed Preadipocytes

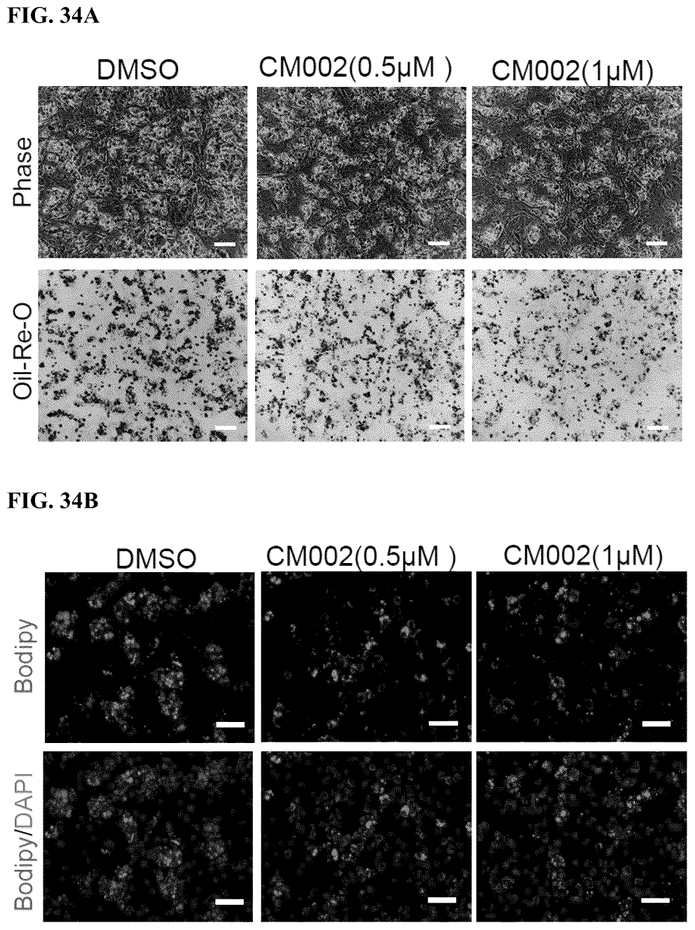

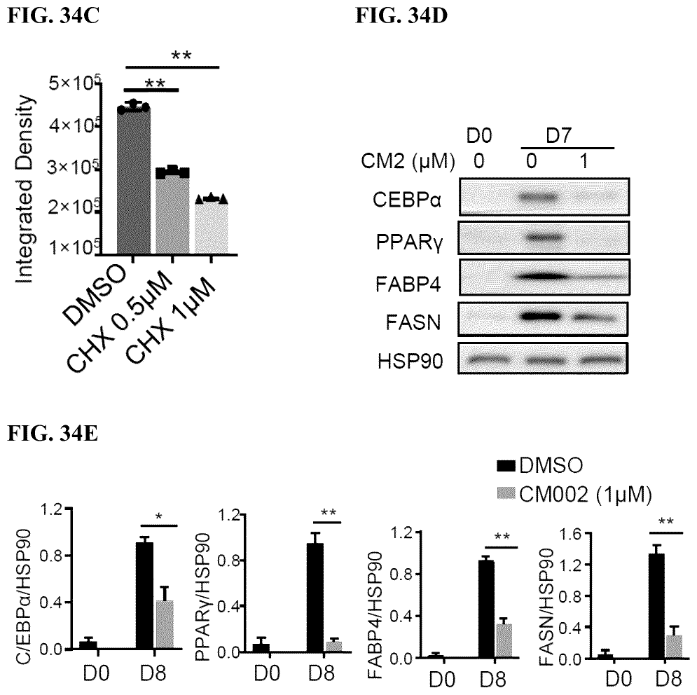

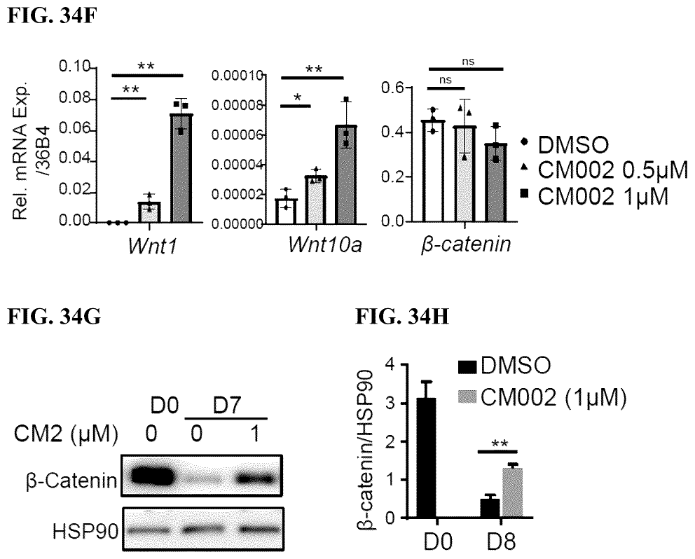

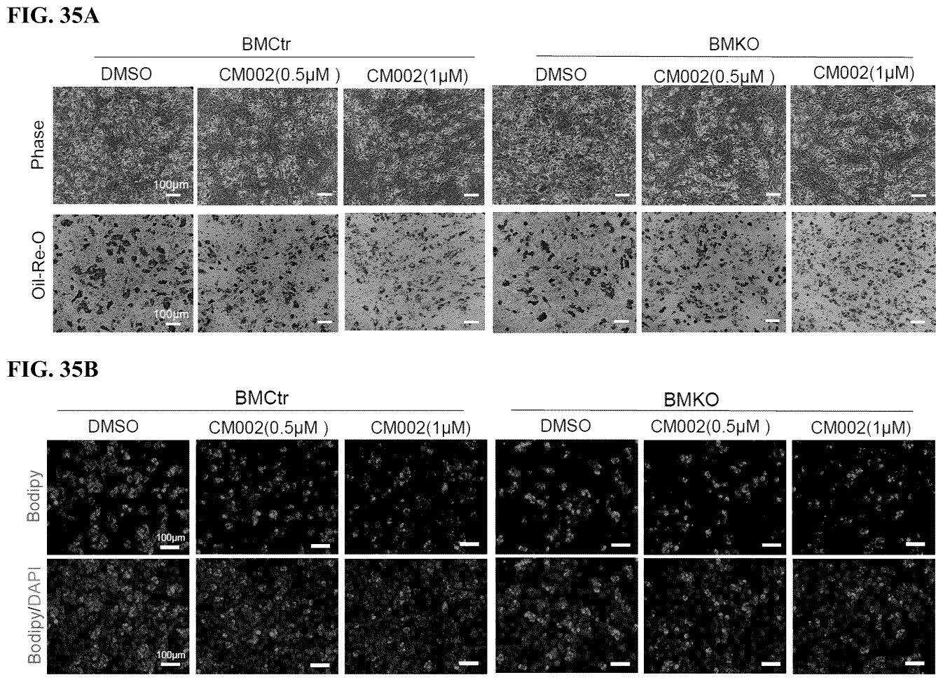

As a CHX analog with clock-activating property and stimulation of Wnt signaling, we postulated that CM002 may have anti-adipogenic effects as shown for CHX. Indeed, in C3H10T1/2 adipogenic precursor cells, CM002 markedly suppressed their differentiation in a dose-dependent manner as shown by oil-red-O (FIG. 34A) or Bodipy staining of lipids (FIGS. 34B-34C). Analysis of adipogenic gene induction at day 7 of differentiation revealed strong inhibition of adipogenic factors C/EBPα and PPARγ, suggesting a block of differentiation induction (FIGS. 34D-34E). Expression of mature adipocyte markers were also substantially lower in CM002-treated cells as compared to controls, indicating impaired maturation. Surveying key Wnt signaling components revealed significant induction of Wnt ligands including Wnt1 and Wnt10a transcripts, although the key signal transducer β-catenin was not modulated (FIG. 34F). β-catenin protein levels before and after adipogenic induction demonstrate a marked decline, as expected (FIGS. 34G-34H). Notably, CM002 was able to partially reverse the decline of β-catenin level at the differentiated state, suggesting its role in mediating the suppression of adipogenesis by CM002.

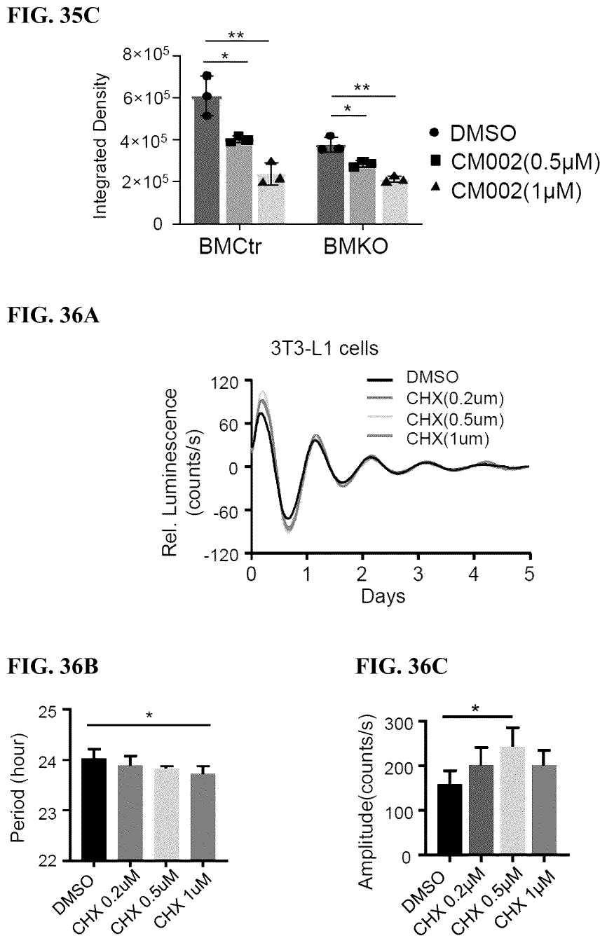

We further tested effect of CM002 on primary adipocyte differentiation and whether its adipogenic-modulatory activity is dependent on clock modulation. As shown in FIG. 35A, the addition of CM002 to differentiating primary preadipocytes, isolated from normal controls (BMCtr), strongly inhibited their differentiation (FIG. 35A). In Bmal1-deficient preadipocyte isolated from BMKO null mice, CM002 effect on reducing adipocyte formation was severely blunted. Bodipy staining further confirmed this finding, with dose-dependent reductions by CM002 at 0.5 and 1 μM concentration, while this inhibition of adipocyte maturation was attenuated in BMKO preadipocytes (FIGS. 35B-35C). Lastly, we tested whether CM002 suppression of preadipocyte maturation is due to terminal differentiation by administering CM002 following initial two days of adipogenic induction. Interestingly, CM002 still retained its efficacy of blocking adipocyte formation largely to the same extent as compared to the normal treatment with its presence throughout the entire duration of differentiation, as shown by lipid staining (FIG. 35D).

Given the established role of circadian clock regulation in modulating adipogenesis and a large body of evidence that clock disruption leads to the development of obesity and insulin resistance, circadian clock could be a potential therapeutic target to counter the current epidemic of metabolic diseases, particularly in light of the current epidemic of “social jetlag”. Employing distinct adipogenic progenitor cellular models, the current study uncovered the effects of a novel clock-activating compound, chlorhexidine in inhibiting adipogenic differentiation, and further demonstrate the anti-adipogenic efficacy of a new analog.

Accumulating studies have established the intimate association between circadian clock disruption and the development of metabolic diseases (Stenvers et al., 2019; Sinturel, Petrenko and Dibner, 2020). Given the pervasive temporal control of circadian clock of rate-limiting enzymes of metabolic pathways and circadian oscillation of metabolites, dysregulation of various clock-controlled metabolic processes contributes to altered metabolic homeostasis. We have reported previously that clock activators exert direct transcriptional control of Wnt signaling pathway components to modulate its activity, a known developmental signal in suppressing adipogenesis (Guo et al., 2012; Xiong et al., 2023). Loss of clock function, due to deficiency of core clock transcription activators CLOCK or Bmal1 in mice, leads to obesity (Paschos et al., 2012; Guo et al., 2012; Turek et al., 2005). In addition, disruption of clock via various environmental clock manipulations predispose to the development of obesity (Xiong, Lin, Lee et al., 2021; Scheer, Hilton, Mantzoros et al., 2009; Kolbe, Leinweber, Brandenburger et al., 2019; Barclay, Husse, Bode et al., 2012), while shiftwork is linked with a strong risk for insulin resistance and type II diabetes (Pan et al., 2011; Barclay et al., 2012; van Amelsvoort, Schouten and Kok, 1999). Thus targeting the specific link of clock with adipogenesis, particularly by maintain or re-enforcing proper circadian clock control in the face of wide-spread circadian misalignment, may lead to new avenues for obesity treatment through clock modulation. We previously established a screening platform for clock modulators that led to the identification of chlorhexidine as a clock activator. In line with its role in promoting myogenesis, via activation of Wnt-controlled signaling, chlorhexidine demonstrated a robust pro-myogenic effect in both C2C12 and primary myoblasts (Kiperman et al., 2023). We found recently that Nobiletin, by activating the positive regulator of the clock RORa, was able to suppress adipogenesis and demonstrated strong anti-obesity effect in mice under both chow and high-fat diet conditions (Xiong et al., 2023).

Through chemical modifications based on the chlorhexidine scaffold, we generated new analogs and screened for compounds with improved clock-modulatory activity. This led to the identification of CM002 as a new clock activating molecule. Results based on a standard clock activity monitoring via Per2:: dLuc reporter cell line revealed that CM002 displayed improved efficacy range on inducing clock period shortening without cellular toxicity. Further findings of its biological activity in promoting Wnt signaling is consistent with improved clock-modulatory efficacy. Importantly, CM002 effect on adipogenic inhibition appears to be more robust, as shown by strong inhibition of the adipogenic gene program. More detailed comparisons between CM002 and CHX is needed in future studies to determine the relative anti-adipogenic efficacy, particularly using in vivo models. We observed certain cellular toxicity of CHX beyond 1 μM concentration, while the viability of cells treated by CM002 at this concentration was maintained, suggesting potential wider concentration range for this compound.

An intriguing finding from our study is the differential modulation of CHX and CM002 on adipogenic gene induction between the mesenchymal and lineage-committed progenitors, the C3H10T1/2 cells as compared to 3T3-L1 and primary preadipocytes. Both CHX and CM002 had robust effect on reducing adipocyte formation in mesenchymal progenitors, with consistent reduction of expression levels of key adipogenic factor and mature adipocyte markers. Interestingly, despite a comparable level of inhibition of adipocyte maturation by these molecules in either 3T3-L1 or primary preadipocytes as indicated by the degree of lipid accumulation, their effects are largely confined to blockade of mature adipocyte marker expression without significant regulation of adipogenic factors. These distinct regulation by clock activators in specific adipogenic progenitor cell types suggest their potential developmental stage-specific modulation of the differentiation process in adipocytes. CHX and CM002 likely affects both the lineage commitment process that was induced at early stage of adipogenic induction in mesenchymal progenitors with consequent impact on terminal differentiation, whereas they impact on lineage-determined preadipocytes appears to be confined to suppressing only terminal differentiation step of adipocyte maturation with minimal impact on adipogenic induction. We specifically addressed this distinction by comparing their efficacy in suppressing adipogenesis before and after adipogenic induction cocktail addition. The comparable efficacy on reducing adipocyte formation by CM002 suggests that clock activation functions to inhibit mostly terminal differentiation in lineage-committed adipocyte progenitors. This mechanism of action of clock-activating molecules suggest that they may have potential therapeutic potentials to prevent preadipocyte conversion to mature adipocyte in vivo for anti-obesity drug development.

The anti-adipogenic effects of CHX and its derivative offers potential for anti-obesity applications. High-fat diet feeding-induced obesity leads to dampening of clock oscillatory amplitude, raising the possibility that promoting clock oscillation could reverse this effect of nutritional overload on clock-controlled metabolic pathways. We recently demonstrated that the RORa-activating compound Nobiletin, by activating the positive regulator of the clock was able to suppress adipogenesis and demonstrated strong anti-obesity effect in mice under both chow and high-fat diet conditions (Xiong et al., 2023). Mediated by a common mechanism of transcriptional modulation of the Wnt signaling components, clock exerts anti-adipogenic effect while promotes myogenesis (Guo et al., 2012; Kiperman et al., 2023; Chatterjee et al., 2013). This raises a possibility that maintaining or promoting proper clock modulation may have applications toward therapies against sarcopenic obesity, characterized by the co-existing conditions of loss of skeletal muscle mass with adipose expansion in the elderly (Batsis and Villareal, 2018; Cleasby, Jamieson and Atherton, 2016). With the growing attention in targeting clock modulation for disease treatment (Chaix, Zarrinpar, Miu et al., 2014), clock activators and more diverse compounds that augment clock modulations could be applicable to a diverse array of human pathologies associated with clock disruption.

### REFERENCES FOR EXAMPLE 3

1. Takahashi, J. S., 2017. Nat Rev Genet. 18, 164-179. 2. Bass, J. and Takahashi, J. S., 2010. Science. 330, 1349-54. 3. Stenvers, D. J., Scheer, F., Schrauwen, P., la Fleur, S. E. and Kalsbeek, A., 2019. Nat Rev Endocrinol. 15, 75-89. 4. Pan, A., Schernhammer, E. S., Sun, Q. and Hu, F. B., 2011. PLoS Med. 8, e1001141. 5. Fonken, L. K., Workman, J. L., Walton, J. C., Weil, Z. M., Morris, J. S., Haim, A. and Nelson, R. J., 2010. Proc Natl Acad Sci USA. 107, 18664-9. 6. Sulli, G., Manoogian, E. N. C., Taub, P. R. and Panda, S., 2018. Trends Pharmacol Sci. 39, 812-827. 7. Cederroth, C. R., Albrecht, U., Bass, J., Brown, S. A., Dyhrfjeld-Johnsen, J., Gachon, F., Green, C. B., Hastings, M. H., Helfrich-Forster, C., Hogenesch, J. B., Levi, F., Loudon, A., Lundkvist, G. B., Meijer, J. H., Rosbash, M., Takahashi, J. S., Young, M. and Canlon, B., 2019. Cell Metab. 30, 238-250. 8. Janich, P., Pascual, G., Merlos-Suarez, A., Batlle, E., Ripperger, J., Albrecht, U., Cheng, H. Y., Obrietan, K., Di Croce, L. and Benitah, S. A., 2011. Nature. 480, 209-14. 9. Janich, P., Toufighi, K., Solanas, G., Luis, N. M., Minkwitz, S., Serrano, L., Lehner, B. and Benitah, S. A., 2013. Cell Stem Cell. 13, 745-53. 10. Qian, J., Yeh, B., Rakshit, K., Colwell, C. S. and Matveyenko, A. V., 2015. Endocrinology. 156, 4426-36. 11. Kohsaka, A., Laposky, A. D., Ramsey, K. M., Estrada, C., Joshu, C., Kobayashi, Y., Turek, F. W. and Bass, J., 2007. Cell Metab. 6, 414-21. 12. Solanas, G., Peixoto, F. O., Perdiguero, E., Jardi, M., Ruiz-Bonilla, V., Datta, D., Symeonidi, A., Castellanos, A., Welz, P. S., Caballero, J. M., Sassone-Corsi, P., Munoz-Canoves, P. and Benitah, S. A., 2017. Cell. 170, 678-692 e20. 13. Paschos, G. K., Ibrahim, S., Song, W. L., Kunieda, T., Grant, G., Reyes, T. M., Bradfield, C. A., Vaughan, C. H., Eiden, M., Masoodi, M., Griffin, J. L., Wang, F., Lawson, J. A. and Fitzgerald, G. A., 2012. Nat Med. 18, 1768-77. 14. Guo, B., Chatterjee, S., Li, L., Kim, J. M., Lee, J., Yechoor, V. K., Minze, L. J., Hsueh, W. and Ma, K., 2012. FASEB J. 26, 3453-63. 15. Turek, F. W., Joshu, C., Kohsaka, A., Lin, E., Ivanova, G., McDearmon, E., Laposky, A., Losee-Olson, S., Easton, A., Jensen, D. R., Eckel, R. H., Takahashi, J. S. and Bass, J., 2005. Science. 308, 1043-5. 16. Grimaldi, B., Bellet, M. M., Katada, S., Astarita, G., Hirayama, J., Amin, R. H., Granneman, J. G., Piomelli, D., Leff, T. and Sassone-Corsi, P., 2010. Cell Metab. 12, 509-20. 17. Solt, L. A., Wang, Y., Banerjee, S., Hughes, T., Kojetin, D. J., Lundasen, T., Shin, Y., Liu, J., Cameron, M. D., Noel, R., Yoo, S. H., Takahashi, J. S., Butler, A. A., Kamenecka, T. M. and Burris, T. P., 2012. Nature. 485, 62-8. 18. Xiong, X., Kiperman, T., Li, W., Dhawan, S., Lee, J., Yechoor, V. and Ma, K., 2023. Endocrinology. 164. 19. Kiperman, T., Li, W., Xiong, X., Li, H., Horne, D. and Ma, K., 2023. Stem Cell Res Ther. 14, 190. 20. Nam, D., Guo, B., Chatterjee, S., Chen, M. H., Nelson, D., Yechoor, V. K. and Ma, K., 2015. J Cell Sci. 128, 1835-47. 21. Liu, R., Xiong, X., Nam, D., Yechoor, V. and Ma, K., 2020. Mol Cell Endocrinol. 515, 110920. 22. Chatterjee, S., Nam, D., Guo, B., Kim, J. M., Winnier, G. E., Lee, J., Berdeaux, R., Yechoor, V. K. and Ma, K., 2013. J Cell Sci. 126, 2213-24. 23. Xiong, X., Li, W., Nam, J., Qu, M., Kay, S. A. and Ma, K., 2022. J Cell Sci. 135. 24. Veeman, M. T., Slusarski, D. C., Kaykas, A., Louie, S. H. and Moon, R. T., 2003. Curr Biol. 13, 680-5. 25. Sinturel, F., Petrenko, V. and Dibner, C., 2020. J Mol Biol. 432, 3680-3699. 26. Xiong, X., Lin, Y., Lee, J., Paul, A., Yechoor, V., Figueiro, M. and Ma, K., 2021. Mol Cell Endocrinol. 521, 111110. 27. Scheer, F. A., Hilton, M. F., Mantzoros, C. S. and Shea, S. A., 2009. Proc Natl Acad Sci USA. 106, 4453-8. 28. Kolbe, I., Leinweber, B., Brandenburger, M. and Oster, H., 2019. Mol Metab. 30, 140-151. 29. Barclay, J. L., Husse, J., Bode, B., Naujokat, N., Meyer-Kovac, J., Schmid, S. M., Lehnert, H. and Oster, H., 2012. PLoS One. 7, e37150. 30. van Amelsvoort, L. G., Schouten, E. G. and Kok, F. J., 1999. Int J Obes Relat Metab Disord. 23, 973-8. 31. Batsis, J. A. and Villareal, D. T., 2018. Nat Rev Endocrinol. 14, 513-537. 32. Cleasby, M. E., Jamieson, P. M. and Atherton, P. J., 2016. J Endocrinol. 229, R67-81. 33. Chaix, A., Zarrinpar, A., Miu, P. and Panda, S., 2014. Cell Metab. 20, 991-1005.

### Example 4: Clock-Activating Compounds Ameliorate Muscular Dystrophy by Promoting Regenerative Repair

The circadian clock plays a key role in modulating muscle stem cell behavior and regenerative myogenesis. Recent studies implicate the involvement of circadian clock dysregulation in the etiology of muscular dystrophy, and key clock regulators are required to protect against dystrophic muscle damage. To date, whether clock function in promoting myogenic repair could be targeted for muscular dystrophy therapy remains unexplored. Based on our discovery of novel clock-activating compounds with pro-myogenic properties, the current study investigated their efficacy in promoting myogenic repair in muscular dystrophy disease models.

The effect of clock activators, chlorhexidine (CHX) and a new analog CM002, on myogenesis were determined in normal and disease myoblasts harboring dystrophin mutation from mice and Duchene Muscular Dystrophin (DMD) patients. Furthermore, their effects on regenerative repair were examined using acute cardiotoxin-induced muscle injury in vivo. Lastly, potential protective effects of clock activators on dystrophic pathophysiology were explored in dystrophin-deficient mdx mice.

CHX and CM002 displayed clock-activating properties and promoted myogenic differentiation of normal murine primary myoblasts. We further demonstrate their pro-myogenic efficacy in myoblasts of mdx mice harboring dystrophin mutation, as well as primary myoblasts derived from DMD patients. Upon acute muscle injury, CHX and CM002 treatment induced prolonged nascent myofiber formation with elevated regenerative myogenesis. Direct intramuscular injections of these compounds in mdx mice elicited clock activation, augmented myogenic response, and promoted satellite cell proliferative expansion with new myofiber regeneration under dystrophic condition. These effects of CHX and CM002 are accompanied by robust protection against muscle injury and attenuated inflammatory response. Furthermore, guided by PK analysis of CM002, we show that its systemic administration significantly improved muscle contractile function in mdx mice, accompanied with enhanced myogenic repair and attenuated dystrophic muscle injury.

Collectively, our findings from cellular and mouse models of muscular dystrophy revealed the efficacy of clock-activating compounds, CHX and CM002, in promoting myogenic repair to ameliorate dystrophic muscle damage. The discovery of these clock activators provides new avenues for therapeutic targeting of clock modulation to ameliorate muscular dystrophy or muscle wasting conditions.

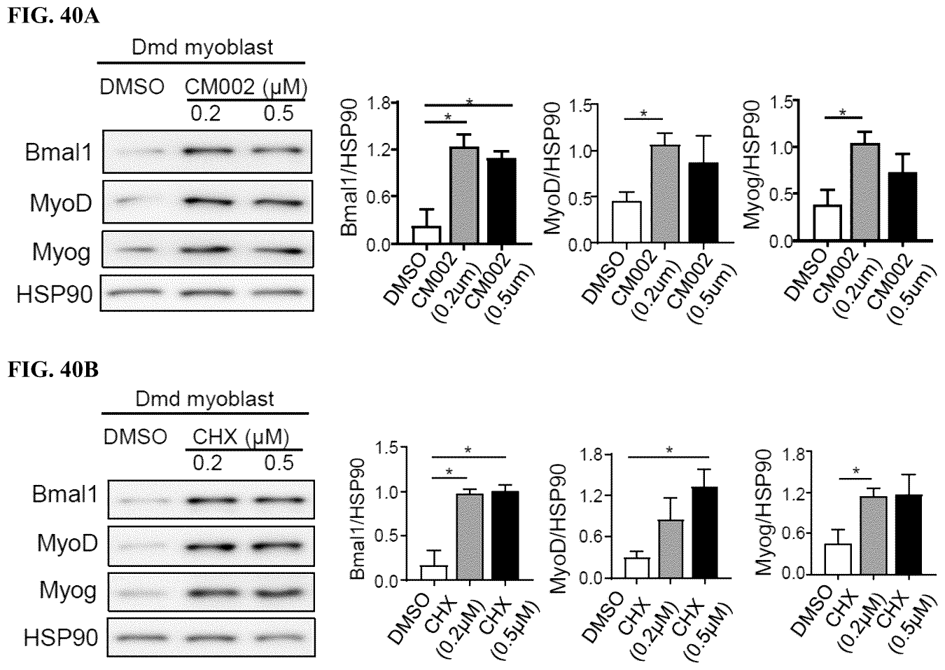

FIGS. 40A-40B. Chlorhexidine and CM002 promote myogenic induction in human DMD primary myoblasts. Using primary myoblasts derived from patients with Duchene Muscular Dystrophy, we determined the effect of Chlorhexidine and CM002 on promoting myogenic induction. In DMD myoblasts subjected to differentiation for 3 days, CM002 treatment at 0.2 and 0.5 μM significantly elevated protein expression of Bmal1, suggesting activation of clock (FIG. 40A). Robust inductions of MyoD and Myogenin protein were also evident, indicative of enhanced myogenic differentiation. Chlorhexidine (CHX) treatment of these cells resulted in similar effects on inducing Bmal1 and myogenic factors (FIG. 40B).

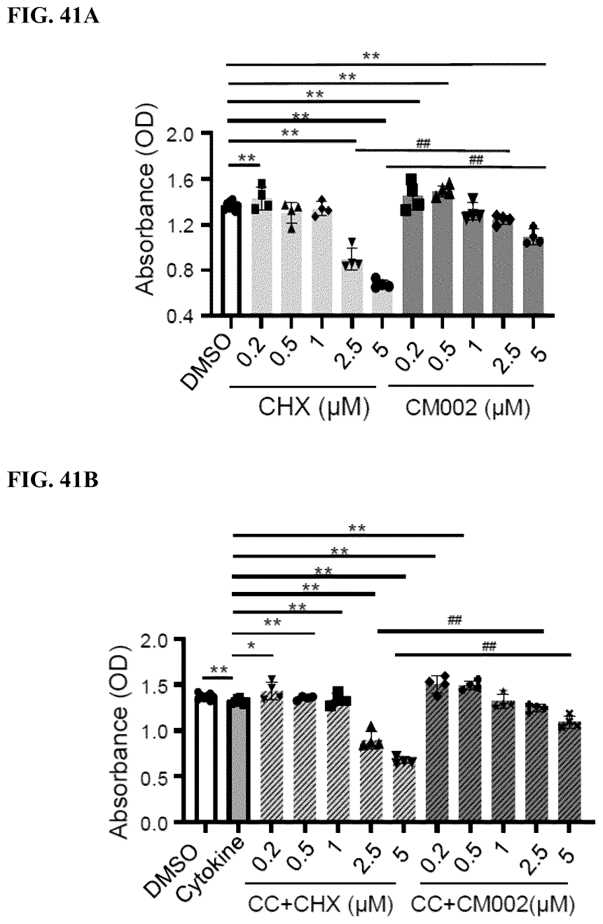

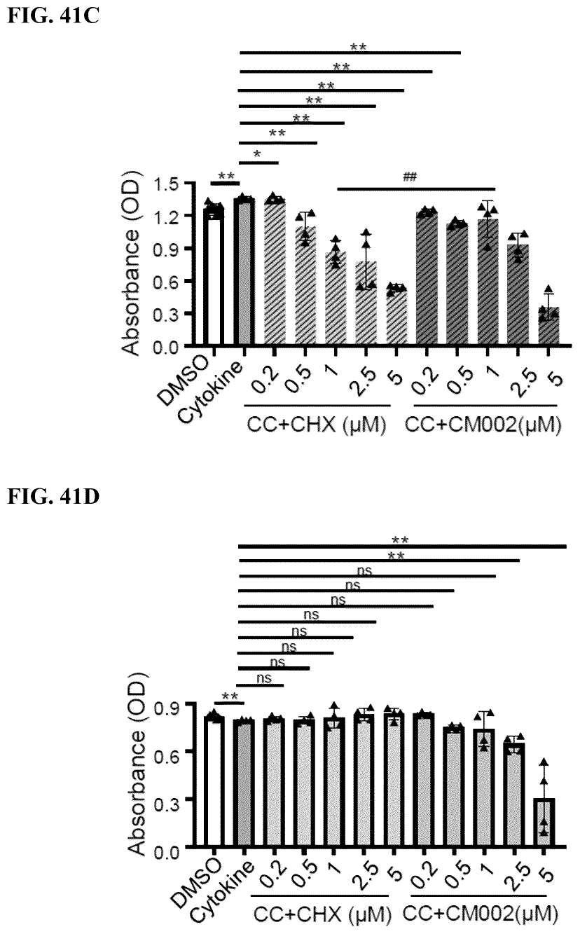

FIGS. 41A-41D. Effect of Chlorhexidine (CHX) and CM002 treatment on viability of mouse and human myoblasts. To determine potential toxic effects of CHX and CM002, we treated mouse C2C12 myoblast cell line with increasing concentrations from 0.2-5 μM for 48 hours and determined cell viability using the MTT assay (FIG. 41A). No significant drop of number of viable cells were observed until CHX concentration increased to 2.5 μM, while cells were susceptible to 5 μM of CM002 treatment only, suggesting reduced cellular toxicity of CM002. Considering the inflammatory milieus in dystrophic muscles, we further tested the tolerance of myoblasts to these molecules in the presence of inflammatory stimuli, a cytokine cocktail (CC) consisted of 10 ng/mL IL-1β, 100 ng/mL IFN-γ and 25 ng/mL of TNF-α (FIG. 41B). Similarly differential responses were observed between CHX and CM002 under this condition, with cells displaying better tolerance to CM002 at 2.5 and 5 μM than CHX. Using primary myoblasts isolated from mdx mouse (FIG. 41C), we found that they are more sensitive to cell death when treated at 0.5 μM or higher concentrations of CHX, while remaining viable up to 1 μM of CM002 with significantly less cell death at 2.5 μM. Lastly, human primary myoblasts derived from Duchene Muscular Dystrophy (DMD) patients were used to determine their tolerance to these compounds. Interestingly, these cells tolerated CHX treatment better without significantly compromised viability up to 5 μM, although displaying sensitivity to cell death by CM002 treatment at this concentration (FIG. 41D).

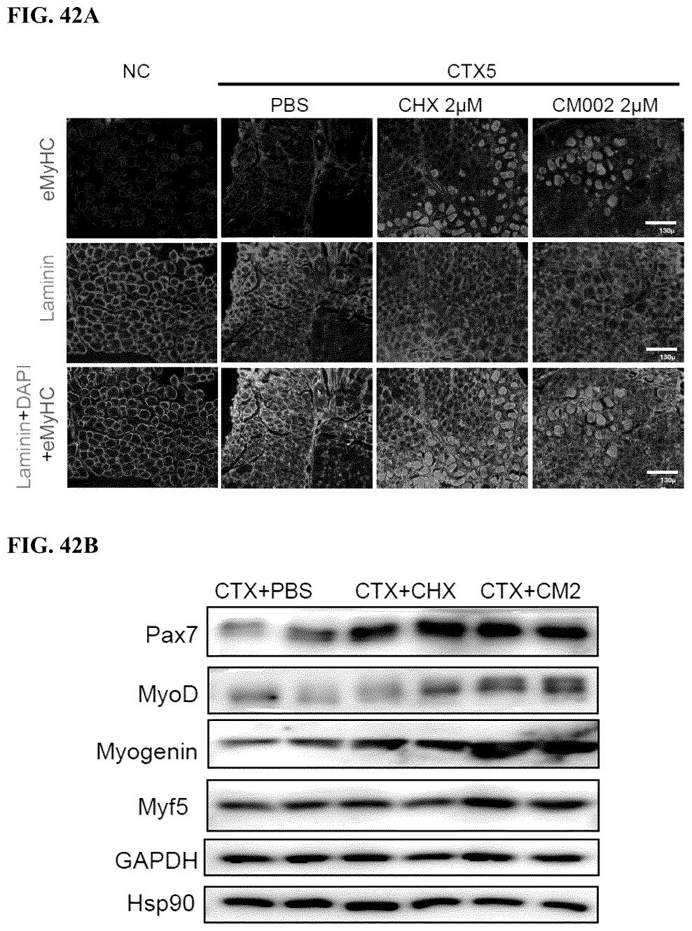

FIGS. 42A-42B. CHX and CM002 promote nascent myofiber formation upon acute muscle injury. To determine whether the effect of CHX and CM002 on promoting myogenesis leads to improved muscle regeneration in vivo, we tested local intramuscular injection into TA muscle following acute injury by cardiotoxin in normal C57/BL6 wild-type mice. As shown by immunofluorescence staining of embryonic myosin heavy chain (eMyHC) to examine the extent of new myofiber formation, while controls displayed barely detectable levels of new myofiber as expected at 5 days after cardiotoxin injury, CHX and CM002-treated muscles demonstrated substantial amount of eMyHC staining indicative of sustained nascent myofiber regeneration (FIG. 42A). Analysis of myogenic response revealed elevated levels of MyoD, Myf5 and Myogenein evident in CM002-treated muscle (FIG. 42B), while Pax7 protein were robustly increased by both CHX and CM002 administration. Collectively, these findings indicate that these clock-activating molecules are capable of inducing myogenic response via local administration leading to sustained new myofiber formation following acute muscle injury.

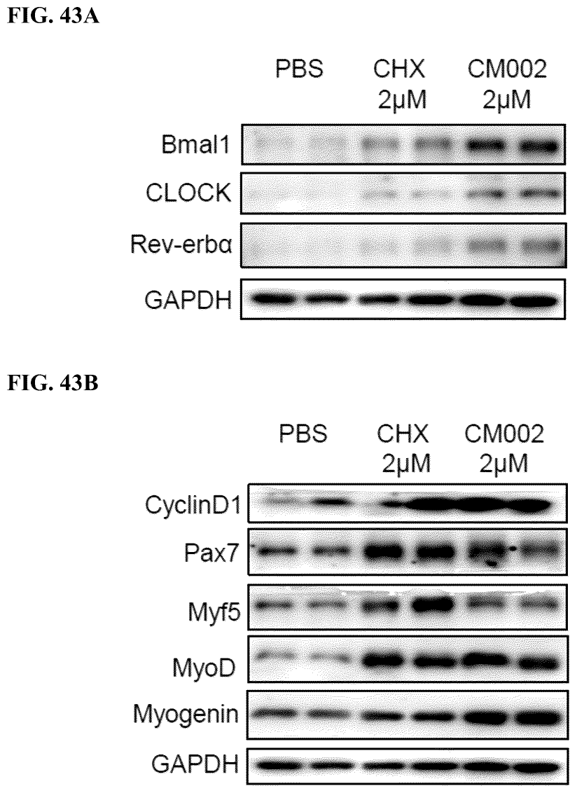

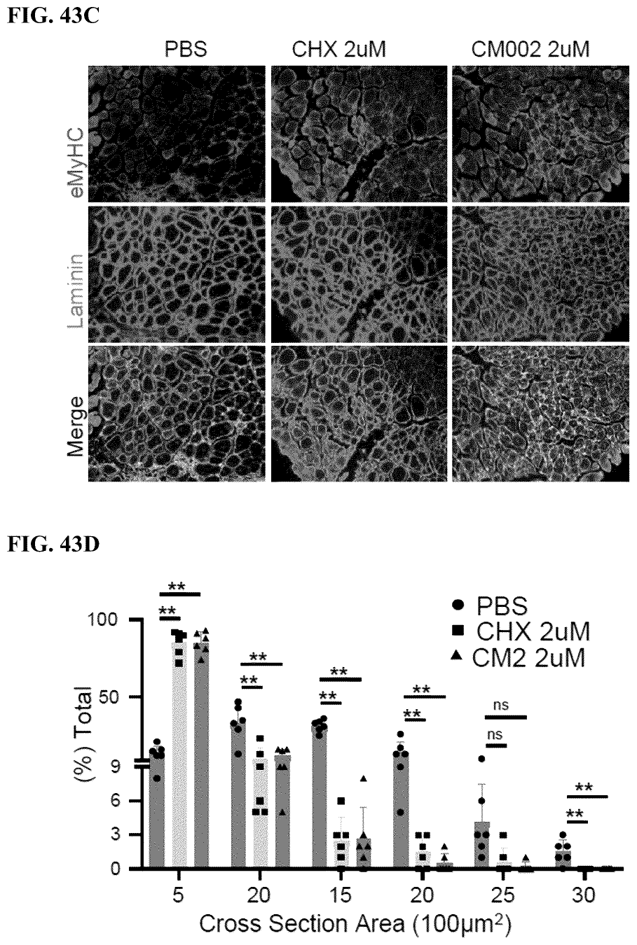

FIGS. 43A-43D. CHX and CM002 promote regenerative myogenesis in dystrophic mdx mice. Based on the effect of CHX and CM002 on promoting regenerative myogenesis, we next tested whether they exert protective effects in dystrophic disease conditions via local intramuscular delivery in the mdx mice, an established model for muscular dystrophy. Following three daily dosages of Chlorhexidine and CM002 injection into TA muscle, protein levels of clock regulators, Bmal1, CLOCK and its direct target gene Rev-erbα, were significantly elevated with stronger effect by CM002 (FIG. 43A), indicating their clock-activating effect in dystrophic muscle. Importantly, local delivery of these molecules in mdx TA muscles were sufficient to induce the myogenic response, as indicated by elevated protein levels of myogenic factors (FIG. 43B). Pax7 and CyclinD1 were also induced, suggesting satellite cell cycle activation. eMyHC staining revealed increased new myofiber formation in Chlorhexidine and CM002-treated muscles (FIG. 43C). Analysis of myofiber size distribution via quantification of cross section area indicate a striking left shift toward smaller myofibers in these muscles, consistent with the emergence of nascent myofiber following treatment (FIG. 43D).

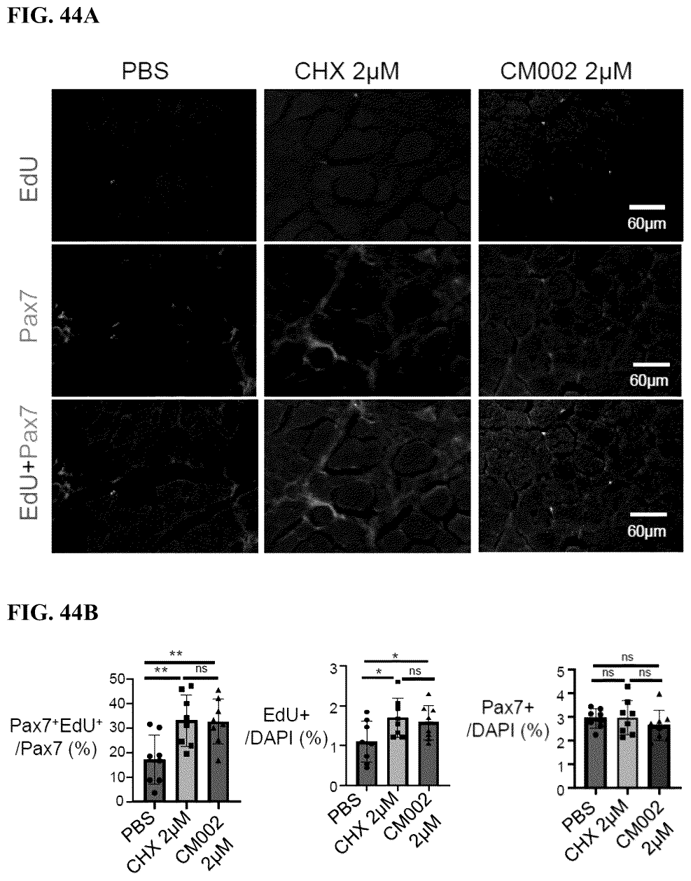

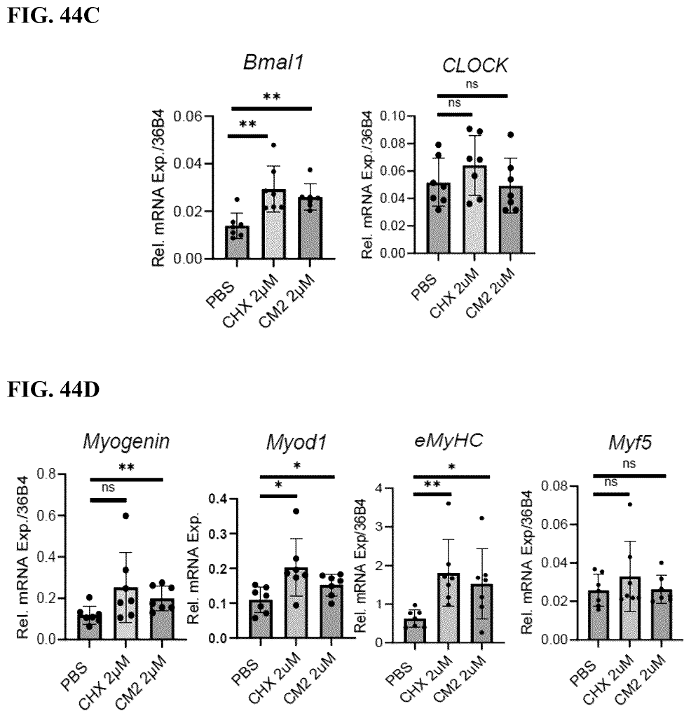

FIGS. 44A-44D. CM002 and CHX stimulate satellite cell proliferative expansion and myogenic gene induction in mdx mice. As Pax7 and CyclinD1 levels were increased by CM002 and CHX in mdx TA, we directly examined the proliferation of satellite cells via EdU labeling with co-staining for Pax7 to identify satellite cells. As shown in FIG. 44A and FIG. 44B, the increased percentage of EdU+/Pax7+ within Pax7+ satellite cell pool indicate that indeed CM002 and CHX are sufficient to stimulate satellite cells proliferative expansion in dystrophic muscle to a similar degree. Although total EdU+ cells were increased as a percentage of the cellular pool, the amount of satellite cells was not significantly altered by these treatments (FIG. 44B). Consistent with the findings of elevated protein expression of clock regulators and myogenic factors, corresponding increase of their transcript levels by CM002 and CHX administration were observed in mdx muscle (FIGS. 44C-44D).

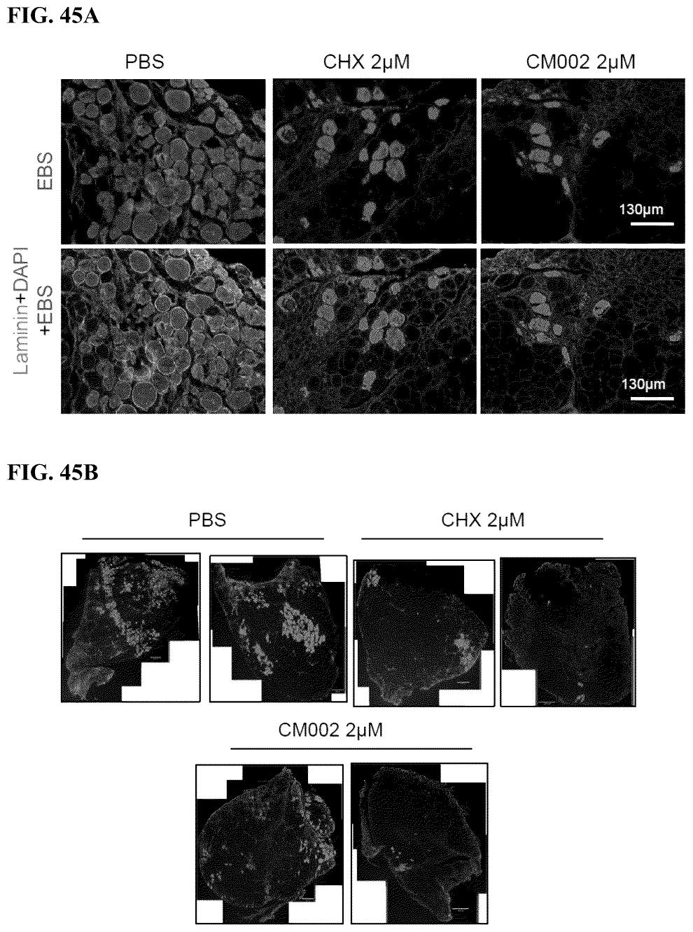

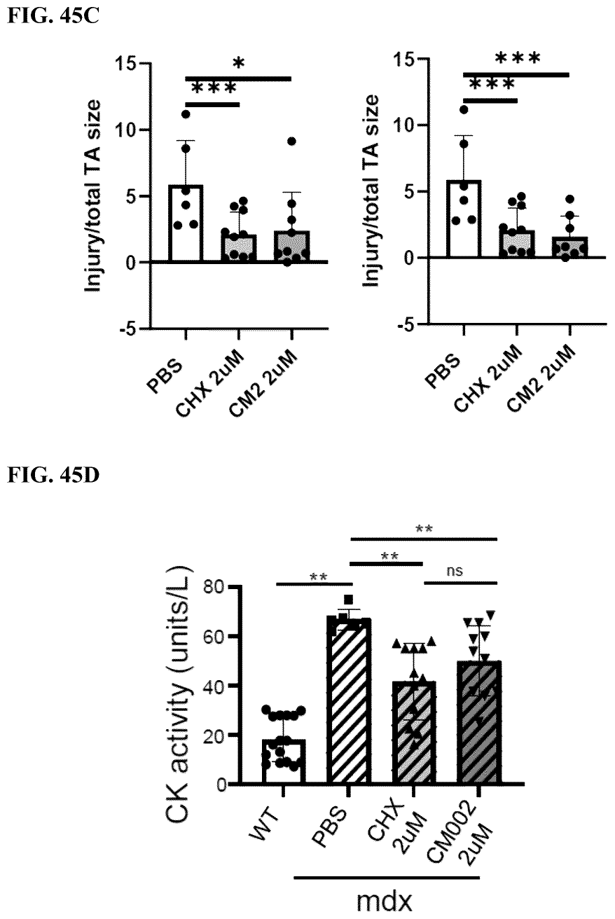

FIGS. 45A-45D. CM002 and CHX protects against dystrophic muscle damage in mdx mice. Given that local delivery of CHX and CM002 stimulated new myofiber formation to promote regenerative repair, we next examined whether these effects lead to protection against dystrophic injury. As shown by Evans Blue Dye (EBD) staining to determine the degree of muscle damage, CM002 and CHX treatment significantly reduced the areas stained positive for EBD (FIG. 45A), with further validation by examination of the degree of injury in entire TA cross sections (FIGS. 45B-45C). Most importantly, using ex vivo TA explants, we determined creatine kinase secretion from treated TA muscles as a direct indicator to assess muscle injury. Following local CM002 and CHX injection, mdx TA displayed significantly lower CK levels indicative of an overall reduction of muscle injury (FIG. 45D). Surprisingly, CHX effect on reducing CK levels, approximately 50%, appears to be more robust than that of CM002 (˜35%) under this condition (FIG. 45D).

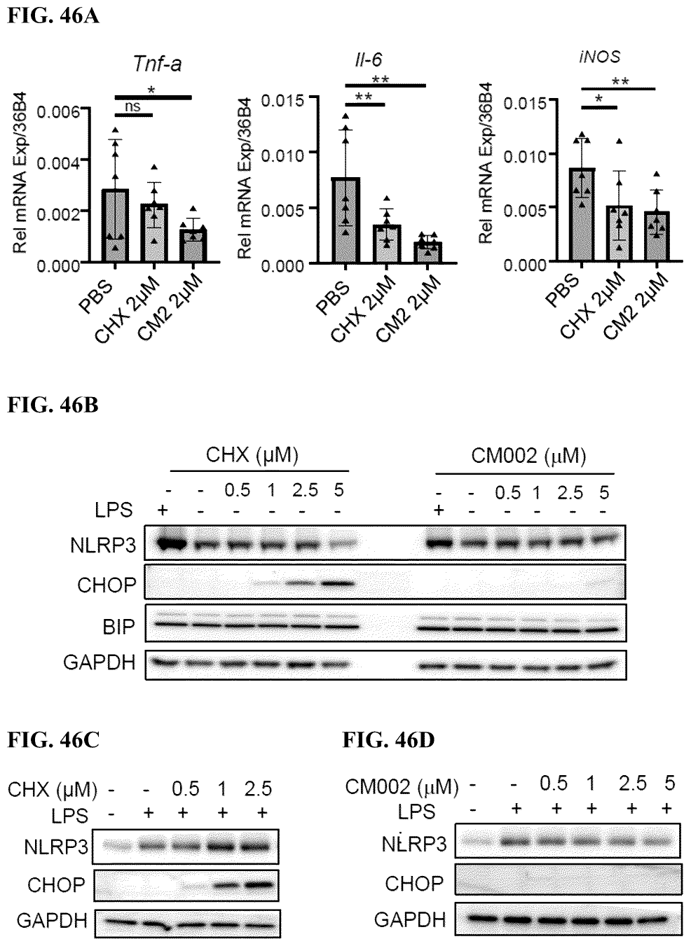

FIGS. 46A-46D. CHX and CM002 ameliorate inflammatory milieu of muscular dystrophy. Chronic inflammation in dystrophic muscle, due to the persistent muscle damage with sustained degeneration and regeneration cycles, is a significant component of the pathogenic characteristics of muscular dystrophy contributing to muscle wasting. We thus determined potential ameliorative effects of CHX and CM002 on muscle inflammation in the mdx mice. Both compounds were able to reduce the inflammatory milieu in the dystrophic muscle, as indicated by consistent reduction of expression of the pro-inflammatory cytokines examined, TNF-α, IL-6 and iNOS (FIG. 46A). To further test whether the reduction of inflammation in muscle could be due to direct modulation of macrophage pro-inflammatory activities, we determined CHX and CM002 effects in primary bone marrow-derived macrophages. Examination of NLRP3 level, a major component of inflammasome (FIG. 46B), revealed that at basal unstimulated condition, both molecules were able to attenuate its expression. However, CHX concentrations at ≥1 μM, but not CM002, also induced CHOP, a ER stress marker, suggesting its higher toxicity. Furthermore, by challenging macrophages with LPS to mimic the inflammatory milieu of dystrophic disease condition, we found that CHX failed to inhibit NLRP3 inflammasome and displayed stimulatory effect at high concentrations accompanied with CHOP induction (FIG. 46C). In contrast, CM002 suppressed NLRP3 in the presence of LPS without inducing CHOP (FIG. 46D), suggesting its anti-inflammasome activity in the presence of inflammatory stimuli without provoking ER stress response. These findings revealed potential direct actions of clock-activating molecules, particularly CM002, on attenuating macrophage inflammatory response that may contribute to protective effects against chronic inflammation in dystrophic disease conditions.

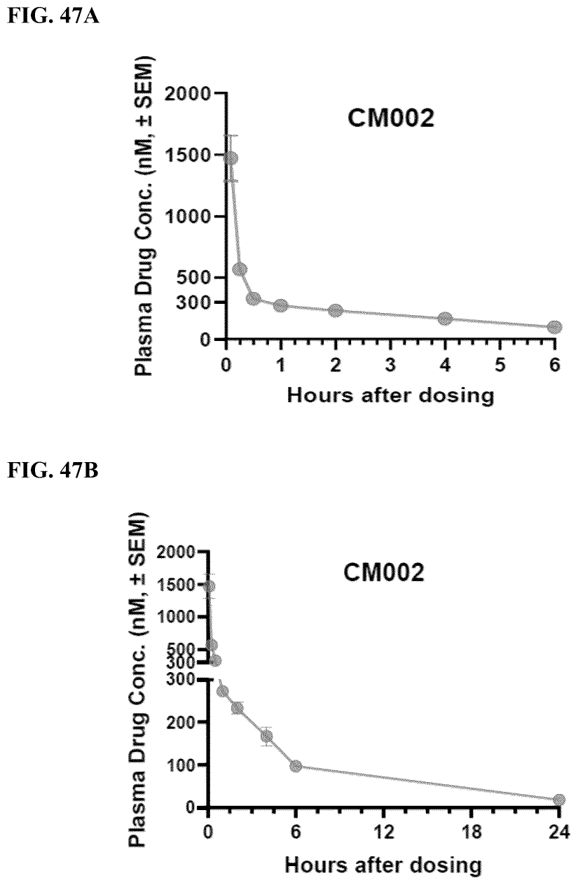

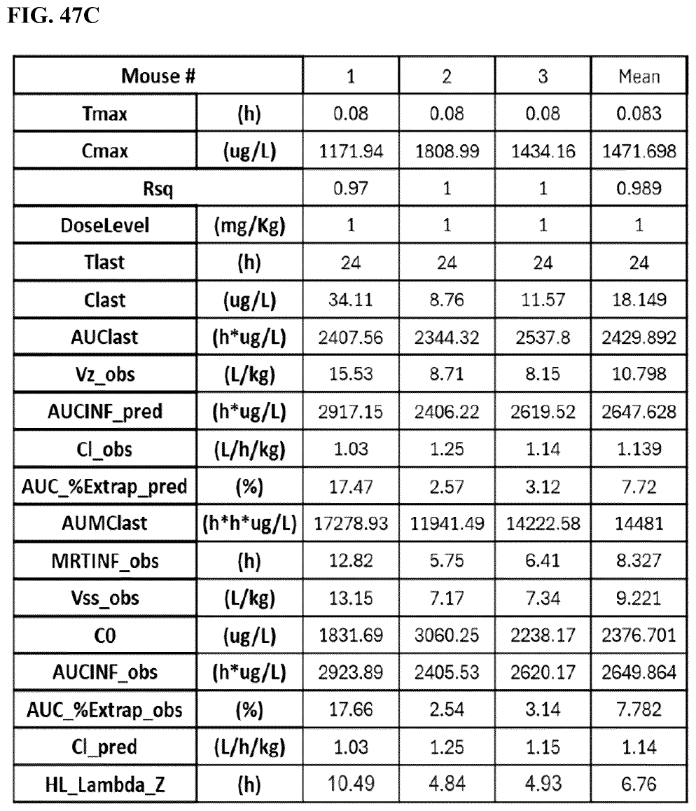

FIGS. 47A-47C. In vivo PK study of CM002 by intravenous administration in C57/BL6 mice. We next performed pharmacokinetic analysis of CM002 in normal wild-type C57/BL6 mice to guide in vivo studies. Following intravenous delivery of CM002 via tail vein injection at 1 mg/kg of body weight in three mice of 12 weeks of age, we monitored its plasma concentrations at 6 hours (FIG. 47A) and 24 hours (FIG. 47B). The clearance of CM002 follows that of a typical kinetic curve of a small molecule, with tissue re-distribution occurring within 30 minutes and largely cleared within 24 hours. Its maximal plasma concentration reached ˜1.47 mg/L, and in vivo half-life was determined at 6.76±1.33 hours with plasma level of ˜0.1 mg/L (FIG. 47C). Quantitative analysis of key pharmacokinetic parameters is shown in FIG. 47C, based on 1 mg/kg/day intravenous administration.

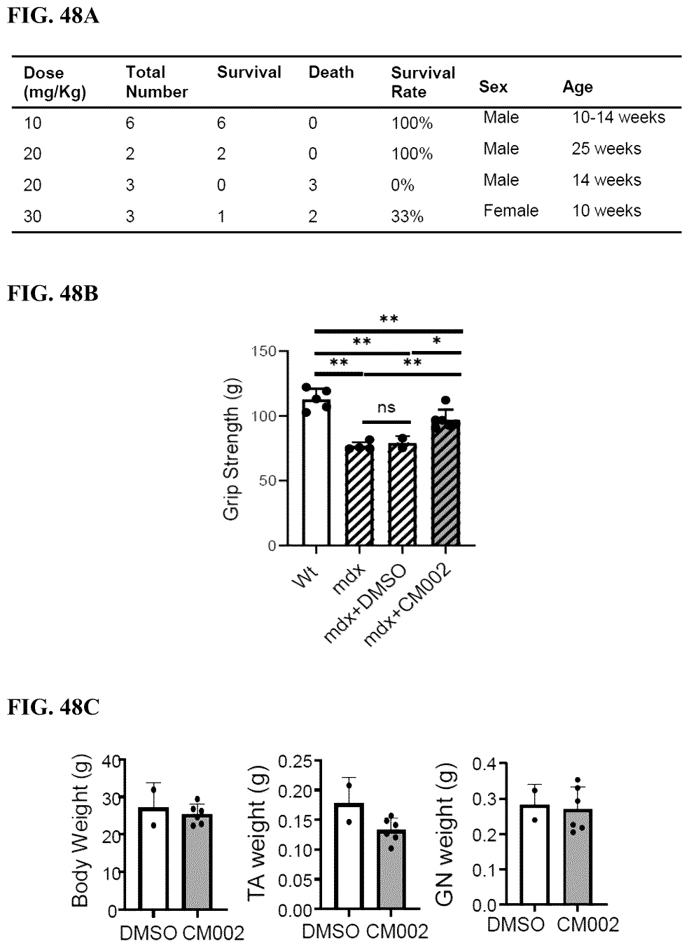

FIGS. 48A-48C. Systemic delivery of CM002 by intraperitoneal injection improves muscle strength in dystrophic mdx mice. Based on the PK analysis, we determined the maximal lethal dose in mdx mice via daily intraperitoneal injections using escalating doses. Starting doses were tested at 10, 20 and 30 mg/Kg/day, for 3 consecutive days in male or female mdx mice (FIG. 48A). Male mice of 10-14 weeks tolerated 10 mg/kg well without any lethality. At 20 mg/kg IP injection, young adult mice at 14 weeks of age were succumbed to death, while older mice over 6 months of age survived. The lethal dose is estimated at over 20 mg/kg/day as most mice injected at 30 mg/kg deceased. These results determined 10 mg/kg/day dose by intraperitoneal injection as most suitable for in vivo studies in 10-12 week old mdx male mice, or normal wild-type controls. Following 3 doses of injections of CM002, grip strength measured in the treated groups were significantly increased by ˜16% as compared to non-treated or DMSO-injected groups, suggesting improved muscle contractile function (FIG. 48B). As anticipated, the improvement in grip strength in CM002-treated mdx mice remained significantly lower than that of the normal controls. Analysis of total body weight and muscle weight, including Tibialis Anterior and Gastrocnemius, did not show significant effect following 3 days of CM002 IP administration (FIG. 48C).

### Example 5: Effect of Clock Activators on Regulation of Food Intake and Activity Levels

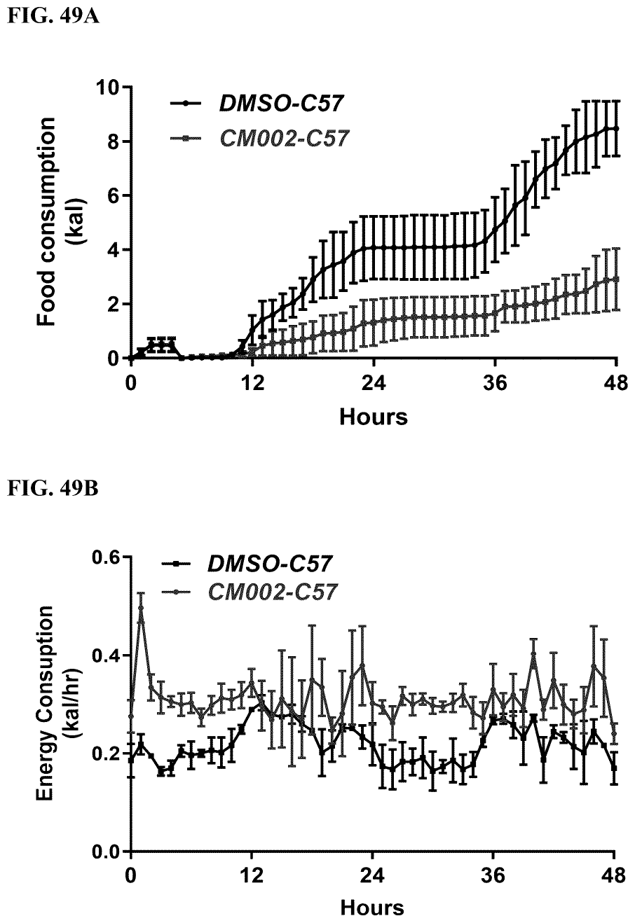

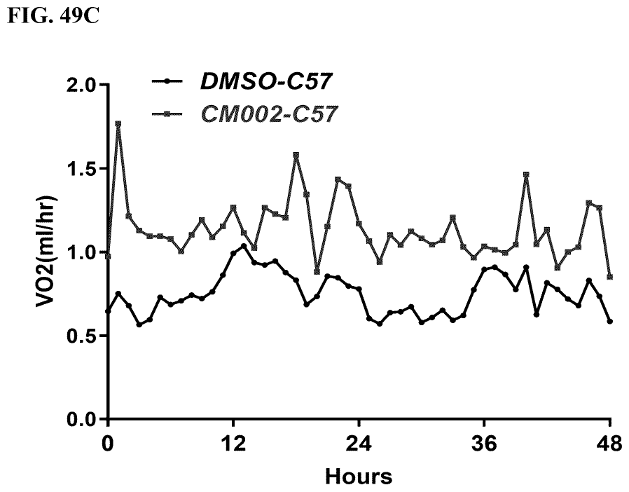

To examine potential effects of CM002 on central clock located in suprachiasmatic nuclei that controls food intake, activity and sleep rhythms, we administered CM002 (5 mg/Kg) together with DMSO as vehicle control using daily IP injections in normal C57/BL6 mice. Mice received daily injections for 3 doses and their food and water intake, activity level and energy expenditure were monitored using metabolic cages for 3 days. As shown in FIG. 49A, there was a significant reduction of accumulative food consumption in mice treated with CM002 as compared to vehicle control, reaching only ˜25% of the normal food intake level after 3 days of monitoring. Interestingly, energy expenditure based on calculation from oxygen consumption in CM002-treated mice were persistently elevated as compared to controls across both light and dark cycles (FIG. 49B), with markedly higher levels most evident during the daytime resting phase. The average tracing of oxygen consumption in CM002-treated mice and DMSO controls were shown in FIG. 49C. In contrast, daily ambulatory activity levels showed tendency toward reduction although not significantly altered, while water consumption remains comparable between groups. Thus, these findings suggest that CM002 may cross the blood-brain barrier to modulate central clock output regulations in appetite control, sleep duration, activity level and energy expenditure. The elevated energy consumption despite moderate reduction of activity levels suggest increased efficiency for energy metabolism with improved basal metabolic rate.

What is claimed is:

1. A method of increasing myogenesis in a subject in need thereof, said method comprising administering a therapeutically effective amount of a clock activator, or a pharmaceutically acceptable salt thereof.

2. A method of treating a muscle degenerative disease in a subject in need thereof, said method comprising administering a therapeutically effective amount of a clock activator, or a pharmaceutically acceptable salt thereof.

3. The method of claim 2, wherein the muscle degenerative disease is muscular dystrophy, aging-induced sarcopenia, cachexia, or cardiomyopathy.

4. A method of treating a muscle injury in a subject in need thereof, said method comprising administering a therapeutically effective amount of a clock activator, or a pharmaceutically acceptable salt thereof.

5. The method of claim 4, wherein the muscle injury is a cardiac muscle injury.

6. A method of reducing adipogenesis in a subject in need thereof, said method comprising administering a therapeutically effective amount of a clock activator, or a pharmaceutically acceptable salt thereof.

7. A method of treating a metabolic disorder in a subject in need thereof, said method comprising administering a therapeutically effective amount of a clock activator, or a pharmaceutically acceptable salt thereof.

8. The method of claim 7, wherein the metabolic disorder is obesity or type 2 diabetes.

9. A method of activating a CLOCK protein, said method comprising contacting said CLOCK protein with a clock activator, or a pharmaceutically acceptable salt thereof, which contacts at least one amino acid residue forming a palmitoylation site of said CLOCK protein, wherein said at least one amino acid residue is selected from E116, Q261, F262, E270, G345, Q112, F262, and P260.

10. The method of claim 1, wherein the clock activator has the formula: 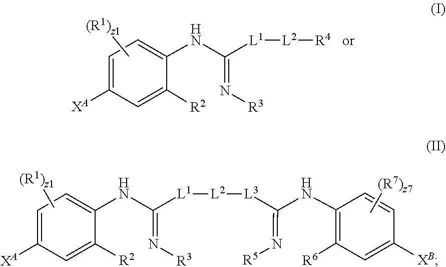
wherein
X^{A} and X^{B} are independently —Cl, —Br, —I, or —F;
L¹ is a bond, —C(O)—, —C(O)O—, —OC(O)—, —O—, —S—, —NR¹⁰—, —C(O)NR¹⁰—,
—NR¹⁰C(O)—, —NR′OC(O)O—, —OC(O)NR¹⁰—, —NR¹⁰C(O)NR¹⁰—, —NR¹⁰C(NH)NR¹⁰—, —S(O)₂—,
—NR¹⁰S(O)₂—, —S(O)₂NR¹⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —C(O)—, —C(O)O—, —OC(O)—, —O—, —S—, —NR²⁰—, —C(O)NR²⁰—,
—NR²⁰C(O)—, —NR²⁰C(O)O—, —OC(O)NR²⁰—, —NR²⁰C(O)NR²⁰—, —NR²⁰C(NH)NR²⁰, —S(O)₂—,
—NR²⁰S(O)₂—, —S(O)₂NR²⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —C(O)—, —C(O)O—, —OC(O)—, —O—, —S—, —NR³⁰—, —C(O)NR³⁰—,
—NR³⁰C(O)—, —NR³⁰C(O)O—, —OC(O)NR³⁰—, —NR³⁰C(O)NR³⁰—, —NR³⁰C(NH)NR³⁰—, —S(O)₂—,
—NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹,
—OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B},
—N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B},
—OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C},
—N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂,
—CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₙ₂,
—NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B},
—OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, —CCl₃, —CBr₃, —CF₃, —CI₃, —CHCl₂, —CHBr₂, —CHF₂, —CHI₂, —CH₂Cl,
—CH₂Br, —CH₂F, —CH₂I, —CN, —OH, —NH₂, —COOH, —CONH₂, —OCCl₃, —OCF₃, —OCBr₃, —OCI₃,
—OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted heterocycloalkyl or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂,
—CN, —SOₙ₄R^{4D}, —SOₙ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄,
—NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B},
—OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, —CCl₃, —CBr₃, —CF₃, —CI₃, —CHCl₂, —CHBr₂, —CHF₂, —CHI₂, —CH₂Cl,
—CH₂Br, —CH₂F, —CH₂I, —CN, —OH, —NH₂, —COOH, —CONH₂, —OCCl₃, —OCF₃, —OCBr₃, —OCI₃,
—OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCH₂X⁶, —OCHX⁶₂,
—CN, —SOₙ₆R^{6D}, —SOᵥ₆NR^{6A}R^{6B}, —NR^{6C}NR^{6A}R^{6B}, —ONR^{6A}R^{6B}, —NR^{6C}C(O)NR^{6A}R^{6B}, —N(O)ₘ₆,
—NR^{6A}R^{6B}, —C(O)R^{6C}, —C(O)OR^{6C}, —OC(O)R^{6C}, —OC(O)OR^{6C}, —C(O)NR^{6A}R^{6B}, —OC(O)NR^{6A}R^{6B},
—OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ and R⁶ substituents may optionally be joined to form a substituted or unsubstituted heterocycloalkyl or substituted or unsubstituted heteroaryl;
R⁷ is independently halogen, —CX⁷₃, —CHX⁷₂, —CH₂X⁷, —OCX⁷₃, —OCH₂X⁷,
—OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NR^{7C}C(O)NR^{7A}R^{7B},
—N(O)ₘ₇, —NR^{7A}R^{7B}, —C(O)R^{7C}, —C(O)OR^{7C}, —OC(O)R^{7C}, —OC(O)OR^{7C}, —C(O)NR^{7A}R^{7B},
—OC(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C},
—N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰, R²⁰, and R³⁰ is independently hydrogen, —CCl₃, —CBr₃, —CF₃, —Cl₃,
—CH₂Cl, —CH₂Br, —CH₂F, —CH₂I, —CHCl₂, —CHBr₂, —CHF₂, —CHI₂, —CN, —OH, —NH₂, —COOH,
—CONH₂, —OCCl₃, —OCBr₃, —OCF₃, —OCI₃, —OCH₂Cl, —OCH₂Br, —OCH₂F, —OCH₂I, —OCHCl₂,
—OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{4A}, R^{4B}, R^{4C}, R^{4D}, R^{6A}, R^{6B}, R^{6C}, R^{6D}, R^{7A}, R^{7B}, R^{7C}, and R^{7D} are independently hydrogen, —CCl₃, —CBr₃, —CF₃, —CI₃, —CHCl₂, —CHBr₂,
—CHF₂, —CHI₂, —CH₂Cl, —CH₂Br, —CH₂F, —CH₂I, —CN, —OH, —NH₂, —COOH, —CONH₂, —OCCl₃,
—OCF₃, —OCBr₃, —OCI₃, —OCHCl₂, —OCHBr₂, —OCHI₂, —OCHF₂, —OCH₂Cl, —OCH₂Br, —OCH₂I,
—OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl;
z1 is an integer from 0 to 3;
z7 is an integer from 0 to 3;
each X¹, X², X⁴, X⁶, and X⁷ is independently —Cl, —Br, —I, or —F;
n1, n2, n4, n6, and n7 are independently an integer from 0 to 4; and
m1, m2, m4, m6, m7, v1, v2, v4, v6, and v7 are independently 1 or 2.

11. The method of claim 10, wherein L¹ and L³ are independently a bond, —NH—, —NHC(NH)NH—, or substituted or unsubstituted 2 to 10 membered heteroalkylene.

12. The method of claim 10, wherein the clock activator has the formula:

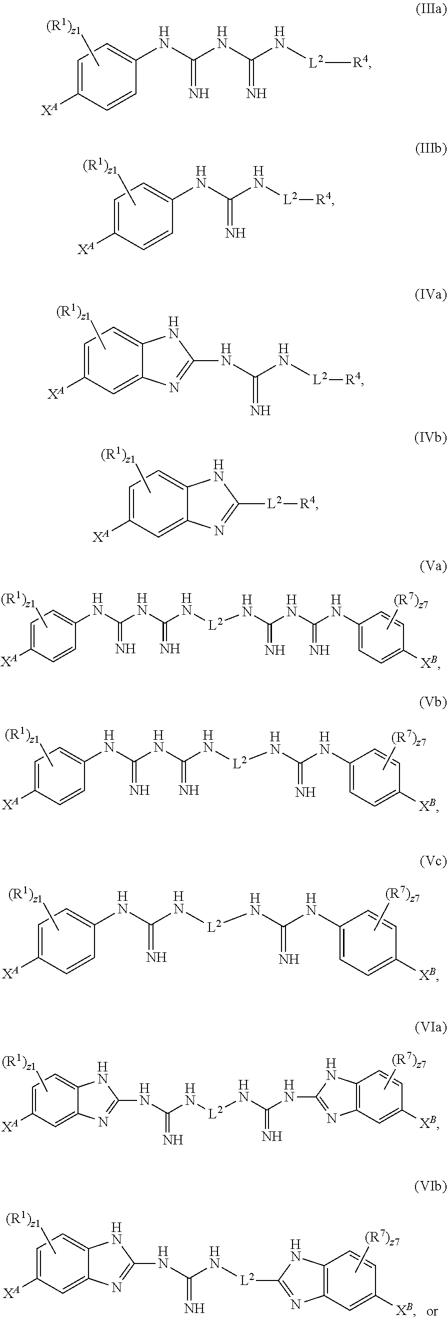

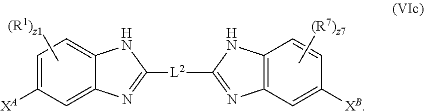

13. The method of claim 10, wherein L² is unsubstituted C₂-C₂₀ alkylene.

14. The method of claim 10, wherein R⁴ is hydrogen, —NHC(O)NH₂, or unsubstituted C₁-C₄ alkylene.

15. The method of claim 1, wherein the clock activator is chlorhexidine.

16. The method of claim 1, wherein the clock activator has the formula:

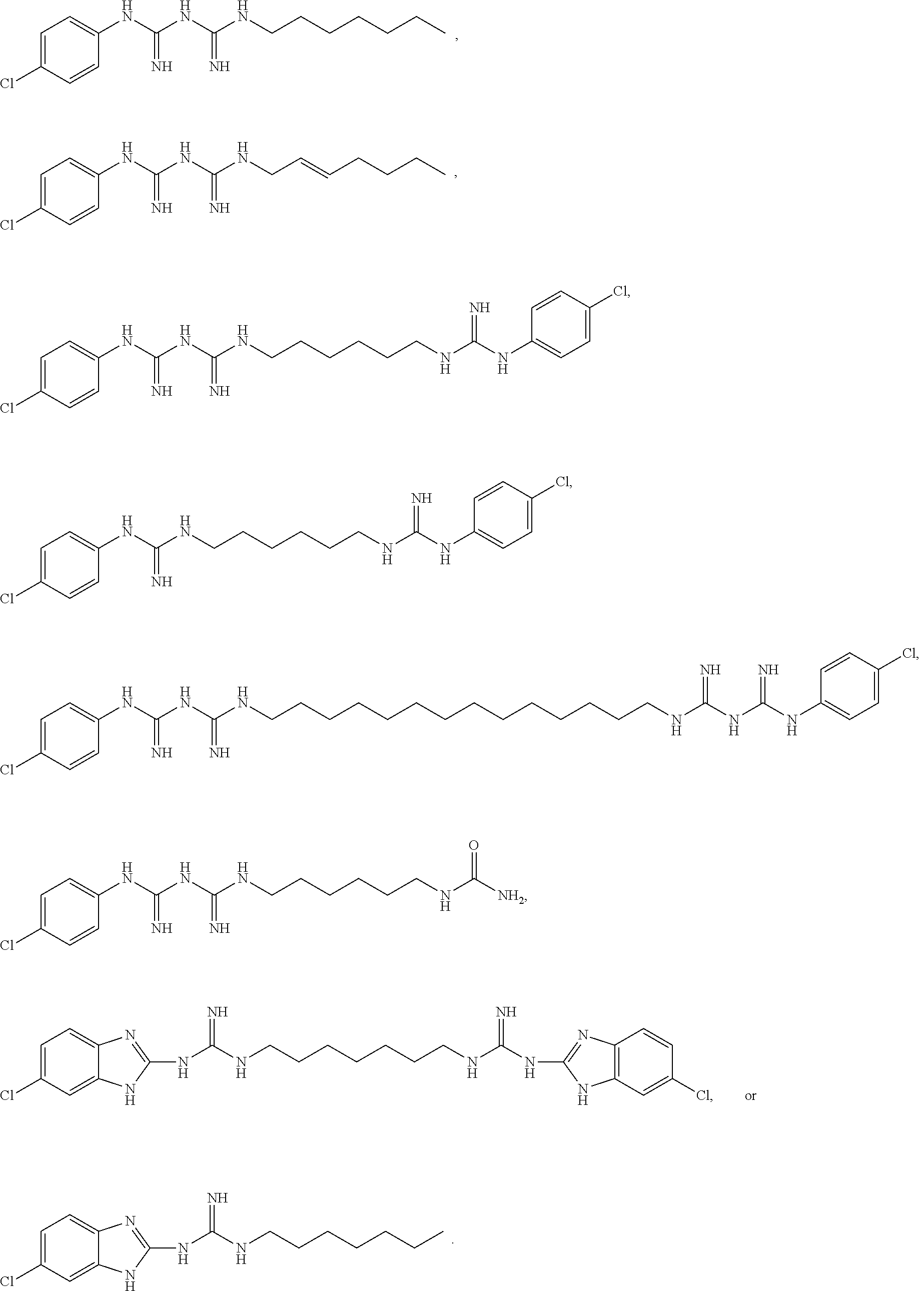

17. A compound, having the formula: 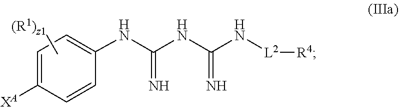
wherein
X^{A} is —Cl, —Br, —I, or —F;
L² is a bond;
R⁴ is unsubstituted C₄-C₂₀ alkenyl;
R¹ is independently halogen, —CX¹₃, —CHX¹₂, —CH₂X¹, —OCX¹₃, —OCH₂X¹,
—OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B},
—N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B},
—OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C},
—N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CCl₃, —CBr₃, —CF₃, —Cl₃,
—CHCl₂, —CHBr₂, —CHF₂, —CHI₂, —CH₂Cl, —CH₂Br, —CH₂F, —CH₂I, —CN, —OH, —NH₂, —COOH,
—CONH₂, —OCCl₃, —OCF₃, —OCBr₃, —OCI₃, —OCHCl₂, —OCHBr₂, —OCHI₂, —OCHF₂, —OCH₂Cl,
—OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
z1 is an integer from 0 to 3;
each X¹ is independently —Cl, —Br, —I, or —F;
n1 is independently an integer from 0 to 4; and
m1 and v1 are independently 1 or 2.

18. The compound of claim 17, wherein R⁴ is unsubstituted C₄-C₈ alkenyl.

19. The compound of claim 17, having the formula:

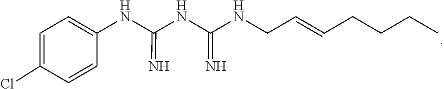 [SMILES: CCCC/C=C/CNC(=N)NC(=N)Nc1ccc(Cl)cc1]

20. A pharmaceutical composition comprising a pharmaceutically acceptable excipient and a compound, or a pharmaceutically acceptable salt thereof, having the formula: 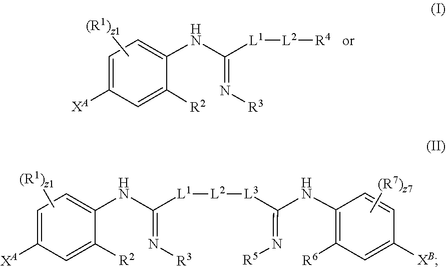
wherein
X^{A} and X^{B} are independently —Cl, —Br, —I, or —F;
L¹ is a bond, —C(O)—, —C(O)O—, —OC(O)—, —O—, —S—, —NR¹⁰—, —C(O)NR¹⁰—,
—NR¹⁰C(O)—, —NR¹⁰C(O)O—, —OC(O)NR¹⁰—, —NR¹⁰C(O)NR¹⁰—, —NR¹⁰C(NH)NR¹⁰—, —S(O)₂—,
—NR¹⁰S(O)₂—, —S(O)₂NR¹⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —C(O)—, —C(O)O—, —OC(O)—, —O—, —S—, —NR²⁰—, —C(O)NR²⁰—,
—NR²⁰C(O)—, —NR²⁰C(O)O—, —OC(O)NR²⁰—, —NR²⁰C(O)NR²⁰—, —NR²⁰C(NH)NR²⁰—, —S(O)₂—,
—NR²⁰S(O)₂—, —S(O)₂NR²⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —C(O)—, —C(O)O—, —OC(O)—, —O—, —S—, —NR³⁰—, —C(O)NR³⁰—,
—NR³⁰C(O)—, —NR³⁰C(O)O—, —OC(O)NR³⁰—, —NR³⁰C(O)NR³⁰—, —NR³⁰C(NH)NR³⁰—, —S(O)₂—,
—NR³⁰S(O)₂—, —S(O)₂NR³⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹,
—OCHX¹², —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B},
—N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B},
—OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C},
—N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂,
—CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂,
—NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B},
—OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃,
—OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted heterocycloalkyl or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂,
—CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄,
—NR^{4A}R^{4B}, —C(O)R^{4C}, —C(O)OR^{4C}, —OC(O)R^{4C}, —OC(O)OR^{4C}, —C(O)NR^{4A}R^{4B}, —OC(O)NR^{4A}R^{4B},
—OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, —CCl₃, —CBr₃, —CF₃, —CI₃, —CHCl₂, —CHBr₂, —CHF₂, —CHI₂, —CH₂Cl,
—CH₂Br, —CH₂F, —CH₂I, —CN, —OH, —NH₂, —COOH, —CONH₂, —OCCl₃, —OCF₃, —OCBr₃, —OCI₃,
—OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCH₂X⁶, —OCHX⁶₂,
—CN, —SOₙ₆R^{6D}, —SOᵥ₆NR^{6A}R^{6B}, —NR^{6C}NR^{6A}R^{6B}, —ONR^{6A}R^{6B}, —NR^{6C}C(O)NR^{6A}R^{6B}, —N(O)ₘ₆,
—NR^{6A}R^{6B}, —C(O)R^{6C}, —C(O)OR^{6C}, —OC(O)R^{6C}, —OC(O)OR^{6C}, —C(O)NR^{6A}R^{6B}, —OC(O)NR^{6A}R^{6B},
—OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ and R⁶ substituents may optionally be joined to form a substituted or unsubstituted heterocycloalkyl or substituted or unsubstituted heteroaryl;
R⁷ is independently halogen, —CX⁷₃, —CHX⁷₂, —CH₂X⁷, —OCX⁷₃, —OCH₂X⁷,
—OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NR^{7C}C(O)NR^{7A}R^{7B},
—N(O)ₘ₇, —NR^{7A}R^{7B}, —C(O)R^{7C}, —C(O)OR^{7C}, —OC(O)R^{7C}, —OC(O)OR^{7C}, —C(O)NR^{7A}R^{7B},
—OC(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C},
—N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R¹⁰, R²⁰, and R³⁰ is independently hydrogen, —CCl₃, —CBr₃, —CF₃, —Cl₃,
—CH₂Cl, —CH₂Br, —CH₂F, —CH₂I, —CHCl₂, —CHBr₂, —CHF₂, —CHI₂, —CN, —OH, —NH₂, —COOH,
—CONH₂, —OCCl₃, —OCBr₃, —OCF₃, —OCI₃, —OCH₂Cl, —OCH₂Br, —OCH₂F, —OCH₂I, —OCHCl₂,
—OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}R^{1C}, R^{1D}, R^{2A}, R^{2B}, R^{2C}, R^{2D}, R^{4A}, R^{4B}, R^{4C}, R^{4D}, R^{6A}, R^{6B}, R^{6C}, R^{6D}, R^{7A}, R^{7B}, R^{7C}, and R^{7D} are independently hydrogen, —CCl₃, —CBr₃, —CF₃, —CI₃, —CHCl₂, —CHBr₂,
—CHF₂, —CHI₂, —CH₂Cl, —CH₂Br, —CH₂F, —CH₂I, —CN, —OH, —NH₂, —COOH, —CONH₂, —OCCl₃,
—OCF₃, —OCBr₃, —OCI₃, —OCHCl₂, —OCHBr₂, —OCHI₂, —OCHF₂, —OCH₂Cl, —OCH₂Br, —OCH₂I,
—OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl;
z1 is an integer from 0 to 3;
z7 is an integer from 0 to 3;
each X¹, X², X⁴, X⁶, and X⁷ is independently —Cl, —Br, —I, or —F;
n1, n2, n4, n6, and n7 are independently an integer from 0 to 4; and
m1, m2, m4, m6, m7, v1, v2, v4, v6, and v7 are independently 1 or 2.
